SEQUENCE LISTING (200 sequences)

SEQ ID NO: 1 - DNA, 6627 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcaggtaaggagaccagtcccctgagggagcgtggagtgcctccccatctacagcactgcttctacatatcctggtcatcagaaccactactggggcctcttttgtgggtacactttccctttagtaaaggcttatgcagtatttcctttgacttctaatgctatgtaagtttacctaacaccttcacgggtctcttttatccacacagtgtttcagcctaccatcttggagtgctgagatactacatggtttgcccaaagtcacccagcaagtcttagaagcagggttcaagtcttcctgattggtgtagctctgctacttcctcaccaagagctgacaggctatatctcaagaaattccaaggaagcaccaaactgtaacagctgttcctctggaagcaaagttttgccagaaacagttctctggtgttcctaagatttaccaggaatgagcattaatggaattttgtgtcctctctctgtaaacgtaactcttctcattggctcagagttaagtgtagagacacataaccatgtgaagagtccctttgtgttcaggaaggatgcggctccttaaggttcctcaattgtgatacgtctatttttttccatggtcttaaatgaatttctccgaatacaggattttttaaatgcaatgctgaaatatagacttaataggccaaaaataagataaatttaatctttcttttgcaaaataacttttatttctggttagctcagctcaggtgggccaacatgaatttacggtttagagataaaaatttggttttctgaaattatcaggaaaatattagttgtaaggagcatatcctatagacatgtcatttcttgctgatataaaaaccattggtcccattataaactacatgaagaacaaagacatgatcagcttctactgactaagtcaatggttaacctcagctcaaattaagaaaaagttttaacatgaaaccaagcttgaaaattctgttacctgaaccaacatgtatcaatcactttctaagcatggacttccgggccctcagtttgggattagaaaggtattctcaggccattttccagacaagtgagtcctgatttggtctgtgagatgaaaccagacatgcggaagaccaggccagacagaggaatctgaccgtgccacttcctgctcatccaaacaggaggctttctcaccatcctgcaaggaggttcttggggtcaagtgcagctctcccaccaggtctcttgctcttcttgcccaggacatcattccttatttttcttctctatgaccaagtgctcagttacccttatattctataagtaggtagtcccttagaggaagcagtaagttggtgctttcaccactaagacgaaatgaagaatagtgatggcgaaggcacacgtactctacctccctttcccaaggtgctctgcaagagaacctatgtgcctcagacaactcccatctgccatcttggtgctcctctctaaggtcccagtgcagtggtcaccaagaaaagcaccccgagacatagcaggcaggaagcttctcttggatagtaagggccgcagtctctgaatcctatcagaaaaggctgtctcttccactatgctctttgatatttagaatacagagcttaaatcctgcataaagtagcagctccatggccctagagtaaaaaaactggccagtctgatgctctcatttcattgttttaacaaaacttctgggaggaaggcctcaaaggttcttctgagtgttttgaggtgctagctggatggaaggggaaaatatgtgataataaaatctatctcccttaattatggtctcaggtggcagtagccaccatctctgaacaacaacaaaaacaaccaaccaggaaacatcaacaaaaccagactctatgagatattcacgactgatttgttatagtggcggctgtctaagaagtctgaatctatctgacaggagtatctgttacgtggccctcatacactgtaacatttctagaattcatggcccagctatagcagaataatttatttcagagttaacctgaaaccacctgttggaacgtcccactaatgctatccaggtgaagggcttccctacccctctgctccaccgctagtaaagccaaaatacaccccctctggatctccccatatccacctctcccaaatgcagacactgatgggtaattaacaccactgagaatcccagggtagaaataaaggctcagtctctaaacactcaactcagatggagccactgggtctaaatgctcaccctgtggtttgttctcttgtagatgccatctacgactgctcttccctctaccagaagaactaccgcatctctggagtgtataagcttcctcctgatgacttcctgggcagccctgaactggaggtgaggtcattacagtcactggccatgccctaatacctgtccttcaccccctcaaggggactacaacaacagggccattcacagtttaaagaaaggaaaattcggctgggcgcagtggctcacacctgtaatcccagcactatgggaggccgaggcaggtggatcacttcaggtcaggagtttaagaccagcctggccaacatggtgaaaccctgtctctactaaaaatacaaaaaaattagccaggcatggtggtgggcacctgtaatccctgctacacaggaggattgcttgaactcaggaggcagaggttgcagtgagccgagatcacgccactgcactataatctgggagacaaagtgagactccatttcaattaaaaaaaaaaaaaaaaaaaaggaaaactcaaacacaagcaaacacaccaaacaccacagagctatgcaaacactcagtttatgccctgcactccaaacccaggcatctgtttggccccttcaaatcattatcagtcaaacaacaagccttctaacatagatcagatcattcttataaccaccacataacttagtttaaatctcttgccatgtcctagaacagctattccttgggggaggagaaaagaaaacacgaaggcagcatcaaattatctggattttcacccaggcatggtggctcacacctgtaatcccaagttttttgggaggtgaggtgggcggaacaatcacctgaggtcaggactttgagaccagcctggccaacatgctgaaacccagtctctactaaaaatacaaaaattagcccagtgtggtgacaggcactctggtcccagctactaggaaggcaggagaatcactggaactcaggaggtggaggttgcagtgagccgagattgcaccactgtactctagcctgggcaacaagagtgaaattctgcttcaaaaaaaaaaaaagtatctggatttttccctccaagcttcatgtgcactcacccccgggcccaatttgcatcgttcttccagagcaatgcaccacccaccccagctcaccagcagtggggcagcatcactgcccgagtgagccagtgtgactgcgggagtgcacacatctactggctctgcagggacaggaacaggttgggaagcctgccctcttgctcctgccttctgcccctgcaagtccctcaccagagtatcccctctgcttcaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggtaagcacaaggccaggggccccatgactggaccagtgccaccacacatgaccgcgtacaactccgggggtgccattcctattctgattcaagacaaatctgtatattcattgtgatggttttcctgcaagttgtaatggagttgaggaaaaataggtatttttcctttctgcaacccccccaaccccccgacaaaagtggggctgcaggtgggacaggaagaggccagacccaggccagagtagagcaaattcaacagtcagctgtgccgaacactagtctctgctctggccgagcatgaggtcctttaggtgcaaatcttactgatactgtttggggacccttgctgaaggtctgaaagcactcactatatcctcatgtttctcttacagcagctctgtgtgggattcagcaaaaacatagctgcaccttataagcaggaaagtgaggaatatagaaagagagactaatcaaggccatatggtgaatcaggaaagaagttcgagccttgttttctgattcccaggttaacacagtaaactggaggtaaacaagtaataaagtcttattagattcacacctataaaaagatgtttggctatgggactgtcaggagagaaggggtatagagacagcatgaaatggagcctgctgcactttctttaaggctctgctcctcctgacaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtgagatttggggggaccggaaaggagaagttcaggtacaagctcataatcccacttgaggagaaagagtgaattataactgtacagttgatattccggttttggtattctttctgaccctggctctaactccttacctgatgtctggtctatcacagtcaacttactagcactgggtctgtttctcatgccaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 2 - DNA, 6627 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcaggtaaggagaccagtcccctgagggagcgtggagtgcctccccatctacagcactgcttctacatatcctggtcatcagaaccactactggggcctcttttgtgggtacactttccctttagtaaaggcttatgcagtatttcctttgacttctaatgctatgtaagtttacctaacaccttcacgggtctcttttatccacacagtgtttcagcctaccatcttggagtgctgagatactacatggtttgcccaaagtcacccagcaagtcttagaagcagggttcaagtcttcctgattggtgtagctctgctacttcctcaccaagagctgacaggctatatctcaagaaattccaaggaagcaccaaactgtaacagctgttcctctggaagcaaagttttgccagaaacagttctctggtgttcctaagatttaccaggaatgagcattaatggaattttgtgtcctctctctgtaaacgtaactcttctcattggctcagagttaagtgtagagacacataaccatgtgaagagtccctttgtgttcaggaaggatgcggctccttaaggttcctcaattgtgatacgtctatttttttccatggtcttaaatgaatttctccgaatacaggattttttaaatgcaatgctgaaatatagacttaataggccaaaaataagataaatttaatctttcttttgcaaaataacttttatttctggttagctcagctcaggtgggccaacatgaatttacggtttagagataaaaatttggttttctgaaattatcaggaaaatattagttgtaaggagcatatcctatagacatgtcatttcttgctgatataaaaaccattggtcccattataaactacatgaagaacaaagacatgatcagcttctactgactaagtcaatggttaacctcagctcaaattaagaaaaagttttaacatgaaaccaagcttgaaaattctgttacctgaaccaacatgtatcaatcactttctaagcatggacttccgggccctcagtttgggattagaaaggtattctcaggccattttccagacaagtgagtcctgatttggtctgtgagatgaaaccagacatgcggaagaccaggccagacagaggaatctgaccgtgccacttcctgctcatccaaacaggaggctttctcaccatcctgcaaggaggttcttggggtcaagtgcagctctcccaccaggtctcttgctcttcttgcccaggacatcattccttatttttcttctctatgaccaagtgctcagttacccttatattctataagtaggtagtcccttagaggaagcagtaagttggtgctttcaccactaagacgaaatgaagaatagtgatggcgaaggcacacgtactctacctccctttcccaaggtgctctgcaagagaacctatgtgcctcagacaactcccatctgccatcttggtgctcctctctaaggtcccagtgcagtggtcaccaagaaaagcaccccgagacatagcaggcaggaagcttctcttggatagtaagggccgcagtctctgaatcctatcagaaaaggctgtctcttccactatgctctttgatatttagaatacagagcttaaatcctgcataaagtagcagctccatggccctagagtaaaaaaactggccagtctgatgctctcatttcattgttttaacaaaacttctgggaggaaggcctcaaaggttcttctgagtgttttgaggtgctagctggatggaaggggaaaatatgtgataataaaatctatctcccttaattatggtctcaggtggcagtagccaccatctctgaacaacaacaaaaacaaccaaccaggaaacatcaacaaaaccagactctatgagatattcacgactgatttgttatagtggcggctgtctaagaagtctgaatctatctgacaggagtatctgttacgtggccctcatacactgtaacatttctagaattcatggcccagctatagcagaataatttatttcagagttaacctgaaaccacctgttggaacgtcccactaatgctatccaggtgaagggcttccctacccctctgctccaccgctagtaaagccaaaatacaccccctctggatctccccatatccacctctcccaaatgcagacactgatgggtaattaacaccactgagaatcccagggtagaaataaaggctcagtctctaaacactcaactcagatggagccactgggtctaaatgctcaccctgtggtttgttctcttgtagatgccatctacgactgctcttccctctaccagaagaactaccgcatctctggagtgtataagcttcctcctgatgacttcctgggcagccctgaactggaggtgaggtcattacagtcactggccatgccctaatacctgtccttcaccccctcaaggggactacaacaacagggccattcacagtttaaagaaaggaaaattcggctgggcgcagtggctcacacctgtaatcccagcactatgggaggccgaggcaggtggatcacttcaggtcaggagtttaagaccagcctggccaacatggtgaaaccctgtctctactaaaaatacaaaaaaattagccaggcatggtggtgggcacctgtaatccctgctacacaggaggattgcttgaactcaggaggcagaggttgcagtgagccgagatcacgccactgcactataatctgggagacaaagtgagactccatttcaattaaaaaaaaaaaaaaaaaaaaggaaaactcaaacacaagcaaacacaccaaacaccacagagctatgcaaacactcagtttatgccctgcactccaaacccaggcatctgtttggccccttcaaatcattatcagtcaaacaacaagccttctaacatagatcagatcattcttataaccaccacataacttagtttaaatctcttgccatgtcctagaacagctattccttgggggaggagaaaagaaaacacgaaggcagcatcaaattatctggattttcacccaggcatggtggctcacacctgtaatcccaagttttttgggaggtgaggtgggcggaacaatcacctgaggtcaggactttgagaccagcctggccaacatgctgaaacccagtctctactaaaaatacaaaaattagcccagtgtggtgacaggcactctggtcccagctactaggaaggcaggagaatcactggaactcaggaggtggaggttgcagtgagccgagattgcaccactgtactctagcctgggcaacaagagtgaaattctgcttcaaaaaaaaaaaaagtatctggatttttccctccaagcttcatgtgcactcacccccgggcccaatttgcatcgttcttccagagcaatgcaccacccaccccagctcaccagcagtggggcagcatcactgcccgagtgagccagtgtgactgcgggagtgcacacatctactggctctgcagggacaggaacaggttgggaagcctgccctcttgctcctgccttctgcccctgcaagtccctcaccagagtatcccctctgcttcaggtgatctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggtaagcacaaggccaggggccccatgactggaccagtgccaccacacatgaccgcgtacaactccgggggtgccattcctattctgattcaagacaaatctgtatattcattgtgatggttttcctgcaagttgtaatggagttgaggaaaaataggtatttttcctttctgcaacccccccaaccccccgacaaaagtggggctgcaggtgggacaggaagaggccagacccaggccagagtagagcaaattcaacagtcagctgtgccgaacactagtctctgctctggccgagcatgaggtcctttaggtgcaaatcttactgatactgtttggggacccttgctgaaggtctgaaagcactcactatatcctcatgtttctcttacagcagctctgtgtgggattcagcaaaaacatagctgcaccttataagcaggaaagtgaggaatatagaaagagagactaatcaaggccatatggtgaatcaggaaagaagttcgagccttgttttctgattcccaggttaacacagtaaactggaggtaaacaagtaataaagtcttattagattcacacctataaaaagatgtttggctatgggactgtcaggagagaaggggtatagagacagcatgaaatggagcctgctgcactttctttaaggctctgctcctcctgacaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtgagatttggggggaccggaaaggagaagttcaggtacaagctcataatcccacttgaggagaaagagtgaattataactgtacagttgatattccggttttggtattctttctgaccctggctctaactccttacctgatgtctggtctatcacagtcaacttactagcactgggtctgtttctcatgccaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 3 - DNA, 6627 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcaggtaaggagaccagtcccctgagggagcgtggagtgcctccccatctacagcactgcttctacatatcctggtcatcagaaccactactggggcctcttttgtgggtacactttccctttagtaaaggcttatgcagtatttcctttgacttctaatgctatgtaagtttacctaacaccttcacgggtctcttttatccacacagtgtttcagcctaccatcttggagtgctgagatactacatggtttgcccaaagtcacccagcaagtcttagaagcagggttcaagtcttcctgattggtgtagctctgctacttcctcaccaagagctgacaggctatatctcaagaaattccaaggaagcaccaaactgtaacagctgttcctctggaagcaaagttttgccagaaacagttctctggtgttcctaagatttaccaggaatgagcattaatggaattttgtgtcctctctctgtaaacgtaactcttctcattggctcagagttaagtgtagagacacataaccatgtgaagagtccctttgtgttcaggaaggatgcggctccttaaggttcctcaattgtgatacgtctatttttttccatggtcttaaatgaatttctccgaatacaggattttttaaatgcaatgctgaaatatagacttaataggccaaaaataagataaatttaatctttcttttgcaaaataacttttatttctggttagctcagctcaggtgggccaacatgaatttacggtttagagataaaaatttggttttctgaaattatcaggaaaatattagttgtaaggagcatatcctatagacatgtcatttcttgctgatataaaaaccattggtcccattataaactacatgaagaacaaagacatgatcagcttctactgactaagtcaatggttaacctcagctcaaattaagaaaaagttttaacatgaaaccaagcttgaaaattctgttacctgaaccaacatgtatcaatcactttctaagcatggacttccgggccctcagtttgggattagaaaggtattctcaggccattttccagacaagtgagtcctgatttggtctgtgagatgaaaccagacatgcggaagaccaggccagacagaggaatctgaccgtgccacttcctgctcatccaaacaggaggctttctcaccatcctgcaaggaggttcttggggtcaagtgcagctctcccaccaggtctcttgctcttcttgcccaggacatcattccttatttttcttctctatgaccaagtgctcagttacccttatattctataagtaggtagtcccttagaggaagcagtaagttggtgctttcaccactaagacgaaatgaagaatagtgatggcgaaggcacacgtactctacctccctttcccaaggtgctctgcaagagaacctatgtgcctcagacaactcccatctgccatcttggtgctcctctctaaggtcccagtgcagtggtcaccaagaaaagcaccccgagacatagcaggcaggaagcttctcttggatagtaagggccgcagtctctgaatcctatcagaaaaggctgtctcttccactatgctctttgatatttagaatacagagcttaaatcctgcataaagtagcagctccatggccctagagtaaaaaaactggccagtctgatgctctcatttcattgttttaacaaaacttctgggaggaaggcctcaaaggttcttctgagtgttttgaggtgctagctggatggaaggggaaaatatgtgataataaaatctatctcccttaattatggtctcaggtggcagtagccaccatctctgaacaacaacaaaaacaaccaaccaggaaacatcaacaaaaccagactctatgagatattcacgactgatttgttatagtggcggctgtctaagaagtctgaatctatctgacaggagtatctgttacgtggccctcatacactgtaacatttctagaattcatggcccagctatagcagaataatttatttcagagttaacctgaaaccacctgttggaacgtcccactaatgctatccaggtgaagggcttccctacccctctgctccaccgctagtaaagccaaaatacaccccctctggatctccccatatccacctctcccaaatgcagacactgatgggtaattaacaccactgagaatcccagggtagaaataaaggctcagtctctaaacactcaactcagatggagccactgggtctaaatgctcaccctgtggtttgttctcttgtagatgccatctacgactgctcttccctctaccagaagaactaccgcatctctggagtgtataagcttcctcctgatgacttcctgggcagccctgaactggaggtgaggtcattacagtcactggccatgccctaatacctgtccttcaccccctcaaggggactacaacaacagggccattcacagtttaaagaaaggaaaattcggctgggcgcagtggctcacacctgtaatcccagcactatgggaggccgaggcaggtggatcacttcaggtcaggagtttaagaccagcctggccaacatggtgaaaccctgtctctactaaaaatacaaaaaaattagccaggcatggtggtgggcacctgtaatccctgctacacaggaggattgcttgaactcaggaggcagaggttgcagtgagccgagatcacgccactgcactataatctgggagacaaagtgagactccatttcaattaaaaaaaaaaaaaaaaaaaaggaaaactcaaacacaagcaaacacaccaaacaccacagagctatgcaaacactcagtttatgccctgcactccaaacccaggcatctgtttggccccttcaaatcattatcagtcaaacaacaagccttctaacatagatcagatcattcttataaccaccacataacttagtttaaatctcttgccatgtcctagaacagctattccttgggggaggagaaaagaaaacacgaaggcagcatcaaattatctggattttcacccaggcatggtggctcacacctgtaatcccaagttttttgggaggtgaggtgggcggaacaatcacctgaggtcaggactttgagaccagcctggccaacatgctgaaacccagtctctactaaaaatacaaaaattagcccagtgtggtgacaggcactctggtcccagctactaggaaggcaggagaatcactggaactcaggaggtggaggttgcagtgagccgagattgcaccactgtactctagcctgggcaacaagagtgaaattctgcttcaaaaaaaaaaaaagtatctggatttttccctccaagcttcatgtgcactcacccccgggcccaatttgcatcgttcttccagagcaatgcaccacccaccccagctcaccagcagtggggcagcatcactgcccgagtgagccagtgtgactgcgggagtgcacacatctactggctctgcagggacaggaacaggttgggaagcctgccctcttgctcctgccttctgcccctgcaagtccctcaccagagtatcccctctgcttcaggtgttctgtgacatggagacttcaggcggaggctggaccatcaaccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggtaagcacaaggccaggggccccatgactggaccagtgccaccacacatgaccgcgtacaactccgggggtgccattcctattctgattcaagacaaatctgtatattcattgtgatggttttcctgcaagttgtaatggagttgaggaaaaataggtatttttcctttctgcaacccccccaaccccccgacaaaagtggggctgcaggtgggacaggaagaggccagacccaggccagagtagagcaaattcaacagtcagctgtgccgaacactagtctctgctctggccgagcatgaggtcctttaggtgcaaatcttactgatactgtttggggacccttgctgaaggtctgaaagcactcactatatcctcatgtttctcttacagcagctctgtgtgggattcagcaaaaacatagctgcaccttataagcaggaaagtgaggaatatagaaagagagactaatcaaggccatatggtgaatcaggaaagaagttcgagccttgttttctgattcccaggttaacacagtaaactggaggtaaacaagtaataaagtcttattagattcacacctataaaaagatgtttggctatgggactgtcaggagagaaggggtatagagacagcatgaaatggagcctgctgcactttctttaaggctctgctcctcctgacaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtgagatttggggggaccggaaaggagaagttcaggtacaagctcataatcccacttgaggagaaagagtgaattataactgtacagttgatattccggttttggtattctttctgaccctggctctaactccttacctgatgtctggtctatcacagtcaacttactagcactgggtctgtttctcatgccaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 4 - DNA, 6627 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcaggtaaggagaccagtcccctgagggagcgtggagtgcctccccatctacagcactgcttctacatatcctggtcatcagaaccactactggggcctcttttgtgggtacactttccctttagtaaaggcttatgcagtatttcctttgacttctaatgctatgtaagtttacctaacaccttcacgggtctcttttatccacacagtgtttcagcctaccatcttggagtgctgagatactacatggtttgcccaaagtcacccagcaagtcttagaagcagggttcaagtcttcctgattggtgtagctctgctacttcctcaccaagagctgacaggctatatctcaagaaattccaaggaagcaccaaactgtaacagctgttcctctggaagcaaagttttgccagaaacagttctctggtgttcctaagatttaccaggaatgagcattaatggaattttgtgtcctctctctgtaaacgtaactcttctcattggctcagagttaagtgtagagacacataaccatgtgaagagtccctttgtgttcaggaaggatgcggctccttaaggttcctcaattgtgatacgtctatttttttccatggtcttaaatgaatttctccgaatacaggattttttaaatgcaatgctgaaatatagacttaataggccaaaaataagataaatttaatctttcttttgcaaaataacttttatttctggttagctcagctcaggtgggccaacatgaatttacggtttagagataaaaatttggttttctgaaattatcaggaaaatattagttgtaaggagcatatcctatagacatgtcatttcttgctgatataaaaaccattggtcccattataaactacatgaagaacaaagacatgatcagcttctactgactaagtcaatggttaacctcagctcaaattaagaaaaagttttaacatgaaaccaagcttgaaaattctgttacctgaaccaacatgtatcaatcactttctaagcatggacttccgggccctcagtttgggattagaaaggtattctcaggccattttccagacaagtgagtcctgatttggtctgtgagatgaaaccagacatgcggaagaccaggccagacagaggaatctgaccgtgccacttcctgctcatccaaacaggaggctttctcaccatcctgcaaggaggttcttggggtcaagtgcagctctcccaccaggtctcttgctcttcttgcccaggacatcattccttatttttcttctctatgaccaagtgctcagttacccttatattctataagtaggtagtcccttagaggaagcagtaagttggtgctttcaccactaagacgaaatgaagaatagtgatggcgaaggcacacgtactctacctccctttcccaaggtgctctgcaagagaacctatgtgcctcagacaactcccatctgccatcttggtgctcctctctaaggtcccagtgcagtggtcaccaagaaaagcaccccgagacatagcaggcaggaagcttctcttggatagtaagggccgcagtctctgaatcctatcagaaaaggctgtctcttccactatgctctttgatatttagaatacagagcttaaatcctgcataaagtagcagctccatggccctagagtaaaaaaactggccagtctgatgctctcatttcattgttttaacaaaacttctgggaggaaggcctcaaaggttcttctgagtgttttgaggtgctagctggatggaaggggaaaatatgtgataataaaatctatctcccttaattatggtctcaggtggcagtagccaccatctctgaacaacaacaaaaacaaccaaccaggaaacatcaacaaaaccagactctatgagatattcacgactgatttgttatagtggcggctgtctaagaagtctgaatctatctgacaggagtatctgttacgtggccctcatacactgtaacatttctagaattcatggcccagctatagcagaataatttatttcagagttaacctgaaaccacctgttggaacgtcccactaatgctatccaggtgaagggcttccctacccctctgctccaccgctagtaaagccaaaatacaccccctctggatctccccatatccacctctcccaaatgcagacactgatgggtaattaacaccactgagaatcccagggtagaaataaaggctcagtctctaaacactcaactcagatggagccactgggtctaaatgctcaccctgtggtttgttctcttgtagatgccatctacgactgctcttccctctaccagaagaactaccgcatctctggagtgtataagcttcctcctgatgacttcctgggcagccctgaactggaggtgaggtcattacagtcactggccatgccctaatacctgtccttcaccccctcaaggggactacaacaacagggccattcacagtttaaagaaaggaaaattcggctgggcgcagtggctcacacctgtaatcccagcactatgggaggccgaggcaggtggatcacttcaggtcaggagtttaagaccagcctggccaacatggtgaaaccctgtctctactaaaaatacaaaaaaattagccaggcatggtggtgggcacctgtaatccctgctacacaggaggattgcttgaactcaggaggcagaggttgcagtgagccgagatcacgccactgcactataatctgggagacaaagtgagactccatttcaattaaaaaaaaaaaaaaaaaaaaggaaaactcaaacacaagcaaacacaccaaacaccacagagctatgcaaacactcagtttatgccctgcactccaaacccaggcatctgtttggccccttcaaatcattatcagtcaaacaacaagccttctaacatagatcagatcattcttataaccaccacataacttagtttaaatctcttgccatgtcctagaacagctattccttgggggaggagaaaagaaaacacgaaggcagcatcaaattatctggattttcacccaggcatggtggctcacacctgtaatcccaagttttttgggaggtgaggtgggcggaacaatcacctgaggtcaggactttgagaccagcctggccaacatgctgaaacccagtctctactaaaaatacaaaaattagcccagtgtggtgacaggcactctggtcccagctactaggaaggcaggagaatcactggaactcaggaggtggaggttgcagtgagccgagattgcaccactgtactctagcctgggcaacaagagtgaaattctgcttcaaaaaaaaaaaaagtatctggatttttccctccaagcttcatgtgcactcacccccgggcccaatttgcatcgttcttccagagcaatgcaccacccaccccagctcaccagcagtggggcagcatcactgcccgagtgagccagtgtgactgcgggagtgcacacatctactggctctgcagggacaggaacaggttgggaagcctgccctcttgctcctgccttctgcccctgcaagtccctcaccagagtatcccctctgcttcaggtgttctgtgacatggagacttcaggcggaggctggaccatcatccatagacgaaaaagtggccttgtctccttctaccgggactggaagcagtacaagcagggctttggcagcatccgtggggacttctggctggggaacgaacacatccaccggctctccagacagccaacccggctgcgtgtagagatggaggtaagcacaaggccaggggccccatgactggaccagtgccaccacacatgaccgcgtacaactccgggggtgccattcctattctgattcaagacaaatctgtatattcattgtgatggttttcctgcaagttgtaatggagttgaggaaaaataggtatttttcctttctgcaacccccccaaccccccgacaaaagtggggctgcaggtgggacaggaagaggccagacccaggccagagtagagcaaattcaacagtcagctgtgccgaacactagtctctgctctggccgagcatgaggtcctttaggtgcaaatcttactgatactgtttggggacccttgctgaaggtctgaaagcactcactatatcctcatgtttctcttacagcagctctgtgtgggattcagcaaaaacatagctgcaccttataagcaggaaagtgaggaatatagaaagagagactaatcaaggccatatggtgaatcaggaaagaagttcgagccttgttttctgattcccaggttaacacagtaaactggaggtaaacaagtaataaagtcttattagattcacacctataaaaagatgtttggctatgggactgtcaggagagaaggggtatagagacagcatgaaatggagcctgctgcactttctttaaggctctgctcctcctgacaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtgagatttggggggaccggaaaggagaagttcaggtacaagctcataatcccacttgaggagaaagagtgaattataactgtacagttgatattccggttttggtattctttctgaccctggctctaactccttacctgatgtctggtctatcacagtcaacttactagcactgggtctgtttctcatgccaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 5 - DNA, 6627 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcaggtaaggagaccagtcccctgagggagcgtggagtgcctccccatctacagcactgcttctacatatcctggtcatcagaaccactactggggcctcttttgtgggtacactttccctttagtaaaggcttatgcagtatttcctttgacttctaatgctatgtaagtttacctaacaccttcacgggtctcttttatccacacagtgtttcagcctaccatcttggagtgctgagatactacatggtttgcccaaagtcacccagcaagtcttagaagcagggttcaagtcttcctgattggtgtagctctgctacttcctcaccaagagctgacaggctatatctcaagaaattccaaggaagcaccaaactgtaacagctgttcctctggaagcaaagttttgccagaaacagttctctggtgttcctaagatttaccaggaatgagcattaatggaattttgtgtcctctctctgtaaacgtaactcttctcattggctcagagttaagtgtagagacacataaccatgtgaagagtccctttgtgttcaggaaggatgcggctccttaaggttcctcaattgtgatacgtctatttttttccatggtcttaaatgaatttctccgaatacaggattttttaaatgcaatgctgaaatatagacttaataggccaaaaataagataaatttaatctttcttttgcaaaataacttttatttctggttagctcagctcaggtgggccaacatgaatttacggtttagagataaaaatttggttttctgaaattatcaggaaaatattagttgtaaggagcatatcctatagacatgtcatttcttgctgatataaaaaccattggtcccattataaactacatgaagaacaaagacatgatcagcttctactgactaagtcaatggttaacctcagctcaaattaagaaaaagttttaacatgaaaccaagcttgaaaattctgttacctgaaccaacatgtatcaatcactttctaagcatggacttccgggccctcagtttgggattagaaaggtattctcaggccattttccagacaagtgagtcctgatttggtctgtgagatgaaaccagacatgcggaagaccaggccagacagaggaatctgaccgtgccacttcctgctcatccaaacaggaggctttctcaccatcctgcaaggaggttcttggggtcaagtgcagctctcccaccaggtctcttgctcttcttgcccaggacatcattccttatttttcttctctatgaccaagtgctcagttacccttatattctataagtaggtagtcccttagaggaagcagtaagttggtgctttcaccactaagacgaaatgaagaatagtgatggcgaaggcacacgtactctacctccctttcccaaggtgctctgcaagagaacctatgtgcctcagacaactcccatctgccatcttggtgctcctctctaaggtcccagtgcagtggtcaccaagaaaagcaccccgagacatagcaggcaggaagcttctcttggatagtaagggccgcagtctctgaatcctatcagaaaaggctgtctcttccactatgctctttgatatttagaatacagagcttaaatcctgcataaagtagcagctccatggccctagagtaaaaaaactggccagtctgatgctctcatttcattgttttaacaaaacttctgggaggaaggcctcaaaggttcttctgagtgttttgaggtgctagctggatggaaggggaaaatatgtgataataaaatctatctcccttaattatggtctcaggtggcagtagccaccatctctgaacaacaacaaaaacaaccaaccaggaaacatcaacaaaaccagactctatgagatattcacgactgatttgttatagtggcggctgtctaagaagtctgaatctatctgacaggagtatctgttacgtggccctcatacactgtaacatttctagaattcatggcccagctatagcagaataatttatttcagagttaacctgaaaccacctgttggaacgtcccactaatgctatccaggtgaagggcttccctacccctctgctccaccgctagtaaagccaaaatacaccccctctggatctccccatatccacctctcccaaatgcagacactgatgggtaattaacaccactgagaatcccagggtagaaataaaggctcagtctctaaacactcaactcagatggagccactgggtctaaatgctcaccctgtggtttgttctcttgtagatgccatctacgactgctcttccctctaccagaagaactaccgcatctctggagtgtataagcttcctcctgatgacttcctgggcagccctgaactggaggtgaggtcattacagtcactggccatgccctaatacctgtccttcaccccctcaaggggactacaacaacagggccattcacagtttaaagaaaggaaaattcggctgggcgcagtggctcacacctgtaatcccagcactatgggaggccgaggcaggtggatcacttcaggtcaggagtttaagaccagcctggccaacatggtgaaaccctgtctctactaaaaatacaaaaaaattagccaggcatggtggtgggcacctgtaatccctgctacacaggaggattgcttgaactcaggaggcagaggttgcagtgagccgagatcacgccactgcactataatctgggagacaaagtgagactccatttcaattaaaaaaaaaaaaaaaaaaaaggaaaactcaaacacaagcaaacacaccaaacaccacagagctatgcaaacactcagtttatgccctgcactccaaacccaggcatctgtttggccccttcaaatcattatcagtcaaacaacaagccttctaacatagatcagatcattcttataaccaccacataacttagtttaaatctcttgccatgtcctagaacagctattccttgggggaggagaaaagaaaacacgaaggcagcatcaaattatctggattttcacccaggcatggtggctcacacctgtaatcccaagttttttgggaggtgaggtgggcggaacaatcacctgaggtcaggactttgagaccagcctggccaacatgctgaaacccagtctctactaaaaatacaaaaattagcccagtgtggtgacaggcactctggtcccagctactaggaaggcaggagaatcactggaactcaggaggtggaggttgcagtgagccgagattgcaccactgtactctagcctgggcaacaagagtgaaattctgcttcaaaaaaaaaaaaagtatctggatttttccctccaagcttcatgtgcactcacccccgggcccaatttgcatcgttcttccagagcaatgcaccacccaccccagctcaccagcagtggggcagcatcactgcccgagtgagccagtgtgactgcgggagtgcacacatctactggctctgcagggacaggaacaggttgggaagcctgccctcttgctcctgccttctgcccctgcaagtccctcaccagagtatcccctctgcttcaggtgttctgtgacatggagacttcaggcggaggctggaccatcatccagagatgaaaaagtggccttgtctccttctaccgggactggaagcagtacaagcagggctttggcagcatccgtggggacttctggctggggaacgaacacatccaccggctctccagacagccaacccggctgcgtgtagagatggaggtaagcacaaggccaggggccccatgactggaccagtgccaccacacatgaccgcgtacaactccgggggtgccattcctattctgattcaagacaaatctgtatattcattgtgatggttttcctgcaagttgtaatggagttgaggaaaaataggtatttttcctttctgcaacccccccaaccccccgacaaaagtggggctgcaggtgggacaggaagaggccagacccaggccagagtagagcaaattcaacagtcagctgtgccgaacactagtctctgctctggccgagcatgaggtcctttaggtgcaaatcttactgatactgtttggggacccttgctgaaggtctgaaagcactcactatatcctcatgtttctcttacagcagctctgtgtgggattcagcaaaaacatagctgcaccttataagcaggaaagtgaggaatatagaaagagagactaatcaaggccatatggtgaatcaggaaagaagttcgagccttgttttctgattcccaggttaacacagtaaactggaggtaaacaagtaataaagtcttattagattcacacctataaaaagatgtttggctatgggactgtcaggagagaaggggtatagagacagcatgaaatggagcctgctgcactttctttaaggctctgctcctcctgacaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtgagatttggggggaccggaaaggagaagttcaggtacaagctcataatcccacttgaggagaaagagtgaattataactgtacagttgatattccggttttggtattctttctgaccctggctctaactccttacctgatgtctggtctatcacagtcaacttactagcactgggtctgtttctcatgccaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 6 - DNA, 6627 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcaggtaaggagaccagtcccctgagggagcgtggagtgcctccccatctacagcactgcttctacatatcctggtcatcagaaccactactggggcctcttttgtgggtacactttccctttagtaaaggcttatgcagtatttcctttgacttctaatgctatgtaagtttacctaacaccttcacgggtctcttttatccacacagtgtttcagcctaccatcttggagtgctgagatactacatggtttgcccaaagtcacccagcaagtcttagaagcagggttcaagtcttcctgattggtgtagctctgctacttcctcaccaagagctgacaggctatatctcaagaaattccaaggaagcaccaaactgtaacagctgttcctctggaagcaaagttttgccagaaacagttctctggtgttcctaagatttaccaggaatgagcattaatggaattttgtgtcctctctctgtaaacgtaactcttctcattggctcagagttaagtgtagagacacataaccatgtgaagagtccctttgtgttcaggaaggatgcggctccttaaggttcctcaattgtgatacgtctatttttttccatggtcttaaatgaatttctccgaatacaggattttttaaatgcaatgctgaaatatagacttaataggccaaaaataagataaatttaatctttcttttgcaaaataacttttatttctggttagctcagctcaggtgggccaacatgaatttacggtttagagataaaaatttggttttctgaaattatcaggaaaatattagttgtaaggagcatatcctatagacatgtcatttcttgctgatataaaaaccattggtcccattataaactacatgaagaacaaagacatgatcagcttctactgactaagtcaatggttaacctcagctcaaattaagaaaaagttttaacatgaaaccaagcttgaaaattctgttacctgaaccaacatgtatcaatcactttctaagcatggacttccgggccctcagtttgggattagaaaggtattctcaggccattttccagacaagtgagtcctgatttggtctgtgagatgaaaccagacatgcggaagaccaggccagacagaggaatctgaccgtgccacttcctgctcatccaaacaggaggctttctcaccatcctgcaaggaggttcttggggtcaagtgcagctctcccaccaggtctcttgctcttcttgcccaggacatcattccttatttttcttctctatgaccaagtgctcagttacccttatattctataagtaggtagtcccttagaggaagcagtaagttggtgctttcaccactaagacgaaatgaagaatagtgatggcgaaggcacacgtactctacctccctttcccaaggtgctctgcaagagaacctatgtgcctcagacaactcccatctgccatcttggtgctcctctctaaggtcccagtgcagtggtcaccaagaaaagcaccccgagacatagcaggcaggaagcttctcttggatagtaagggccgcagtctctgaatcctatcagaaaaggctgtctcttccactatgctctttgatatttagaatacagagcttaaatcctgcataaagtagcagctccatggccctagagtaaaaaaactggccagtctgatgctctcatttcattgttttaacaaaacttctgggaggaaggcctcaaaggttcttctgagtgttttgaggtgctagctggatggaaggggaaaatatgtgataataaaatctatctcccttaattatggtctcaggtggcagtagccaccatctctgaacaacaacaaaaacaaccaaccaggaaacatcaacaaaaccagactctatgagatattcacgactgatttgttatagtggcggctgtctaagaagtctgaatctatctgacaggagtatctgttacgtggccctcatacactgtaacatttctagaattcatggcccagctatagcagaataatttatttcagagttaacctgaaaccacctgttggaacgtcccactaatgctatccaggtgaagggcttccctacccctctgctccaccgctagtaaagccaaaatacaccccctctggatctccccatatccacctctcccaaatgcagacactgatgggtaattaacaccactgagaatcccagggtagaaataaaggctcagtctctaaacactcaactcagatggagccactgggtctaaatgctcaccctgtggtttgttctcttgtagatgccatctacgactgctcttccctctaccagaagaactaccgcatctctggagtgtataagcttcctcctgatgacttcctgggcagccctgaactggaggtgaggtcattacagtcactggccatgccctaatacctgtccttcaccccctcaaggggactacaacaacagggccattcacagtttaaagaaaggaaaattcggctgggcgcagtggctcacacctgtaatcccagcactatgggaggccgaggcaggtggatcacttcaggtcaggagtttaagaccagcctggccaacatggtgaaaccctgtctctactaaaaatacaaaaaaattagccaggcatggtggtgggcacctgtaatccctgctacacaggaggattgcttgaactcaggaggcagaggttgcagtgagccgagatcacgccactgcactataatctgggagacaaagtgagactccatttcaattaaaaaaaaaaaaaaaaaaaaggaaaactcaaacacaagcaaacacaccaaacaccacagagctatgcaaacactcagtttatgccctgcactccaaacccaggcatctgtttggccccttcaaatcattatcagtcaaacaacaagccttctaacatagatcagatcattcttataaccaccacataacttagtttaaatctcttgccatgtcctagaacagctattccttgggggaggagaaaagaaaacacgaaggcagcatcaaattatctggattttcacccaggcatggtggctcacacctgtaatcccaagttttttgggaggtgaggtgggcggaacaatcacctgaggtcaggactttgagaccagcctggccaacatgctgaaacccagtctctactaaaaatacaaaaattagcccagtgtggtgacaggcactctggtcccagctactaggaaggcaggagaatcactggaactcaggaggtggaggttgcagtgagccgagattgcaccactgtactctagcctgggcaacaagagtgaaattctgcttcaaaaaaaaaaaaagtatctggatttttccctccaagcttcatgtgcactcacccccgggcccaatttgcatcgttcttccagagcaatgcaccacccaccccagctcaccagcagtggggcagcatcactgcccgagtgagccagtgtgactgcgggagtgcacacatctactggctctgcagggacaggaacaggttgggaagcctgccctcttgctcctgccttctgcccctgcaagtccctcaccagagtatcccctctgcttcaggtgttctgtgacatggagacttcaggcggaggctggaccatcatccagagacgaaaaagtggccttgtctccttctaccgggactagaagcagtacaagcagggctttggcagcatccgtggggacttctggctggggaacgaacacatccaccggctctccagacagccaacccggctgcgtgtagagatggaggtaagcacaaggccaggggccccatgactggaccagtgccaccacacatgaccgcgtacaactccgggggtgccattcctattctgattcaagacaaatctgtatattcattgtgatggttttcctgcaagttgtaatggagttgaggaaaaataggtatttttcctttctgcaacccccccaaccccccgacaaaagtggggctgcaggtgggacaggaagaggccagacccaggccagagtagagcaaattcaacagtcagctgtgccgaacactagtctctgctctggccgagcatgaggtcctttaggtgcaaatcttactgatactgtttggggacccttgctgaaggtctgaaagcactcactatatcctcatgtttctcttacagcagctctgtgtgggattcagcaaaaacatagctgcaccttataagcaggaaagtgaggaatatagaaagagagactaatcaaggccatatggtgaatcaggaaagaagttcgagccttgttttctgattcccaggttaacacagtaaactggaggtaaacaagtaataaagtcttattagattcacacctataaaaagatgtttggctatgggactgtcaggagagaaggggtatagagacagcatgaaatggagcctgctgcactttctttaaggctctgctcctcctgacaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtgagatttggggggaccggaaaggagaagttcaggtacaagctcataatcccacttgaggagaaagagtgaattataactgtacagttgatattccggttttggtattctttctgaccctggctctaactccttacctgatgtctggtctatcacagtcaacttactagcactgggtctgtttctcatgccaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 7 - DNA, 6627 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcaggtaaggagaccagtcccctgagggagcgtggagtgcctccccatctacagcactgcttctacatatcctggtcatcagaaccactactggggcctcttttgtgggtacactttccctttagtaaaggcttatgcagtatttcctttgacttctaatgctatgtaagtttacctaacaccttcacgggtctcttttatccacacagtgtttcagcctaccatcttggagtgctgagatactacatggtttgcccaaagtcacccagcaagtcttagaagcagggttcaagtcttcctgattggtgtagctctgctacttcctcaccaagagctgacaggctatatctcaagaaattccaaggaagcaccaaactgtaacagctgttcctctggaagcaaagttttgccagaaacagttctctggtgttcctaagatttaccaggaatgagcattaatggaattttgtgtcctctctctgtaaacgtaactcttctcattggctcagagttaagtgtagagacacataaccatgtgaagagtccctttgtgttcaggaaggatgcggctccttaaggttcctcaattgtgatacgtctatttttttccatggtcttaaatgaatttctccgaatacaggattttttaaatgcaatgctgaaatatagacttaataggccaaaaataagataaatttaatctttcttttgcaaaataacttttatttctggttagctcagctcaggtgggccaacatgaatttacggtttagagataaaaatttggttttctgaaattatcaggaaaatattagttgtaaggagcatatcctatagacatgtcatttcttgctgatataaaaaccattggtcccattataaactacatgaagaacaaagacatgatcagcttctactgactaagtcaatggttaacctcagctcaaattaagaaaaagttttaacatgaaaccaagcttgaaaattctgttacctgaaccaacatgtatcaatcactttctaagcatggacttccgggccctcagtttgggattagaaaggtattctcaggccattttccagacaagtgagtcctgatttggtctgtgagatgaaaccagacatgcggaagaccaggccagacagaggaatctgaccgtgccacttcctgctcatccaaacaggaggctttctcaccatcctgcaaggaggttcttggggtcaagtgcagctctcccaccaggtctcttgctcttcttgcccaggacatcattccttatttttcttctctatgaccaagtgctcagttacccttatattctataagtaggtagtcccttagaggaagcagtaagttggtgctttcaccactaagacgaaatgaagaatagtgatggcgaaggcacacgtactctacctccctttcccaaggtgctctgcaagagaacctatgtgcctcagacaactcccatctgccatcttggtgctcctctctaaggtcccagtgcagtggtcaccaagaaaagcaccccgagacatagcaggcaggaagcttctcttggatagtaagggccgcagtctctgaatcctatcagaaaaggctgtctcttccactatgctctttgatatttagaatacagagcttaaatcctgcataaagtagcagctccatggccctagagtaaaaaaactggccagtctgatgctctcatttcattgttttaacaaaacttctgggaggaaggcctcaaaggttcttctgagtgttttgaggtgctagctggatggaaggggaaaatatgtgataataaaatctatctcccttaattatggtctcaggtggcagtagccaccatctctgaacaacaacaaaaacaaccaaccaggaaacatcaacaaaaccagactctatgagatattcacgactgatttgttatagtggcggctgtctaagaagtctgaatctatctgacaggagtatctgttacgtggccctcatacactgtaacatttctagaattcatggcccagctatagcagaataatttatttcagagttaacctgaaaccacctgttggaacgtcccactaatgctatccaggtgaagggcttccctacccctctgctccaccgctagtaaagccaaaatacaccccctctggatctccccatatccacctctcccaaatgcagacactgatgggtaattaacaccactgagaatcccagggtagaaataaaggctcagtctctaaacactcaactcagatggagccactgggtctaaatgctcaccctgtggtttgttctcttgtagatgccatctacgactgctcttccctctaccagaagaactaccgcatctctggagtgtataagcttcctcctgatgacttcctgggcagccctgaactggaggtgaggtcattacagtcactggccatgccctaatacctgtccttcaccccctcaaggggactacaacaacagggccattcacagtttaaagaaaggaaaattcggctgggcgcagtggctcacacctgtaatcccagcactatgggaggccgaggcaggtggatcacttcaggtcaggagtttaagaccagcctggccaacatggtgaaaccctgtctctactaaaaatacaaaaaaattagccaggcatggtggtgggcacctgtaatccctgctacacaggaggattgcttgaactcaggaggcagaggttgcagtgagccgagatcacgccactgcactataatctgggagacaaagtgagactccatttcaattaaaaaaaaaaaaaaaaaaaaggaaaactcaaacacaagcaaacacaccaaacaccacagagctatgcaaacactcagtttatgccctgcactccaaacccaggcatctgtttggccccttcaaatcattatcagtcaaacaacaagccttctaacatagatcagatcattcttataaccaccacataacttagtttaaatctcttgccatgtcctagaacagctattccttgggggaggagaaaagaaaacacgaaggcagcatcaaattatctggattttcacccaggcatggtggctcacacctgtaatcccaagttttttgggaggtgaggtgggcggaacaatcacctgaggtcaggactttgagaccagcctggccaacatgctgaaacccagtctctactaaaaatacaaaaattagcccagtgtggtgacaggcactctggtcccagctactaggaaggcaggagaatcactggaactcaggaggtggaggttgcagtgagccgagattgcaccactgtactctagcctgggcaacaagagtgaaattctgcttcaaaaaaaaaaaaagtatctggatttttccctccaagcttcatgtgcactcacccccgggcccaatttgcatcgttcttccagagcaatgcaccacccaccccagctcaccagcagtggggcagcatcactgcccgagtgagccagtgtgactgcgggagtgcacacatctactggctctgcagggacaggaacaggttgggaagcctgccctcttgctcctgccttctgcccctgcaagtccctcaccagagtatcccctctgcttcaggtgttctgtgacatggagacttcaggcggaggctggaccatcatccagagacgaaaaagtggccttgtctccttctaccgggactggaagcagtaccagcagggctttggcagcatccgtggggacttctggctggggaacgaacacatccaccggctctccagacagccaacccggctgcgtgtagagatggaggtaagcacaaggccaggggccccatgactggaccagtgccaccacacatgaccgcgtacaactccgggggtgccattcctattctgattcaagacaaatctgtatattcattgtgatggttttcctgcaagttgtaatggagttgaggaaaaataggtatttttcctttctgcaacccccccaaccccccgacaaaagtggggctgcaggtgggacaggaagaggccagacccaggccagagtagagcaaattcaacagtcagctgtgccgaacactagtctctgctctggccgagcatgaggtcctttaggtgcaaatcttactgatactgtttggggacccttgctgaaggtctgaaagcactcactatatcctcatgtttctcttacagcagctctgtgtgggattcagcaaaaacatagctgcaccttataagcaggaaagtgaggaatatagaaagagagactaatcaaggccatatggtgaatcaggaaagaagttcgagccttgttttctgattcccaggttaacacagtaaactggaggtaaacaagtaataaagtcttattagattcacacctataaaaagatgtttggctatgggactgtcaggagagaaggggtatagagacagcatgaaatggagcctgctgcactttctttaaggctctgctcctcctgacaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtgagatttggggggaccggaaaggagaagttcaggtacaagctcataatcccacttgaggagaaagagtgaattataactgtacagttgatattccggttttggtattctttctgaccctggctctaactccttacctgatgtctggtctatcacagtcaacttactagcactgggtctgtttctcatgccaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 8 - DNA, 6627 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcaggtaaggagaccagtcccctgagggagcgtggagtgcctccccatctacagcactgcttctacatatcctggtcatcagaaccactactggggcctcttttgtgggtacactttccctttagtaaaggcttatgcagtatttcctttgacttctaatgctatgtaagtttacctaacaccttcacgggtctcttttatccacacagtgtttcagcctaccatcttggagtgctgagatactacatggtttgcccaaagtcacccagcaagtcttagaagcagggttcaagtcttcctgattggtgtagctctgctacttcctcaccaagagctgacaggctatatctcaagaaattccaaggaagcaccaaactgtaacagctgttcctctggaagcaaagttttgccagaaacagttctctggtgttcctaagatttaccaggaatgagcattaatggaattttgtgtcctctctctgtaaacgtaactcttctcattggctcagagttaagtgtagagacacataaccatgtgaagagtccctttgtgttcaggaaggatgcggctccttaaggttcctcaattgtgatacgtctatttttttccatggtcttaaatgaatttctccgaatacaggattttttaaatgcaatgctgaaatatagacttaataggccaaaaataagataaatttaatctttcttttgcaaaataacttttatttctggttagctcagctcaggtgggccaacatgaatttacggtttagagataaaaatttggttttctgaaattatcaggaaaatattagttgtaaggagcatatcctatagacatgtcatttcttgctgatataaaaaccattggtcccattataaactacatgaagaacaaagacatgatcagcttctactgactaagtcaatggttaacctcagctcaaattaagaaaaagttttaacatgaaaccaagcttgaaaattctgttacctgaaccaacatgtatcaatcactttctaagcatggacttccgggccctcagtttgggattagaaaggtattctcaggccattttccagacaagtgagtcctgatttggtctgtgagatgaaaccagacatgcggaagaccaggccagacagaggaatctgaccgtgccacttcctgctcatccaaacaggaggctttctcaccatcctgcaaggaggttcttggggtcaagtgcagctctcccaccaggtctcttgctcttcttgcccaggacatcattccttatttttcttctctatgaccaagtgctcagttacccttatattctataagtaggtagtcccttagaggaagcagtaagttggtgctttcaccactaagacgaaatgaagaatagtgatggcgaaggcacacgtactctacctccctttcccaaggtgctctgcaagagaacctatgtgcctcagacaactcccatctgccatcttggtgctcctctctaaggtcccagtgcagtggtcaccaagaaaagcaccccgagacatagcaggcaggaagcttctcttggatagtaagggccgcagtctctgaatcctatcagaaaaggctgtctcttccactatgctctttgatatttagaatacagagcttaaatcctgcataaagtagcagctccatggccctagagtaaaaaaactggccagtctgatgctctcatttcattgttttaacaaaacttctgggaggaaggcctcaaaggttcttctgagtgttttgaggtgctagctggatggaaggggaaaatatgtgataataaaatctatctcccttaattatggtctcaggtggcagtagccaccatctctgaacaacaacaaaaacaaccaaccaggaaacatcaacaaaaccagactctatgagatattcacgactgatttgttatagtggcggctgtctaagaagtctgaatctatctgacaggagtatctgttacgtggccctcatacactgtaacatttctagaattcatggcccagctatagcagaataatttatttcagagttaacctgaaaccacctgttggaacgtcccactaatgctatccaggtgaagggcttccctacccctctgctccaccgctagtaaagccaaaatacaccccctctggatctccccatatccacctctcccaaatgcagacactgatgggtaattaacaccactgagaatcccagggtagaaataaaggctcagtctctaaacactcaactcagatggagccactgggtctaaatgctcaccctgtggtttgttctcttgtagatgccatctacgactgctcttccctctaccagaagaactaccgcatctctggagtgtataagcttcctcctgatgacttcctgggcagccctgaactggaggtgaggtcattacagtcactggccatgccctaatacctgtccttcaccccctcaaggggactacaacaacagggccattcacagtttaaagaaaggaaaattcggctgggcgcagtggctcacacctgtaatcccagcactatgggaggccgaggcaggtggatcacttcaggtcaggagtttaagaccagcctggccaacatggtgaaaccctgtctctactaaaaatacaaaaaaattagccaggcatggtggtgggcacctgtaatccctgctacacaggaggattgcttgaactcaggaggcagaggttgcagtgagccgagatcacgccactgcactataatctgggagacaaagtgagactccatttcaattaaaaaaaaaaaaaaaaaaaaggaaaactcaaacacaagcaaacacaccaaacaccacagagctatgcaaacactcagtttatgccctgcactccaaacccaggcatctgtttggccccttcaaatcattatcagtcaaacaacaagccttctaacatagatcagatcattcttataaccaccacataacttagtttaaatctcttgccatgtcctagaacagctattccttgggggaggagaaaagaaaacacgaaggcagcatcaaattatctggattttcacccaggcatggtggctcacacctgtaatcccaagttttttgggaggtgaggtgggcggaacaatcacctgaggtcaggactttgagaccagcctggccaacatgctgaaacccagtctctactaaaaatacaaaaattagcccagtgtggtgacaggcactctggtcccagctactaggaaggcaggagaatcactggaactcaggaggtggaggttgcagtgagccgagattgcaccactgtactctagcctgggcaacaagagtgaaattctgcttcaaaaaaaaaaaaagtatctggatttttccctccaagcttcatgtgcactcacccccgggcccaatttgcatcgttcttccagagcaatgcaccacccaccccagctcaccagcagtggggcagcatcactgcccgagtgagccagtgtgactgcgggagtgcacacatctactggctctgcagggacaggaacaggttgggaagcctgccctcttgctcctgccttctgcccctgcaagtccctcaccagagtatcccctctgcttcaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggtaagcacaaggccaggggccccatgactggaccagtgccaccacacatgaccgcgtacaactccgggggtgccattcctattctgattcaagacaaatctgtatattcattgtgatggttttcctgcaagttgtaatggagttgaggaaaaataggtatttttcctttctgcaacccccccaaccccccgacaaaagtggggctgcaggtgggacaggaagaggccagacccaggccagagtagagcaaattcaacagtcagctgtgccgaacactagtctctgctctggccgagcatgaggtcctttaggtgcaaatcttactgatactgtttggggacccttgctgaaggtctgaaagcactcactatatcctcatgtttctcttacagcagctctgtgtgggattcagcaaaaacatagctgcaccttataagcaggaaagtgaggaatatagaaagagagactaatcaaggccatatggtgaatcaggaaagaagttcgagccttgttttctgattcccaggttaacacagtaaactggaggtaaacaagtaataaagtcttattagattcacacctataaaaagatgtttggctatgggactgtcaggagagaaggggtatagagacagcatgaaatggagcctgctgcactttctttaaggctctgctcctcctgacaggactgggagggcaacctgtgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtgagatttggggggaccggaaaggagaagttcaggtacaagctcataatcccacttgaggagaaagagtgaattataactgtacagttgatattccggttttggtattctttctgaccctggctctaactccttacctgatgtctggtctatcacagtcaacttactagcactgggtctgtttctcatgccaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 9 - DNA, 6627 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcaggtaaggagaccagtcccctgagggagcgtggagtgcctccccatctacagcactgcttctacatatcctggtcatcagaaccactactggggcctcttttgtgggtacactttccctttagtaaaggcttatgcagtatttcctttgacttctaatgctatgtaagtttacctaacaccttcacgggtctcttttatccacacagtgtttcagcctaccatcttggagtgctgagatactacatggtttgcccaaagtcacccagcaagtcttagaagcagggttcaagtcttcctgattggtgtagctctgctacttcctcaccaagagctgacaggctatatctcaagaaattccaaggaagcaccaaactgtaacagctgttcctctggaagcaaagttttgccagaaacagttctctggtgttcctaagatttaccaggaatgagcattaatggaattttgtgtcctctctctgtaaacgtaactcttctcattggctcagagttaagtgtagagacacataaccatgtgaagagtccctttgtgttcaggaaggatgcggctccttaaggttcctcaattgtgatacgtctatttttttccatggtcttaaatgaatttctccgaatacaggattttttaaatgcaatgctgaaatatagacttaataggccaaaaataagataaatttaatctttcttttgcaaaataacttttatttctggttagctcagctcaggtgggccaacatgaatttacggtttagagataaaaatttggttttctgaaattatcaggaaaatattagttgtaaggagcatatcctatagacatgtcatttcttgctgatataaaaaccattggtcccattataaactacatgaagaacaaagacatgatcagcttctactgactaagtcaatggttaacctcagctcaaattaagaaaaagttttaacatgaaaccaagcttgaaaattctgttacctgaaccaacatgtatcaatcactttctaagcatggacttccgggccctcagtttgggattagaaaggtattctcaggccattttccagacaagtgagtcctgatttggtctgtgagatgaaaccagacatgcggaagaccaggccagacagaggaatctgaccgtgccacttcctgctcatccaaacaggaggctttctcaccatcctgcaaggaggttcttggggtcaagtgcagctctcccaccaggtctcttgctcttcttgcccaggacatcattccttatttttcttctctatgaccaagtgctcagttacccttatattctataagtaggtagtcccttagaggaagcagtaagttggtgctttcaccactaagacgaaatgaagaatagtgatggcgaaggcacacgtactctacctccctttcccaaggtgctctgcaagagaacctatgtgcctcagacaactcccatctgccatcttggtgctcctctctaaggtcccagtgcagtggtcaccaagaaaagcaccccgagacatagcaggcaggaagcttctcttggatagtaagggccgcagtctctgaatcctatcagaaaaggctgtctcttccactatgctctttgatatttagaatacagagcttaaatcctgcataaagtagcagctccatggccctagagtaaaaaaactggccagtctgatgctctcatttcattgttttaacaaaacttctgggaggaaggcctcaaaggttcttctgagtgttttgaggtgctagctggatggaaggggaaaatatgtgataataaaatctatctcccttaattatggtctcaggtggcagtagccaccatctctgaacaacaacaaaaacaaccaaccaggaaacatcaacaaaaccagactctatgagatattcacgactgatttgttatagtggcggctgtctaagaagtctgaatctatctgacaggagtatctgttacgtggccctcatacactgtaacatttctagaattcatggcccagctatagcagaataatttatttcagagttaacctgaaaccacctgttggaacgtcccactaatgctatccaggtgaagggcttccctacccctctgctccaccgctagtaaagccaaaatacaccccctctggatctccccatatccacctctcccaaatgcagacactgatgggtaattaacaccactgagaatcccagggtagaaataaaggctcagtctctaaacactcaactcagatggagccactgggtctaaatgctcaccctgtggtttgttctcttgtagatgccatctacgactgctcttccctctaccagaagaactaccgcatctctggagtgtataagcttcctcctgatgacttcctgggcagccctgaactggaggtgaggtcattacagtcactggccatgccctaatacctgtccttcaccccctcaaggggactacaacaacagggccattcacagtttaaagaaaggaaaattcggctgggcgcagtggctcacacctgtaatcccagcactatgggaggccgaggcaggtggatcacttcaggtcaggagtttaagaccagcctggccaacatggtgaaaccctgtctctactaaaaatacaaaaaaattagccaggcatggtggtgggcacctgtaatccctgctacacaggaggattgcttgaactcaggaggcagaggttgcagtgagccgagatcacgccactgcactataatctgggagacaaagtgagactccatttcaattaaaaaaaaaaaaaaaaaaaaggaaaactcaaacacaagcaaacacaccaaacaccacagagctatgcaaacactcagtttatgccctgcactccaaacccaggcatctgtttggccccttcaaatcattatcagtcaaacaacaagccttctaacatagatcagatcattcttataaccaccacataacttagtttaaatctcttgccatgtcctagaacagctattccttgggggaggagaaaagaaaacacgaaggcagcatcaaattatctggattttcacccaggcatggtggctcacacctgtaatcccaagttttttgggaggtgaggtgggcggaacaatcacctgaggtcaggactttgagaccagcctggccaacatgctgaaacccagtctctactaaaaatacaaaaattagcccagtgtggtgacaggcactctggtcccagctactaggaaggcaggagaatcactggaactcaggaggtggaggttgcagtgagccgagattgcaccactgtactctagcctgggcaacaagagtgaaattctgcttcaaaaaaaaaaaaagtatctggatttttccctccaagcttcatgtgcactcacccccgggcccaatttgcatcgttcttccagagcaatgcaccacccaccccagctcaccagcagtggggcagcatcactgcccgagtgagccagtgtgactgcgggagtgcacacatctactggctctgcagggacaggaacaggttgggaagcctgccctcttgctcctgccttctgcccctgcaagtccctcaccagagtatcccctctgcttcaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggtaagcacaaggccaggggccccatgactggaccagtgccaccacacatgaccgcgtacaactccgggggtgccattcctattctgattcaagacaaatctgtatattcattgtgatggttttcctgcaagttgtaatggagttgaggaaaaataggtatttttcctttctgcaacccccccaaccccccgacaaaagtggggctgcaggtgggacaggaagaggccagacccaggccagagtagagcaaattcaacagtcagctgtgccgaacactagtctctgctctggccgagcatgaggtcctttaggtgcaaatcttactgatactgtttggggacccttgctgaaggtctgaaagcactcactatatcctcatgtttctcttacagcagctctgtgtgggattcagcaaaaacatagctgcaccttataagcaggaaagtgaggaatatagaaagagagactaatcaaggccatatggtgaatcaggaaagaagttcgagccttgttttctgattcccaggttaacacagtaaactggaggtaaacaagtaataaagtcttattagattcacacctataaaaagatgtttggctatgggactgtcaggagagaaggggtatagagacagcatgaaatggagcctgctgcactttctttaaggctctgctcctcctgacaggactgggagggcaacctgcgctacgctgagtatagccactttgttttgggcaatgaactcaacagctattgcctcttcctggggaactacactggcaatgtggggaacgacgccctccagtatcataacaacacagccttcagcaccaaggacaaggacaatgacaactgcttggacaagtgtgcacagctccgcaaaggtgagatttggggggaccggaaaggagaagttcaggtacaagctcataatcccacttgaggagaaagagtgaattataactgtacagttgatattccggttttggtattctttctgaccctggctctaactccttacctgatgtctggtctatcacagtcaacttactagcactgggtctgtttctcatgccaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 10 - DNA, 6627 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcaggtaaggagaccagtcccctgagggagcgtggagtgcctccccatctacagcactgcttctacatatcctggtcatcagaaccactactggggcctcttttgtgggtacactttccctttagtaaaggcttatgcagtatttcctttgacttctaatgctatgtaagtttacctaacaccttcacgggtctcttttatccacacagtgtttcagcctaccatcttggagtgctgagatactacatggtttgcccaaagtcacccagcaagtcttagaagcagggttcaagtcttcctgattggtgtagctctgctacttcctcaccaagagctgacaggctatatctcaagaaattccaaggaagcaccaaactgtaacagctgttcctctggaagcaaagttttgccagaaacagttctctggtgttcctaagatttaccaggaatgagcattaatggaattttgtgtcctctctctgtaaacgtaactcttctcattggctcagagttaagtgtagagacacataaccatgtgaagagtccctttgtgttcaggaaggatgcggctccttaaggttcctcaattgtgatacgtctatttttttccatggtcttaaatgaatttctccgaatacaggattttttaaatgcaatgctgaaatatagacttaataggccaaaaataagataaatttaatctttcttttgcaaaataacttttatttctggttagctcagctcaggtgggccaacatgaatttacggtttagagataaaaatttggttttctgaaattatcaggaaaatattagttgtaaggagcatatcctatagacatgtcatttcttgctgatataaaaaccattggtcccattataaactacatgaagaacaaagacatgatcagcttctactgactaagtcaatggttaacctcagctcaaattaagaaaaagttttaacatgaaaccaagcttgaaaattctgttacctgaaccaacatgtatcaatcactttctaagcatggacttccgggccctcagtttgggattagaaaggtattctcaggccattttccagacaagtgagtcctgatttggtctgtgagatgaaaccagacatgcggaagaccaggccagacagaggaatctgaccgtgccacttcctgctcatccaaacaggaggctttctcaccatcctgcaaggaggttcttggggtcaagtgcagctctcccaccaggtctcttgctcttcttgcccaggacatcattccttatttttcttctctatgaccaagtgctcagttacccttatattctataagtaggtagtcccttagaggaagcagtaagttggtgctttcaccactaagacgaaatgaagaatagtgatggcgaaggcacacgtactctacctccctttcccaaggtgctctgcaagagaacctatgtgcctcagacaactcccatctgccatcttggtgctcctctctaaggtcccagtgcagtggtcaccaagaaaagcaccccgagacatagcaggcaggaagcttctcttggatagtaagggccgcagtctctgaatcctatcagaaaaggctgtctcttccactatgctctttgatatttagaatacagagcttaaatcctgcataaagtagcagctccatggccctagagtaaaaaaactggccagtctgatgctctcatttcattgttttaacaaaacttctgggaggaaggcctcaaaggttcttctgagtgttttgaggtgctagctggatggaaggggaaaatatgtgataataaaatctatctcccttaattatggtctcaggtggcagtagccaccatctctgaacaacaacaaaaacaaccaaccaggaaacatcaacaaaaccagactctatgagatattcacgactgatttgttatagtggcggctgtctaagaagtctgaatctatctgacaggagtatctgttacgtggccctcatacactgtaacatttctagaattcatggcccagctatagcagaataatttatttcagagttaacctgaaaccacctgttggaacgtcccactaatgctatccaggtgaagggcttccctacccctctgctccaccgctagtaaagccaaaatacaccccctctggatctccccatatccacctctcccaaatgcagacactgatgggtaattaacaccactgagaatcccagggtagaaataaaggctcagtctctaaacactcaactcagatggagccactgggtctaaatgctcaccctgtggtttgttctcttgtagatgccatctacgactgctcttccctctaccagaagaactaccgcatctctggagtgtataagcttcctcctgatgacttcctgggcagccctgaactggaggtgaggtcattacagtcactggccatgccctaatacctgtccttcaccccctcaaggggactacaacaacagggccattcacagtttaaagaaaggaaaattcggctgggcgcagtggctcacacctgtaatcccagcactatgggaggccgaggcaggtggatcacttcaggtcaggagtttaagaccagcctggccaacatggtgaaaccctgtctctactaaaaatacaaaaaaattagccaggcatggtggtgggcacctgtaatccctgctacacaggaggattgcttgaactcaggaggcagaggttgcagtgagccgagatcacgccactgcactataatctgggagacaaagtgagactccatttcaattaaaaaaaaaaaaaaaaaaaaggaaaactcaaacacaagcaaacacaccaaacaccacagagctatgcaaacactcagtttatgccctgcactccaaacccaggcatctgtttggccccttcaaatcattatcagtcaaacaacaagccttctaacatagatcagatcattcttataaccaccacataacttagtttaaatctcttgccatgtcctagaacagctattccttgggggaggagaaaagaaaacacgaaggcagcatcaaattatctggattttcacccaggcatggtggctcacacctgtaatcccaagttttttgggaggtgaggtgggcggaacaatcacctgaggtcaggactttgagaccagcctggccaacatgctgaaacccagtctctactaaaaatacaaaaattagcccagtgtggtgacaggcactctggtcccagctactaggaaggcaggagaatcactggaactcaggaggtggaggttgcagtgagccgagattgcaccactgtactctagcctgggcaacaagagtgaaattctgcttcaaaaaaaaaaaaagtatctggatttttccctccaagcttcatgtgcactcacccccgggcccaatttgcatcgttcttccagagcaatgcaccacccaccccagctcaccagcagtggggcagcatcactgcccgagtgagccagtgtgactgcgggagtgcacacatctactggctctgcagggacaggaacaggttgggaagcctgccctcttgctcctgccttctgcccctgcaagtccctcaccagagtatcccctctgcttcaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggtaagcacaaggccaggggccccatgactggaccagtgccaccacacatgaccgcgtacaactccgggggtgccattcctattctgattcaagacaaatctgtatattcattgtgatggttttcctgcaagttgtaatggagttgaggaaaaataggtatttttcctttctgcaacccccccaaccccccgacaaaagtggggctgcaggtgggacaggaagaggccagacccaggccagagtagagcaaattcaacagtcagctgtgccgaacactagtctctgctctggccgagcatgaggtcctttaggtgcaaatcttactgatactgtttggggacccttgctgaaggtctgaaagcactcactatatcctcatgtttctcttacagcagctctgtgtgggattcagcaaaaacatagctgcaccttataagcaggaaagtgaggaatatagaaagagagactaatcaaggccatatggtgaatcaggaaagaagttcgagccttgttttctgattcccaggttaacacagtaaactggaggtaaacaagtaataaagtcttattagattcacacctataaaaagatgtttggctatgggactgtcaggagagaaggggtatagagacagcatgaaatggagcctgctgcactttctttaaggctctgctcctcctgacaggactgggagggcaacctgcgctacgctgagtatagccactttgttttgggcaatgaactcaacagctatcgcctcttcctggggaactacactggcaatgtggggaacgacgccctccagtatcaaaacaacacagccttcagcaccaaggacaaggacaatgacaactgcttggacaagtgtgcacagctccgcaaaggtgagatttggggggaccggaaaggagaagttcaggtacaagctcataatcccacttgaggagaaagagtgaattataactgtacagttgatattccggttttggtattctttctgaccctggctctaactccttacctgatgtctggtctatcacagtcaacttactagcactgggtctgtttctcatgccaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 11 - RNA, 2224 nt, homo sapien
gggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 12 - RNA, 2245 nt, homo sapien
gcagccaugguagggguggagguacaggcagcaaacaauauuuaagauacugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaac

SEQ ID NO: 13 - RNA, 1298 nt, homo sapien
aauauuuaagaugcugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcuguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaa

SEQ ID NO: 14 - RNA, 2255 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugugaaaaaaaaaaaaaaaaaa

SEQ ID NO: 15 - RNA, 2212 nt, homo sapien
gaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 16 - RNA, 2006 nt, homo sapien
acaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagacccauauuauuuugauucucaaaggauaggccuuugaguguuagagaaaggagugaaggagccaggugggaaaugguauuucuauuuuuaaacuccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaggcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 17 - RNA, 1041 nt, homo sapien
augcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuag

SEQ ID NO: 18 - RNA, 2238 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 19 - RNA, 2224 nt, homo sapien
gggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggaggugaucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 20 - RNA, 2245 nt, homo sapien
gcagccaugguagggguggagguacaggcagcaaacaauauuuaagauacugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggaggugaucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaac

SEQ ID NO: 21 - RNA, 1298 nt, homo sapien
aauauuuaagaugcugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcuguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggaggugaucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaa

SEQ ID NO: 22 - RNA, 2255 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggaggugaucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugugaaaaaaaaaaaaaaaaaa

SEQ ID NO: 23 - RNA, 2212 nt, homo sapien
gaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggaggugaucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 24 - RNA, 2006 nt, homo sapien
acaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggaggugaucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagacccauauuauuuugauucucaaaggauaggccuuugaguguuagagaaaggagugaaggagccaggugggaaaugguauuucuauuuuuaaacuccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaggcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 25 - RNA, 1041 nt, homo sapien
augcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggaggugaucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuag

SEQ ID NO: 26 - RNA, 2238 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggaggugaucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 27 - RNA, 2224 nt, homo sapien
gggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucaaccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 28 - RNA, 2245 nt, homo sapien
gcagccaugguagggguggagguacaggcagcaaacaauauuuaagauacugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucaaccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaac

SEQ ID NO: 29 - RNA, 1298 nt, homo sapien
aauauuuaagaugcugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcuguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucaaccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaa

SEQ ID NO: 30 - RNA, 2255 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucaaccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugugaaaaaaaaaaaaaaaaaa

SEQ ID NO: 31 - RNA, 2212 nt, homo sapien
gaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucaaccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 32 - RNA, 2006 nt, homo sapien
acaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucaaccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagacccauauuauuuugauucucaaaggauaggccuuugaguguuagagaaaggagugaaggagccaggugggaaaugguauuucuauuuuuaaacuccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaggcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 33 - RNA, 1041 nt, homo sapien
augcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucaaccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuag

SEQ ID NO: 34 - RNA, 2238 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucaaccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 35 - RNA, 2224 nt, homo sapien
gggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccauagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 36 - RNA, 2245 nt, homo sapien
gcagccaugguagggguggagguacaggcagcaaacaauauuuaagauacugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccauagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaac

SEQ ID NO: 37 - RNA, 1298 nt, homo sapien
aauauuuaagaugcugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcuguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccauagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaa

SEQ ID NO: 38 - RNA, 2255 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccauagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugugaaaaaaaaaaaaaaaaaa

SEQ ID NO: 39 - RNA, 2212 nt, homo sapien
gaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccauagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 40 - RNA, 2006 nt, homo sapien
acaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccauagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagacccauauuauuuugauucucaaaggauaggccuuugaguguuagagaaaggagugaaggagccaggugggaaaugguauuucuauuuuuaaacuccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaggcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 41 - RNA, 1041 nt, homo sapien
augcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccauagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuag

SEQ ID NO: 42 - RNA, 2238 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccauagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 43 - RNA, 2224 nt, homo sapien
gggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagaugaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 44 - RNA, 2245 nt, homo sapien
gcagccaugguagggguggagguacaggcagcaaacaauauuuaagauacugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagaugaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaac

SEQ ID NO: 45 - RNA, 1298 nt, homo sapien
aauauuuaagaugcugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcuguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagaugaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaa

SEQ ID NO: 46 - RNA, 2255 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagaugaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugugaaaaaaaaaaaaaaaaaa

SEQ ID NO: 47 - RNA, 2212 nt, homo sapien
gaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagaugaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 48 - RNA, 2006 nt, homo sapien
acaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagaugaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagacccauauuauuuugauucucaaaggauaggccuuugaguguuagagaaaggagugaaggagccaggugggaaaugguauuucuauuuuuaaacuccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaggcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 49 - RNA, 1041 nt, homo sapien
augcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagaugaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuag

SEQ ID NO: 50 - RNA, 2238 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagaugaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 51 - RNA, 2224 nt, homo sapien
gggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuagaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 52 - RNA, 2245 nt, homo sapien
gcagccaugguagggguggagguacaggcagcaaacaauauuuaagauacugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuagaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaac

SEQ ID NO: 53 - RNA, 1298 nt, homo sapien
aauauuuaagaugcugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcuguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuagaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaa

SEQ ID NO: 54 - RNA, 2255 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuagaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugugaaaaaaaaaaaaaaaaaa

SEQ ID NO: 55 - RNA, 2212 nt, homo sapien
gaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuagaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 56 - RNA, 2006 nt, homo sapien
acaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuagaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagacccauauuauuuugauucucaaaggauaggccuuugaguguuagagaaaggagugaaggagccaggugggaaaugguauuucuauuuuuaaacuccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaggcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 57 - RNA, 1041 nt, homo sapien
augcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuagaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuag

SEQ ID NO: 58 - RNA, 2238 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuagaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 59 - RNA, 2224 nt, homo sapien
gggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguaccagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 60 - RNA, 2245 nt, homo sapien
gcagccaugguagggguggagguacaggcagcaaacaauauuuaagauacugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguaccagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaac

SEQ ID NO: 61 - RNA, 1298 nt, homo sapien
aauauuuaagaugcugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcuguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguaccagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaa

SEQ ID NO: 62 - RNA, 2255 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguaccagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugugaaaaaaaaaaaaaaaaaa

SEQ ID NO: 63 - RNA, 2212 nt, homo sapien
gaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguaccagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 64 - RNA, 2006 nt, homo sapien
acaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguaccagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagacccauauuauuuugauucucaaaggauaggccuuugaguguuagagaaaggagugaaggagccaggugggaaaugguauuucuauuuuuaaacuccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaggcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 65 - RNA, 1041 nt, homo sapien
augcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguaccagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuag

SEQ ID NO: 66 - RNA, 2238 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguaccagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 67 - RNA, 2224 nt, homo sapien
gggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugugcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 68 - RNA, 2245 nt, homo sapien
gcagccaugguagggguggagguacaggcagcaaacaauauuuaagauacugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugugcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaac

SEQ ID NO: 69 - RNA, 1298 nt, homo sapien
aauauuuaagaugcugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcuguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugugcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaa

SEQ ID NO: 70 - RNA, 2255 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugugcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugugaaaaaaaaaaaaaaaaaa

SEQ ID NO: 71 - RNA, 2212 nt, homo sapien
gaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugugcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 72 - RNA, 2006 nt, homo sapien
acaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugugcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagacccauauuauuuugauucucaaaggauaggccuuugaguguuagagaaaggagugaaggagccaggugggaaaugguauuucuauuuuuaaacuccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaggcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 73 - RNA, 1041 nt, homo sapien
augcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugugcuacgcugaguauagccacuuuguuuugggcaaugaacucaacagcuaucgccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuag

SEQ ID NO: 74 - RNA, 2238 nt, homo sapien; identical to SEQ ID NO: 18

SEQ ID NO: 75 - RNA, 2224 nt, homo sapien
gggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuauugccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 76 - RNA, 2245 nt, homo sapien
gcagccaugguagggguggagguacaggcagcaaacaauauuuaagauacugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuauugccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaac

SEQ ID NO: 77 - RNA, 1298 nt, homo sapien
aauauuuaagaugcugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcuguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuauugccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaa

SEQ ID NO: 78 - RNA, 2255 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuauugccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugugaaaaaaaaaaaaaaaaaa

SEQ ID NO: 79 - RNA, 2212 nt, homo sapien
gaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuauugccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 80 - RNA, 2006 nt, homo sapien
acaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuauugccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagacccauauuauuuugauucucaaaggauaggccuuugaguguuagagaaaggagugaaggagccaggugggaaaugguauuucuauuuuuaaacuccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaggcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 81 - RNA, 1041 nt, homo sapien
augcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuauugccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuag

SEQ ID NO: 82 - RNA, 2238 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuauugccucuuccuggggaacuacacuggcaauguggggaacgacgcccuccaguaucauaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 83 - RNA, 2224 nt, homo sapien
gggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucaaaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 84 - RNA, 2245 nt, homo sapien
gcagccaugguagggguggagguacaggcagcaaacaauauuuaagauacugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucaaaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaac

SEQ ID NO: 85 - RNA, 1298 nt, homo sapien
aauauuuaagaugcugacuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcuguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucaaaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaa

SEQ ID NO: 86 - RNA, 2255 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucaaaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugugaaaaaaaaaaaaaaaaaa

SEQ ID NO: 87 - RNA, 2212 nt, homo sapien
gaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucaaaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 88 - RNA, 2006 nt, homo sapien
acaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucaaaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagacccauauuauuuugauucucaaaggauaggccuuugaguguuagagaaaggagugaaggagccaggugggaaaugguauuucuauuuuuaaacuccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaggcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaaugug

SEQ ID NO: 89 - RNA, 1041 nt, homo sapien
augcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucaaaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuag

SEQ ID NO: 90 - RNA, 2238 nt, homo sapien
cuuguggagcauucgggcuuggaaggaaagcuauaggcuacccauucagcuccccugucagagacucaagcuuugagaaaggcuagcaaagagcaaggaaagagagaaaacaacaaaguggcgaggcccucagagugaaagcguaagguucagucagccugcugcagcuuugcagaccucagcugggcaucuccagacuccccugaaggaagagccuuccucacccaaacccacaaaagaugcugaaaaagccucucucagcugugaccuggcucugcauuuucaucguggccuuugucagccacccagcguggcugcagaagcucucuaagcacaagacaccagcacagccacagcucaaagcggccaacugcugugaggaggugaaggagcucaaggcccaaguugccaaccuuagcagccugcugagugaacugaacaagaagcaggagagggacugggucagcguggucaugcaggugauggagcuggagagcaacagcaagcgcauggagucgcggcucacagaugcugagagcaaguacuccgagaugaacaaccaaauugacaucaugcagcugcaggcagcacagacggucacucagaccuccgcagaugccaucuacgacugcucuucccucuaccagaagaacuaccgcaucucuggaguguauaagcuuccuccugaugacuuccugggcagcccugaacuggagguguucugugacauggagacuucaggcggaggcuggaccaucauccagagacgaaaaaguggccuugucuccuucuaccgggacuggaagcaguacaagcagggcuuuggcagcauccguggggacuucuggcuggggaacgaacacauccaccggcucuccagacagccaacccggcugcguguagagauggaggacugggagggcaaccugcgcuacgcugaguauagccacuuuguuuugggcaaugaacucaacagcuaucgccucuuccuggggaacuacacuggcaauguggggaacgacgcccuccaguaucaaaacaacacagccuucagcaccaaggacaaggacaaugacaacugcuuggacaagugugcacagcuccgcaaagguggcuacugguacaacugcugcacagacuccaaccucaauggaguguacuaccgccugggugagcacaauaagcaccuggauggcaucaccugguauggcuggcauggaucuaccuacucccucaaacggguggagaugaaaauccgcccagaagacuucaagccuuaaaaggaggcugccguggagcacggauacagaaacugagacacguggagacuggaugagggcagaugaggacaggaagagaguguuagaaaggguaggacugagaaacagccuauaaucuccaaagaaagaauaagucuccaaggagcacaaaaaaaucauauguaccaaggauguuacaguaaacaggaugaacuauuuaaacccacuggguccugccacauccuucucaaggugguagacugaguggggucucucugcccaagaucccugacauagcaguagcuugucuuuuccacaugauuugucugugaaagaaaauaauuuugagaucguuuuaucuauuuucucuacggcuuaggcuaugugagggcaaaacacaaaucccuuugcuaaaaagaaccauauuauuuugauucucaaaggauaggccuuugaguguuagagaaaggagugaaggaggcaggugggaaaugguauuucuauuuuuaaauccagugaaauuaucuugagucuacacauuauuuuuaaaacacaaaaauuguucggcuggaacugacccaggcuggacuugcggggaggaaacuccagggcacugcaucuggcgaucagacucugagcacugccccugcucgccuuggucauguacagcacugaaaggaaugaagcaccagcaggagguggacagagucucucauggaugccggcacaaaacugccuuaaaauauucauaguuaauacagguauaucuauuuuuauuuacuuuguaagaaacaagcucaaggagcuuccuuuuaaauuuugucuguaggaaaugguugaaaacugaagguagaugguguuauaguuaauaauaaaugcuguaaauaagcaucucacuuuguaaaaauaaaauauugugguuuuguuuuaaacauucaacguuucuuuuccuucuacaauaaacacuuucaaaauguga

SEQ ID NO: 91 - DNA, 2224 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 92 - DNA, 2245 bp, homo sapien
gcagccatggtaggggtggaggtacaggcagcaaacaatatttaagatactgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaac

SEQ ID NO: 93 - DNA, 1298 bp, homo sapien
aatatttaagatgctgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagctgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaa

SEQ ID NO: 94 - DNA, 2255 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtgaaaaaaaaaaaaaaaaaa

SEQ ID NO: 95 - DNA, 2212 bp, homo sapien
gaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 96 - DNA, 2006 bp, homo sapien
acaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagacccatattattttgattctcaaaggataggcctttgagtgttagagaaaggagtgaaggagccaggtgggaaatggtatttctatttttaaactccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaggcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 97 - DNA, 1041 bp, homo sapien
atgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttag

SEQ ID NO: 98 - DNA, 2238 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 99 - DNA, 2224 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgatctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 100 - DNA, 2245 bp, homo sapien
gcagccatggtaggggtggaggtacaggcagcaaacaatatttaagatactgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgatctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaac

SEQ ID NO: 101 - DNA, 1298 bp, homo sapien
aatatttaagatgctgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagctgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgatctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaa

SEQ ID NO: 102 - DNA, 2255 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgatctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtgaaaaaaaaaaaaaaaaaa

SEQ ID NO: 103 - DNA, 2212 bp, homo sapien
gaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgatctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 104 - DNA, 2006 bp, homo sapien
acaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgatctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagacccatattattttgattctcaaaggataggcctttgagtgttagagaaaggagtgaaggagccaggtgggaaatggtatttctatttttaaactccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaggcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 105 - DNA, 1041 bp, homo sapien
atgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgatctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttag

SEQ ID NO: 106 - DNA, 2238 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgatctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 107 - DNA, 2224 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcaaccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 108 - DNA, 2245 bp, homo sapien
gcagccatggtaggggtggaggtacaggcagcaaacaatatttaagatactgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcaaccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaac

SEQ ID NO: 109 - DNA, 1298 bp, homo sapien
aatatttaagatgctgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagctgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcaaccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaa

SEQ ID NO: 110 - DNA, 2255 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcaaccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtgaaaaaaaaaaaaaaaaaa

SEQ ID NO: 111 - DNA, 2212 bp, homo sapien
gaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcaaccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 112 - DNA, 2006 bp, homo sapien
acaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcaaccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagacccatattattttgattctcaaaggataggcctttgagtgttagagaaaggagtgaaggagccaggtgggaaatggtatttctatttttaaactccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaggcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 113 - DNA, 1041 bp, homo sapien
atgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcaaccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttag

SEQ ID NO: 114 - DNA, 2238 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcaaccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 115 - DNA, 2224 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccatagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 116 - DNA, 2245 bp, homo sapien
gcagccatggtaggggtggaggtacaggcagcaaacaatatttaagatactgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccatagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaac

SEQ ID NO: 117 - DNA, 1298 bp, homo sapien
aatatttaagatgctgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagctgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccatagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaa

SEQ ID NO: 118 - DNA, 2255 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccatagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtgaaaaaaaaaaaaaaaaaa

SEQ ID NO: 119 - DNA, 2212 bp, homo sapien
gaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccatagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 120 - DNA, 2006 bp, homo sapien
acaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccatagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagacccatattattttgattctcaaaggataggcctttgagtgttagagaaaggagtgaaggagccaggtgggaaatggtatttctatttttaaactccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaggcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 121 - DNA, 1041 bp, homo sapien
atgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccatagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttag

SEQ ID NO: 122 - DNA, 2238 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccatagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 123 - DNA, 2224 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagatgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 124 - DNA, 2245 bp, homo sapien
gcagccatggtaggggtggaggtacaggcagcaaacaatatttaagatactgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagatgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaac

SEQ ID NO: 125 - DNA, 1298 bp, homo sapien
aatatttaagatgctgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagctgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagatgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaa

SEQ ID NO: 126 - DNA, 2255 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagatgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtgaaaaaaaaaaaaaaaaaa

SEQ ID NO: 127 - DNA, 2212 bp, homo sapien
gaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagatgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 128 - DNA, 2006 bp, homo sapien
acaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagatgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagacccatattattttgattctcaaaggataggcctttgagtgttagagaaaggagtgaaggagccaggtgggaaatggtatttctatttttaaactccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaggcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 129 - DNA, 1041 bp, homo sapien
atgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagatgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttag

SEQ ID NO: 130 - DNA, 2238 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagatgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 131 - DNA, 2224 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactagaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 132 - DNA, 2245 bp, homo sapien
gcagccatggtaggggtggaggtacaggcagcaaacaatatttaagatactgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactagaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaac

SEQ ID NO: 133 - DNA, 1298 bp, homo sapien
aatatttaagatgctgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagctgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactagaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaa

SEQ ID NO: 134 - DNA, 2255 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactagaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtgaaaaaaaaaaaaaaaaaa

SEQ ID NO: 135 - DNA, 2212 bp, homo sapien
gaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactagaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 136 - DNA, 2006 bp, homo sapien
acaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactagaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagacccatattattttgattctcaaaggataggcctttgagtgttagagaaaggagtgaaggagccaggtgggaaatggtatttctatttttaaactccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaggcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 137 - DNA, 1041 bp, homo sapien
atgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactagaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttag

SEQ ID NO: 138 - DNA, 2238 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactagaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 139 - DNA, 2224 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtaccagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 140 - DNA, 2245 bp, homo sapien
gcagccatggtaggggtggaggtacaggcagcaaacaatatttaagatactgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtaccagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaac

SEQ ID NO: 141 - DNA, 1298 bp, homo sapien
aatatttaagatgctgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagctgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtaccagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaa

SEQ ID NO: 142 - DNA, 2255 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtaccagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtgaaaaaaaaaaaaaaaaaa

SEQ ID NO: 143 - DNA, 2212 bp, homo sapien
gaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtaccagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 144 - DNA, 2006 bp, homo sapien
acaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtaccagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagacccatattattttgattctcaaaggataggcctttgagtgttagagaaaggagtgaaggagccaggtgggaaatggtatttctatttttaaactccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaggcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 145 - DNA, 1041 bp, homo sapien
atgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtaccagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttag

SEQ ID NO: 146 - DNA, 2238 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtaccagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 147 - DNA, 2224 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgtgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 148 - DNA, 2245 bp, homo sapien
gcagccatggtaggggtggaggtacaggcagcaaacaatatttaagatactgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgtgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaac

SEQ ID NO: 149 - DNA, 1298 bp, homo sapien
aatatttaagatgctgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagctgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgtgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaa

SEQ ID NO: 150 - DNA, 2255 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgtgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtgaaaaaaaaaaaaaaaaaa

SEQ ID NO: 151 - DNA, 2212 bp, homo sapien
gaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgtgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 152 - DNA, 2006 bp, homo sapien
acaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgtgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagacccatattattttgattctcaaaggataggcctttgagtgttagagaaaggagtgaaggagccaggtgggaaatggtatttctatttttaaactccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaggcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 153 - DNA, 1041 bp, homo sapien
atgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgtgctacgctgagtatagccactttgttttgggcaatgaactcaacagctatcgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttag

SEQ ID NO: 154 - DNA, 2238 bp, homo sapien; identical to SEQ ID NO: 98

SEQ ID NO: 155 - DNA, 2224 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctattgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 156 - DNA, 2245 bp, homo sapien
gcagccatggtaggggtggaggtacaggcagcaaacaatatttaagatactgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctattgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaac

SEQ ID NO: 157 - DNA, 1298 bp, homo sapien
aatatttaagatgctgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagctgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctattgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaa

SEQ ID NO: 158 - DNA, 2255 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctattgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtgaaaaaaaaaaaaaaaaaa

SEQ ID NO: 159 - DNA, 2212 bp, homo sapien
gaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctattgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 160 - DNA, 2006 bp, homo sapien
acaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctattgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagacccatattattttgattctcaaaggataggcctttgagtgttagagaaaggagtgaaggagccaggtgggaaatggtatttctatttttaaactccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaggcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 161 - DNA, 1041 bp, homo sapien
atgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctattgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttag

SEQ ID NO: 162 - DNA, 2238 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctattgcctcttcctggggaactacactggcaatgtggggaacgacgccctccagtatcataacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 163 - DNA, 2224 bp, homo sapien
gggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcaaaacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 164 - DNA, 2245 bp, homo sapien
gcagccatggtaggggtggaggtacaggcagcaaacaatatttaagatactgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcaaaacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaac

SEQ ID NO: 165 - DNA, 1298 bp, homo sapien
aatatttaagatgctgacttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagctgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcaaaacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaa

SEQ ID NO: 166 - DNA, 2255 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcaaaacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtgaaaaaaaaaaaaaaaaaa

SEQ ID NO: 167 - DNA, 2212 bp, homo sapien
gaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcaaaacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 168 - DNA, 2006 bp, homo sapien
acaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcaaaacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagacccatattattttgattctcaaaggataggcctttgagtgttagagaaaggagtgaaggagccaggtgggaaatggtatttctatttttaaactccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaggcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtg

SEQ ID NO: 169 - DNA, 1041 bp, homo sapien
atgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcaaaacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttag

SEQ ID NO: 170 - DNA, 2238 bp, homo sapien
cttgtggagcattcgggcttggaaggaaagctataggctacccattcagctcccctgtcagagactcaagctttgagaaaggctagcaaagagcaaggaaagagagaaaacaacaaagtggcgaggccctcagagtgaaagcgtaaggttcagtcagcctgctgcagctttgcagacctcagctgggcatctccagactcccctgaaggaagagccttcctcacccaaacccacaaaagatgctgaaaaagcctctctcagctgtgacctggctctgcattttcatcgtggcctttgtcagccacccagcgtggctgcagaagctctctaagcacaagacaccagcacagccacagctcaaagcggccaactgctgtgaggaggtgaaggagctcaaggcccaagttgccaaccttagcagcctgctgagtgaactgaacaagaagcaggagagggactgggtcagcgtggtcatgcaggtgatggagctggagagcaacagcaagcgcatggagtcgcggctcacagatgctgagagcaagtactccgagatgaacaaccaaattgacatcatgcagctgcaggcagcacagacggtcactcagacctccgcagatgccatctacgactgctcttccctctaccagaagaactaccgcatctctggagtgtataagcttcctcctgatgacttcctgggcagccctgaactggaggtgttctgtgacatggagacttcaggcggaggctggaccatcatccagagacgaaaaagtggccttgtctccttctaccgggactggaagcagtacaagcagggctttggcagcatccgtggggacttctggctggggaacgaacacatccaccggctctccagacagccaacccggctgcgtgtagagatggaggactgggagggcaacctgcgctacgctgagtatagccactttgttttgggcaatgaactcaacagctatcgcctcttcctggggaactacactggcaatgtggggaacgacgccctccagtatcaaaacaacacagccttcagcaccaaggacaaggacaatgacaactgcttggacaagtgtgcacagctccgcaaaggtggctactggtacaactgctgcacagactccaacctcaatggagtgtactaccgcctgggtgagcacaataagcacctggatggcatcacctggtatggctggcatggatctacctactccctcaaacgggtggagatgaaaatccgcccagaagacttcaagccttaaaaggaggctgccgtggagcacggatacagaaactgagacacgtggagactggatgagggcagatgaggacaggaagagagtgttagaaagggtaggactgagaaacagcctataatctccaaagaaagaataagtctccaaggagcacaaaaaaatcatatgtaccaaggatgttacagtaaacaggatgaactatttaaacccactgggtcctgccacatccttctcaaggtggtagactgagtggggtctctctgcccaagatccctgacatagcagtagcttgtcttttccacatgatttgtctgtgaaagaaaataattttgagatcgttttatctattttctctacggcttaggctatgtgagggcaaaacacaaatccctttgctaaaaagaaccatattattttgattctcaaaggataggcctttgagtgttagagaaaggagtgaaggaggcaggtgggaaatggtatttctatttttaaatccagtgaaattatcttgagtctacacattatttttaaaacacaaaaattgttcggctggaactgacccaggctggacttgcggggaggaaactccagggcactgcatctggcgatcagactctgagcactgcccctgctcgccttggtcatgtacagcactgaaaggaatgaagcaccagcaggaggtggacagagtctctcatggatgccggcacaaaactgccttaaaatattcatagttaatacaggtatatctatttttatttactttgtaagaaacaagctcaaggagcttccttttaaattttgtctgtaggaaatggttgaaaactgaaggtagatggtgttatagttaataataaatgctgtaaataagcatctcactttgtaaaaataaaatattgtggttttgttttaaacattcaacgtttcttttccttctacaataaacactttcaaaatgtga

SEQ ID NO: 171 - protein, 346 aa, homo sapien
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIQRRKSGLVSFYRDWKQYKQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 172 - protein, 346 aa, homo sapien
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVICDMETSGGGWTIIQRRKSGLVSFYRDWKQYKQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 173 - protein, 346 aa, homo sapien
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTINQRRKSGLVSFYRDWKQYKQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 174 - protein, 346 aa, homo sapien
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIHRRKSGLVSFYRDWKQYKQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 175 - protein, 176 aa, homo sapien
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIQR

SEQ ID NO: 176 - protein, 187 aa, homo sapien
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIQRRKSGLVSFYRD

SEQ ID NO: 177 - protein, 346 aa, homo sapien
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIQRRKSGLVSFYRDWKQYQQGFGSIRGDFWLGNEHIHRLSRQPTRLRVEMEDWEGNLRYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 178 - protein, 346 aa, homo sapien
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIQRRKSGLVSFYRDWKQYKQGFGSIRGDFWLGNEHIHRLSRQPTRLRVEMEDWEGNLCYAEYSHFVLGNELNSYRLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 179 - protein, 346 aa, homo sapien
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIQRRKSGLVSFYRDWKQYKQGFGSIRGDFWLGNEHIHRLSRQPTRLRVEMEDWEGNLRYAEYSHFVLGNELNSYCLFLGNYTGNVGNDALQYHNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 180 - protein, 346 aa, homo sapien
MLKKPLSAVTWLCIFIVAFVSHPAWLQKLSKHKTPAQPQLKAANCCEEVKELKAQVANLSSLLSELNKKQERDWVSVVMQVMELESNSKRMESRLTDAESKYSEMNNQIDIMQLQAAQTVTQTSADAIYDCSSLYQKNYRISGVYKLPPDDFLGSPELEVFCDMETSGGGWTIIQRRKSGLVSFYRDWKQYKQGFGSIRGDFWLGNEHIHRLSRQPTRLRVEMEDWEGNLRYAEYSHFVLGNELNSYRLFLGNYTGNVGNDALQYQNNTAFSTKDKDNDNCLDKCAQLRKGGYWYNCCTDSNLNGVYYRLGEHNKHLDGITWYGWHGSTYSLKRVEMKIRPEDFKP

SEQ ID NO: 181 - DNA, 20 bp, artificial sequence
  Synthetic sequence; gRNA Recognition Sequence
gcttatacactccagagatg

SEQ ID NO: 182 - DNA, 20 bp, artificial sequence
  Synthetic sequence; gRNA Recognition Sequence
cctttgtcagccacccagcg

SEQ ID NO: 183 - DNA, 20 bp, artificial sequence
  Synthetic sequence; gRNA Recognition Sequence
gactggaagcagtacaagca

SEQ ID NO: 184 - DNA, 20 bp, artificial sequence
  Synthetic sequence; gRNA Recognition Sequence
caaagcggccaactgctgtg

SEQ ID NO: 185 - DNA, 20 bp, artificial sequence
  Synthetic sequence; gRNA Recognition Sequence
ggagagggactgggtcagcg

SEQ ID NO: 186 - DNA, 20 bp, artificial sequence
  Synthetic sequence; gRNA Recognition Sequence
acggtcactcagacctccgc

SEQ ID NO: 187 - DNA, 20 bp, artificial sequence
  Synthetic sequence; gRNA Recognition Sequence
gggctttggcagcatccgtg

SEQ ID NO: 188 - DNA, 20 bp, artificial sequence
  Synthetic sequence; gRNA Recognition Sequence
tgtgacatggagacttcagg

SEQ ID NO: 189 - DNA, 20 bp, artificial sequence
  Synthetic sequence; gRNA Recognition Sequence
actcagcaggctgctaaggt

SEQ ID NO: 190 - DNA, 20 bp, artificial sequence
  Synthetic sequence; gRNA Recognition Sequence
gggactggtctccttacctg

SEQ ID NO: 191 - DNA, 20 bp, artificial sequence
  Synthetic sequence; gRNA Recognition Sequence
tggggaacgaacacatccac

SEQ ID NO: 192 - DNA, 20 bp, artificial sequence
  Synthetic sequence; gRNA Recognition Sequence
ggactggaagcagtacaagc

SEQ ID NO: 193 - DNA, 20 bp, artificial sequence
  Synthetic sequence; gRNA Recognition Sequence
acagcaagcgcatggagtcg

SEQ ID NO: 194 - DNA, 20 bp, artificial sequence
  Synthetic sequence; gRNA Recognition Sequence
agggctttggcagcatccgt

SEQ ID NO: 195 - DNA, 20 bp, artificial sequence
  Synthetic sequence; gRNA Recognition Sequence
ctccatctctacacgcagcc

SEQ ID NO: 196 - DNA, 20 bp, artificial sequence
  Synthetic sequence; gRNA Recognition Sequence
agtcccggtagaaggagaca

SEQ ID NO: 197 - DNA, 20 bp, artificial sequence
  Synthetic sequence; gRNA Recognition Sequence
ccacgctgggtggctgacaa

SEQ ID NO: 198 - DNA, 20 bp, artificial sequence
  Synthetic sequence; gRNA Recognition Sequence
ggctctccagacagccaacc

SEQ ID NO: 199 - DNA, 20 bp, artificial sequence
  Synthetic sequence; gRNA Recognition Sequence
atctctacacgcagccgggt

SEQ ID NO: 200 - DNA, 20 bp, artificial sequence
  Synthetic sequence; gRNA Recognition Sequence
gtcaatttggttgttcatct

(19) United States
(12) Patent Application Publication
(10) Pub. No.: US 2022/0089664 A1
(43) Pub. Date: Mar. 24, 2022

(54) Treatment Of Ophthalmic Conditions With Angiopoietin-Like 7 (ANGPTL7) Inhibitors

(71) Applicant: Regeneron Pharmaceuticals, Inc., Tarrytown, NY (US)

(72) Inventors: Kavita Praveen, Tarrytown, NY (US); Claudia Schurmann, Tarrytown, NY (US); Lauren Gurski, Tarrytown, NY (US); Tanya Teslovich Dostal, Tarrytown, NY (US); Goncalo Abecasis, Tarrytown, NY (US); Aris Baras, Tarrytown, NY (US); Giovanni Coppola, Tarrytown, NY (US); Gaurang Patel, Tarrytown, NY (US); Ying Hu, Tarrytown, NY (US); Carmelo Romano, Tarrytown, NY (US); Giusy Della Gatta, Tarrytown, NY (US); Trikaldarshi Persaud, Tarrytown, NY (US)

(21) Appl. No.: 17/443,702

(22) Filed: Jul. 27, 2021

Related U.S. Application Data
(63) Continuation-in-part of application No. 17/031,523, filed on Sep. 24, 2020.
Application No. 17/031,523 is a continuation-in-part of application No. 16/748,006, filed on Jan. 21, 2020.
(60) Provisional application No. 62/909,573, filed on Oct. 2, 2019.
(60) Provisional application No. 62/902,683, filed on Sep. 19, 2019.
(60) Provisional application No. 62/880,609, filed on Jul. 30, 2019.
(60) Provisional application No. 62/795,665, filed on Jan. 23, 2019.

(51) Int. Cl. C07K 14/47; C12N 9/22; G01N 33/74

(52) U.S. Cl. CPC C07K 14/4703; A61K 38/00; G01N 33/74; C12N 9/22

(57) ABSTRACT

The present disclosure provides methods of treating subjects having an ophthalmic condition, methods of identifying subjects having an increased risk of developing an ophthalmic condition, and methods of detecting Angiopoietin-Like 7 (ANGPTL7) variant nucleic acid molecules and variant polypeptides.

### REFERENCE TO SEQUENCE LISTING

This application includes a Sequence Listing submitted electronically as a text file named 18923801631SEQ, created on Jul. 27, 2021, with a size of 529 kilobytes. The Sequence Listing is incorporated herein by reference.

### FIELD

The present disclosure relates generally to the treatment of subjects having an ophthalmic condition with Angiopoietin-Like 7 (ANGPTL7) inhibitors, methods of identifying subjects having an increased risk of developing an ophthalmic condition, and methods of detecting ANGPTL7 variant nucleic acid molecules and variant polypeptides.

### BACKGROUND

Glaucoma is the leading cause of irreversible blindness, with a global prevalence of 3.54% in individuals 40-80 years of age, and is projected to affect more than 111.8 million people by 2040. Classified as a neurodegenerative disease, glaucoma is characterized by the progressive loss of retinal ganglion cells in the eye and thinning of the neuroretinal rim of the optic nerve head. Affected individuals present with visual field loss that is accompanied by increased intraocular pressure (IOP) to a level that may damage the optic nerve in the majority of cases. Several types of glaucoma exist, the primary form being open angle glaucoma (POAG) is the most common glaucoma subtype and has highest prevalence in individuals of African ancestry (4.2% prevalence in Africa). In low-tension or normal-tension glaucoma, optic nerve damage and narrowed side vision occur in people with normal ocular pressure. In angle-closure glaucoma, the fluid at the front of the eye cannot drain properly, which may lead to a sudden increase in ocular pressure. In congenital glaucoma, children are born with a defect in the eye that slows the normal drainage of fluid. Glaucoma treatments include drug therapy, laser trabeculoplasty, and conventional surgery. While these treatments may save remaining vision, they do not improve sight already lost from glaucoma.

ANGPTL7 is a secreted glycoprotein structurally related to the angiopoietin family of growth factors. ANGPTL7 contains C-terminal (fibrinogen-like) and N-terminal (coiled) domains. ANGPTL7 is predominantly found in the stromal layer of the cornea and the extracellular matrix of the trabecular meshwork.

### SUMMARY

The present disclosure provides methods of treating a subject having an ophthalmic condition, the methods comprising administering an ANGPTL7 inhibitor to the subject.

The present disclosure also provides methods of treating a subject having increased IOP, the methods comprising administering an ANGPTL7 inhibitor to the subject.

The present disclosure also provides methods of treating a subject having glaucoma, the methods comprising administering an ANGPTL7 inhibitor to the subject.

The present disclosure also provides methods of treating a subject having open angle glaucoma, the methods comprising administering an ANGPTL7 inhibitor to the subject.

The present disclosure also provides methods of treating a subject having angle-closure glaucoma, the methods comprising administering an ANGPTL7 inhibitor to the subject.

The present disclosure also provides methods of treating a subject with a therapeutic agent that treats or inhibits an ophthalmic condition, the methods comprising the steps of: determining whether the subject has an ANGPTL7 missense variant nucleic acid molecule encoding an ANGPTL7 predicted loss-of-function polypeptide by: obtaining or having obtained a biological sample from the subject; and performing or having performed a sequence analysis on the biological sample to determine if the subject has a genotype comprising an ANGPTL7 missense variant nucleic acid molecule encoding the ANGPTL7 predicted loss-of-function polypeptide; and i) administering or continuing to administer the therapeutic agent that treats or inhibits the ophthalmic condition in a standard dosage amount to a subject that is ANGPTL7 reference, and administering an ANGPTL7 inhibitor to the subject; or ii) administering or continuing to administer the therapeutic agent that treats or inhibits the ophthalmic condition in an amount that is the same as or less than a standard dosage amount to a subject that is heterozygous for an ANGPTL7 missense variant nucleic acid molecule encoding the ANGPTL7 predicted loss-of-function polypeptide, and administering an ANGPTL7 inhibitor to the subject; wherein the presence of a genotype having an ANGPTL7 missense variant nucleic acid molecule encoding the ANGPTL7 predicted loss-of-function polypeptide indicates the subject has a decreased risk of developing the ophthalmic condition.

The present disclosure also provides methods of identifying a subject having an increased risk of developing an ophthalmic condition, the methods comprising: determining or having determined the presence or absence of an ANGPTL7 missense variant nucleic acid molecule encoding an ANGPTL7 predicted loss-of-function polypeptide in a biological sample obtained from the subject; wherein the subject has an increased risk of developing the ophthalmic condition when the subject is ANGPTL7 reference, and the subject has a decreased risk of developing the ophthalmic condition when the subject is heterozygous or homozygous for the ANGPTL7 missense variant nucleic acid molecule encoding the ANGPTL7 predicted loss-of-function polypeptide.

The present disclosure also provides methods of detecting an ANGPTL7 missense variant nucleic acid molecule, or the complement thereof, encoding an ANGPTL7 predicted loss-of-function polypeptide in a subject, the methods comprising assaying a biological sample obtained from the subject to determine whether a nucleic acid molecule in the biological sample is: i) a genomic nucleic acid molecule having a nucleotide sequence comprising: an adenine at a position corresponding to position 4,229 according to SEQ ID NO:2, or the complement thereof; an adenine at a position corresponding to position 4,269 according to SEQ ID NO:3, or the complement thereof; a thymine at a position corresponding to position 4,273 according to SEQ ID NO:4, or the complement thereof; a thymine at a position corresponding to position 4,277 according to SEQ ID NO:5, or the complement thereof; an adenine at a position corresponding to position 4,311 according to SEQ ID NO:6, or the complement thereof; a cytosine at a position corresponding to position 4,322 according to SEQ ID NO:7, or the complement thereof; a thymine at a position corresponding to position 5,125 according to SEQ ID NO:8, or the complement thereof; a thymine at a position corresponding to position 5,176 according to SEQ ID NO:9, or the complement thereof; or an adenine at a position corresponding to position 5,232 according to SEQ ID NO:10, or the complement thereof; ii) an mRNA molecule having a nucleotide sequence comprising: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof; a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a uracil at a position corresponding to position 768 according to SEQ ID NO:50, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; a uracil at a position corresponding to position 981 according to SEQ ID NO:82, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; an adenine at a position corresponding to position 1,037 according to SEQ ID NO:90, or the complement thereof; or iii) a cDNA molecule produced from an mRNA molecule in the biological sample, wherein the cDNA molecule has a nucleotide sequence comprising: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; an adenine at a position corresponding to position 760 according to SEQ ID NO:114, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; a thymine at a position corresponding to position 764 according to SEQ ID NO:122, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; a thymine at a position corresponding to position 768 according to SEQ ID NO:130, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; an adenine at a position corresponding to position 802 according to SEQ ID NO:138, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; a cytosine at a position corresponding to position 813 according to SEQ ID NO:146, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; a thymine at a position corresponding to position 930 according to SEQ ID NO:154, or the complement thereof; a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; a thymine at a position corresponding to position 981 according to SEQ ID NO:162, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof.

The present disclosure also provides therapeutic agents that treat or inhibit an ophthalmic condition for use in the treatment of an ophthalmic conditions (or for use in the preparation of a medicament for treating an ophthalmic condition) in a subject identified as having: i) a genomic nucleic acid molecule encoding an ANGPTL7 predicted loss-of-function polypeptide, or the complement thereof, wherein the genomic nucleic acid molecule has a nucleotide sequence comprising: an adenine at a position corresponding to position 4,229 according to SEQ ID NO:2, or the complement thereof; an adenine at a position corresponding to position 4,269 according to SEQ ID NO:3, or the complement thereof; a thymine at a position corresponding to position 4,273 according to SEQ ID NO:4, or the complement thereof; a thymine at a position corresponding to position 4,277 according to SEQ ID NO:5, or the complement thereof; an adenine at a position corresponding to position 4,311 according to SEQ ID NO:6, or the complement thereof; a cytosine at a position corresponding to position 4,322 according to SEQ ID NO:7, or the complement thereof; a thymine at a position corresponding to position 5,125 according to SEQ ID NO:8, or the complement thereof; a thymine at a position corresponding to position 5,176 according to SEQ ID NO:9, or the complement thereof; or an adenine at a position corresponding to position 5,232 according to SEQ ID NO:10, or the complement thereof; ii) an mRNA molecule having a nucleotide sequence encoding an ANGPTL7 predicted loss-of-function polypeptide, wherein the nucleotide sequence comprises: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof; a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a uracil at a position corresponding to position 768 according to SEQ ID NO:50, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof; a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; a uracil at a position corresponding to position 981 according to SEQ ID NO:82, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; an adenine at a position corresponding to position 1,037 according to SEQ ID NO:90, or the complement thereof; or iii) a cDNA molecule having a nucleotide sequence encoding an ANGPTL7 predicted loss-of-function polypeptide, wherein the nucleotide sequence comprises: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; an adenine at a position corresponding to position 760 according to SEQ ID NO:114, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; a thymine at a position corresponding to position 764 according to SEQ ID NO:122, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; a thymine at a position corresponding to position 768 according to SEQ ID NO:130, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; an adenine at a position corresponding to position 802 according to SEQ ID NO:138, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; a cytosine at a position corresponding to position 813 according to SEQ ID NO:146, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; a thymine at a position corresponding to position 930 according to SEQ ID NO:154, or the complement thereof; a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; a thymine at a position corresponding to position 981 according to SEQ ID NO:162, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof.

The present disclosure also provides ANGPTL7 inhibitors for use in the treatment of an ophthalmic condition (or for use in the preparation of a medicament for treating an ophthalmic condition) in a subject that: a) is reference for an ANGPTL7 genomic nucleic acid molecule, an ANGPTL7 mRNA molecule, or an ANGPTL7 cDNA molecule; or b) is heterozygous for: i) a genomic nucleic acid molecule encoding an ANGPTL7 predicted loss-of-function polypeptide, or the complement thereof, wherein the genomic nucleic acid molecule has a nucleotide sequence comprising: an adenine at a position corresponding to position 4,229 according to SEQ ID NO:2, or the complement thereof; an adenine at a position corresponding to position 4,269 according to SEQ ID NO:3, or the complement thereof; a thymine at a position corresponding to position 4,273 according to SEQ ID NO:4, or the complement thereof; a thymine at a position corresponding to position 4,277 according to SEQ ID NO:5, or the complement thereof; an adenine at a position corresponding to position 4,311 according to SEQ ID NO:6, or the complement thereof; a cytosine at a position corresponding to position 4,322 according to SEQ ID NO:7, or the complement thereof; a thymine at a position corresponding to position 5,125 according to SEQ ID NO:8, or the complement thereof; a thymine at a position corresponding to position 5,176 according to SEQ ID NO:9, or the complement thereof; or an adenine at a position corresponding to position 5,232 according to SEQ ID NO:10, or the complement thereof; ii) an mRNA molecule encoding an ANGPTL7 predicted loss-of-function polypeptide, or the complement thereof, wherein the mRNA molecule has a nucleotide sequence comprising: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof; a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a uracil at a position corresponding to position 768 according to SEQ ID NO:50, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof; a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; a uracil at a position corresponding to position 981 according to SEQ ID NO:82, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof; or iii) a cDNA molecule encoding an ANGPTL7 predicted loss-of-function polypeptide, or the complement thereof, wherein the cDNA molecule has a nucleotide sequence comprising: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; an adenine at a position corresponding to position 760 according to SEQ ID NO:114, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; a thymine at a position corresponding to position 764 according to SEQ ID NO:122, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; a thymine at a position corresponding to position 768 according to SEQ ID NO:130, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; an adenine at a position corresponding to position 802 according to SEQ ID NO:138, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; a cytosine at a position corresponding to position 813 according to SEQ ID NO:146, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; a thymine at a position corresponding to position 930 according to SEQ ID NO:154, or the complement thereof; a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; a thymine at a position corresponding to position 981 according to SEQ ID NO:162, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof.

### BRIEF DESCRIPTION OF THE DRAWINGS

The accompanying figures, which are incorporated in and constitute a part of this specification, illustrate several features of the present disclosure.

The patent or application file contains at least one drawing executed in color. Copies of this patent or patent application publication with color drawing(s) will be provided by the Office upon request and payment of the necessary fee.

FIG. 1 shows association of an aggregate of 99 pLOF and deleterious (based on 5 prediction algorithms) missense variants in ANGPTL7 with reduced IOP in 129,207 individuals of European descent.

FIG. 2 shows missense and predicted loss-of-function (pLOF) variants in ANGPTL7 and IOP levels in individuals of European descent. The plots represent Goldmann-correlated IOP (mean of both eyes) levels in carriers of 1 pLOF and 10 missense variants in ANGPTL7 that are predicted deleterious by five different algorithms and have at least five carriers amongst the 101,678 exome-sequenced individuals with IOP measurements in the UK Biobank. The median IOP level across carriers of all 99 pLOF and predicted-deleterious missense ANGPTL7 variants (14.74 mmHg) is indicated by the red line, and the median IOP in non-variant carriers (15.57 mmHg) is indicated by the blue line. Magenta diamonds mark the median IOP in carriers of each variant. Beneath the plots is the median and interquartile range of IOP and the numbers of variant carriers diagnosed with glaucoma or controls in the UK Biobank cohort (n=385,040).

FIG. 3 shows missense and predicted loss-of-function (pLOF) variants in ANGPTL7 and IOP levels in individuals of European descent in GHS. The plots represent Goldmann-correlated IOP (mean of both eyes) levels in carriers of 1 pLOF and 4 missense variants in ANGPTL7 that are predicted deleterious by five different algorithms and have at least five carriers amongst the 27,529 exome-sequenced individuals with IOP measurements in GHS. The median IOP level across carriers of all 33 pLOF and predicted-deleterious missense ANGPTL7 variants (15.25 mmHg) is indicated by the red line, and the median IOP in non-variant carriers (15.50 mmHg) is indicated by the blue line. Magenta diamonds mark the median IOP in carriers of each variant. Beneath the plots is the median and interquartile range of IOP and the numbers of variant carriers diagnosed with glaucoma or controls in GHS (n=118,164).

FIG. 4 shows Meta-analysis of ANGPTL7 aggregate of predicted loss-of-function and deleterious missense variants (MAF<1%), excluding Gln175His and Arg177*, with IOP. A total of 97 variants were present in the burden test.

FIG. 5 shows association of Gln175His variant in ANGPTL7 with IOP, effect measured in standard deviation units, in individuals of European descent.

FIG. 6 shows boxplot representing Goldmann-correlated (IOPg) in the UK Biobank across genotypes, showing that Gln175His heterozygous and homozygous carriers have a 0.8-mmHg and 4.1-mmHg lower median IOPg, respectively, compared to non-carriers.

FIG. 7 shows association of Arg177* variant in ANGPTL7 with IOP, effect measured in standard deviation units, in individuals of European descent.

FIG. 8 shows boxplot representing Goldmann-correlated (IOPg) in the UK Biobank across genotypes, showing that Arg177* heterozygous carriers have a 1.4-mmHg lower IOPg compared to non-carriers. GHS: Geisinger DiscovEHR, UKB: UK Biobank.

FIG. 9 shows meta-analysis of ANGPTL7 aggregate of predicted loss-of-function variants only (MAF<1%) with IOP. Arg177* is included in this aggregate of 12 variants.

FIG. 10 cross-ancestry meta-analysis of Arg177* and Trp188* with IOP. AFR=African ancestry cohorts.

FIG. 11 shows meta-analysis results for Gln175His with glaucoma across 8 different cohorts. Geisinger DiscovEHR, UKB: UK Biobank, SINAI: Mt. Sinai Medical School BioMe Biobank, MALMO: Malmo Diet and Cancer Study, EstBB: the Estonia Biobank at the University of Tartu; HUNT: the HUNT study from Nord-Trøndelag; CGPS-CCHS: the Copenhagen General Population Study and the Copenhagen City Heart Study, POAAGG: Primary Open Angle African-American Glaucoma Genetics, AAF=alternative allele frequency.

FIG. 12 shows cross-ancestry meta-analysis of Arg177* and Trp188* across 5 EUR and 3 AFR cohorts. The variants in the meta-analysis of EUR and AFR cohorts were Arg177* and Trp188*, respectively. GHS: Geisinger DiscovEHR, UKB: UK Biobank, SINAI: Mt. Sinai Medical School BioMe Biobank, MALMO: Malmo Diet and Cancer Study, EstBB: the Estonia Biobank at the University of Tartu; HUNT: the HUNT study from Nord-Trondelag; CGPS-CCHS: the Copenhagen General Population Study and the Copenhagen City Heart Study, POAAGG: Primary Open Angle African-American Glaucoma Genetics, AAF=alternative allele frequency.

FIG. 13 shows relative ANGPTL7 mRNA expression in a HEK293 cell line. qPCR shows mRNA levels of ANGPTL7 wild type, ANGPTL7 Gln175His, ANGPTL7 Arg177* and Trp188*, after transfection in HEK293. The experiment shows the average expression of ANGPTL7 and its variants in three biological replicates. Technical replicates (n=3) were run for all three qPCR replicates.

FIG. 14 shows western blotting shows intra-cellular protein levels of ANGPTL7 wild-type, Gln175His, Arg177*, and Trp188*.

FIG. 15 shows western blotting shows extra-cellular protein levels of ANGPTL7 wild-type, Gln175His, Arg177*, and Trp188*.

FIG. 16 shows ELISA assay quantifying intra- and extra-cellular protein levels of ANGPTL7 wild-type, Gln175His, Arg177*, and Trp188* transiently transfected in HEK293 cells (whole-cell lysate was diluted 1:1,000; supernatant was diluted 1:10,000. Both whole-cell lysate and supernatant were normalized against the total amount of protein from the whole-cell lysate).

FIG. 17 shows ratio of secreted versus intracellular ANGPTL7 wild-type, Gln175His, Arg177*, and Trp188* protein levels. Raw ANGPTL7 wild-type, Gln175His, Arg177*, and Trp188* protein levels were normalized to the whole-lysate protein concentration. Western blotting and ELISA analysis were repeated on three independent biological replicates. Technical replicates (n=3) were run for the ELISA analysis.

FIG. 18 shows RNA-sequencing-based expression levels (measured in transcripts per million, TPM) are highest in cornea, trabecular meshwork (TM), and sclera in human eyes.

FIG. 19 shows RNA-sequencing-based expression levels (measured in transcripts per million, TPM) are highest in cornea, trabecular meshwork (TM), and sclera in African green monkey eyes.

FIG. 20 shows RNA-sequencing-based expression levels (measured in transcripts per million, TPM) in cornea, TM, sclera, optic nerve, and choroid/RPE in C57BL/6J mice.

FIG. 21 shows in situ hybridization (RNAScope) shows ANGPTL7/Angptl7 (red) expression in TM, cornea and sclera in human eyes. DAPI staining (blue) counterstains cell nuclei. RPE: retinal pigmented epithelium; CB: ciliary body; SC: Schlemm's canal; CM: ciliary muscle; AC: anterior chamber; RGC: retinal ganglion cell; INL: inner nuclear layer; ONL: outer nuclear layer.

FIG. 22 shows in situ hybridization (RNAScope) shows ANGPTL7/Angptl7 (red) expression in TM, cornea and sclera in murine eyes. DAPI staining (blue) counterstains cell nuclei. RPE: retinal pigmented epithelium; CB: ciliary body; SC: Schlemm's canal; CM: ciliary muscle; AC: anterior chamber; RGC: retinal ganglion cell; INL: inner nuclear layer; ONL: outer nuclear layer.

FIG. 23 shows murine Angptl7 (mAngptl7) protein was injected into mouse eyes via intravitreal route, and IOP was measured over time. After an initial drop, IOP was elevated in Angptl7-treated eyes compared to control eyes. Data are presented as means and error bars represent SD.

FIG. 24 shows Murine Angptl7 (mAngptl7) protein was injected into mouse eyes via intracameral route, and IOP was measured over time. After an initial drop, IOP was elevated in Angptl7-treated eyes compared to control eyes. Data are presented as means and error bars represent SD.

FIG. 25 shows that no ocular changes were observed between Angptl7 KO (ii, iv) and WT (i, iii) mouse eyes on anterior segment optical coherence tomography (OCT).

FIG. 26 shows Angptl7 KO and WT mice having similar corneal thickness. Error bars represent the standard deviation.

FIG. 27 shows that IOP was significantly lowered in Angptl7 KO compared to Het and WT mice (P<0.0001).

FIG. 28 shows that Angptl7 mRNA is not expressed in any ocular tissue in Angptl7 KO mice whereas it was expressed in TM, cornea, and sclera of WT mice as shown by in situ hybridization (RNAScope). Brightfield images showing following probes (upper right) Negative control—DapB, (upper left) Positive control—Ubc (red), (lower right) WT mice—Angptl7 (red), and (lower left) Angptl7 KO mice—Angptl7 (no signal). Scale bar: 100 μm.

FIG. 29 shows that intravitreal injection with 15 ug of Angptl7-siRNA significantly lowered IOP in two of the six siRNAs (n=6-8/group) compared to the PBS (n=6) and Naïve (no injection, n=5) groups and remained lowered throughout the end of the study. siRNA #3 and 5 lowered IOP between 2-4 mmHg starting at week 2 compared to PBS-treated mice.

FIG. 30 shows qPCR results from micro-dissected limbal ring showed the highest level of knockdown (>50%) of Angptl7 mRNA with siRNAs #3 and #5 compared to PBS-treated mice, which is highly consistent with the IOP lowering observed in mice injected with these two siRNAs. Error bars represent the standard deviation.

FIG. 31 shows dexamethasone (DEX)-induced gene expression changes in three human trabecular meshwork (hTM) primary cell lines from three independent human eyes, measured with quantitative PCR (qPCR); hTM cells were treated with DEX for 72 hours followed by qPCR analysis; DEX treatment increased ANGPTL7 expression in two out of three HTM cell lines; Ctrl represents untreated cells and EtOH represents ethanol treatment; data are presented as means±standard error across two replicates, one-way A NOVA, * p=0.01, **p=0.001, ***p=0.0001.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician.

A variant in the ANGPTL7 gene associated with a decreased risk of developing an ophthalmic conditions, such as glaucoma, in humans has been identified in accordance with the present disclosure. For example, a genetic alteration that changes the thymine at position 4,229 in the ANGPTL7 reference genomic nucleic acid molecule (see, SEQ ID NO:1) to an adenine, or changes the thymine at position 4,269 in the ANGPTL7 reference genomic nucleic acid molecule (see, SEQ ID NO:1) to an adenine, or changes the guanine at position 4,273 in the ANGPTL7 reference genomic nucleic acid molecule (see, SEQ ID NO:1) to a thymine, or changes the cytosine at position 4,277 in the ANGPTL7 reference genomic nucleic acid molecule (see, SEQ ID NO:1) to a thymine, or changes the guanine at position 4,311 in the ANGPTL7 reference genomic nucleic acid molecule (see, SEQ ID NO:1) to an adenine, or changes the adenine at position 4,322 in the ANGPTL7 reference genomic nucleic acid molecule (see, SEQ ID NO:1) to a cytosine, or changes the cytosine at position 5,125 in the ANGPTL7 reference genomic nucleic acid molecule (see, SEQ ID NO:1) to a thymine, changes the cytosine at position 5,176 in the ANGPTL7 reference genomic nucleic acid molecule (see, SEQ ID NO:1) to a thymine, or changes the thymine at position 5,232 in the ANGPTL7 reference genomic nucleic acid molecule (see, SEQ ID NO:1) to an adenine has been observed to indicate that the subject having such an alteration may have a decreased risk of developing an ophthalmic condition, such as glaucoma. It is believed that no variants of the ANGPTL7 gene or protein have any known association with an ophthalmic condition, such as glaucoma. Altogether, the genetic analyses described herein surprisingly indicate that the ANGPTL7 gene and, in particular, a variant in the ANGPTL7 gene, associates with a decreased risk of developing an ophthalmic condition. Moreover, the identification by the present disclosure of the association between additional variants and gene burden masks indicates that ANGPTL7 itself (rather than linkage disequilibrium with variants in another gene) is responsible for a protective effect in ophthalmic conditions. Therefore, subjects that are ANGPTL7 reference that have an increased risk of developing an ophthalmic condition, such as glaucoma, including open-angle glaucoma, angle-closure glaucoma, normal-tension glaucoma, congenital glaucoma, secondary glaucoma, pigmentary glaucoma, pseudoexfoliative glaucoma, traumatic glaucoma, neovascular glaucoma, uveitic glaucoma, or irido corneal endothelial syndrome, may be treated such that the ophthalmic condition is prevented, the symptoms thereof are reduced, and/or development of symptoms is repressed. Accordingly, the present disclosure provides methods of leveraging the identification of such variants in subjects to identify or stratify risk in such subjects of developing an ophthalmic conditions, such as glaucoma, including open-angle glaucoma, angle-closure glaucoma, normal-tension glaucoma, congenital glaucoma, secondary glaucoma, pigmentary glaucoma, pseudoexfoliative glaucoma, traumatic glaucoma, neovascular glaucoma, uveitic glaucoma, or irido corneal endothelial syndrome, or to diagnose subjects as having an increased risk of developing an ophthalmic condition, such as glaucoma, including open-angle glaucoma, angle-closure glaucoma, normal-tension glaucoma, congenital glaucoma, secondary glaucoma, pigmentary glaucoma, pseudoexfoliative glaucoma, traumatic glaucoma, neovascular glaucoma, uveitic glaucoma, or irido corneal endothelial syndrome, such that subjects at risk or subjects with active disease may be treated accordingly.

For purposes of the present disclosure, any particular subject can be categorized as having one of three ANGPTL7 genotypes: i) ANGPTL7 reference; ii) heterozygous for an ANGPTL7 missense variant nucleic acid molecule encoding an ANGPTL7 predicted loss-of-function polypeptide; or iii) homozygous for an ANGPTL7 missense variant nucleic acid molecule encoding an ANGPTL7 predicted loss-of-function polypeptide. A subject is ANGPTL7 reference when the subject does not have a copy of an ANGPTL7 missense variant nucleic acid molecule encoding an ANGPTL7 predicted loss-of-function polypeptide. A subject is heterozygous for an ANGPTL7 missense variant nucleic acid molecule encoding an ANGPTL7 predicted loss-of-function polypeptide when the subject has a single copy of an ANGPTL7 missense variant nucleic acid molecule encoding an ANGPTL7 predicted loss-of-function polypeptide. As used herein, an ANGPTL7 missense variant nucleic acid molecule is any ANGPTL7 nucleic acid molecule (such as, a genomic nucleic acid molecule, an mRNA molecule, or a cDNA molecule) encoding an ANGPTL7 polypeptide having a partial loss-of-function, a complete loss-of-function, a predicted partial loss-of-function, or a predicted complete loss-of-function. A subject who has an ANGPTL7 missense variant nucleic acid molecule encoding an ANGPTL7 predicted loss-of-function polypeptide having a partial loss-of-function (or predicted partial loss-of-function) is hypomorphic for ANGPTL7. The ANGPTL7 missense variant nucleic acid molecule encoding an ANGPTL7 predicted loss-of-function polypeptide can be any nucleic acid molecule encoding an ANGPTL7 Phe161Ile, Ile174Asn, Gln175His, Arg177STOP, Trp188STOP, Lys192Gln, Arg220Cys, Arg220His, Arg231Cys, Arg248Cys, His266Gln, Asn302Lys, or Arg340His. In some embodiments, the ANGPTL7 missense variant nucleic acid molecule encodes an ANGPTL7 Gln175His, Arg177STOP, or Trp188STOP. A subject is homozygous for an ANGPTL7 missense variant nucleic acid molecule encoding an ANGPTL7 predicted loss-of-function polypeptide when the subject has two copies of an ANGPTL7 missense variant nucleic acid molecule encoding an ANGPTL7 predicted loss-of-function polypeptide.

For subjects that are genotyped or determined to be ANGPTL7 reference, such subjects have an increased risk of developing an ophthalmic condition, such as glaucoma, including open-angle glaucoma, angle-closure glaucoma, normal-tension glaucoma, congenital glaucoma, secondary glaucoma, pigmentary glaucoma, pseudoexfoliative glaucoma, traumatic glaucoma, neovascular glaucoma, uveitic glaucoma, or irido corneal endothelial syndrome. For subjects that are genotyped or determined to be either ANGPTL7 reference or heterozygous for an ANGPTL7 missense variant nucleic acid molecule encoding an ANGPTL7 predicted loss-of-function polypeptide, such subjects can be treated with an ANGPTL7 inhibitor.

In any of the embodiments described throughout the present disclosure, the ANGPTL7 missense variant nucleic acid molecule encoding an ANGPTL7 predicted loss-of-function polypeptide can be any ANGPTL7 nucleic acid molecule (such as, for example, genomic nucleic acid molecule, mRNA molecule, or cDNA molecule) encoding an ANGPTL7 polypeptide having a partial loss-of-function, a complete loss-of-function, a predicted partial loss-of-function, or a predicted complete loss-of-function. For example, the ANGPTL7 missense variant nucleic acid molecule can be any nucleic acid molecule encoding ANGPTL7 Phe161Ile, Ile174Asn, Gln175His, Arg177STOP, Trp188STOP, Lys192Gln, Arg220Cys, Arg220His, Arg231Cys, Arg248Cys, His266Gln, Asn302Lys, or Arg340His. In some embodiments, the ANGPTL7 missense variant nucleic acid molecule encodes ANGPTL7 Gln175His, Arg177STOP, or Trp188STOP.

In any of the embodiments described throughout the present disclosure, the ANGPTL7 predicted loss-of-function polypeptide can be any ANGPTL7 polypeptide having a partial loss-of-function, a complete loss-of-function, a predicted partial loss-of-function, or a predicted complete loss-of-function. In any of the embodiments described throughout the present disclosure, the ANGPTL7 predicted loss-of-function polypeptide can be any of the ANGPTL7 polypeptides described herein including, for example, ANGPTL7 Phe161Ile, Ile174Asn, Gln175His, Arg177STOP, Trp188STOP, Lys192Gln, Arg220Cys, Arg220His, Arg231Cys, Arg248Cys, His266Gln, Asn302Lys, or Arg340His. In some embodiments, the ANGPTL7 predicted loss-of-function polypeptide is ANGPTL7 Gln175His, Arg177STOP, or Trp188STOP.

In any of the embodiments described throughout the present disclosure, the ophthalmic condition is glaucoma, including open-angle glaucoma, angle-closure glaucoma, normal-tension glaucoma, congenital glaucoma, secondary glaucoma, pigmentary glaucoma, pseudoexfoliative glaucoma, traumatic glaucoma, neovascular glaucoma, uveitic glaucoma, or irido corneal endothelial syndrome. In any of the embodiments described throughout the present disclosure, the ophthalmic condition is increased IOP. In any of the embodiments described throughout the present disclosure, the ophthalmic condition is glaucoma. In any of the embodiments described throughout the present disclosure, the ophthalmic condition is open angle glaucoma. In any of the embodiments described throughout the present disclosure, the ophthalmic condition is angle-closure glaucoma. In any of the embodiments described throughout the present disclosure, the ophthalmic condition is normal-tension glaucoma. In any of the embodiments described throughout the present disclosure, the ophthalmic condition is congenital glaucoma. In any of the embodiments described throughout the present disclosure, the ophthalmic condition is secondary glaucoma. In any of the embodiments described throughout the present disclosure, the ophthalmic condition is pigmentary glaucoma. In any of the embodiments described throughout the present disclosure, the ophthalmic condition is pseudoexfoliative glaucoma. In any of the embodiments described throughout the present disclosure, the ophthalmic condition is traumatic glaucoma. In any of the embodiments described throughout the present disclosure, the ophthalmic condition is neovascular glaucoma. In any of the embodiments described throughout the present disclosure, the ophthalmic condition is uveitic glaucoma. In any of the embodiments described throughout the present disclosure, the ophthalmic condition is irido corneal endothelial syndrome.

Symptoms of ophthalmic conditions include, but are not limited to, severe eye pain, nausea, vomiting, redness in eye, sudden vision disturbances, seeing colored rings around lights, and sudden blurred vision.

The present disclosure provides methods of treating a subject having an ophthalmic condition, the methods comprising administering an ANGPTL7 inhibitor to the subject.

The present disclosure also provides methods of treating a subject having increased IOP, the methods comprising administering an ANGPTL7 inhibitor to the subject.

The present disclosure also provides methods of treating a subject having glaucoma, the methods comprising administering an ANGPTL7 inhibitor to the subject.

The present disclosure also provides methods of treating a subject having open angle glaucoma, the methods comprising administering an ANGPTL7 inhibitor to the subject.

The present disclosure also provides methods of treating a subject having angle-closure glaucoma, the methods comprising administering an ANGPTL7 inhibitor to the subject.

In some embodiments, the ANGPTL7 inhibitor comprises an inhibitory nucleic acid molecule. In some embodiments, the inhibitory nucleic acid molecule comprises an antisense molecule, a small interfering RNA (siRNA) molecule, or a short hairpin RNA (shRNA) molecule. In some embodiments, the inhibitory nucleic acid molecule comprises an antisense molecule. In some embodiments, the inhibitory nucleic acid molecule comprises an siRNA molecule. In some embodiments, the inhibitory nucleic acid molecule comprises an shRNA molecule. Such inhibitory nucleic acid molecules can be designed to target any region of an ANGPTL7 nucleic acid molecule, such as an mRNA molecule. In some embodiments, the inhibitory nucleic acid molecule hybridizes to a sequence within an ANGPTL7 genomic nucleic acid molecule or mRNA molecule and decreases expression of the ANGPTL7 polypeptide in a cell in the subject. In some embodiments, the ANGPTL7 inhibitor comprises an antisense molecule that hybridizes to an ANGPTL7 genomic nucleic acid molecule or mRNA molecule and decreases expression of the ANGPTL7 polypeptide in a cell in the subject. In some embodiments, the ANGPTL7 inhibitor comprises an siRNA that hybridizes to an ANGPTL7 genomic nucleic acid molecule or mRNA molecule and decreases expression of the ANGPTL7 polypeptide in a cell in the subject. In some embodiments, the ANGPTL7 inhibitor comprises an shRNA that hybridizes to an ANGPTL7 genomic nucleic acid molecule or mRNA molecule and decreases expression of the ANGPTL7 polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules disclosed herein can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher).

Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (1), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the ANGPTL7 inhibitor comprises a nuclease agent that induces one or more nicks or double-strand breaks at a recognition sequence(s) or a DNA-binding protein that binds to a recognition sequence within an ANGPTL7 genomic nucleic acid molecule. The recognition sequence can be located within a coding region of the ANGPTL7 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the ANGPTL7 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an ANGPTL7 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of ANGPTL7 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an ANGPTL7 genomic nucleic acid molecule or it can be a nickase that creates a single-strand break in an ANGPTL7 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of an ANGPTL7 genomic nucleic acid molecules can be generated by contacting a cell with a Cas protein and one or more gRNAs that hybridize to one or more gRNA recognition sequences within a target genomic locus in the ANGPTL7 genomic nucleic acid molecule. For example, a gRNA recognition sequence can be located within a region of SEQ ID NO:1. The gRNA recognition sequence can also include or be proximate to a position corresponding to: position 4,229, position 4,269, position 4,273, position 4,277, position 4,311, position 4,322, position 5,125, position 5,176, or position 5,232 according to SEQ ID NO:1. For example, the gRNA recognition sequence can be located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to: position 4,229, position 4,269, position 4,273, position 4,277, position 4,311, position 4,322, position 5,125, position 5,176, or position 5,232 according to SEQ ID NO:1. The gRNA recognition sequence can include or be proximate to the start codon of an ANGPTL7 genomic nucleic acid molecule or the stop codon of an ANGPTL7 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an ANGPTL7 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2 to about 6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10 base pairs, about 2 to about 5 base pairs, or 3 base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an ANGPTL7 genomic nucleic acid molecule. An exemplary gRNA is a gRNA effective to direct a Cas enzyme to bind to or cleave an ANGPTL7 genomic nucleic acid molecule, wherein the gRNA comprises a DNA-targeting segment that hybridizes to a gRNA recognition sequence within the ANGPTL7 genomic nucleic acid molecule that includes or is proximate to a position corresponding to: position 4,229, position 4,269, position 4,273, position 4,277, position 4,311, position 4,322, position 5,125, position 5,176, or position 5,232 according to SEQ ID NO:1. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides from a position corresponding to: position 4,229, position 4,269, position 4,273, position 4,277, position 4,311, position 4,322, position 5,125 position 5,176, or position 5,232 according to SEQ ID NO:1. Other exemplary gRNAs comprise a DNA-targeting segment that hybridizes to a gRNA recognition sequence present within an ANGPTL7 genomic nucleic acid molecule that includes or is proximate to the start codon or the stop codon. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides of the start codon or located about 5, about 10, about 15, about 20, about 25, about 30, about 35, about 40, about 45, about 50, about 100, about 200, about 300, about 400, about 500, or about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the ANGPTL7 reference gene are set forth in Table 1 as SEQ ID NOs:181-200.

TABLE 1
Guide RNA Recognition Sequences
Near ANGPTL7 Variation(s)

| Strand | gRNA Recognition Sequence | SEQ ID NO: |
|---|---|---|
| − | GCTTATACACTCCAGAGATG | 181 |
| + | CCTTTGTCAGCCACCCAGCG | 182 |
| + | GACTGGAAGCAGTACAAGCA | 183 |
| + | CAAAGCGGCCAACTGCTGTG | 184 |
| + | GGAGAGGGACTGGGTCAGCG | 185 |
| + | ACGGTCACTCAGACCTCCGC | 186 |
| + | GGGCTTTGGCAGCATCCGTG | 187 |
| + | TGTGACATGGAGACTTCAGG | 188 |
| − | ACTCAGCAGGCTGCTAAGGT | 189 |
| − | GGGACTGGTCTCCTTACCTG | 190 |
| + | TGGGGAACGAACACATCCAC | 191 |
| + | GGACTGGAAGCAGTACAAGC | 192 |
| + | ACAGCAAGCGCATGGAGTCG | 193 |
| + | AGGGCTTTGGCAGCATCCGT | 194 |
| − | CTCCATCTCTACACGCAGCC | 195 |
| − | AGTCCCGGTAGAAGGAGACA | 196 |
| − | CCACGCTGGGTGGCTGACAA | 197 |
| + | GGCTCTCCAGACAGCCAACC | 198 |
| − | ATCTCTACACGCAGCCGGGT | 199 |
| − | GTCAATTTGGTTGTTCATCT | 200 |

The Cas protein and the gRNA form a complex, and the Cas protein cleaves the target ANGPTL7 genomic nucleic acid molecule. The Cas protein can cleave the nucleic acid molecule at a site within or outside of the nucleic acid sequence present in the target ANGPTL7 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the ANGPTL7 genomic nucleic acid molecule to which a DNA-targeting segment of a gRNA will bind.

Such methods can result, for example, in an ANGPTL7 genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the ANGPTL7 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the ANGPTL7 inhibitor comprises a small molecule. In some embodiments, the ANGPTL7 inhibitor is 6,11-dihydro[1]benzothiopyrano[4,3-b]indole (PD146176), 2-bromophenol, 2,4-dibromophenol, 2-(1-thienyl)ethyl-3,4-dihydroxybenzylidenecyanoacetate (TEDC), 4,4′-(2,3-dimethyl-1,4-butanediyl)bis-1,2-benzenediol (nordihydroguaiaretic acid), or cinnamyl-3,4-dihydroxy-a-cyanocinnamate (CDC). In some embodiments, the ANGPTL7 inhibitor is 6,11-dihydro[1] benzothiopyrano [4,3-b]indole (PD146176). In some embodiments, the ANGPTL7 inhibitor is 2-bromophenol. In some embodiments, the ANGPTL7 inhibitor is 2,4-dibromophenol. In some embodiments, the ANGPTL7 inhibitor is 2-(1-thienyl)ethyl-3,4-dihydroxybenzylidenecyanoacetate (TEDC). In some embodiments, the ANGPTL7 inhibitor is 4,4′-(2,3-dimethyl-1,4-butanediyl)bis-1,2-benzenediol (nordihydroguaiaretic acid). In some embodiments, the ANGPTL7 inhibitor is cinnamyl-3,4-dihydroxy-a-cyanocinnamate (CDC).

In some embodiments, the methods of treatment further comprise detecting the presence or absence of an ANGPTL7 missense variant nucleic acid molecule encoding an ANGPTL7 predicted loss-of-function polypeptide in a biological sample obtained from the subject. As used throughout the present disclosure, “an ANGPTL7 missense variant nucleic acid molecule” is any ANGPTL7 nucleic acid molecule (such as, for example, genomic nucleic acid molecule, mRNA molecule, or cDNA molecule) encoding an ANGPTL7 polypeptide having a partial loss-of-function, a complete loss-of-function, a predicted partial loss-of-function, or a predicted complete loss-of-function.

The present disclosure also provides methods of treating a subject with a therapeutic agent that treats or inhibits an ophthalmic condition. In some embodiments, the subject has an ophthalmic condition. In some embodiments, the methods comprise determining whether the subject has an ANGPTL7 missense variant nucleic acid molecule encoding an ANGPTL7 predicted loss-of-function polypeptide by obtaining or having obtained a biological sample from the subject, and performing or having performed a sequence analysis on the biological sample to determine if the subject has a genotype comprising the ANGPTL7 missense variant nucleic acid molecule. When the subject is ANGPTL7 reference, the therapeutic agent that treats or inhibits the ophthalmic condition is administered or continued to be administered to the subject in a standard dosage amount, and an ANGPTL7 inhibitor is administered to the subject. When the subject is heterozygous for an ANGPTL7 missense variant nucleic acid molecule, the therapeutic agent that treats or inhibits the ophthalmic condition is administered or continued to be administered to the subject in an amount that is the same as or less than a standard dosage amount, and an ANGPTL7 inhibitor is administered to the subject. The presence of a genotype having the ANGPTL7 missense variant nucleic acid molecule encoding an ANGPTL7 predicted loss-of-function polypeptide indicates the subject has a decreased risk of developing an ophthalmic condition. In some embodiments, the subject is ANGPTL7 reference. In some embodiments, the subject is heterozygous for the ANGPTL7 missense variant nucleic acid molecule encoding an ANGPTL7 predicted loss-of-function polypeptide.

For subjects that are genotyped or determined to be either ANGPTL7 reference or heterozygous for the ANGPTL7 missense variant nucleic acid molecule encoding an ANGPTL7 predicted loss-of-function polypeptide, such subjects can be treated with an ANGPTL7 inhibitor, as described herein.

Detecting the presence or absence of an ANGPTL7 missense variant nucleic acid molecule encoding an ANGPTL7 predicted loss-of-function polypeptide in a biological sample from a subject and/or determining whether a subject has an ANGPTL7 missense variant nucleic acid molecule encoding an ANGPTL7 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the ANGPTL7 missense variant nucleic acid molecule encoding an ANGPTL7 predicted loss-of-function polypeptide can be present within a cell obtained from the subject.

In some embodiments, when the subject is ANGPTL7 reference, the subject is also administered a therapeutic agent that treats or inhibits an ophthalmic condition in a standard dosage amount. In some embodiments, when the subject is heterozygous for an ANGPTL7 missense variant nucleic acid molecule encoding an ANGPTL7 predicted loss-of-function polypeptide, the subject is also administered a therapeutic agent that treats or inhibits an ophthalmic condition in a dosage amount that is the same as or less than a standard dosage amount.

In some embodiments, the treatment methods further comprise detecting the presence or absence of an ANGPTL7 predicted loss-of-function polypeptide in a biological sample from the subject. In some embodiments, when the subject does not have an ANGPTL7 predicted loss-of-function polypeptide, the subject is also administered a therapeutic agent that treats or inhibits an ophthalmic condition in a standard dosage amount. In some embodiments, when the subject has an ANGPTL7 predicted loss-of-function polypeptide, the subject is also administered a therapeutic agent that treats or inhibits an ophthalmic condition in a dosage amount that is the same as or less than a standard dosage amount.

The present disclosure also provides methods of treating a subject with a therapeutic agent that treats or inhibits an ophthalmic condition. In some embodiments, the subject has an ophthalmic conditions. In some embodiments, the method comprises determining whether the subject has an ANGPTL7 predicted loss-of-function polypeptide by obtaining or having obtained a biological sample from the subject, and performing or having performed an assay on the biological sample to determine if the subject has an ANGPTL7 predicted loss-of-function polypeptide. When the subject does not have an ANGPTL7 predicted loss-of-function polypeptide, the therapeutic agent that treats or inhibits the ophthalmic condition is administered or continued to be administered to the subject in a standard dosage amount, and an ANGPTL7 inhibitor is administered to the subject. When the subject has an ANGPTL7 predicted loss-of-function polypeptide, the therapeutic agent that treats or inhibits the ophthalmic condition is administered or continued to be administered to the subject in an amount that is the same as or less than a standard dosage amount, and an ANGPTL7 inhibitor is administered to the subject. The presence of an ANGPTL7 predicted loss-of-function polypeptide indicates the subject has a decreased risk of developing an ophthalmic condition. In some embodiments, the subject has an ANGPTL7 predicted loss-of-function polypeptide. In some embodiments, the subject does not have an ANGPTL7 predicted loss-of-function polypeptide.

Detecting the presence or absence of an ANGPTL7 predicted loss-of-function polypeptide in a biological sample from a subject and/or determining whether a subject has an ANGPTL7 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the ANGPTL7 predicted loss-of-function polypeptide can be present within a cell obtained from the subject.

Examples of therapeutic agents that treat or inhibit an ophthalmic condition include, but are not limited to: a prostaglandin, a beta blocker, an alpha-adrenergic agonist, a carbonic anhydrase inhibitor, a rho kinase inhibitor, or a miotic or cholinergic agent.

In some embodiments, the therapeutic agent that treats or inhibits the ophthalmic condition is a prostaglandin. In some embodiments, the prostaglandin is XALATAN® (latanoprost), TRAVATAN Z® (travoprost), ZIOPTAN® (tafluprost), LUMIGAN® (bimatoprost), or VYZULTA® (latanoprostene bunod). In some embodiments, the prostaglandin is latanoprost, travoprost, tafluprost, bimatoprost, or latanoprostene bunod.

In some embodiments, the therapeutic agent that treats or inhibits the ophthalmic condition is a beta blocker. In some embodiments, the beta blocker is BETIMOL®, ISTALOL®, or TIMOPTIC® (timolol) or BETOPTIC® (betaxolol). In some embodiments, the beta blocker is timolol or betaxolol.

In some embodiments, the therapeutic agent that treats or inhibits the ophthalmic condition is an alpha-adrenergic agonist. In some embodiments, the alpha-adrenergic agonist is IOPIDINE® (apraclonidine) or ALPHAGAN® or QOLIANA® (brimonidine). In some embodiments, the alpha-adrenergic agonist is apraclonidine or brimonidine.

In some embodiments, the therapeutic agent that treats or inhibits the ophthalmic condition is a carbonic anhydrase inhibitor. In some embodiments, the carbonic anhydrase inhibitor is TRUSOPT® (dorzolamide) or AZOPT® (brinzolamide). In some embodiments, the carbonic anhydrase inhibitor is dorzolamide or brinzolamide.

In some embodiments, the therapeutic agent that treats or inhibits the ophthalmic condition is a rho kinase inhibitor. In some embodiments, the rho kinase inhibitor is RHOPRESSA® (netarsudil). In some embodiments, the rho kinase inhibitor is netarsudil.

In some embodiments, the therapeutic agent that treats or inhibits the ophthalmic condition is a miotic or cholinergic agent. In some embodiments, the miotic or cholinergic agent is ISOPTO® Carpine (pilocarpine). In some embodiments, the miotic or cholinergic agent is pilocarpine.

In some embodiments, the dose of the therapeutic agents that treat or inhibit an ophthalmic condition can be reduced by about 10%, by about 20%, by about 30%, by about 40%, by about 50%, by about 60%, by about 70%, by about 80%, or by about 90% for subjects that are heterozygous for an ANGPTL7 missense variant nucleic acid molecule encoding an ANGPTL7 predicted loss-of-function polypeptide (i.e., a less than the standard dosage amount) compared to subjects that are ANGPTL7 reference (who may receive a standard dosage amount). In some embodiments, the dose of the therapeutic agents that treat or inhibit an ophthalmic condition can be reduced by about 10%, by about 20%, by about 30%, by about 40%, or by about 50%. In addition, the dose of therapeutic agents that treat or inhibit an ophthalmic condition in subjects that are heterozygous for an ANGPTL7 missense variant nucleic acid molecule encoding an ANGPTL7 predicted loss-of-function polypeptide can be administered less frequently compared to subjects that are ANGPTL7 reference.

Administration of the therapeutic agents that treat or inhibit an ophthalmic condition and/or ANGPTL7 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more. In addition, the therapeutic agents that treat or inhibit an ophthalmic condition and/or ANGPTL7 inhibitors can be administered sequentially or at the same time. In addition, the therapeutic agents that treat or inhibit an ophthalmic condition and/or ANGPTL7 inhibitors can be administered in separate compositions or can be administered together in the same composition.

Administration of the therapeutic agents that treat or inhibit an ophthalmic condition and/or ANGPTL7 inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in ophthalmic conditions, a decrease/reduction in the severity of ophthalmic conditions (such as, for example, a reduction or inhibition of development of ophthalmic conditions), a decrease/reduction in symptoms and ophthalmic condition-related effects, delaying the onset of symptoms and ophthalmic condition-related effects, reducing the severity of symptoms of ophthalmic condition-related effects, reducing the severity of an acute episode, reducing the number of symptoms and ophthalmic condition-related effects, reducing the latency of symptoms and ophthalmic condition-related effects, an amelioration of symptoms and ophthalmic condition-related effects, reducing secondary symptoms, reducing secondary infections, preventing relapse to ophthalmic conditions, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of ophthalmic conditions development/progression (such as, for example, a complete or partial avoidance/inhibition or a delay), and an increased survival time of the affected host animal, following administration of a therapeutic protocol. Treatment of ophthalmic conditions encompasses the treatment of subjects already diagnosed as having any form of ophthalmic conditions at any clinical stage or manifestation, the delay of the onset or evolution or aggravation or deterioration of the symptoms or signs of ophthalmic conditions, and/or preventing and/or reducing the severity of ophthalmic conditions.

The present disclosure also provides methods of identifying a subject having an increased risk of developing an ophthalmic condition. In some embodiments, the methods comprise determining or having determined the presence or absence of an ANGPTL7 missense variant nucleic acid molecule (such as a genomic nucleic acid molecule, mRNA molecule, and/or cDNA molecule) encoding an ANGPTL7 predicted loss-of-function polypeptide in a biological sample obtained from the subject. When the subject lacks an ANGPTL7 missense variant nucleic acid molecule encoding an ANGPTL7 predicted loss-of-function polypeptide (i.e., the subject is genotypically categorized as ANGPTL7 reference), then the subject has an increased risk of developing an ophthalmic condition. When the subject has an ANGPTL7 missense variant nucleic acid molecule encoding an ANGPTL7 predicted loss-of-function polypeptide (i.e., the subject is heterozygous or homozygous for an ANGPTL7 missense variant nucleic acid molecule), then the subject has a decreased risk of developing an ophthalmic condition compared to a subject that is ANGPTL7 reference.

Having a single copy of an ANGPTL7 missense variant nucleic acid molecule encoding an ANGPTL7 predicted loss-of-function polypeptide is more protective of a subject from developing an ophthalmic condition than having no copies of an ANGPTL7 missense variant nucleic acid molecule encoding an ANGPTL7 predicted loss-of-function polypeptide. Without intending to be limited to any particular theory or mechanism of action, it is believed that a single copy of an ANGPTL7 missense variant nucleic acid molecule encoding an ANGPTL7 predicted loss-of-function polypeptide (i.e., heterozygous for an ANGPTL7 missense variant nucleic acid molecule) is protective of a subject from developing an ophthalmic condition, and it is also believed that having two copies of an ANGPTL7 missense variant nucleic acid molecule encoding an ANGPTL7 predicted loss-of-function polypeptide (i.e., homozygous for an ANGPTL7 missense variant nucleic acid molecule) may be more protective of a subject from developing an ophthalmic condition, relative to a subject with a single copy. Thus, in some embodiments, a single copy of an ANGPTL7 missense variant nucleic acid molecule encoding an ANGPTL7 predicted loss-of-function polypeptide may not be completely protective, but instead, may be partially or incompletely protective of a subject from developing an ophthalmic condition. While not desiring to be bound by any particular theory, there may be additional factors or molecules involved in the development of an ophthalmic condition that are still present in a subject having a single copy of an ANGPTL7 missense variant nucleic acid molecule encoding an ANGPTL7 predicted loss-of-function polypeptide, thus resulting in less than complete protection from the development of an ophthalmic condition.

Detecting the presence or absence of an ANGPTL7 missense variant nucleic acid molecule encoding an ANGPTL7 predicted loss-of-function polypeptide in a biological sample from the subject and/or determining whether a subject has an ANGPTL7 missense variant nucleic acid molecule encoding an ANGPTL7 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the ANGPTL7 missense variant nucleic acid molecule encoding an ANGPTL7 predicted loss-of-function polypeptide can be present within a cell obtained from the subject.

In some embodiments, when a subject is identified as having an increased risk of developing an ophthalmic condition, the subject is further treated with a therapeutic agent that treats or inhibits an ophthalmic condition and/or an ANGPTL7 inhibitor, as described herein. For example, when the subject is ANGPTL7 reference, and therefore has an increased risk of developing an ophthalmic condition, the subject is administered an ANGPTL7 inhibitor. In some embodiments, such a subject is also administered a therapeutic agent that treats or inhibits an ophthalmic condition. In some embodiments, when the subject is heterozygous for an ANGPTL7 missense variant nucleic acid molecule encoding an ANGPTL7 predicted loss-of-function polypeptide, the subject is administered the therapeutic agent that treats or inhibits an ophthalmic condition in a dosage amount that is the same as or less than a standard dosage amount, and is also administered an ANGPTL7 inhibitor. In some embodiments, the subject is ANGPTL7 reference. In some embodiments, the subject is heterozygous for an ANGPTL7 missense variant nucleic acid molecule encoding an ANGPTL7 predicted loss-of-function polypeptide.

The present disclosure also provides methods of detecting the presence or absence of an ANGPTL7 missense variant genomic nucleic acid molecule encoding an ANGPTL7 predicted loss-of-function polypeptide in a biological sample obtained from a subject, and/or an ANGPTL7 missense variant mRNA molecule encoding an ANGPTL7 predicted loss-of-function polypeptide in a biological sample obtained from a subject, and/or an ANGPTL7 missense variant cDNA molecule encoding an ANGPTL7 predicted loss-of-function polypeptide produced from an mRNA molecule in a biological sample obtained from a subject. It is understood that gene sequences within a population and mRNA molecules encoded by such genes can vary due to polymorphisms such as single-nucleotide polymorphisms. The sequences provided herein for the ANGPTL7 variant genomic nucleic acid molecule, ANGPTL7 variant mRNA molecule, and ANGPTL7 variant cDNA molecule are only exemplary sequences. Other sequences for the ANGPTL7 variant genomic nucleic acid molecule, variant mRNA molecule, and variant cDNA molecule are also possible.

The biological sample can be derived from any cell, tissue, or biological fluid from the subject. The biological sample may comprise any clinically relevant tissue such as, for example, a bone marrow sample, a tumor biopsy, a fine needle aspirate, or a sample of bodily fluid, such as blood, gingival crevicular fluid, plasma, serum, lymph, ascitic fluid, cystic fluid, or urine. In some embodiments, the biological sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any ANGPTL7 variant nucleic acid molecule, preliminary processing designed to isolate or enrich the biological sample for the ANGPTL7 variant nucleic acid molecule can be employed. A variety of techniques may be used for this purpose. When detecting the level of any ANGPTL7 variant mRNA molecule, different techniques can be used enrich the biological sample with mRNA molecules. Various methods to detect the presence or level of an mRNA molecule or the presence of a particular variant genomic DNA locus can be used.

The present disclosure also provides methods of detecting an ANGPTL7 missense variant nucleic acid molecule, or the complement thereof, encoding an ANGPTL7 predicted loss-of-function polypeptide in a subject. The methods comprise assaying a biological sample obtained from the subject to determine whether a nucleic acid molecule in the biological sample is an ANGPTL7 missense variant nucleic acid molecule encoding an ANGPTL7 predicted loss-of-function polypeptide.

In some embodiments, the ANGPTL7 missense variant nucleic acid molecule encoding the ANGPTL7 predicted loss-of-function polypeptide, or the complement thereof, is a genomic nucleic acid molecule having a nucleotide sequence comprising: an adenine at a position corresponding to position 4,229 according to SEQ ID NO:2, or the complement thereof; an adenine at a position corresponding to position 4,269 according to SEQ ID NO:3, or the complement thereof; a thymine at a position corresponding to position 4,273 according to SEQ ID NO:4, or the complement thereof; a thymine at a position corresponding to position 4,277 according to SEQ ID NO:5, or the complement thereof; an adenine at a position corresponding to position 4,311 according to SEQ ID NO:6, or the complement thereof; a cytosine at a position corresponding to position 4,322 according to SEQ ID NO:7, or the complement thereof; a thymine at a position corresponding to position 5,125 according to SEQ ID NO:8, or the complement thereof; a thymine at a position corresponding to position 5,176 according to SEQ ID NO:9, or the complement thereof; or an adenine at a position corresponding to position 5,232 according to SEQ ID NO:10, or the complement thereof.

In some embodiments, the ANGPTL7 missense variant nucleic acid molecule encoding the ANGPTL7 predicted loss-of-function polypeptide, or the complement thereof, is an mRNA molecule having a nucleotide sequence comprising: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof; a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a uracil at a position corresponding to position 768 according to SEQ ID NO:50, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof; a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; a uracil at a position corresponding to position 981 according to SEQ ID NO:82, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof.

In some embodiments, the ANGPTL7 missense variant nucleic acid molecule encoding the ANGPTL7 predicted loss-of-function polypeptide, or the complement thereof, is a cDNA molecule produced from an mRNA molecule in the biological sample having a nucleotide sequence comprising: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; an adenine at a position corresponding to position 760 according to SEQ ID NO:114, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; a thymine at a position corresponding to position 764 according to SEQ ID NO:122, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; a thymine at a position corresponding to position 768 according to SEQ ID NO:130, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; an adenine at a position corresponding to position 802 according to SEQ ID NO:138, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; a cytosine at a position corresponding to position 813 according to SEQ ID NO:146, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; a thymine at a position corresponding to position 930 according to SEQ ID NO:154, or the complement thereof; a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; a thymine at a position corresponding to position 981 according to SEQ ID NO:162, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof.

In some embodiments, the ANGPTL7 missense variant nucleic acid molecule has a nucleotide sequence comprising: i) an adenine at a position corresponding to position 4,229 according to SEQ ID NO:2 (for genomic nucleic acid molecules); ii) an adenine at a position corresponding to position 706 according to SEQ ID NO:19; an adenine at a position corresponding to position 773 according to SEQ ID NO:20; an adenine at a position corresponding to position 738 according to SEQ ID NO:21; an adenine at a position corresponding to position 720 according to SEQ ID NO:22; an adenine at a position corresponding to position 695 according to SEQ ID NO:23; an adenine at a position corresponding to position 488 according to SEQ ID NO:24; an adenine at a position corresponding to position 481 according to SEQ ID NO:25; or an adenine at a position corresponding to position 720 according to SEQ ID NO:26 (for mRNA molecules); or iii) an adenine at a position corresponding to position 706 according to SEQ ID NO:99; an adenine at a position corresponding to position 773 according to SEQ ID NO:100; an adenine at a position corresponding to position 738 according to SEQ ID NO:101; an adenine at a position corresponding to position 720 according to SEQ ID NO:102; an adenine at a position corresponding to position 695 according to SEQ ID NO:103; an adenine at a position corresponding to position 488 according to SEQ ID NO:104; an adenine at a position corresponding to position 481 according to SEQ ID NO:105; or an adenine at a position corresponding to position 720 according to SEQ ID NO:106 (for cDNA molecules obtained from mRNA molecules).

In some embodiments, the ANGPTL7 missense variant nucleic acid molecule has a nucleotide sequence comprising: i) an adenine at a position corresponding to position 4,269 according to SEQ ID NO:3 (for genomic nucleic acid molecules); ii) an adenine at a position corresponding to position 746 according to SEQ ID NO:27; an adenine at a position corresponding to position 812 according to SEQ ID NO:28; an adenine at a position corresponding to position 778 according to SEQ ID NO:29; an adenine at a position corresponding to position 760 according to SEQ ID NO:30; an adenine at a position corresponding to position 735 according to SEQ ID NO:31; an adenine at a position corresponding to position 528 according to SEQ ID NO:32; an adenine at a position corresponding to position 521 according to SEQ ID NO:33; or an adenine at a position corresponding to position 760 according to SEQ ID NO:34 (for mRNA molecules); or iii) an adenine at a position corresponding to position 746 according to SEQ ID NO:107; an adenine at a position corresponding to position 812 according to SEQ ID NO:108; an adenine at a position corresponding to position 778 according to SEQ ID NO:109; an adenine at a position corresponding to position 760 according to SEQ ID NO:110; an adenine at a position corresponding to position 735 according to SEQ ID NO:111; an adenine at a position corresponding to position 528 according to SEQ ID NO:112; an adenine at a position corresponding to position 529 according to SEQ ID NO:113; or an adenine at a position corresponding to position 760 according to SEQ ID NO:114 (for cDNA molecules obtained from mRNA molecules).

In some embodiments, the ANGPTL7 missense variant nucleic acid molecule has a nucleotide sequence comprising: i) a thymine at a position corresponding to position 4,273 according to SEQ ID NO:4 (for genomic nucleic acid molecules); ii) a uracil at a position corresponding to position 750 according to SEQ ID NO:35; a uracil at a position corresponding to position 817 according to SEQ ID NO:36; a uracil at a position corresponding to position 782 according to SEQ ID NO:37; a uracil at a position corresponding to position 764 according to SEQ ID NO:38; a uracil at a position corresponding to position 739 according to SEQ ID NO:39; a uracil at a position corresponding to position 532 according to SEQ ID NO:40; a uracil at a position corresponding to position 525 according to SEQ ID NO:41; or a uracil at a position corresponding to position 764 according to SEQ ID NO:42 (for mRNA molecules); or iii) a thymine at a position corresponding to position 750 according to SEQ ID NO:115; a thymine at a position corresponding to position 817 according to SEQ ID NO:116; a thymine at a position corresponding to position 782 according to SEQ ID NO:117; a thymine at a position corresponding to position 764 according to SEQ ID NO:118; a thymine at a position corresponding to position 739 according to SEQ ID NO:119; a thymine at a position corresponding to position 532 according to SEQ ID NO:120; a thymine at a position corresponding to position 525 according to SEQ ID NO:121; or a thymine at a position corresponding to position 764 according to SEQ ID NO:122 (for cDNA molecules obtained from mRNA molecules).

In some embodiments, the ANGPTL7 missense variant nucleic acid molecule has a nucleotide sequence comprising: i) a thymine at a position corresponding to position 4,277 according to SEQ ID NO:5 (for genomic nucleic acid molecules); ii) a uracil at a position corresponding to position 754 according to SEQ ID NO:43; a uracil at a position corresponding to position 821 according to SEQ ID NO:44; a uracil at a position corresponding to position 786 according to SEQ ID NO:45; a uracil at a position corresponding to position 768 according to SEQ ID NO:46; a uracil at a position corresponding to position 743 according to SEQ ID NO:47; a uracil at a position corresponding to position 536 according to SEQ ID NO:48; a uracil at a position corresponding to position 529 according to SEQ ID NO:49; or a uracil at a position corresponding to position 768 according to SEQ ID NO:50 (for mRNA molecules); or iii) a thymine at a position corresponding to position 754 according to SEQ ID NO:123; a thymine at a position corresponding to position 821 according to SEQ ID NO:124; a thymine at a position corresponding to position 786 according to SEQ ID NO:125; a thymine at a position corresponding to position 768 according to SEQ ID NO:126; a thymine at a position corresponding to position 743 according to SEQ ID NO:127; a thymine at a position corresponding to position 536 according to SEQ ID NO:128; a thymine at a position corresponding to position 529 according to SEQ ID NO:129; or a thymine at a position corresponding to position 768 according to SEQ ID NO:130 (for cDNA molecules obtained from mRNA molecules).

In some embodiments, the ANGPTL7 missense variant nucleic acid molecule has a nucleotide sequence comprising: i) an adenine at a position corresponding to position 4,311 according to SEQ ID NO:6 (for genomic nucleic acid molecules); ii) an adenine at a position corresponding to position 788 according to SEQ ID NO:51; an adenine at a position corresponding to position 855 according to SEQ ID NO:52; an adenine at a position corresponding to position 820 according to SEQ ID NO:53; an adenine at a position corresponding to position 802 according to SEQ ID NO:54; an adenine at a position corresponding to position 777 according to SEQ ID NO:55; an adenine at a position corresponding to position 570 according to SEQ ID NO:56; an adenine at a position corresponding to position 563 according to SEQ ID NO:57; or an adenine at a position corresponding to position 802 according to SEQ ID NO:58 (for mRNA molecules); or iii) an adenine at a position corresponding to position 788 according to SEQ ID NO:131; an adenine at a position corresponding to position 855 according to SEQ ID NO:132; an adenine at a position corresponding to position 820 according to SEQ ID NO:133; an adenine at a position corresponding to position 802 according to SEQ ID NO:134; an adenine at a position corresponding to position 777 according to SEQ ID NO:135; an adenine at a position corresponding to position 570 according to SEQ ID NO:136; an adenine at a position corresponding to position 563 according to SEQ ID NO:137; or an adenine at a position corresponding to position 802 according to SEQ ID NO:138 (for cDNA molecules obtained from mRNA molecules).

In some embodiments, the ANGPTL7 missense variant nucleic acid molecule has a nucleotide sequence comprising: i) a cytosine at a position corresponding to position 4,322 according to SEQ ID NO:7 (for genomic nucleic acid molecules); ii) a cytosine at a position corresponding to position 799 according to SEQ ID NO:59; a cytosine at a position corresponding to position 866 according to SEQ ID NO:60; a cytosine at a position corresponding to position 831 according to SEQ ID NO:61; a cytosine at a position corresponding to position 813 according to SEQ ID NO:62; a cytosine at a position corresponding to position 788 according to SEQ ID NO:63; a cytosine at a position corresponding to position 581 according to SEQ ID NO:64; a cytosine at a position corresponding to position 574 according to SEQ ID NO:65; or a cytosine at a position corresponding to position 813 according to SEQ ID NO:66 (for mRNA molecules); or iii) a cytosine at a position corresponding to position 799 according to SEQ ID NO:139; a cytosine at a position corresponding to position 866 according to SEQ ID NO:140; a cytosine at a position corresponding to position 831 according to SEQ ID NO:141; a cytosine at a position corresponding to position 813 according to SEQ ID NO:142; a cytosine at a position corresponding to position 788 according to SEQ ID NO:143; a cytosine at a position corresponding to position 581 according to SEQ ID NO:144; a cytosine at a position corresponding to position 574 according to SEQ ID NO:145; or a cytosine at a position corresponding to position 813 according to SEQ ID NO:146 (for cDNA molecules obtained from mRNA molecules).

In some embodiments, the ANGPTL7 missense variant nucleic acid molecule has a nucleotide sequence comprising: i) a thymine at a position corresponding to position 5,125 according to SEQ ID NO:8 (for genomic nucleic acid molecules); ii) a uracil at a position corresponding to position 916 according to SEQ ID NO:67; a uracil at a position corresponding to position 983 according to SEQ ID NO:68; a uracil at a position corresponding to position 948 according to SEQ ID NO:69; a uracil at a position corresponding to position 930 according to SEQ ID NO:70; a uracil at a position corresponding to position 905 according to SEQ ID NO:71; a uracil at a position corresponding to position 698 according to SEQ ID NO:72; a uracil at a position corresponding to position 691 according to SEQ ID NO:73; or a uracil at a position corresponding to position 930 according to SEQ ID NO:74 (for mRNA molecules); or iii) a thymine at a position corresponding to position 916 according to SEQ ID NO:147; a thymine at a position corresponding to position 983 according to SEQ ID NO:148; a thymine at a position corresponding to position 948 according to SEQ ID NO:149; a thymine at a position corresponding to position 930 according to SEQ ID NO:150; a thymine at a position corresponding to position 905 according to SEQ ID NO:151; a thymine at a position corresponding to position 698 according to SEQ ID NO:152; a thymine at a position corresponding to position 691 according to SEQ ID NO:153; or a thymine at a position corresponding to position 930 according to SEQ ID NO:154 (for cDNA molecules obtained from mRNA molecules).

In some embodiments, the ANGPTL7 missense variant nucleic acid molecule has a nucleotide sequence comprising: i) a thymine at a position corresponding to position 5,176 according to SEQ ID NO:9 (for genomic nucleic acid molecules); ii) a uracil at a position corresponding to position 967 according to SEQ ID NO:75; a uracil at a position corresponding to position 1,034 according to SEQ ID NO:76; a uracil at a position corresponding to position 999 according to SEQ ID NO:77; a uracil at a position corresponding to position 981 according to SEQ ID NO:78; a uracil at a position corresponding to position 956 according to SEQ ID NO:79; a uracil at a position corresponding to position 749 according to SEQ ID NO:80; a uracil at a position corresponding to position 742 according to SEQ ID NO:81; or a uracil at a position corresponding to position 981 according to SEQ ID NO:82 (for mRNA molecules); or iii) a thymine at a position corresponding to position 967 according to SEQ ID NO:155; a thymine at a position corresponding to position 1,034 according to SEQ ID NO:156; a thymine at a position corresponding to position 999 according to SEQ ID NO:157; a thymine at a position corresponding to position 981 according to SEQ ID NO:158; a thymine at a position corresponding to position 956 according to SEQ ID NO:159; a thymine at a position corresponding to position 749 according to SEQ ID NO:160; a thymine at a position corresponding to position 742 according to SEQ ID NO:161; or a thymine at a position corresponding to position 981 according to SEQ ID NO:162 (for cDNA molecules obtained from mRNA molecules).

In some embodiments, the ANGPTL7 missense variant nucleic acid molecule has a nucleotide sequence comprising: i) an adenine at a position corresponding to position 5,232 according to SEQ ID NO:10 (for genomic nucleic acid molecules); ii) an adenine at a position corresponding to position 1,023 according to SEQ ID NO:83; an adenine at a position corresponding to position 1,090 according to SEQ ID NO:84; an adenine at a position corresponding to position 1,055 according to SEQ ID NO:85; an adenine at a position corresponding to position 1,037 according to SEQ ID NO:86; an adenine at a position corresponding to position 1,012 according to SEQ ID NO:87; an adenine at a position corresponding to position 805 according to SEQ ID NO:88; an adenine at a position corresponding to position 798 according to SEQ ID NO:89; or an adenine at a position corresponding to position 1,037 according to SEQ ID NO:90; (for mRNA molecules); or iii) an adenine at a position corresponding to position 1,023 according to SEQ ID NO:163; an adenine at a position corresponding to position 1,090 according to SEQ ID NO:164; an adenine at a position corresponding to position 1,055 according to SEQ ID NO:165; an adenine at a position corresponding to position 1,037 according to SEQ ID NO:166; an adenine at a position corresponding to position 1,012 according to SEQ ID NO:167; an adenine at a position corresponding to position 805 according to SEQ ID NO:168; an adenine at a position corresponding to position 798 according to SEQ ID NO:169; or an adenine at a position corresponding to position 1,037 according to SEQ ID NO:170 (for cDNA molecules obtained from mRNA molecules).

In some embodiments, the biological sample comprises a cell or cell lysate. Such methods can further comprise, for example, obtaining a biological sample from the subject comprising an ANGPTL7 genomic nucleic acid molecule or mRNA molecule, and if mRNA, optionally reverse transcribing the mRNA into cDNA. Such assays can comprise, for example determining the identity of these positions of the particular ANGPTL7 nucleic acid molecule. In some embodiments, the method is an in vitro method.

In some embodiments, the determining step, detecting step, or sequence analysis comprises sequencing at least a portion of the nucleotide sequence of the ANGPTL7 genomic nucleic acid molecule, the ANGPTL7 mRNA molecule, or the ANGPTL7 cDNA molecule produced from the mRNA molecule in the biological sample, wherein the sequenced portion comprises one or more variations that cause a loss-of-function (partial or complete) or are predicted to cause a loss-of-function (partial or complete).

In some embodiments, the determining step, detecting step, or sequence analysis comprises sequencing at least a portion of: i) the nucleotide sequence of the ANGPTL7 genomic nucleic acid molecule in the biological sample, wherein the sequenced portion comprises a position corresponding to position 4,229 according to SEQ ID NO:2, or the complement thereof; ii) the nucleotide sequence of the ANGPTL7 mRNA molecule in the biological sample, wherein the sequenced portion comprises a position corresponding to: position 706 according to SEQ ID NO:19, or the complement thereof; position 773 according to SEQ ID NO:20, or the complement thereof; position 738 according to SEQ ID NO:21, or the complement thereof; position 720 according to SEQ ID NO:22, or the complement thereof; position 695 according to SEQ ID NO:23, or the complement thereof; position 488 according to SEQ ID NO:24, or the complement thereof; position 481 according to SEQ ID NO:25, or the complement thereof; or position 720 according to SEQ ID NO:26, or the complement thereof; and/or iii) the nucleotide sequence of the ANGPTL7 cDNA molecule produced from the mRNA in the biological sample, wherein the sequenced portion comprises a position corresponding to: position 706 according to SEQ ID NO:99, or the complement thereof; position 773 according to SEQ ID NO:100, or the complement thereof; position 738 according to SEQ ID NO:101, or the complement thereof; position 720 according to SEQ ID NO:102, or the complement thereof; position 695 according to SEQ ID NO:103, or the complement thereof; position 488 according to SEQ ID NO:104, or the complement thereof; position 481 according to SEQ ID NO:105, or the complement thereof; or position 720 according to SEQ ID NO:106, or the complement thereof. When the sequenced portion of the ANGPTL7 nucleic acid molecule in the biological sample comprises: an adenine at a position corresponding to position 4,229 according to SEQ ID NO:2, an adenine at a position corresponding to position 706 according to SEQ ID NO:19, an adenine at a position corresponding to position 773 according to SEQ ID NO:20, an adenine at a position corresponding to position 738 according to SEQ ID NO:21, an adenine at a position corresponding to position 720 according to SEQ ID NO:22, an adenine at a position corresponding to position 695 according to SEQ ID NO:23, an adenine at a position corresponding to position 488 according to SEQ ID NO:24, an adenine at a position corresponding to position 481 according to SEQ ID NO:25, an adenine at a position corresponding to position 720 according to SEQ ID NO:26, an adenine at a position corresponding to position 706 according to SEQ ID NO:99, an adenine at a position corresponding to position 773 according to SEQ ID NO:100, an adenine at a position corresponding to position 738 according to SEQ ID NO:101, an adenine at a position corresponding to position 720 according to SEQ ID NO:102, an adenine at a position corresponding to position 695 according to SEQ ID NO:103, an adenine at a position corresponding to position 488 according to SEQ ID NO:104, an adenine at a position corresponding to position 481 according to SEQ ID NO:105, or an adenine at a position corresponding to position 720 according to SEQ ID NO:106, then the ANGPTL7 nucleic acid molecule in the biological sample is an ANGPTL7 missense variant nucleic acid molecule encoding an ANGPTL7 predicted loss-of-function polypeptide.

In some embodiments, the determining step, detecting step, or sequence analysis comprises sequencing at least a portion of: i) the nucleotide sequence of the ANGPTL7 genomic nucleic acid molecule in the biological sample, wherein the sequenced portion comprises a position corresponding to position 4,269 according to SEQ ID NO:3, or the complement thereof; ii) the nucleotide sequence of the ANGPTL7 mRNA molecule in the biological sample, wherein the sequenced portion comprises a position corresponding to: position 746 according to SEQ ID NO:27, or the complement thereof; position 812 according to SEQ ID NO:28, or the complement thereof; position 778 according to SEQ ID NO:29, or the complement thereof; position 760 according to SEQ ID NO:30, or the complement thereof; position 735 according to SEQ ID NO:31, or the complement thereof; position 528 according to SEQ ID NO:32, or the complement thereof; position 521 according to SEQ ID NO:33, or the complement thereof; position 760 according to SEQ ID NO:34, or the complement thereof; and/or iii) the nucleotide sequence of the ANGPTL7 cDNA molecule produced from the mRNA in the biological sample, wherein the sequenced portion comprises a position corresponding to: position 746 according to SEQ ID NO:107, or the complement thereof; position 812 according to SEQ ID NO:108, or the complement thereof; position 778 according to SEQ ID NO:109, or the complement thereof; position 760 according to SEQ ID NO:110, or the complement thereof; position 735 according to SEQ ID NO:111, or the complement thereof; position 528 according to SEQ ID NO:112, or the complement thereof; position 529 according to SEQ ID NO:113, or the complement thereof; position 760 according to SEQ ID NO:114, or the complement thereof. When the sequenced portion of the ANGPTL7 nucleic acid molecule in the biological sample comprises: an adenine at a position corresponding to position 4,269 according to SEQ ID NO:3, an adenine at a position corresponding to position 746 according to SEQ ID NO:27, an adenine at a position corresponding to position 812 according to SEQ ID NO:28, an adenine at a position corresponding to position 778 according to SEQ ID NO:29, an adenine at a position corresponding to position 760 according to SEQ ID NO:30, an adenine at a position corresponding to position 735 according to SEQ ID NO:31, an adenine at a position corresponding to position 528 according to SEQ ID NO:32, an adenine at a position corresponding to position 521 according to SEQ ID NO:33, an adenine at a position corresponding to position 760 according to SEQ ID NO:34, an adenine at a position corresponding to position 746 according to SEQ ID NO:107, an adenine at a position corresponding to position 812 according to SEQ ID NO:108, an adenine at a position corresponding to position 778 according to SEQ ID NO:109, an adenine at a position corresponding to position 760 according to SEQ ID NO:110, an adenine at a position corresponding to position 735 according to SEQ ID NO:111, an adenine at a position corresponding to position 528 according to SEQ ID NO:112, an adenine at a position corresponding to position 529 according to SEQ ID NO:113, an adenine at a position corresponding to position 760 according to SEQ ID NO:114, then the ANGPTL7 nucleic acid molecule in the biological sample is an ANGPTL7 missense variant nucleic acid molecule encoding an ANGPTL7 predicted loss-of-function polypeptide.

In some embodiments, the determining step, detecting step, or sequence analysis comprises sequencing at least a portion of: i) the nucleotide sequence of the ANGPTL7 genomic nucleic acid molecule in the biological sample, wherein the sequenced portion comprises a position corresponding to position 4,273 according to SEQ ID NO:4, or the complement thereof; ii) the nucleotide sequence of the ANGPTL7 mRNA molecule in the biological sample, wherein the sequenced portion comprises a position corresponding to: position 750 according to SEQ ID NO:35, or the complement thereof; position 817 according to SEQ ID NO:36, or the complement thereof; position 782 according to SEQ ID NO:37, or the complement thereof; position 764 according to SEQ ID NO:38, or the complement thereof; position 739 according to SEQ ID NO:39, or the complement thereof; position 532 according to SEQ ID NO:40, or the complement thereof; position 525 according to SEQ ID NO:41, or the complement thereof; position 764 according to SEQ ID NO:42, or the complement thereof; and/or iii) the nucleotide sequence of the ANGPTL7 cDNA molecule produced from the mRNA in the biological sample, wherein the sequenced portion comprises a position corresponding to: position 750 according to SEQ ID NO:115, or the complement thereof; position 817 according to SEQ ID NO:116, or the complement thereof; position 782 according to SEQ ID NO:117, or the complement thereof; position 764 according to SEQ ID NO:118, or the complement thereof; position 739 according to SEQ ID NO:119, or the complement thereof; position 532 according to SEQ ID NO:120, or the complement thereof; position 525 according to SEQ ID NO:121, or the complement thereof; position 764 according to SEQ ID NO:122, or the complement thereof. When the sequenced portion of the ANGPTL7 nucleic acid molecule in the biological sample comprises: a thymine at a position corresponding to position 4,273 according to SEQ ID NO:4, a uracil at a position corresponding to position 750 according to SEQ ID NO:35, a uracil at a position corresponding to position 817 according to SEQ ID NO:36, a uracil at a position corresponding to position 782 according to SEQ ID NO:37, a uracil at a position corresponding to position 764 according to SEQ ID NO:38, a uracil at a position corresponding to position 739 according to SEQ ID NO:39, a uracil at a position corresponding to position 532 according to SEQ ID NO:40, a uracil at a position corresponding to position 525 according to SEQ ID NO:41, a uracil at a position corresponding to position 764 according to SEQ ID NO:42, a thymine at a position corresponding to position 750 according to SEQ ID NO:115, a thymine at a position corresponding to position 817 according to SEQ ID NO:116, a thymine at a position corresponding to position 782 according to SEQ ID NO:117, a thymine at a position corresponding to position 764 according to SEQ ID NO:118, a thymine at a position corresponding to position 739 according to SEQ ID NO:119, a thymine at a position corresponding to position 532 according to SEQ ID NO:120, a thymine at a position corresponding to position 525 according to SEQ ID NO:121, a thymine at a position corresponding to position 764 according to SEQ ID NO:122, then the ANGPTL7 nucleic acid molecule in the biological sample is an ANGPTL7 missense variant nucleic acid molecule encoding an ANGPTL7 predicted loss-of-function polypeptide.

In some embodiments, the determining step, detecting step, or sequence analysis comprises sequencing at least a portion of: i) the nucleotide sequence of the ANGPTL7 genomic nucleic acid molecule in the biological sample, wherein the sequenced portion comprises a position corresponding to position 4,277 according to SEQ ID NO:5, or the complement thereof; ii) the nucleotide sequence of the ANGPTL7 mRNA molecule in the biological sample, wherein the sequenced portion comprises a position corresponding to: position 754 according to SEQ ID NO:43, or the complement thereof; position 821 according to SEQ ID NO:44, or the complement thereof; position 786 according to SEQ ID NO:45, or the complement thereof; position 768 according to SEQ ID NO:46, or the complement thereof; position 743 according to SEQ ID NO:47, or the complement thereof; position 536 according to SEQ ID NO:48, or the complement thereof; position 529 according to SEQ ID NO:49, or the complement thereof; position 768 according to SEQ ID NO:50, or the complement thereof; and/or iii) the nucleotide sequence of the ANGPTL7 cDNA molecule produced from the mRNA in the biological sample, wherein the sequenced portion comprises a position corresponding to: position 754 according to SEQ ID NO:123, or the complement thereof; position 821 according to SEQ ID NO:124, or the complement thereof; position 786 according to SEQ ID NO:125, or the complement thereof; position 768 according to SEQ ID NO:126, or the complement thereof; position 743 according to SEQ ID NO:127, or the complement thereof; position 536 according to SEQ ID NO:128, or the complement thereof; position 529 according to SEQ ID NO:129, or the complement thereof; position 768 according to SEQ ID NO:130, or the complement thereof. When the sequenced portion of the ANGPTL7 nucleic acid molecule in the biological sample comprises: a thymine at a position corresponding to position 4,277 according to SEQ ID NO:5, a uracil at a position corresponding to position 754 according to SEQ ID NO:43, a uracil at a position corresponding to position 821 according to SEQ ID NO:44, a uracil at a position corresponding to position 786 according to SEQ ID NO:45, a uracil at a position corresponding to position 768 according to SEQ ID NO:46, a uracil at a position corresponding to position 743 according to SEQ ID NO:47, a uracil at a position corresponding to position 536 according to SEQ ID NO:48, a uracil at a position corresponding to position 529 according to SEQ ID NO:49, a uracil at a position corresponding to position 768 according to SEQ ID NO:50, a thymine at a position corresponding to position 754 according to SEQ ID NO:123, a thymine at a position corresponding to position 821 according to SEQ ID NO:124, a thymine at a position corresponding to position 786 according to SEQ ID NO:125, a thymine at a position corresponding to position 768 according to SEQ ID NO:126, a thymine at a position corresponding to position 743 according to SEQ ID NO:127, a thymine at a position corresponding to position 536 according to SEQ ID NO:128, a thymine at a position corresponding to position 529 according to SEQ ID NO:129, a thymine at a position corresponding to position 768 according to SEQ ID NO:130, then the ANGPTL7 nucleic acid molecule in the biological sample is an ANGPTL7 missense variant nucleic acid molecule encoding an ANGPTL7 predicted loss-of-function polypeptide.

In some embodiments, the determining step, detecting step, or sequence analysis comprises sequencing at least a portion of: i) the nucleotide sequence of the ANGPTL7 genomic nucleic acid molecule in the biological sample, wherein the sequenced portion comprises a position corresponding to position 4,311 according to SEQ ID NO:6, or the complement thereof; ii) the nucleotide sequence of the ANGPTL7 mRNA molecule in the biological sample, wherein the sequenced portion comprises a position corresponding to: position 788 according to SEQ ID NO:51, or the complement thereof; position 855 according to SEQ ID NO:52, or the complement thereof; position 820 according to SEQ ID NO:53, or the complement thereof; position 802 according to SEQ ID NO:54, or the complement thereof; position 777 according to SEQ ID NO:55, or the complement thereof; position 570 according to SEQ ID NO:56, or the complement thereof; position 563 according to SEQ ID NO:57, or the complement thereof; position 802 according to SEQ ID NO:58, or the complement thereof; and/or iii) the nucleotide sequence of the ANGPTL7 cDNA molecule produced from the mRNA in the biological sample, wherein the sequenced portion comprises a position corresponding to: position 788 according to SEQ ID NO:131, or the complement thereof; position 855 according to SEQ ID NO:132, or the complement thereof; position 820 according to SEQ ID NO:133, or the complement thereof; position 802 according to SEQ ID NO:134, or the complement thereof; position 777 according to SEQ ID NO:135, or the complement thereof; position 570 according to SEQ ID NO:136, or the complement thereof; position 563 according to SEQ ID NO:137, or the complement thereof; position 802 according to SEQ ID NO:138, or the complement thereof. When the sequenced portion of the ANGPTL7 nucleic acid molecule in the biological sample comprises: an adenine at a position corresponding to position 4,311 according to SEQ ID NO:6, an adenine at a position corresponding to position 788 according to SEQ ID NO:51, an adenine at a position corresponding to position 855 according to SEQ ID NO:52, an adenine at a position corresponding to position 820 according to SEQ ID NO:53, an adenine at a position corresponding to position 802 according to SEQ ID NO:54, an adenine at a position corresponding to position 777 according to SEQ ID NO:55, an adenine at a position corresponding to position 570 according to SEQ ID NO:56, an adenine at a position corresponding to position 563 according to SEQ ID NO:57, an adenine at a position corresponding to position 802 according to SEQ ID NO:58, an adenine at a position corresponding to position 788 according to SEQ ID NO:131, an adenine at a position corresponding to position 855 according to SEQ ID NO:132, an adenine at a position corresponding to position 820 according to SEQ ID NO:133, an adenine at a position corresponding to position 802 according to SEQ ID NO:134, an adenine at a position corresponding to position 777 according to SEQ ID NO:135, an adenine at a position corresponding to position 570 according to SEQ ID NO:136, an adenine at a position corresponding to position 563 according to SEQ ID NO:137, an adenine at a position corresponding to position 802 according to SEQ ID NO:138, then the ANGPTL7 nucleic acid molecule in the biological sample is an ANGPTL7 missense variant nucleic acid molecule encoding an ANGPTL7 predicted loss-of-function polypeptide.

In some embodiments, the determining step, detecting step, or sequence analysis comprises sequencing at least a portion of: i) the nucleotide sequence of the ANGPTL7 genomic nucleic acid molecule in the biological sample, wherein the sequenced portion comprises a position corresponding to position 4,322 according to SEQ ID NO:7, or the complement thereof; ii) the nucleotide sequence of the ANGPTL7 mRNA molecule in the biological sample, wherein the sequenced portion comprises a position corresponding to: position 799 according to SEQ ID NO:59, or the complement thereof; position 866 according to SEQ ID NO:60, or the complement thereof; position 831 according to SEQ ID NO:61, or the complement thereof; position 813 according to SEQ ID NO:62, or the complement thereof; position 788 according to SEQ ID NO:63, or the complement thereof; position 581 according to SEQ ID NO:64, or the complement thereof; position 574 according to SEQ ID NO:65, or the complement thereof; position 813 according to SEQ ID NO:66, or the complement thereof; and/or iii) the nucleotide sequence of the ANGPTL7 cDNA molecule produced from the mRNA in the biological sample, wherein the sequenced portion comprises a position corresponding to: position 799 according to SEQ ID NO:139, or the complement thereof; position 866 according to SEQ ID NO:140, or the complement thereof; position 831 according to SEQ ID NO:141, or the complement thereof; position 813 according to SEQ ID NO:142, or the complement thereof; position 788 according to SEQ ID NO:143, or the complement thereof; position 581 according to SEQ ID NO:144, or the complement thereof; position 574 according to SEQ ID NO:145, or the complement thereof; position 813 according to SEQ ID NO:146, or the complement thereof. When the sequenced portion of the ANGPTL7 nucleic acid molecule in the biological sample comprises: a cytosine at a position corresponding to position 4,322 according to SEQ ID NO:7, a cytosine at a position corresponding to position 799 according to SEQ ID NO:59, a cytosine at a position corresponding to position 866 according to SEQ ID NO:60, a cytosine at a position corresponding to position 831 according to SEQ ID NO:61, a cytosine at a position corresponding to position 813 according to SEQ ID NO:62, a cytosine at a position corresponding to position 788 according to SEQ ID NO:63, a cytosine at a position corresponding to position 581 according to SEQ ID NO:64, a cytosine at a position corresponding to position 574 according to SEQ ID NO:65, a cytosine at a position corresponding to position 813 according to SEQ ID NO:66, a cytosine at a position corresponding to position 799 according to SEQ ID NO:139, a cytosine at a position corresponding to position 866 according to SEQ ID NO:140, a cytosine at a position corresponding to position 831 according to SEQ ID NO:141, a cytosine at a position corresponding to position 813 according to SEQ ID NO:142, a cytosine at a position corresponding to position 788 according to SEQ ID NO:143, a cytosine at a position corresponding to position 581 according to SEQ ID NO:144, a cytosine at a position corresponding to position 574 according to SEQ ID NO:145, a cytosine at a position corresponding to position 813 according to SEQ ID NO:146, then the ANGPTL7 nucleic acid molecule in the biological sample is an ANGPTL7 missense variant nucleic acid molecule encoding an ANGPTL7 predicted loss-of-function polypeptide.

In some embodiments, the determining step, detecting step, or sequence analysis comprises sequencing at least a portion of: i) the nucleotide sequence of the ANGPTL7 genomic nucleic acid molecule in the biological sample, wherein the sequenced portion comprises a position corresponding to position 5,125 according to SEQ ID NO:8, or the complement thereof; ii) the nucleotide sequence of the ANGPTL7 mRNA molecule in the biological sample, wherein the sequenced portion comprises a position corresponding to: position 916 according to SEQ ID NO:67, or the complement thereof; position 983 according to SEQ ID NO:68, or the complement thereof; position 948 according to SEQ ID NO:69, or the complement thereof; position 930 according to SEQ ID NO:70, or the complement thereof; position 905 according to SEQ ID NO:71, or the complement thereof; position 698 according to SEQ ID NO:72, or the complement thereof; position 691 according to SEQ ID NO:73, or the complement thereof; position 930 according to SEQ ID NO:74, or the complement thereof; and/or iii) the nucleotide sequence of the ANGPTL7 cDNA molecule produced from the mRNA in the biological sample, wherein the sequenced portion comprises a position corresponding to: position 916 according to SEQ ID NO:147, or the complement thereof; position 983 according to SEQ ID NO:148, or the complement thereof; position 948 according to SEQ ID NO:149, or the complement thereof; position 930 according to SEQ ID NO:150, or the complement thereof; position 905 according to SEQ ID NO:151, or the complement thereof; position 698 according to SEQ ID NO:152, or the complement thereof; position 691 according to SEQ ID NO:153, or the complement thereof; position 930 according to SEQ ID NO:154, or the complement thereof. When the sequenced portion of the ANGPTL7 nucleic acid molecule in the biological sample comprises: a thymine at a position corresponding to position 5,125 according to SEQ ID NO:8, a uracil at a position corresponding to position 916 according to SEQ ID NO:67, a uracil at a position corresponding to position 983 according to SEQ ID NO:68, a uracil at a position corresponding to position 948 according to SEQ ID NO:69, a uracil at a position corresponding to position 930 according to SEQ ID NO:70, a uracil at a position corresponding to position 905 according to SEQ ID NO:71, a uracil at a position corresponding to position 698 according to SEQ ID NO:72, a uracil at a position corresponding to position 691 according to SEQ ID NO:73, a uracil at a position corresponding to position 930 according to SEQ ID NO:74, a thymine at a position corresponding to position 916 according to SEQ ID NO:147, a thymine at a position corresponding to position 983 according to SEQ ID NO:148, a thymine at a position corresponding to position 948 according to SEQ ID NO:149, a thymine at a position corresponding to position 930 according to SEQ ID NO:150, a thymine at a position corresponding to position 905 according to SEQ ID NO:151, a thymine at a position corresponding to position 698 according to SEQ ID NO:152, a thymine at a position corresponding to position 691 according to SEQ ID NO:153, a thymine at a position corresponding to position 930 according to SEQ ID NO:154, then the ANGPTL7 nucleic acid molecule in the biological sample is an ANGPTL7 missense variant nucleic acid molecule encoding an ANGPTL7 predicted loss-of-function polypeptide.

In some embodiments, the determining step, detecting step, or sequence analysis comprises sequencing at least a portion of: i) the nucleotide sequence of the ANGPTL7 genomic nucleic acid molecule in the biological sample, wherein the sequenced portion comprises a position corresponding to position 5,176 according to SEQ ID NO:9, or the complement thereof; ii) the nucleotide sequence of the ANGPTL7 mRNA molecule in the biological sample, wherein the sequenced portion comprises a position corresponding to: position 967 according to SEQ ID NO:75, or the complement thereof; position 1,034 according to SEQ ID NO:76, or the complement thereof; position 999 according to SEQ ID NO:77, or the complement thereof; position 981 according to SEQ ID NO:78, or the complement thereof; position 956 according to SEQ ID NO:79, or the complement thereof; position 749 according to SEQ ID NO:80, or the complement thereof; position 742 according to SEQ ID NO:81, or the complement thereof; position 981 according to SEQ ID NO:82, or the complement thereof; and/or iii) the nucleotide sequence of the ANGPTL7 cDNA molecule produced from the mRNA in the biological sample, wherein the sequenced portion comprises a position corresponding to: position 967 according to SEQ ID NO:155, or the complement thereof; position 1,034 according to SEQ ID NO:156, or the complement thereof; position 999 according to SEQ ID NO:157, or the complement thereof; position 981 according to SEQ ID NO:158, or the complement thereof; position 956 according to SEQ ID NO:159, or the complement thereof; position 749 according to SEQ ID NO:160, or the complement thereof; position 742 according to SEQ ID NO:161, or the complement thereof; position 981 according to SEQ ID NO:162, or the complement thereof. When the sequenced portion of the ANGPTL7 nucleic acid molecule in the biological sample comprises: a thymine at a position corresponding to position 5,176 according to SEQ ID NO:9, a uracil at a position corresponding to position 967 according to SEQ ID NO:75, a uracil at a position corresponding to position 1,034 according to SEQ ID NO:76, a uracil at a position corresponding to position 999 according to SEQ ID NO:77, a uracil at a position corresponding to position 981 according to SEQ ID NO:78, a uracil at a position corresponding to position 956 according to SEQ ID NO:79, a uracil at a position corresponding to position 749 according to SEQ ID NO:80, a uracil at a position corresponding to position 742 according to SEQ ID NO:81, a uracil at a position corresponding to position 981 according to SEQ ID NO:82, a thymine at a position corresponding to position 967 according to SEQ ID NO:155, a thymine at a position corresponding to position 1,034 according to SEQ ID NO:156, a thymine at a position corresponding to position 999 according to SEQ ID NO:157, a thymine at a position corresponding to position 981 according to SEQ ID NO:158, a thymine at a position corresponding to position 956 according to SEQ ID NO:159, a thymine at a position corresponding to position 749 according to SEQ ID NO:160, a thymine at a position corresponding to position 742 according to SEQ ID NO:161, a thymine at a position corresponding to position 981 according to SEQ ID NO:162, then the ANGPTL7 nucleic acid molecule in the biological sample is an ANGPTL7 missense variant nucleic acid molecule encoding an ANGPTL7 predicted loss-of-function polypeptide.

In some embodiments, the determining step, detecting step, or sequence analysis comprises sequencing at least a portion of: i) the nucleotide sequence of the ANGPTL7 genomic nucleic acid molecule in the biological sample, wherein the sequenced portion comprises a position corresponding to position 5,232 according to SEQ ID NO:10, or the complement thereof; ii) the nucleotide sequence of the ANGPTL7 mRNA molecule in the biological sample, wherein the sequenced portion comprises a position corresponding to: position 1,023 according to SEQ ID NO:83, or the complement thereof; position 1,090 according to SEQ ID NO:84, or the complement thereof; position 1,055 according to SEQ ID NO:85, or the complement thereof; position 1,037 according to SEQ ID NO:86, or the complement thereof; position 1,012 according to SEQ ID NO:87, or the complement thereof; position 805 according to SEQ ID NO:88, or the complement thereof; position 798 according to SEQ ID NO:89, or the complement thereof; or position 1,037 according to SEQ ID NO:90, or the complement thereof; and/or iii) the nucleotide sequence of the ANGPTL7 cDNA molecule produced from the mRNA in the biological sample, wherein the sequenced portion comprises a position corresponding to: position 1,023 according to SEQ ID NO:163, or the complement thereof; position 1,090 according to SEQ ID NO:164, or the complement thereof; position 1,055 according to SEQ ID NO:165, or the complement thereof; position 1,037 according to SEQ ID NO:166, or the complement thereof; position 1,012 according to SEQ ID NO:167, or the complement thereof; position 805 according to SEQ ID NO:168, or the complement thereof; position 798 according to SEQ ID NO:169, or the complement thereof; or position 1,037 according to SEQ ID NO:170, or the complement thereof. When the sequenced portion of the ANGPTL7 nucleic acid molecule in the biological sample comprises: an adenine at a position corresponding to position 1,023 according to SEQ ID NO:83, an adenine at a position corresponding to position 1,090 according to SEQ ID NO:84, an adenine at a position corresponding to position 1,055 according to SEQ ID NO:85, an adenine at a position corresponding to position 1,037 according to SEQ ID NO:86, an adenine at a position corresponding to position 1,012 according to SEQ ID NO:87, an adenine at a position corresponding to position 805 according to SEQ ID NO:88, an adenine at a position corresponding to position 798 according to SEQ ID NO:89, or an adenine at a position corresponding to position 1,037 according to SEQ ID NO:90, an adenine at a position corresponding to position 1,023 according to SEQ ID NO:163, an adenine at a position corresponding to position 1,090 according to SEQ ID NO:164, an adenine at a position corresponding to position 1,055 according to SEQ ID NO:165, an adenine at a position corresponding to position 1,037 according to SEQ ID NO:166, an adenine at a position corresponding to position 1,012 according to SEQ ID NO:167, an adenine at a position corresponding to position 805 according to SEQ ID NO:168, an adenine at a position corresponding to position 798 according to SEQ ID NO:169, or an adenine at a position corresponding to position 1,037 according to SEQ ID NO:170, or the complement thereof, then the ANGPTL7 nucleic acid molecule in the biological sample is an ANGPTL7 missense variant nucleic acid molecule encoding an ANGPTL7 predicted loss-of-function polypeptide.

In some embodiments, the determining step, detecting step, or sequence analysis comprises sequencing at least a portion of the nucleotide sequence of the ANGPTL7 genomic nucleic acid molecule in the biological sample, wherein the sequenced portion comprises a position corresponding to: position 4,229 according to SEQ ID NO:2, or the complement thereof; position 4,269 according to SEQ ID NO:3, or the complement thereof; position 4,273 according to SEQ ID NO:4, or the complement thereof; position 4,277 according to SEQ ID NO:5, or the complement thereof; position 4,311 according to SEQ ID NO:6, or the complement thereof; position 4,322 according to SEQ ID NO:7, or the complement thereof; position 5,125 according to SEQ ID NO:8, or the complement thereof; or position 5,176 according to SEQ ID NO:9, or the complement thereof, or a position corresponding to position 5,232 according to SEQ ID NO:10. When the sequenced portion of the ANGPTL7 nucleic acid molecule in the biological sample comprises: an adenine at a position corresponding to position 4,229 according to SEQ ID NO:2, an adenine at a position corresponding to position 4,269 according to SEQ ID NO:3, a thymine at a position corresponding to position 4,273 according to SEQ ID NO:4, a thymine at a position corresponding to position 4,277 according to SEQ ID NO:5, an adenine at a position corresponding to position 4,311 according to SEQ ID NO:6, a cytosine at a position corresponding to position 4,322 according to SEQ ID NO:7, a thymine at a position corresponding to position 5,125 according to SEQ ID NO:8, a thymine at a position corresponding to position 5,176 according to SEQ ID NO:9, or an adenine at a position corresponding to position 5,232 according to SEQ ID NO:10, then the ANGPTL7 nucleic acid molecule in the biological sample is an ANGPTL7 missense variant genomic nucleic acid molecule encoding an ANGPTL7 predicted loss-of-function polypeptide.

In some embodiments, the determining step, detecting step, or sequence analysis comprises sequencing at least a portion of the nucleotide sequence of the ANGPTL7 mRNA molecule in the biological sample, wherein the sequenced portion comprises a position corresponding to: position 706 according to SEQ ID NO:19, or the complement thereof; position 773 according to SEQ ID NO:20, or the complement thereof; position 738 according to SEQ ID NO:21, or the complement thereof; position 720 according to SEQ ID NO:22, or the complement thereof; position 695 according to SEQ ID NO:23, or the complement thereof; position 488 according to SEQ ID NO:24, or the complement thereof; position 481 according to SEQ ID NO:25, or the complement thereof; position 720 according to SEQ ID NO:26, or the complement thereof; position 746 according to SEQ ID NO:27, or the complement thereof; position 812 according to SEQ ID NO:28, or the complement thereof; position 778 according to SEQ ID NO:29, or the complement thereof; position 760 according to SEQ ID NO:30, or the complement thereof; position 735 according to SEQ ID NO:31, or the complement thereof; position 528 according to SEQ ID NO:32, or the complement thereof; position 521 according to SEQ ID NO:33, or the complement thereof; position 760 according to SEQ ID NO:34, or the complement thereof; position 750 according to SEQ ID NO:35, or the complement thereof; position 817 according to SEQ ID NO:36, or the complement thereof; position 782 according to SEQ ID NO:37, or the complement thereof; position 764 according to SEQ ID NO:38, or the complement thereof; position 739 according to SEQ ID NO:39, or the complement thereof; position 532 according to SEQ ID NO:40, or the complement thereof; position 525 according to SEQ ID NO:41, or the complement thereof; position 764 according to SEQ ID NO:42, or the complement thereof; position 754 according to SEQ ID NO:43, or the complement thereof; position 821 according to SEQ ID NO:44, or the complement thereof; position 786 according to SEQ ID NO:45, or the complement thereof; position 768 according to SEQ ID NO:46, or the complement thereof; position 743 according to SEQ ID NO:47, or the complement thereof; position 536 according to SEQ ID NO:48, or the complement thereof; position 529 according to SEQ ID NO:49, or the complement thereof; position 768 according to SEQ ID NO:50, or the complement thereof; position 788 according to SEQ ID NO:51, or the complement thereof; position 855 according to SEQ ID NO:52, or the complement thereof; position 820 according to SEQ ID NO:53, or the complement thereof; position 802 according to SEQ ID NO:54, or the complement thereof; position 777 according to SEQ ID NO:55, or the complement thereof; position 570 according to SEQ ID NO:56, or the complement thereof; position 563 according to SEQ ID NO:57, or the complement thereof; position 802 according to SEQ ID NO:58, or the complement thereof; position 799 according to SEQ ID NO:59, or the complement thereof; position 866 according to SEQ ID NO:60, or the complement thereof; position 831 according to SEQ ID NO:61, or the complement thereof; position 813 according to SEQ ID NO:62, or the complement thereof; position 788 according to SEQ ID NO:63, or the complement thereof; position 581 according to SEQ ID NO:64, or the complement thereof; position 574 according to SEQ ID NO:65, or the complement thereof; position 813 according to SEQ ID NO:66, or the complement thereof; position 916 according to SEQ ID NO:67, or the complement thereof; position 983 according to SEQ ID NO:68, or the complement thereof; position 948 according to SEQ ID NO:69, or the complement thereof; position 930 according to SEQ ID NO:70, or the complement thereof; position 905 according to SEQ ID NO:71, or the complement thereof; position 698 according to SEQ ID NO:72, or the complement thereof; position 691 according to SEQ ID NO:73, or the complement thereof; position 930 according to SEQ ID NO:74, or the complement thereof; position 967 according to SEQ ID NO:75, or the complement thereof; position 1,034 according to SEQ ID NO:76, or the complement thereof; position 999 according to SEQ ID NO:77, or the complement thereof; position 981 according to SEQ ID NO:78, or the complement thereof; position 956 according to SEQ ID NO:79, or the complement thereof; position 749 according to SEQ ID NO:80, or the complement thereof; position 742 according to SEQ ID NO:81, or the complement thereof; position 981 according to SEQ ID NO:82, or the complement thereof; position 1,023 according to SEQ ID NO:83, or the complement thereof; position 1,090 according to SEQ ID NO:84, or the complement thereof; position 1,055 according to SEQ ID NO:85, or the complement thereof; position 1,037 according to SEQ ID NO:86, or the complement thereof; position 1,012 according to SEQ ID NO:87, or the complement thereof; position 805 according to SEQ ID NO:88, or the complement thereof; position 798 according to SEQ ID NO:89, or the complement thereof; or position 1,037 according to SEQ ID NO:90, or the complement thereof. When the sequenced portion of the ANGPTL7 mRNA molecule in the biological sample comprises: an adenine at a position corresponding to position 706 according to SEQ ID NO:19, an adenine at a position corresponding to position 773 according to SEQ ID NO:20, an adenine at a position corresponding to position 738 according to SEQ ID NO:21, an adenine at a position corresponding to position 720 according to SEQ ID NO:22, an adenine at a position corresponding to position 695 according to SEQ ID NO:23, an adenine at a position corresponding to position 488 according to SEQ ID NO:24, an adenine at a position corresponding to position 481 according to SEQ ID NO:25, an adenine at a position corresponding to position 720 according to SEQ ID NO:26, an adenine at a position corresponding to position 746 according to SEQ ID NO:27, an adenine at a position corresponding to position 812 according to SEQ ID NO:28, an adenine at a position corresponding to position 778 according to SEQ ID NO:29, an adenine at a position corresponding to position 760 according to SEQ ID NO:30, an adenine at a position corresponding to position 735 according to SEQ ID NO:31, an adenine at a position corresponding to position 528 according to SEQ ID NO:32, an adenine at a position corresponding to position 521 according to SEQ ID NO:33, an adenine at a position corresponding to position 760 according to SEQ ID NO:34, a uracil at a position corresponding to position 750 according to SEQ ID NO:35, a uracil at a position corresponding to position 817 according to SEQ ID NO:36, a uracil at a position corresponding to position 782 according to SEQ ID NO:37, a uracil at a position corresponding to position 764 according to SEQ ID NO:38, a uracil at a position corresponding to position 739 according to SEQ ID NO:39, a uracil at a position corresponding to position 532 according to SEQ ID NO:40, a uracil at a position corresponding to position 525 according to SEQ ID NO:41, a uracil at a position corresponding to position 764 according to SEQ ID NO:42, a uracil at a position corresponding to position 754 according to SEQ ID NO:43, a uracil at a position corresponding to position 821 according to SEQ ID NO:44, a uracil at a position corresponding to position 786 according to SEQ ID NO:45, a uracil at a position corresponding to position 768 according to SEQ ID NO:46, a uracil at a position corresponding to position 743 according to SEQ ID NO:47, a uracil at a position corresponding to position 536 according to SEQ ID NO:48, a uracil at a position corresponding to position 529 according to SEQ ID NO:49, a uracil at a position corresponding to position 768 according to SEQ ID NO:50, an adenine at a position corresponding to position 788 according to SEQ ID NO:51, an adenine at a position corresponding to position 855 according to SEQ ID NO:52, an adenine at a position corresponding to position 820 according to SEQ ID NO:53, an adenine at a position corresponding to position 802 according to SEQ ID NO:54, an adenine at a position corresponding to position 777 according to SEQ ID NO:55, an adenine at a position corresponding to position 570 according to SEQ ID NO:56, an adenine at a position corresponding to position 563 according to SEQ ID NO:57, an adenine at a position corresponding to position 802 according to SEQ ID NO:58, a cytosine at a position corresponding to position 799 according to SEQ ID NO:59, a cytosine at a position corresponding to position 866 according to SEQ ID NO:60, a cytosine at a position corresponding to position 831 according to SEQ ID NO:61, a cytosine at a position corresponding to position 813 according to SEQ ID NO:62, a cytosine at a position corresponding to position 788 according to SEQ ID NO:63, a cytosine at a position corresponding to position 581 according to SEQ ID NO:64, a cytosine at a position corresponding to position 574 according to SEQ ID NO:65, a cytosine at a position corresponding to position 813 according to SEQ ID NO:66, a uracil at a position corresponding to position 916 according to SEQ ID NO:67, a uracil at a position corresponding to position 983 according to SEQ ID NO:68, a uracil at a position corresponding to position 948 according to SEQ ID NO:69, a uracil at a position corresponding to position 930 according to SEQ ID NO:70, a uracil at a position corresponding to position 905 according to SEQ ID NO:71, a uracil at a position corresponding to position 698 according to SEQ ID NO:72, a uracil at a position corresponding to position 691 according to SEQ ID NO:73, a uracil at a position corresponding to position 930 according to SEQ ID NO:74, a uracil at a position corresponding to position 967 according to SEQ ID NO:75, a uracil at a position corresponding to position 1,034 according to SEQ ID NO:76, a uracil at a position corresponding to position 999 according to SEQ ID NO:77, a uracil at a position corresponding to position 981 according to SEQ ID NO:78, a uracil at a position corresponding to position 956 according to SEQ ID NO:79, a uracil at a position corresponding to position 749 according to SEQ ID NO:80, a uracil at a position corresponding to position 742 according to SEQ ID NO:81, a uracil at a position corresponding to position 981 according to SEQ ID NO:82, an adenine at a position corresponding to position 1,023 according to SEQ ID NO:83, an adenine at a position corresponding to position 1,090 according to SEQ ID NO:84, an adenine at a position corresponding to position 1,055 according to SEQ ID NO:85, an adenine at a position corresponding to position 1,037 according to SEQ ID NO:86, an adenine at a position corresponding to position 1,012 according to SEQ ID NO:87, an adenine at a position corresponding to position 805 according to SEQ ID NO:88, an adenine at a position corresponding to position 798 according to SEQ ID NO:89, or an adenine at a position corresponding to position 1,037 according to SEQ ID NO:90, then the ANGPTL7 nucleic acid molecule in the biological sample is an ANGPTL7 missense variant mRNA molecule encoding an ANGPTL7 predicted loss-of-function polypeptide.

In some embodiments, the determining step, detecting step, or sequence analysis comprises sequencing at least a portion of the nucleotide sequence of the ANGPTL7 cDNA molecule produced from the mRNA molecule in the biological sample, wherein the sequenced portion comprises a position corresponding to: position 706 according to SEQ ID NO:99, or the complement thereof; position 773 according to SEQ ID NO:100, or the complement thereof; position 738 according to SEQ ID NO:101, or the complement thereof; position 720 according to SEQ ID NO:102, or the complement thereof; position 695 according to SEQ ID NO:103, or the complement thereof; position 488 according to SEQ ID NO:104, or the complement thereof; position 481 according to SEQ ID NO:105, or the complement thereof; position 720 according to SEQ ID NO:106, or the complement thereof; position 746 according to SEQ ID NO:107, or the complement thereof; position 812 according to SEQ ID NO:108, or the complement thereof; position 778 according to SEQ ID NO:109, or the complement thereof; position 760 according to SEQ ID NO:110, or the complement thereof; position 735 according to SEQ ID NO:111, or the complement thereof; position 528 according to SEQ ID NO:112, or the complement thereof; position 529 according to SEQ ID NO:113, or the complement thereof; position 760 according to SEQ ID NO:114, or the complement thereof; position 750 according to SEQ ID NO:115, or the complement thereof; position 817 according to SEQ ID NO:116, or the complement thereof; position 782 according to SEQ ID NO:117, or the complement thereof; position 764 according to SEQ ID NO:118, or the complement thereof; position 739 according to SEQ ID NO:119, or the complement thereof; position 532 according to SEQ ID NO:120, or the complement thereof; position 525 according to SEQ ID NO:121, or the complement thereof; position 764 according to SEQ ID NO:122, or the complement thereof; position 754 according to SEQ ID NO:123, or the complement thereof; position 821 according to SEQ ID NO:124, or the complement thereof; position 786 according to SEQ ID NO:125, or the complement thereof; position 768 according to SEQ ID NO:126, or the complement thereof; position 743 according to SEQ ID NO:127, or the complement thereof; position 536 according to SEQ ID NO:128, or the complement thereof; position 529 according to SEQ ID NO:129, or the complement thereof; position 768 according to SEQ ID NO:130, or the complement thereof; position 788 according to SEQ ID NO:131, or the complement thereof; position 855 according to SEQ ID NO:132, or the complement thereof; position 820 according to SEQ ID NO:133, or the complement thereof; position 802 according to SEQ ID NO:134, or the complement thereof; position 777 according to SEQ ID NO:135, or the complement thereof; position 570 according to SEQ ID NO:136, or the complement thereof; position 563 according to SEQ ID NO:137, or the complement thereof; position 802 according to SEQ ID NO:138, or the complement thereof; position 799 according to SEQ ID NO:139, or the complement thereof; position 866 according to SEQ ID NO:140, or the complement thereof; position 831 according to SEQ ID NO:141, or the complement thereof; position 813 according to SEQ ID NO:142, or the complement thereof; position 788 according to SEQ ID NO:143, or the complement thereof; position 581 according to SEQ ID NO:144, or the complement thereof; position 574 according to SEQ ID NO:145, or the complement thereof; position 813 according to SEQ ID NO:146, or the complement thereof; position 916 according to SEQ ID NO:147, or the complement thereof; position 983 according to SEQ ID NO:148, or the complement thereof; position 948 according to SEQ ID NO:149, or the complement thereof; position 930 according to SEQ ID NO:150, or the complement thereof; position 905 according to SEQ ID NO:151, or the complement thereof; position 698 according to SEQ ID NO:152, or the complement thereof; position 691 according to SEQ ID NO:153, or the complement thereof; position 930 according to SEQ ID NO:154, or the complement thereof; position 967 according to SEQ ID NO:155, or the complement thereof; position 1,034 according to SEQ ID NO:156, or the complement thereof; position 999 according to SEQ ID NO:157, or the complement thereof; position 981 according to SEQ ID NO:158, or the complement thereof; position 956 according to SEQ ID NO:159, or the complement thereof; position 749 according to SEQ ID NO:160, or the complement thereof; position 742 according to SEQ ID NO:161, or the complement thereof; position 981 according to SEQ ID NO:162, or the complement thereof; position 1,023 according to SEQ ID NO:163, or the complement thereof; position 1,090 according to SEQ ID NO:164, or the complement thereof; position 1,055 according to SEQ ID NO:165, or the complement thereof; position 1,037 according to SEQ ID NO:166, or the complement thereof; position 1,012 according to SEQ ID NO:167, or the complement thereof; position 805 according to SEQ ID NO:168, or the complement thereof; position 798 according to SEQ ID NO:169, or the complement thereof; or position 1,037 according to SEQ ID NO:170, or the complement thereof. When the sequenced portion of the ANGPTL7 cDNA molecule in the biological sample comprises: an adenine at a position corresponding to position 706 according to SEQ ID NO:99, an adenine at a position corresponding to position 773 according to SEQ ID NO:100, an adenine at a position corresponding to position 738 according to SEQ ID NO:101, an adenine at a position corresponding to position 720 according to SEQ ID NO:102, an adenine at a position corresponding to position 695 according to SEQ ID NO:103, an adenine at a position corresponding to position 488 according to SEQ ID NO:104, an adenine at a position corresponding to position 481 according to SEQ ID NO:105, an adenine at a position corresponding to position 720 according to SEQ ID NO:106, an adenine at a position corresponding to position 746 according to SEQ ID NO:107, an adenine at a position corresponding to position 812 according to SEQ ID NO:108, an adenine at a position corresponding to position 778 according to SEQ ID NO:109, an adenine at a position corresponding to position 760 according to SEQ ID NO:110, an adenine at a position corresponding to position 735 according to SEQ ID NO:111, an adenine at a position corresponding to position 528 according to SEQ ID NO:112, an adenine at a position corresponding to position 529 according to SEQ ID NO:113, an adenine at a position corresponding to position 760 according to SEQ ID NO:114, a thymine at a position corresponding to position 750 according to SEQ ID NO:115, a thymine at a position corresponding to position 817 according to SEQ ID NO:116, a thymine at a position corresponding to position 782 according to SEQ ID NO:117, a thymine at a position corresponding to position 764 according to SEQ ID NO:118, a thymine at a position corresponding to position 739 according to SEQ ID NO:119, a thymine at a position corresponding to position 532 according to SEQ ID NO:120, a thymine at a position corresponding to position 525 according to SEQ ID NO:121, a thymine at a position corresponding to position 764 according to SEQ ID NO:122, a thymine at a position corresponding to position 754 according to SEQ ID NO:123, a thymine at a position corresponding to position 821 according to SEQ ID NO:124, a thymine at a position corresponding to position 786 according to SEQ ID NO:125, a thymine at a position corresponding to position 768 according to SEQ ID NO:126, a thymine at a position corresponding to position 743 according to SEQ ID NO:127, a thymine at a position corresponding to position 536 according to SEQ ID NO:128, a thymine at a position corresponding to position 529 according to SEQ ID NO:129, a thymine at a position corresponding to position 768 according to SEQ ID NO:130, an adenine at a position corresponding to position 788 according to SEQ ID NO:131, an adenine at a position corresponding to position 855 according to SEQ ID NO:132, an adenine at a position corresponding to position 820 according to SEQ ID NO:133, an adenine at a position corresponding to position 802 according to SEQ ID NO:134, an adenine at a position corresponding to position 777 according to SEQ ID NO:135, an adenine at a position corresponding to position 570 according to SEQ ID NO:136, an adenine at a position corresponding to position 563 according to SEQ ID NO:137, an adenine at a position corresponding to position 802 according to SEQ ID NO:138, a cytosine at a position corresponding to position 799 according to SEQ ID NO:139, a cytosine at a position corresponding to position 866 according to SEQ ID NO:140, a cytosine at a position corresponding to position 831 according to SEQ ID NO:141, a cytosine at a position corresponding to position 813 according to SEQ ID NO:142, a cytosine at a position corresponding to position 788 according to SEQ ID NO:143, a cytosine at a position corresponding to position 581 according to SEQ ID NO:144, a cytosine at a position corresponding to position 574 according to SEQ ID NO:145, a cytosine at a position corresponding to position 813 according to SEQ ID NO:146, a thymine at a position corresponding to position 916 according to SEQ ID NO:147, a thymine at a position corresponding to position 983 according to SEQ ID NO:148, a thymine at a position corresponding to position 948 according to SEQ ID NO:149, a thymine at a position corresponding to position 930 according to SEQ ID NO:150, a thymine at a position corresponding to position 905 according to SEQ ID NO:151, a thymine at a position corresponding to position 698 according to SEQ ID NO:152, a thymine at a position corresponding to position 691 according to SEQ ID NO:153, a thymine at a position corresponding to position 930 according to SEQ ID NO:154, a thymine at a position corresponding to position 967 according to SEQ ID NO:155, a thymine at a position corresponding to position 1,034 according to SEQ ID NO:156, a thymine at a position corresponding to position 999 according to SEQ ID NO:157, a thymine at a position corresponding to position 981 according to SEQ ID NO:158, a thymine at a position corresponding to position 956 according to SEQ ID NO:159, a thymine at a position corresponding to position 749 according to SEQ ID NO:160, a thymine at a position corresponding to position 742 according to SEQ ID NO:161, a thymine at a position corresponding to position 981 according to SEQ ID NO:162, an adenine at a position corresponding to position 1,023 according to SEQ ID NO:163, an adenine at a position corresponding to position 1,090 according to SEQ ID NO:164, an adenine at a position corresponding to position 1,055 according to SEQ ID NO:165, an adenine at a position corresponding to position 1,037 according to SEQ ID NO:166, an adenine at a position corresponding to position 1,012 according to SEQ ID NO:167, an adenine at a position corresponding to position 805 according to SEQ ID NO:168, an adenine at a position corresponding to position 798 according to SEQ ID NO:169, or an adenine at a position corresponding to position 1,037 according to SEQ ID NO:170, or the complement thereof, then the ANGPTL7 nucleic acid molecule in the biological sample is an ANGPTL7 missense variant cDNA molecule encoding an ANGPTL7 predicted loss-of-function polypeptide.

In some embodiments, the determining step, detecting step, or sequence analysis comprises: a) contacting the biological sample with a primer hybridizing to a portion of the nucleotide sequence of the ANGPTL7: i) genomic nucleic acid molecule, or the complement thereof, that is proximate to a position corresponding to position 4,229 according to SEQ ID NO:2, or the complement thereof; ii) mRNA molecule, or the complement thereof, that is proximate to a position corresponding to: position 706 according to SEQ ID NO:19, or the complement thereof; position 773 according to SEQ ID NO:20, or the complement thereof; position 738 according to SEQ ID NO:21, or the complement thereof; position 720 according to SEQ ID NO:22, or the complement thereof; position 695 according to SEQ ID NO:23, or the complement thereof; position 488 according to SEQ ID NO:24, or the complement thereof; position 481 according to SEQ ID NO:25, or the complement thereof; or position 720 according to SEQ ID NO:26, or the complement thereof; and/or iii) cDNA molecule, or the complement thereof, that is proximate to a position corresponding to: position 706 according to SEQ ID NO:99, or the complement thereof; position 773 according to SEQ ID NO:100, or the complement thereof; position 738 according to SEQ ID NO:101, or the complement thereof; position 720 according to SEQ ID NO:102, or the complement thereof; position 695 according to SEQ ID NO:103, or the complement thereof; position 488 according to SEQ ID NO:104, or the complement thereof; position 481 according to SEQ ID NO:105, or the complement thereof; or position 720 according to SEQ ID NO:106, or the complement thereof; b) extending the primer at least through the position of the nucleotide sequence of the ANGPTL7: i) genomic nucleic acid molecule, or the complement thereof, corresponding to position 4,229 according to SEQ ID NO:2, or the complement thereof; ii) mRNA molecule, or the complement thereof, corresponding to: position 706 according to SEQ ID NO:19, or the complement thereof; position 773 according to SEQ ID NO:20, or the complement thereof; position 738 according to SEQ ID NO:21, or the complement thereof; position 720 according to SEQ ID NO:22, or the complement thereof; position 695 according to SEQ ID NO:23, or the complement thereof; position 488 according to SEQ ID NO:24, or the complement thereof; position 481 according to SEQ ID NO:25, or the complement thereof; or position 720 according to SEQ ID NO:26, or the complement thereof; and/or iii) cDNA molecule, or the complement thereof, corresponding to: position 706 according to SEQ ID NO:99, or the complement thereof; position 773 according to SEQ ID NO:100, or the complement thereof; position 738 according to SEQ ID NO:101, or the complement thereof; position 720 according to SEQ ID NO:102, or the complement thereof; position 695 according to SEQ ID NO:103, or the complement thereof; position 488 according to SEQ ID NO:104, or the complement thereof; position 481 according to SEQ ID NO:105, or the complement thereof; or position 720 according to SEQ ID NO:106, or the complement thereof; and c) determining whether the extension product of the primer comprises: an adenine at a position corresponding to position 4,229 according to SEQ ID NO:2, or the complement thereof; an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; and/or an adenine at a position corresponding to position 720 according to SEQ ID NO:106, or the complement thereof.

In some embodiments, the determining step, detecting step, or sequence analysis comprises: a) contacting the biological sample with a primer hybridizing to a portion of the nucleotide sequence of the ANGPTL7: i) genomic nucleic acid molecule, or the complement thereof, that is proximate to a position corresponding to position 4,269 according to SEQ ID NO:3, or the complement thereof; ii) mRNA molecule, or the complement thereof, that is proximate to a position corresponding to: position 746 according to SEQ ID NO:27, or the complement thereof; position 812 according to SEQ ID NO:28, or the complement thereof; position 778 according to SEQ ID NO:29, or the complement thereof; position 760 according to SEQ ID NO:30, or the complement thereof; position 735 according to SEQ ID NO:31, or the complement thereof; position 528 according to SEQ ID NO:32, or the complement thereof; position 521 according to SEQ ID NO:33, or the complement thereof; or position 760 according to SEQ ID NO:34, or the complement thereof; and/or iii) cDNA molecule, or the complement thereof, that is proximate to a position corresponding to: position 746 according to SEQ ID NO:107, or the complement thereof; position 812 according to SEQ ID NO:108, or the complement thereof; position 778 according to SEQ ID NO:109, or the complement thereof; position 760 according to SEQ ID NO:110, or the complement thereof; position 735 according to SEQ ID NO:111, or the complement thereof; position 528 according to SEQ ID NO:112, or the complement thereof; position 529 according to SEQ ID NO:113, or the complement thereof; or position 760 according to SEQ ID NO:114, or the complement thereof; b) extending the primer at least through the position of the nucleotide sequence of the ANGPTL7: i) genomic nucleic acid molecule, or the complement thereof, corresponding to position 4,269 according to SEQ ID NO:3, or the complement thereof; ii) mRNA molecule, or the complement thereof, corresponding to: position 746 according to SEQ ID NO:27, or the complement thereof; position 812 according to SEQ ID NO:28, or the complement thereof; position 778 according to SEQ ID NO:29, or the complement thereof; position 760 according to SEQ ID NO:30, or the complement thereof; position 735 according to SEQ ID NO:31, or the complement thereof; position 528 according to SEQ ID NO:32, or the complement thereof; position 521 according to SEQ ID NO:33, or the complement thereof; or position 760 according to SEQ ID NO:34, or the complement thereof; and/or iii) cDNA molecule, or the complement thereof, corresponding to: position 746 according to SEQ ID NO:107, or the complement thereof; position 812 according to SEQ ID NO:108, or the complement thereof; position 778 according to SEQ ID NO:109, or the complement thereof; position 760 according to SEQ ID NO:110, or the complement thereof; position 735 according to SEQ ID NO:111, or the complement thereof; position 528 according to SEQ ID NO:112, or the complement thereof; position 529 according to SEQ ID NO:113, or the complement thereof; position 760 according to SEQ ID NO:114, or the complement thereof; and c) determining whether the extension product of the primer comprises: an adenine at a position corresponding to position 4,269 according to SEQ ID NO:3, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; and/or an adenine at a position corresponding to position 760 according to SEQ ID NO:114, or the complement thereof.

In some embodiments, the determining step, detecting step, or sequence analysis comprises: a) contacting the biological sample with a primer hybridizing to a portion of the nucleotide sequence of the ANGPTL7: i) genomic nucleic acid molecule, or the complement thereof, that is proximate to a position corresponding to position 4,273 according to SEQ ID NO:4, or the complement thereof; ii) mRNA molecule, or the complement thereof, that is proximate to a position corresponding to: position 750 according to SEQ ID NO:35, or the complement thereof; position 817 according to SEQ ID NO:36, or the complement thereof; position 782 according to SEQ ID NO:37, or the complement thereof; position 764 according to SEQ ID NO:38, or the complement thereof; position 739 according to SEQ ID NO:39, or the complement thereof; position 532 according to SEQ ID NO:40, or the complement thereof; position 525 according to SEQ ID NO:41, or the complement thereof; or position 764 according to SEQ ID NO:42, or the complement thereof; and/or iii) cDNA molecule, or the complement thereof, that is proximate to a position corresponding to: position 750 according to SEQ ID NO:115, or the complement thereof; position 817 according to SEQ ID NO:116, or the complement thereof; position 782 according to SEQ ID NO:117, or the complement thereof; position 764 according to SEQ ID NO:118, or the complement thereof; position 739 according to SEQ ID NO:119, or the complement thereof; position 532 according to SEQ ID NO:120, or the complement thereof; position 525 according to SEQ ID NO:121, or the complement thereof; or position 764 according to SEQ ID NO:122, or the complement thereof; b) extending the primer at least through the position of the nucleotide sequence of the ANGPTL7: i) genomic nucleic acid molecule, or the complement thereof, corresponding to position 4,273 according to SEQ ID NO:4, or the complement thereof; ii) mRNA molecule, or the complement thereof, corresponding to: position 750 according to SEQ ID NO:35, or the complement thereof; position 817 according to SEQ ID NO:36, or the complement thereof; position 782 according to SEQ ID NO:37, or the complement thereof; position 764 according to SEQ ID NO:38, or the complement thereof; position 739 according to SEQ ID NO:39, or the complement thereof; position 532 according to SEQ ID NO:40, or the complement thereof; position 525 according to SEQ ID NO:41, or the complement thereof; or position 764 according to SEQ ID NO:42, or the complement thereof; and/or iii) cDNA molecule, or the complement thereof, corresponding to: position 750 according to SEQ ID NO:115, or the complement thereof; position 817 according to SEQ ID NO:116, or the complement thereof; position 782 according to SEQ ID NO:117, or the complement thereof; position 764 according to SEQ ID NO:118, or the complement thereof; position 739 according to SEQ ID NO:119, or the complement thereof; position 532 according to SEQ ID NO:120, or the complement thereof; position 525 according to SEQ ID NO:121, or the complement thereof; or position 764 according to SEQ ID NO:122, or the complement thereof and c) determining whether the extension product of the primer comprises: a thymine at a position corresponding to position 4,273 according to SEQ ID NO:4, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; and/or a thymine at a position corresponding to position 764 according to SEQ ID NO:122, or the complement thereof.

In some embodiments, the determining step, detecting step, or sequence analysis comprises: a) contacting the biological sample with a primer hybridizing to a portion of the nucleotide sequence of the ANGPTL7: i) genomic nucleic acid molecule, or the complement thereof, that is proximate to a position corresponding to position 4,277 according to SEQ ID NO:5, or the complement thereof; ii) mRNA molecule, or the complement thereof, that is proximate to a position corresponding to: position 754 according to SEQ ID NO:43, or the complement thereof; position 821 according to SEQ ID NO:44, or the complement thereof; position 786 according to SEQ ID NO:45, or the complement thereof; position 768 according to SEQ ID NO:46, or the complement thereof; position 743 according to SEQ ID NO:47, or the complement thereof; position 536 according to SEQ ID NO:48, or the complement thereof; position 529 according to SEQ ID NO:49, or the complement thereof; or position 768 according to SEQ ID NO:50, or the complement thereof; and/or iii) cDNA molecule, or the complement thereof, that is proximate to a position corresponding to: position 754 according to SEQ ID NO:123, or the complement thereof; position 821 according to SEQ ID NO:124, or the complement thereof; position 786 according to SEQ ID NO:125, or the complement thereof; position 768 according to SEQ ID NO:126, or the complement thereof; position 743 according to SEQ ID NO:127, or the complement thereof; position 536 according to SEQ ID NO:128, or the complement thereof; position 529 according to SEQ ID NO:129, or the complement thereof; or position 768 according to SEQ ID NO:130, or the complement thereof; b) extending the primer at least through the position of the nucleotide sequence of the ANGPTL7: i) genomic nucleic acid molecule, or the complement thereof, corresponding to position 4,277 according to SEQ ID NO:5, or the complement thereof; ii) mRNA molecule, or the complement thereof, corresponding to: position 754 according to SEQ ID NO:43, or the complement thereof; position 821 according to SEQ ID NO:44, or the complement thereof; position 786 according to SEQ ID NO:45, or the complement thereof; position 768 according to SEQ ID NO:46, or the complement thereof; position 743 according to SEQ ID NO:47, or the complement thereof; position 536 according to SEQ ID NO:48, or the complement thereof; position 529 according to SEQ ID NO:49, or the complement thereof; or position 768 according to SEQ ID NO:50, or the complement thereof; and/or iii) cDNA molecule, or the complement thereof, corresponding to: position 754 according to SEQ ID NO:123, or the complement thereof; position 821 according to SEQ ID NO:124, or the complement thereof; position 786 according to SEQ ID NO:125, or the complement thereof; position 768 according to SEQ ID NO:126, or the complement thereof; position 743 according to SEQ ID NO:127, or the complement thereof; position 536 according to SEQ ID NO:128, or the complement thereof; position 529 according to SEQ ID NO:129, or the complement thereof; or position 768 according to SEQ ID NO:130, or the complement thereof; and c) determining whether the extension product of the primer comprises: a thymine at a position corresponding to position 4,277 according to SEQ ID NO:5, or the complement thereof; a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a uracil at a position corresponding to position 768 according to SEQ ID NO:50, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; and/or a thymine at a position corresponding to position 768 according to SEQ ID NO:130, or the complement thereof.

In some embodiments, the determining step, detecting step, or sequence analysis comprises: a) contacting the biological sample with a primer hybridizing to a portion of the nucleotide sequence of the ANGPTL7: i) genomic nucleic acid molecule, or the complement thereof, that is proximate to a position corresponding to position 4,311 according to SEQ ID NO:6, or the complement thereof; ii) mRNA molecule, or the complement thereof, that is proximate to a position corresponding to: position 788 according to SEQ ID NO:51, or the complement thereof; position 855 according to SEQ ID NO:52, or the complement thereof; position 820 according to SEQ ID NO:53, or the complement thereof; position 802 according to SEQ ID NO:54, or the complement thereof; position 777 according to SEQ ID NO:55, or the complement thereof; position 570 according to SEQ ID NO:56, or the complement thereof; position 563 according to SEQ ID NO:57, or the complement thereof; or position 802 according to SEQ ID NO:58, or the complement thereof; and/or iii) cDNA molecule, or the complement thereof, that is proximate to a position corresponding to: position 788 according to SEQ ID NO:131, or the complement thereof; position 855 according to SEQ ID NO:132, or the complement thereof; position 820 according to SEQ ID NO:133, or the complement thereof; position 802 according to SEQ ID NO:134, or the complement thereof; position 777 according to SEQ ID NO:135, or the complement thereof; position 570 according to SEQ ID NO:136, or the complement thereof; position 563 according to SEQ ID NO:137, or the complement thereof; or position 802 according to SEQ ID NO:138, or the complement thereof; b) extending the primer at least through the position of the nucleotide sequence of the ANGPTL7: i) genomic nucleic acid molecule, or the complement thereof, corresponding to position 4,311 according to SEQ ID NO:6, or the complement thereof; ii) mRNA molecule, or the complement thereof, corresponding to: position 788 according to SEQ ID NO:51, or the complement thereof; position 855 according to SEQ ID NO:52, or the complement thereof; position 820 according to SEQ ID NO:53, or the complement thereof; position 802 according to SEQ ID NO:54, or the complement thereof; position 777 according to SEQ ID NO:55, or the complement thereof; position 570 according to SEQ ID NO:56, or the complement thereof; position 563 according to SEQ ID NO:57, or the complement thereof; or position 802 according to SEQ ID NO:58, or the complement thereof; and/or iii) cDNA molecule, or the complement thereof, corresponding to: position 788 according to SEQ ID NO:131, or the complement thereof; position 855 according to SEQ ID NO:132, or the complement thereof; position 820 according to SEQ ID NO:133, or the complement thereof; position 802 according to SEQ ID NO:134, or the complement thereof; position 777 according to SEQ ID NO:135, or the complement thereof; position 570 according to SEQ ID NO:136, or the complement thereof; position 563 according to SEQ ID NO:137, or the complement thereof; or position 802 according to SEQ ID NO:138, or the complement thereof; and c) determining whether the extension product of the primer comprises: an adenine at a position corresponding to position 4,311 according to SEQ ID NO:6, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; and/or an adenine at a position corresponding to position 802 according to SEQ ID NO:138, or the complement thereof.

In some embodiments, the determining step, detecting step, or sequence analysis comprises: a) contacting the biological sample with a primer hybridizing to a portion of the nucleotide sequence of the ANGPTL7: i) genomic nucleic acid molecule, or the complement thereof, that is proximate to a position corresponding to position 4,322 according to SEQ ID NO:7, or the complement thereof; ii) mRNA molecule, or the complement thereof, that is proximate to a position corresponding to: position 799 according to SEQ ID NO:59, or the complement thereof; position 866 according to SEQ ID NO:60, or the complement thereof; position 831 according to SEQ ID NO:61, or the complement thereof; position 813 according to SEQ ID NO:62, or the complement thereof; position 788 according to SEQ ID NO:63, or the complement thereof; position 581 according to SEQ ID NO:64, or the complement thereof; position 574 according to SEQ ID NO:65, or the complement thereof; or position 813 according to SEQ ID NO:66, or the complement thereof; and/or iii) cDNA molecule, or the complement thereof, that is proximate to a position corresponding to: position 799 according to SEQ ID NO:139, or the complement thereof; position 866 according to SEQ ID NO:140, or the complement thereof; position 831 according to SEQ ID NO:141, or the complement thereof; position 813 according to SEQ ID NO:142, or the complement thereof; position 788 according to SEQ ID NO:143, or the complement thereof; position 581 according to SEQ ID NO:144, or the complement thereof; position 574 according to SEQ ID NO:145, or the complement thereof; or position 813 according to SEQ ID NO:146, or the complement thereof; b) extending the primer at least through the position of the nucleotide sequence of the ANGPTL7: i) genomic nucleic acid molecule, or the complement thereof, corresponding to position 4,322 according to SEQ ID NO:7, or the complement thereof; ii) mRNA molecule, or the complement thereof, corresponding to: position 799 according to SEQ ID NO:59, or the complement thereof; position 866 according to SEQ ID NO:60, or the complement thereof; position 831 according to SEQ ID NO:61, or the complement thereof; position 813 according to SEQ ID NO:62, or the complement thereof; position 788 according to SEQ ID NO:63, or the complement thereof; position 581 according to SEQ ID NO:64, or the complement thereof; position 574 according to SEQ ID NO:65, or the complement thereof; or position 813 according to SEQ ID NO:66, or the complement thereof; and/or iii) cDNA molecule, or the complement thereof, corresponding to: position 799 according to SEQ ID NO:139, or the complement thereof; position 866 according to SEQ ID NO:140, or the complement thereof; position 831 according to SEQ ID NO:141, or the complement thereof; position 813 according to SEQ ID NO:142, or the complement thereof; position 788 according to SEQ ID NO:143, or the complement thereof; position 581 according to SEQ ID NO:144, or the complement thereof; position 574 according to SEQ ID NO:145, or the complement thereof; or position 813 according to SEQ ID NO:146, or the complement thereof; and c) determining whether the extension product of the primer comprises: a cytosine at a position corresponding to position 4,322 according to SEQ ID NO:7, or the complement thereof; a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; and/or a cytosine at a position corresponding to position 813 according to SEQ ID NO:146, or the complement thereof.

In some embodiments, the determining step, detecting step, or sequence analysis comprises: a) contacting the biological sample with a primer hybridizing to a portion of the nucleotide sequence of the ANGPTL7: i) genomic nucleic acid molecule, or the complement thereof, that is proximate to a position corresponding to position 5,125 according to SEQ ID NO:8, or the complement thereof; ii) mRNA molecule, or the complement thereof, that is proximate to a position corresponding to: position 916 according to SEQ ID NO:67, or the complement thereof; position 983 according to SEQ ID NO:68, or the complement thereof; position 948 according to SEQ ID NO:69, or the complement thereof; position 930 according to SEQ ID NO:70, or the complement thereof; position 905 according to SEQ ID NO:71, or the complement thereof; position 698 according to SEQ ID NO:72, or the complement thereof; position 691 according to SEQ ID NO:73, or the complement thereof; or position 930 according to SEQ ID NO:74, or the complement thereof; and/or iii) cDNA molecule, or the complement thereof, that is proximate to a position corresponding to: position 916 according to SEQ ID NO:147, or the complement thereof; position 983 according to SEQ ID NO:148, or the complement thereof; position 948 according to SEQ ID NO:149, or the complement thereof; position 930 according to SEQ ID NO:150, or the complement thereof; position 905 according to SEQ ID NO:151, or the complement thereof; position 698 according to SEQ ID NO:152, or the complement thereof; position 691 according to SEQ ID NO:153, or the complement thereof; or position 930 according to SEQ ID NO:154, or the complement thereof; b) extending the primer at least through the position of the nucleotide sequence of the ANGPTL7: i) genomic nucleic acid molecule, or the complement thereof, corresponding to position 5,125 according to SEQ ID NO:8, or the complement thereof; ii) mRNA molecule, or the complement thereof, corresponding to: position 916 according to SEQ ID NO:67, or the complement thereof; position 983 according to SEQ ID NO:68, or the complement thereof; position 948 according to SEQ ID NO:69, or the complement thereof; position 930 according to SEQ ID NO:70, or the complement thereof; position 905 according to SEQ ID NO:71, or the complement thereof; position 698 according to SEQ ID NO:72, or the complement thereof; position 691 according to SEQ ID NO:73, or the complement thereof; or position 930 according to SEQ ID NO:74, or the complement thereof; and/or iii) cDNA molecule, or the complement thereof, corresponding to: position 916 according to SEQ ID NO:147, or the complement thereof; position 983 according to SEQ ID NO:148, or the complement thereof; position 948 according to SEQ ID NO:149, or the complement thereof; position 930 according to SEQ ID NO:150, or the complement thereof; position 905 according to SEQ ID NO:151, or the complement thereof; position 698 according to SEQ ID NO:152, or the complement thereof; position 691 according to SEQ ID NO:153, or the complement thereof; or position 930 according to SEQ ID NO:154, or the complement thereof; and c) determining whether the extension product of the primer comprises: a thymine at a position corresponding to position 5,125 according to SEQ ID NO:8, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; or a thymine at a position corresponding to position 930 according to SEQ ID NO:154, or the complement thereof.

In some embodiments, the determining step, detecting step, or sequence analysis comprises: a) contacting the biological sample with a primer hybridizing to a portion of the nucleotide sequence of the ANGPTL7: i) genomic nucleic acid molecule, or the complement thereof, that is proximate to a position corresponding to position 5,176 according to SEQ ID NO:9, or the complement thereof; ii) mRNA molecule, or the complement thereof, that is proximate to a position corresponding to: position 967 according to SEQ ID NO:75, or the complement thereof; position 1,034 according to SEQ ID NO:76, or the complement thereof; position 999 according to SEQ ID NO:77, or the complement thereof; position 981 according to SEQ ID NO:78, or the complement thereof; position 956 according to SEQ ID NO:79, or the complement thereof; position 749 according to SEQ ID NO:80, or the complement thereof; position 742 according to SEQ ID NO:81, or the complement thereof; or position 981 according to SEQ ID NO:82, or the complement thereof; and/or iii) cDNA molecule, or the complement thereof, that is proximate to a position corresponding to: position 967 according to SEQ ID NO:155, or the complement thereof; position 1,034 according to SEQ ID NO:156, or the complement thereof; position 999 according to SEQ ID NO:157, or the complement thereof; position 981 according to SEQ ID NO:158, or the complement thereof; position 956 according to SEQ ID NO:159, or the complement thereof; position 749 according to SEQ ID NO:160, or the complement thereof; position 742 according to SEQ ID NO:161, or the complement thereof; or position 981 according to SEQ ID NO:162, or the complement thereof; b) extending the primer at least through the position of the nucleotide sequence of the ANGPTL7: i) genomic nucleic acid molecule, or the complement thereof, corresponding to position 5,176 according to SEQ ID NO:9, or the complement thereof; ii) mRNA molecule, or the complement thereof, corresponding to: position 967 according to SEQ ID NO:75, or the complement thereof; position 1,034 according to SEQ ID NO:76, or the complement thereof; position 999 according to SEQ ID NO:77, or the complement thereof; position 981 according to SEQ ID NO:78, or the complement thereof; position 956 according to SEQ ID NO:79, or the complement thereof; position 749 according to SEQ ID NO:80, or the complement thereof; position 742 according to SEQ ID NO:81, or the complement thereof; or position 981 according to SEQ ID NO:82, or the complement thereof; and/or iii) cDNA molecule, or the complement thereof, corresponding to: position 967 according to SEQ ID NO:155, or the complement thereof; position 1,034 according to SEQ ID NO:156, or the complement thereof; position 999 according to SEQ ID NO:157, or the complement thereof; position 981 according to SEQ ID NO:158, or the complement thereof; position 956 according to SEQ ID NO:159, or the complement thereof; position 749 according to SEQ ID NO:160, or the complement thereof; position 742 according to SEQ ID NO:161, or the complement thereof; or position 981 according to SEQ ID NO:162, or the complement thereof; and c) determining whether the extension product of the primer comprises: a thymine at a position corresponding to position 5,176 according to SEQ ID NO:9, or the complement thereof; a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; a uracil at a position corresponding to position 981 according to SEQ ID NO:82, or the complement thereof; a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; and/or a thymine at a position corresponding to position 981 according to SEQ ID NO:162, or the complement thereof.

In some embodiments, the determining step, detecting step, or sequence analysis comprises: a) contacting the biological sample with a primer hybridizing to a portion of the nucleotide sequence of the ANGPTL7: i) genomic nucleic acid molecule, or the complement thereof, that is proximate to a position corresponding to position 5,232 according to SEQ ID NO:10, or the complement thereof; ii) mRNA molecule, or the complement thereof, that is proximate to a position corresponding to: position 1,023 according to SEQ ID NO:83, or the complement thereof; position 1,090 according to SEQ ID NO:84, or the complement thereof; position 1,055 according to SEQ ID NO:85, or the complement thereof; position 1,037 according to SEQ ID NO:86, or the complement thereof; position 1,012 according to SEQ ID NO:87, or the complement thereof; position 805 according to SEQ ID NO:88, or the complement thereof; position 798 according to SEQ ID NO:89, or the complement thereof; or position 1,037 according to SEQ ID NO:90, or the complement thereof; and/or iii) cDNA molecule, or the complement thereof, that is proximate to a position corresponding to: position 1,023 according to SEQ ID NO:163, or the complement thereof; position 1,090 according to SEQ ID NO:164, or the complement thereof; position 1,055 according to SEQ ID NO:165, or the complement thereof; position 1,037 according to SEQ ID NO:166, or the complement thereof; position 1,012 according to SEQ ID NO:167, or the complement thereof; position 805 according to SEQ ID NO:168, or the complement thereof; position 798 according to SEQ ID NO:169, or the complement thereof; or position 1,037 according to SEQ ID NO:170, or the complement thereof; b) extending the primer at least through the position of the nucleotide sequence of the ANGPTL7: i) genomic nucleic acid molecule, or the complement thereof, corresponding to position 5,232 according to SEQ ID NO:10, or the complement thereof; ii) mRNA molecule, or the complement thereof, corresponding to: position 1,023 according to SEQ ID NO:83, or the complement thereof; position 1,090 according to SEQ ID NO:84, or the complement thereof; position 1,055 according to SEQ ID NO:85, or the complement thereof; position 1,037 according to SEQ ID NO:86, or the complement thereof; position 1,012 according to SEQ ID NO:87, or the complement thereof; position 805 according to SEQ ID NO:88, or the complement thereof; position 798 according to SEQ ID NO:89, or the complement thereof; or position 1,037 according to SEQ ID NO:90, or the complement thereof; and/or iii) cDNA molecule, or the complement thereof, corresponding to: position 1,023 according to SEQ ID NO:163, or the complement thereof; position 1,090 according to SEQ ID NO:164, or the complement thereof; position 1,055 according to SEQ ID NO:165, or the complement thereof; position 1,037 according to SEQ ID NO:166, or the complement thereof; position 1,012 according to SEQ ID NO:167, or the complement thereof; position 805 according to SEQ ID NO:168, or the complement thereof; position 798 according to SEQ ID NO:169, or the complement thereof; or position 1,037 according to SEQ ID NO:170, or the complement thereof; and c) determining whether the extension product of the primer comprises: an adenine at a position corresponding to position 5,232 according to SEQ ID NO:10, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; and/or an adenine at a position corresponding to position 1,037 according to SEQ ID NO:170, or the complement thereof.

In some embodiments, the determining step, detecting step, or sequence analysis comprises: a) contacting the biological sample with a primer hybridizing to a portion of the nucleotide sequence of the ANGPTL7 genomic nucleic acid molecule, or the complement thereof, that is proximate to a position corresponding to: position 4,229 according to SEQ ID NO:2, or the complement thereof; position 4,269 according to SEQ ID NO:3, or the complement thereof; position 4,273 according to SEQ ID NO:4, or the complement thereof; position 4,277 according to SEQ ID NO:5, or the complement thereof; position 4,311 according to SEQ ID NO:6, or the complement thereof; position 4,322 according to SEQ ID NO:7, or the complement thereof; position 5,125 according to SEQ ID NO:8, or the complement thereof; position 5,176 according to SEQ ID NO:9, or the complement thereof; or position 5,232 according to SEQ ID NO:10, or the complement thereof; b) extending the primer at least through the position of the nucleotide sequence of the ANGPTL7 genomic nucleic acid molecule, or the complement thereof, corresponding to: position 4,229 according to SEQ ID NO:2, or the complement thereof; position 4,269 according to SEQ ID NO:3, or the complement thereof; position 4,273 according to SEQ ID NO:4, or the complement thereof; position 4,277 according to SEQ ID NO:5, or the complement thereof; position 4,311 according to SEQ ID NO:6, or the complement thereof; position 4,322 according to SEQ ID NO:7, or the complement thereof; position 5,125 according to SEQ ID NO:8, or the complement thereof; position 5,176 according to SEQ ID NO:9, or the complement thereof; or position corresponding to position 5,232 according to SEQ ID NO:10, or the complement thereof and c) determining whether the extension product of the primer comprises: an adenine at a position corresponding to position 4,229 according to SEQ ID NO:2, or the complement thereof; an adenine at a position corresponding to position 4,269 according to SEQ ID NO:3, or the complement thereof; a thymine at a position corresponding to position 4,273 according to SEQ ID NO:4, or the complement thereof; a thymine at a position corresponding to position 4,277 according to SEQ ID NO:5, or the complement thereof; an adenine at a position corresponding to position 4,311 according to SEQ ID NO:6, or the complement thereof; a cytosine at a position corresponding to position 4,322 according to SEQ ID NO:7, or the complement thereof; a thymine at a position corresponding to position 5,125 according to SEQ ID NO:8, or the complement thereof; a thymine at a position corresponding to position 5,176 according to SEQ ID NO:9, or the complement thereof; an adenine at a position corresponding to position 5,232 according to SEQ ID NO:10, or the complement thereof; or an adenine at a position corresponding to position 5,232 according to SEQ ID NO:10, or the complement thereof.

In some embodiments, the determining step, detecting step, or sequence analysis comprises: a) contacting the biological sample with a primer hybridizing to a portion of the nucleotide sequence of the ANGPTL7 mRNA molecule, or the complement thereof, that is proximate to a position corresponding to: position 706 according to SEQ ID NO:19, or the complement thereof; position 773 according to SEQ ID NO:20, or the complement thereof; position 738 according to SEQ ID NO:21, or the complement thereof; position 720 according to SEQ ID NO:22, or the complement thereof; position 695 according to SEQ ID NO:23, or the complement thereof; position 488 according to SEQ ID NO:24, or the complement thereof; position 481 according to SEQ ID NO:25, or the complement thereof; position 720 according to SEQ ID NO:26, or the complement thereof; position 746 according to SEQ ID NO:27, or the complement thereof; position 812 according to SEQ ID NO:28, or the complement thereof; position 778 according to SEQ ID NO:29, or the complement thereof; position 760 according to SEQ ID NO:30, or the complement thereof; position 735 according to SEQ ID NO:31, or the complement thereof; position 528 according to SEQ ID NO:32, or the complement thereof; position 521 according to SEQ ID NO:33, or the complement thereof; position 760 according to SEQ ID NO:34, or the complement thereof; position 750 according to SEQ ID NO:35, or the complement thereof; position 817 according to SEQ ID NO:36, or the complement thereof; position 782 according to SEQ ID NO:37, or the complement thereof; position 764 according to SEQ ID NO:38, or the complement thereof; position 739 according to SEQ ID NO:39, or the complement thereof; position 532 according to SEQ ID NO:40, or the complement thereof; position 525 according to SEQ ID NO:41, or the complement thereof; position 764 according to SEQ ID NO:42, or the complement thereof; position 754 according to SEQ ID NO:43, or the complement thereof; position 821 according to SEQ ID NO:44, or the complement thereof; position 786 according to SEQ ID NO:45, or the complement thereof; position 768 according to SEQ ID NO:46, or the complement thereof; position 743 according to SEQ ID NO:47, or the complement thereof; position 536 according to SEQ ID NO:48, or the complement thereof; position 529 according to SEQ ID NO:49, or the complement thereof; position 768 according to SEQ ID NO:50, or the complement thereof; position 788 according to SEQ ID NO:51, or the complement thereof; position 855 according to SEQ ID NO:52, or the complement thereof; position 820 according to SEQ ID NO:53, or the complement thereof; position 802 according to SEQ ID NO:54, or the complement thereof; position 777 according to SEQ ID NO:55, or the complement thereof; position 570 according to SEQ ID NO:56, or the complement thereof; position 563 according to SEQ ID NO:57, or the complement thereof; position 802 according to SEQ ID NO:58, or the complement thereof; position 799 according to SEQ ID NO:59, or the complement thereof; position 866 according to SEQ ID NO:60, or the complement thereof; position 831 according to SEQ ID NO:61, or the complement thereof; position 813 according to SEQ ID NO:62, or the complement thereof; position 788 according to SEQ ID NO:63, or the complement thereof; position 581 according to SEQ ID NO:64, or the complement thereof; position 574 according to SEQ ID NO:65, or the complement thereof; position 813 according to SEQ ID NO:66, or the complement thereof; position 916 according to SEQ ID NO:67, or the complement thereof; position 983 according to SEQ ID NO:68, or the complement thereof; position 948 according to SEQ ID NO:69, or the complement thereof; position 930 according to SEQ ID NO:70, or the complement thereof; position 905 according to SEQ ID NO:71, or the complement thereof; position 698 according to SEQ ID NO:72, or the complement thereof; position 691 according to SEQ ID NO:73, or the complement thereof; position 930 according to SEQ ID NO:74, or the complement thereof; position 967 according to SEQ ID NO:75, or the complement thereof; position 1,034 according to SEQ ID NO:76, or the complement thereof; position 999 according to SEQ ID NO:77, or the complement thereof; position 981 according to SEQ ID NO:78, or the complement thereof; position 956 according to SEQ ID NO:79, or the complement thereof; position 749 according to SEQ ID NO:80, or the complement thereof; position 742 according to SEQ ID NO:81, or the complement thereof; position 981 according to SEQ ID NO:82, or the complement thereof; position 1,023 according to SEQ ID NO:83, or the complement thereof; position 1,090 according to SEQ ID NO:84, or the complement thereof; position 1,055 according to SEQ ID NO:85, or the complement thereof; position 1,037 according to SEQ ID NO:86, or the complement thereof; position 1,012 according to SEQ ID NO:87, or the complement thereof; position 805 according to SEQ ID NO:88, or the complement thereof; position 798 according to SEQ ID NO:89, or the complement thereof; or position 1,037 according to SEQ ID NO:90, or the complement thereof; b) extending the primer at least through the position of the nucleotide sequence of the ANGPTL7 mRNA molecule corresponding to: position 706 according to SEQ ID NO:19, or the complement thereof; position 773 according to SEQ ID NO:20, or the complement thereof; position 738 according to SEQ ID NO:21, or the complement thereof; position 720 according to SEQ ID NO:22, or the complement thereof; position 695 according to SEQ ID NO:23, or the complement thereof; position 488 according to SEQ ID NO:24, or the complement thereof; position 481 according to SEQ ID NO:25, or the complement thereof; position 720 according to SEQ ID NO:26, or the complement thereof; position 746 according to SEQ ID NO:27, or the complement thereof; position 812 according to SEQ ID NO:28, or the complement thereof; position 778 according to SEQ ID NO:29, or the complement thereof; position 760 according to SEQ ID NO:30, or the complement thereof; position 735 according to SEQ ID NO:31, or the complement thereof; position 528 according to SEQ ID NO:32, or the complement thereof; position 521 according to SEQ ID NO:33, or the complement thereof; position 760 according to SEQ ID NO:34, or the complement thereof; position 750 according to SEQ ID NO:35, or the complement thereof; position 817 according to SEQ ID NO:36, or the complement thereof; position 782 according to SEQ ID NO:37, or the complement thereof; position 764 according to SEQ ID NO:38, or the complement thereof; position 739 according to SEQ ID NO:39, or the complement thereof; position 532 according to SEQ ID NO:40, or the complement thereof; position 525 according to SEQ ID NO:41, or the complement thereof; position 764 according to SEQ ID NO:42, or the complement thereof; position 754 according to SEQ ID NO:43, or the complement thereof; position 821 according to SEQ ID NO:44, or the complement thereof; position 786 according to SEQ ID NO:45, or the complement thereof; position 768 according to SEQ ID NO:46, or the complement thereof; position 743 according to SEQ ID NO:47, or the complement thereof; position 536 according to SEQ ID NO:48, or the complement thereof; position 529 according to SEQ ID NO:49, or the complement thereof; position 768 according to SEQ ID NO:50, or the complement thereof; position 788 according to SEQ ID NO:51, or the complement thereof; position 855 according to SEQ ID NO:52, or the complement thereof; position 820 according to SEQ ID NO:53, or the complement thereof; position 802 according to SEQ ID NO:54, or the complement thereof; position 777 according to SEQ ID NO:55, or the complement thereof; position 570 according to SEQ ID NO:56, or the complement thereof; position 563 according to SEQ ID NO:57, or the complement thereof; position 802 according to SEQ ID NO:58, or the complement thereof; position 799 according to SEQ ID NO:59, or the complement thereof; position 866 according to SEQ ID NO:60, or the complement thereof; position 831 according to SEQ ID NO:61, or the complement thereof; position 813 according to SEQ ID NO:62, or the complement thereof; position 788 according to SEQ ID NO:63, or the complement thereof; position 581 according to SEQ ID NO:64, or the complement thereof; position 574 according to SEQ ID NO:65, or the complement thereof; position 813 according to SEQ ID NO:66, or the complement thereof; position 916 according to SEQ ID NO:67, or the complement thereof; position 983 according to SEQ ID NO:68, or the complement thereof; position 948 according to SEQ ID NO:69, or the complement thereof; position 930 according to SEQ ID NO:70, or the complement thereof; position 905 according to SEQ ID NO:71, or the complement thereof; position 698 according to SEQ ID NO:72, or the complement thereof; position 691 according to SEQ ID NO:73, or the complement thereof; position 930 according to SEQ ID NO:74, or the complement thereof; position 967 according to SEQ ID NO:75, or the complement thereof; position 1,034 according to SEQ ID NO:76, or the complement thereof; position 999 according to SEQ ID NO:77, or the complement thereof; position 981 according to SEQ ID NO:78, or the complement thereof; position 956 according to SEQ ID NO:79, or the complement thereof; position 749 according to SEQ ID NO:80, or the complement thereof; position 742 according to SEQ ID NO:81, or the complement thereof; position 981 according to SEQ ID NO:82, or the complement thereof; position 1,023 according to SEQ ID NO:83, or the complement thereof; position 1,090 according to SEQ ID NO:84, or the complement thereof; position 1,055 according to SEQ ID NO:85, or the complement thereof; position 1,037 according to SEQ ID NO:86, or the complement thereof; position 1,012 according to SEQ ID NO:87, or the complement thereof; position 805 according to SEQ ID NO:88, or the complement thereof; position 798 according to SEQ ID NO:89, or the complement thereof; or position 1,037 according to SEQ ID NO:90, or the complement thereof and c) determining whether the extension product of the primer comprises: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a uracil at a position corresponding to position 768 according to SEQ ID NO:50, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof; a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; a uracil at a position corresponding to position 981 according to SEQ ID NO:82, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof.

In some embodiments, the determining step, detecting step, or sequence analysis comprises: a) contacting the biological sample with a primer hybridizing to a portion of the nucleotide sequence of the ANGPTL7 cDNA molecule, or the complement thereof, that is proximate to a position corresponding to: position 706 according to SEQ ID NO:99, or the complement thereof; position 773 according to SEQ ID NO:100, or the complement thereof; position 738 according to SEQ ID NO:101, or the complement thereof; position 720 according to SEQ ID NO:102, or the complement thereof; position 695 according to SEQ ID NO:103, or the complement thereof; position 488 according to SEQ ID NO:104, or the complement thereof; position 481 according to SEQ ID NO:105, or the complement thereof; position 720 according to SEQ ID NO:106, or the complement thereof; position 746 according to SEQ ID NO:107, or the complement thereof; position 812 according to SEQ ID NO:108, or the complement thereof; position 778 according to SEQ ID NO:109, or the complement thereof; position 760 according to SEQ ID NO:110, or the complement thereof; position 735 according to SEQ ID NO:111, or the complement thereof; position 528 according to SEQ ID NO:112, or the complement thereof; position 529 according to SEQ ID NO:113, or the complement thereof; position 760 according to SEQ ID NO:114, or the complement thereof; position 750 according to SEQ ID NO:115, or the complement thereof; position 817 according to SEQ ID NO:116, or the complement thereof; position 782 according to SEQ ID NO:117, or the complement thereof; position 764 according to SEQ ID NO:118, or the complement thereof; position 739 according to SEQ ID NO:119, or the complement thereof; position 532 according to SEQ ID NO:120, or the complement thereof; position 525 according to SEQ ID NO:121, or the complement thereof; position 764 according to SEQ ID NO:122, or the complement thereof; position 754 according to SEQ ID NO:123, or the complement thereof; position 821 according to SEQ ID NO:124, or the complement thereof; position 786 according to SEQ ID NO:125, or the complement thereof; position 768 according to SEQ ID NO:126, or the complement thereof; position 743 according to SEQ ID NO:127, or the complement thereof; position 536 according to SEQ ID NO:128, or the complement thereof; position 529 according to SEQ ID NO:129, or the complement thereof; position 768 according to SEQ ID NO:130, or the complement thereof; position 788 according to SEQ ID NO:131, or the complement thereof; position 855 according to SEQ ID NO:132, or the complement thereof; position 820 according to SEQ ID NO:133, or the complement thereof; position 802 according to SEQ ID NO:134, or the complement thereof; position 777 according to SEQ ID NO:135, or the complement thereof; position 570 according to SEQ ID NO:136, or the complement thereof; position 563 according to SEQ ID NO:137, or the complement thereof; position 802 according to SEQ ID NO:138, or the complement thereof; position 799 according to SEQ ID NO:139, or the complement thereof; position 866 according to SEQ ID NO:140, or the complement thereof; position 831 according to SEQ ID NO:141, or the complement thereof; position 813 according to SEQ ID NO:142, or the complement thereof; position 788 according to SEQ ID NO:143, or the complement thereof; position 581 according to SEQ ID NO:144, or the complement thereof; position 574 according to SEQ ID NO:145, or the complement thereof; position 813 according to SEQ ID NO:146, or the complement thereof; position 916 according to SEQ ID NO:147, or the complement thereof; position 983 according to SEQ ID NO:148, or the complement thereof; position 948 according to SEQ ID NO:149, or the complement thereof; position 930 according to SEQ ID NO:150, or the complement thereof; position 905 according to SEQ ID NO:151, or the complement thereof; position 698 according to SEQ ID NO:152, or the complement thereof; position 691 according to SEQ ID NO:153, or the complement thereof; position 930 according to SEQ ID NO:154, or the complement thereof; position 1,034 according to SEQ ID NO:156, or the complement thereof; position 999 according to SEQ ID NO:157, or the complement thereof; position 981 according to SEQ ID NO:158, or the complement thereof; position 956 according to SEQ ID NO:159, or the complement thereof; position 749 according to SEQ ID NO:160, or the complement thereof; position 742 according to SEQ ID NO:161, or the complement thereof; position 981 according to SEQ ID NO:162, or the complement thereof; position 1,023 according to SEQ ID NO:163, or the complement thereof; position 1,090 according to SEQ ID NO:164, or the complement thereof; position 1,055 according to SEQ ID NO:165, or the complement thereof; position 1,037 according to SEQ ID NO:166, or the complement thereof; position 1,012 according to SEQ ID NO:167, or the complement thereof; position 805 according to SEQ ID NO:168, or the complement thereof; position 798 according to SEQ ID NO:169, or the complement thereof; or position 1,037 according to SEQ ID NO:170, or the complement thereof; b) extending the primer at least through the position of the nucleotide sequence of the ANGPTL7 cDNA molecule corresponding to: position 706 according to SEQ ID NO:99, or the complement thereof; position 773 according to SEQ ID NO:100, or the complement thereof; position 738 according to SEQ ID NO:101, or the complement thereof; position 720 according to SEQ ID NO:102, or the complement thereof; position 695 according to SEQ ID NO:103, or the complement thereof; position 488 according to SEQ ID NO:104, or the complement thereof; position 481 according to SEQ ID NO:105, or the complement thereof; position 720 according to SEQ ID NO:106, or the complement thereof; position 746 according to SEQ ID NO:107, or the complement thereof; position 812 according to SEQ ID NO:108, or the complement thereof; position 778 according to SEQ ID NO:109, or the complement thereof; position 760 according to SEQ ID NO:110, or the complement thereof; position 735 according to SEQ ID NO:111, or the complement thereof; position 528 according to SEQ ID NO:112, or the complement thereof; position 529 according to SEQ ID NO:113, or the complement thereof; position 760 according to SEQ ID NO:114, or the complement thereof; position 750 according to SEQ ID NO:115, or the complement thereof; position 817 according to SEQ ID NO:116, or the complement thereof; position 782 according to SEQ ID NO:117, or the complement thereof; position 764 according to SEQ ID NO:118, or the complement thereof; position 739 according to SEQ ID NO:119, or the complement thereof; position 532 according to SEQ ID NO:120, or the complement thereof; position 525 according to SEQ ID NO:121, or the complement thereof; position 764 according to SEQ ID NO:122, or the complement thereof; position 754 according to SEQ ID NO:123, or the complement thereof; position 821 according to SEQ ID NO:124, or the complement thereof; position 786 according to SEQ ID NO:125, or the complement thereof; position 768 according to SEQ ID NO:126, or the complement thereof; position 743 according to SEQ ID NO:127, or the complement thereof; position 536 according to SEQ ID NO:128, or the complement thereof; position 529 according to SEQ ID NO:129, or the complement thereof; position 768 according to SEQ ID NO:130, or the complement thereof; position 788 according to SEQ ID NO:131, or the complement thereof; position 855 according to SEQ ID NO:132, or the complement thereof; position 820 according to SEQ ID NO:133, or the complement thereof; position 802 according to SEQ ID NO:134, or the complement thereof; position 777 according to SEQ ID NO:135, or the complement thereof; position 570 according to SEQ ID NO:136, or the complement thereof; position 563 according to SEQ ID NO:137, or the complement thereof; position 802 according to SEQ ID NO:138, or the complement thereof; position 799 according to SEQ ID NO:139, or the complement thereof; position 866 according to SEQ ID NO:140, or the complement thereof; position 831 according to SEQ ID NO:141, or the complement thereof; position 813 according to SEQ ID NO:142, or the complement thereof; position 788 according to SEQ ID NO:143, or the complement thereof; position 581 according to SEQ ID NO:144, or the complement thereof; position 574 according to SEQ ID NO:145, or the complement thereof; position 813 according to SEQ ID NO:146, or the complement thereof; position 916 according to SEQ ID NO:147, or the complement thereof; position 983 according to SEQ ID NO:148, or the complement thereof; position 948 according to SEQ ID NO:149, or the complement thereof; position 930 according to SEQ ID NO:150, or the complement thereof; position 905 according to SEQ ID NO:151, or the complement thereof; position 698 according to SEQ ID NO:152, or the complement thereof; position 691 according to SEQ ID NO:153, or the complement thereof; position 930 according to SEQ ID NO:154, or the complement thereof; position 967 according to SEQ ID NO:155, or the complement thereof; position 1,034 according to SEQ ID NO:156, or the complement thereof; position 999 according to SEQ ID NO:157, or the complement thereof; position 981 according to SEQ ID NO:158, or the complement thereof; position 956 according to SEQ ID NO:159, or the complement thereof; position 749 according to SEQ ID NO:160, or the complement thereof; position 742 according to SEQ ID NO:161, or the complement thereof; position 981 according to SEQ ID NO:162, or the complement thereof; and c) determining whether the extension product of the primer comprises: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; an adenine at a position corresponding to position 760 according to SEQ ID NO:114, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; a thymine at a position corresponding to position 764 according to SEQ ID NO:122, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; a thymine at a position corresponding to position 768 according to SEQ ID NO:130, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; an adenine at a position corresponding to position 802 according to SEQ ID NO:138, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; a cytosine at a position corresponding to position 813 according to SEQ ID NO:146, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; a thymine at a position corresponding to position 930 according to SEQ ID NO:154, or the complement thereof; a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; a thymine at a position corresponding to position 981 according to SEQ ID NO:162, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof.

In some embodiments, the entire nucleic acid molecule is sequenced. In some embodiments, only an ANGPTL7 genomic nucleic acid molecule is analyzed. In some embodiments, only an ANGPTL7 mRNA is analyzed. In some embodiments, only an ANGPTL7 cDNA obtained from ANGPTL7 mRNA is analyzed.

In some embodiments, the determining step, detecting step, or sequence analysis comprises: a) amplifying at least a portion of the ANGPTL7 nucleic acid molecule, or the complement thereof, in the biological sample, wherein the amplified portion comprises: an adenine at a position corresponding to position 4,229 according to SEQ ID NO:2, or the complement thereof; an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 4,229 according to SEQ ID NO:2, or the complement thereof; an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof; and d) detecting the detectable label.

In some embodiments, the determining step, detecting step, or sequence analysis comprises: a) amplifying at least a portion of the ANGPTL7 nucleic acid molecule, or the complement thereof, in the biological sample, wherein the amplified portion comprises: an adenine at a position corresponding to position 4,269 according to SEQ ID NO:3, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; an adenine at a position corresponding to position 760 according to SEQ ID NO:114, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 4,269 according to SEQ ID NO:3, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; an adenine at a position corresponding to position 760 according to SEQ ID NO:114, or the complement thereof; and d) detecting the detectable label.

In some embodiments, the determining step, detecting step, or sequence analysis comprises: a) amplifying at least a portion of the ANGPTL7 nucleic acid molecule, or the complement thereof, in the biological sample, wherein the amplified portion comprises: a thymine at a position corresponding to position 4,273 according to SEQ ID NO:4, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; a thymine at a position corresponding to position 764 according to SEQ ID NO:122, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a thymine at a position corresponding to position 4,273 according to SEQ ID NO:4, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; a thymine at a position corresponding to position 764 according to SEQ ID NO:122, or the complement thereof; and d) detecting the detectable label.

In some embodiments, the determining step, detecting step, or sequence analysis comprises: a) amplifying at least a portion of the ANGPTL7 nucleic acid molecule, or the complement thereof, in the biological sample, wherein the amplified portion comprises: a thymine at a position corresponding to position 4,277 according to SEQ ID NO:5, or the complement thereof; a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; a thymine at a position corresponding to position 768 according to SEQ ID NO:130, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a thymine at a position corresponding to position 4,277 according to SEQ ID NO:5, or the complement thereof; a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a uracil at a position corresponding to position 768 according to SEQ ID NO:50, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; a thymine at a position corresponding to position 768 according to SEQ ID NO:130, or the complement thereof; and d) detecting the detectable label.

In some embodiments, the determining step, detecting step, or sequence analysis comprises: a) amplifying at least a portion of the ANGPTL7 nucleic acid molecule, or the complement thereof, in the biological sample, wherein the amplified portion comprises: an adenine at a position corresponding to position 4,311 according to SEQ ID NO:6, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; an adenine at a position corresponding to position 802 according to SEQ ID NO:138, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 4,311 according to SEQ ID NO:6, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; an adenine at a position corresponding to position 802 according to SEQ ID NO:138, or the complement thereof; and d) detecting the detectable label.

In some embodiments, the determining step, detecting step, or sequence analysis comprises: a) amplifying at least a portion of the ANGPTL7 nucleic acid molecule, or the complement thereof, in the biological sample, wherein the amplified portion comprises: a cytosine at a position corresponding to position 4,322 according to SEQ ID NO:7, or the complement thereof; a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; a cytosine at a position corresponding to position 813 according to SEQ ID NO:146, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a cytosine at a position corresponding to position 4,322 according to SEQ ID NO:7, or the complement thereof; a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; a cytosine at a position corresponding to position 813 according to SEQ ID NO:146, or the complement thereof; and d) detecting the detectable label.

In some embodiments, the determining step, detecting step, or sequence analysis comprises: a) amplifying at least a portion of the ANGPTL7 nucleic acid molecule, or the complement thereof, in the biological sample, wherein the amplified portion comprises: a thymine at a position corresponding to position 5,125 according to SEQ ID NO:8, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; a thymine at a position corresponding to position 930 according to SEQ ID NO:154, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a thymine at a position corresponding to position 5,125 according to SEQ ID NO:8, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; a thymine at a position corresponding to position 930 according to SEQ ID NO:154, or the complement thereof; and d) detecting the detectable label.

In some embodiments, the determining step, detecting step, or sequence analysis comprises: a) amplifying at least a portion of the ANGPTL7 nucleic acid molecule, or the complement thereof, in the biological sample, wherein the amplified portion comprises: a thymine at a position corresponding to position 5,176 according to SEQ ID NO:9, or the complement thereof; a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; a uracil at a position corresponding to position 981 according to SEQ ID NO:82, or the complement thereof; a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; a thymine at a position corresponding to position 981 according to SEQ ID NO:162, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a thymine at a position corresponding to position 5,176 according to SEQ ID NO:9, or the complement thereof; a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; a uracil at a position corresponding to position 981 according to SEQ ID NO:82, or the complement thereof; a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; a thymine at a position corresponding to position 981 according to SEQ ID NO:162, or the complement thereof; and d) detecting the detectable label.

In some embodiments, the determining step, detecting step, or sequence analysis comprises: a) amplifying at least a portion of the ANGPTL7 nucleic acid molecule, or the complement thereof, in the biological sample, wherein the amplified portion comprises: an adenine at a position corresponding to position 5,232 according to SEQ ID NO:10, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a thymine at a position corresponding to position 5,232 according to SEQ ID NO:10, or the complement thereof; a uracil at a position corresponding to position 967 according to SEQ ID NO:75, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof; and d) detecting the detectable label.

In some embodiments, the determining step, detecting step, or sequence analysis comprises: a) amplifying at least a portion of the ANGPTL7 genomic nucleic acid molecule, or the complement thereof, in the biological sample, wherein the portion comprises: an adenine at a position corresponding to position 4,229 according to SEQ ID NO:2, or the complement thereof; an adenine at a position corresponding to position 4,269 according to SEQ ID NO:3, or the complement thereof; a thymine at a position corresponding to position 4,273 according to SEQ ID NO:4, or the complement thereof; a thymine at a position corresponding to position 4,277 according to SEQ ID NO:5, or the complement thereof; an adenine at a position corresponding to position 4,311 according to SEQ ID NO:6, or the complement thereof; a cytosine at a position corresponding to position 4,322 according to SEQ ID NO:7, or the complement thereof; a thymine at a position corresponding to position 5,125 according to SEQ ID NO:8, or the complement thereof; a thymine at a position corresponding to position 5,176 according to SEQ ID NO:9, or the complement thereof; or an adenine at a position corresponding to position 5,232 according to SEQ ID NO:10, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 4,229 according to SEQ ID NO:2, or the complement thereof; an adenine at a position corresponding to position 4,269 according to SEQ ID NO:3, or the complement thereof; a thymine at a position corresponding to position 4,273 according to SEQ ID NO:4, or the complement thereof; a thymine at a position corresponding to position 4,277 according to SEQ ID NO:5, or the complement thereof; an adenine at a position corresponding to position 4,311 according to SEQ ID NO:6, or the complement thereof; a cytosine at a position corresponding to position 4,322 according to SEQ ID NO:7, or the complement thereof; a thymine at a position corresponding to position 5,125 according to SEQ ID NO:8, or the complement thereof; a thymine at a position corresponding to position 5,176 according to SEQ ID NO:9, or the complement thereof; or an adenine at a position corresponding to position 5,232 according to SEQ ID NO:10, or the complement thereof; and d) detecting the detectable label.

In some embodiments, the determining step, detecting step, or sequence analysis comprises: a) amplifying at least a portion of the ANGPTL7 mRNA molecule, or the complement thereof, in the biological sample, wherein the portion comprises: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof; a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a uracil at a position corresponding to position 768 according to SEQ ID NO:50, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof; a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; a uracil at a position corresponding to position 981 according to SEQ ID NO:82, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof; a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a uracil at a position corresponding to position 768 according to SEQ ID NO:50, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof; a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; a uracil at a position corresponding to position 981 according to SEQ ID NO:82, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof; and d) detecting the detectable label.

In some embodiments, the determining step, detecting step, or sequence analysis comprises: a) amplifying at least a portion of the ANGPTL7 cDNA molecule, or the complement thereof, in the biological sample, wherein the portion comprises: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; an adenine at a position corresponding to position 760 according to SEQ ID NO:114, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; a thymine at a position corresponding to position 764 according to SEQ ID NO:122, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; a thymine at a position corresponding to position 768 according to SEQ ID NO:130, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; an adenine at a position corresponding to position 802 according to SEQ ID NO:138, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; a cytosine at a position corresponding to position 813 according to SEQ ID NO:146, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; a thymine at a position corresponding to position 930 according to SEQ ID NO:154, or the complement thereof; a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; a thymine at a position corresponding to position 981 according to SEQ ID NO:162, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; an adenine at a position corresponding to position 760 according to SEQ ID NO:114, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; a thymine at a position corresponding to position 764 according to SEQ ID NO:122, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; a thymine at a position corresponding to position 768 according to SEQ ID NO:130, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; an adenine at a position corresponding to position 802 according to SEQ ID NO:138, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; a cytosine at a position corresponding to position 813 according to SEQ ID NO:146, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; a thymine at a position corresponding to position 930 according to SEQ ID NO:154, or the complement thereof; a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; a thymine at a position corresponding to position 981 according to SEQ ID NO:162, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or sequence analysis comprises: contacting the ANGPTL7 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the ANGPTL7 nucleic acid molecule, or the complement thereof, comprising: an adenine at a position corresponding to position 4,229 according to SEQ ID NO:2, or the complement thereof; an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof; and detecting the detectable label.

In some embodiments, the determining step, detecting step, or sequence analysis comprises: contacting the ANGPTL7 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the ANGPTL7 nucleic acid molecule, or the complement thereof, comprising: an adenine at a position corresponding to position 4,269 according to SEQ ID NO:3, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; an adenine at a position corresponding to position 760 according to SEQ ID NO:114, or the complement thereof; and detecting the detectable label.

In some embodiments, the determining step, detecting step, or sequence analysis comprises: contacting the ANGPTL7 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the ANGPTL7 nucleic acid molecule, or the complement thereof, comprising: a thymine at a position corresponding to position 4,273 according to SEQ ID NO:4, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; a thymine at a position corresponding to position 764 according to SEQ ID NO:122, or the complement thereof; and detecting the detectable label. In some embodiments, the determining step, detecting step, or sequence analysis comprises: contacting the ANGPTL7 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the ANGPTL7 nucleic acid molecule, or the complement thereof, comprising: a thymine at a position corresponding to position 4,277 according to SEQ ID NO:5, or the complement thereof; a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a uracil at a position corresponding to position 768 according to SEQ ID NO:50, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; a thymine at a position corresponding to position 768 according to SEQ ID NO:130, or the complement thereof; and detecting the detectable label.

In some embodiments, the determining step, detecting step, or sequence analysis comprises: contacting the ANGPTL7 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the ANGPTL7 nucleic acid molecule, or the complement thereof, comprising: an adenine at a position corresponding to position 4,311 according to SEQ ID NO:6, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; an adenine at a position corresponding to position 802 according to SEQ ID NO:138, or the complement thereof; and detecting the detectable label.

In some embodiments, the determining step, detecting step, or sequence analysis comprises: contacting the ANGPTL7 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the ANGPTL7 nucleic acid molecule, or the complement thereof, comprising: a cytosine at a position corresponding to position 4,322 according to SEQ ID NO:7, or the complement thereof; a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; a cytosine at a position corresponding to position 813 according to SEQ ID NO:146, or the complement thereof; and detecting the detectable label.

In some embodiments, the determining step, detecting step, or sequence analysis comprises: contacting the ANGPTL7 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the ANGPTL7 nucleic acid molecule, or the complement thereof, comprising: a thymine at a position corresponding to position 5,125 according to SEQ ID NO:8, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; a thymine at a position corresponding to position 930 according to SEQ ID NO:154, or the complement thereof; and detecting the detectable label.

In some embodiments, the determining step, detecting step, or sequence analysis comprises: contacting the ANGPTL7 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the ANGPTL7 nucleic acid molecule, or the complement thereof, comprising: an adenine at a position corresponding to position 5,232 according to SEQ ID NO:10, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof; and detecting the detectable label.

In some embodiments, the determining step, detecting step, or sequence analysis comprises: contacting the ANGPTL7 genomic nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the ANGPTL7 genomic nucleic acid molecule, or the complement thereof, comprising: an adenine at a position corresponding to position 4,229 according to SEQ ID NO:2, or the complement thereof; an adenine at a position corresponding to position 4,269 according to SEQ ID NO:3, or the complement thereof; a thymine at a position corresponding to position 4,273 according to SEQ ID NO:4, or the complement thereof; a thymine at a position corresponding to position 4,277 according to SEQ ID NO:5, or the complement thereof; an adenine at a position corresponding to position 4,311 according to SEQ ID NO:6, or the complement thereof; a cytosine at a position corresponding to position 4,322 according to SEQ ID NO:7, or the complement thereof; a thymine at a position corresponding to position 5,125 according to SEQ ID NO:8, or the complement thereof; a thymine at a position corresponding to position 5,176 according to SEQ ID NO:9, or the complement thereof; or an adenine at a position corresponding to position 5,232 according to SEQ ID NO:10, or the complement thereof; and detecting the detectable label.

In some embodiments, the determining step, detecting step, or sequence analysis comprises: contacting the ANGPTL7 mRNA molecule, or the complement thereof, in the biological sample with an alteration-specific probe comprising a detectable label, wherein the alteration-specific probe comprises a nucleotide sequence which hybridizes under stringent conditions to the nucleotide sequence of the ANGPTL7 mRNA molecule, or the complement thereof, comprising: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof; a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a uracil at a position corresponding to position 768 according to SEQ ID NO:50, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof; a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; a uracil at a position corresponding to position 981 according to SEQ ID NO:82, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof; and detecting the detectable label.

In some embodiments, the determining step, detecting step, or sequence analysis comprises: contacting the ANGPTL7 cDNA molecule, or the complement thereof, produced from an mRNA molecule in the biological sample with an alteration-specific probe comprising a detectable label, wherein the alteration-specific probe comprises a nucleotide sequence which hybridizes under stringent conditions to the nucleotide sequence of the ANGPTL7 cDNA molecule, or the complement thereof, comprising: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; an adenine at a position corresponding to position 760 according to SEQ ID NO:114, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; a thymine at a position corresponding to position 764 according to SEQ ID NO:122, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; a thymine at a position corresponding to position 768 according to SEQ ID NO:130, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; an adenine at a position corresponding to position 802 according to SEQ ID NO:138, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; a cytosine at a position corresponding to position 813 according to SEQ ID NO:146, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; a thymine at a position corresponding to position 930 according to SEQ ID NO:154, or the complement thereof; a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; a thymine at a position corresponding to position 981 according to SEQ ID NO:162, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof; and detecting the detectable label.

In some embodiments, the ANGPTL7 nucleic acid molecule is present within a cell obtained from the subject.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the determining step, detecting step, or sequence analysis comprises contacting the biological sample with a primer or probe, such as an alteration-specific primer or alteration-specific probe, that specifically hybridizes to an ANGPTL7 variant genomic sequence, variant mRNA sequence, or variant cDNA sequence and not the corresponding ANGPTL7 reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an ANGPTL7 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether an ANGPTL7 nucleic acid molecule (genomic nucleic acid molecule, mRNA molecule, or cDNA molecule), or complement thereof, within a biological sample comprises a nucleotide sequence comprising an adenine at a position corresponding to position 4,229 according to SEQ ID NO:2, an adenine at a position corresponding to position 706 according to SEQ ID NO:19, an adenine at a position corresponding to position 773 according to SEQ ID NO:20, an adenine at a position corresponding to position 738 according to SEQ ID NO:21, an adenine at a position corresponding to position 720 according to SEQ ID NO:22, an adenine at a position corresponding to position 695 according to SEQ ID NO:23, an adenine at a position corresponding to position 488 according to SEQ ID NO:24, an adenine at a position corresponding to position 481 according to SEQ ID NO:25, an adenine at a position corresponding to position 720 according to SEQ ID NO:26, an adenine at a position corresponding to position 706 according to SEQ ID NO:99, an adenine at a position corresponding to position 773 according to SEQ ID NO:100, an adenine at a position corresponding to position 738 according to SEQ ID NO:101, an adenine at a position corresponding to position 720 according to SEQ ID NO:102, an adenine at a position corresponding to position 695 according to SEQ ID NO:103, an adenine at a position corresponding to position 488 according to SEQ ID NO:104, an adenine at a position corresponding to position 481 according to SEQ ID NO:105, an adenine at a position corresponding to position 720 according to SEQ ID NO:106, the biological sample can be subjected to an amplification method using a primer pair that includes a first primer derived from the 5′ flanking sequence adjacent to an adenine at a position corresponding to position 4,229 according to SEQ ID NO:2, an adenine at a position corresponding to position 706 according to SEQ ID NO:19, an adenine at a position corresponding to position 773 according to SEQ ID NO:20, an adenine at a position corresponding to position 738 according to SEQ ID NO:21, an adenine at a position corresponding to position 720 according to SEQ ID NO:22, an adenine at a position corresponding to position 695 according to SEQ ID NO:23, an adenine at a position corresponding to position 488 according to SEQ ID NO:24, an adenine at a position corresponding to position 481 according to SEQ ID NO:25, an adenine at a position corresponding to position 720 according to SEQ ID NO:26, an adenine at a position corresponding to position 706 according to SEQ ID NO:99, an adenine at a position corresponding to position 773 according to SEQ ID NO:100, an adenine at a position corresponding to position 738 according to SEQ ID NO:101, an adenine at a position corresponding to position 720 according to SEQ ID NO:102, an adenine at a position corresponding to position 695 according to SEQ ID NO:103, an adenine at a position corresponding to position 488 according to SEQ ID NO:104, an adenine at a position corresponding to position 481 according to SEQ ID NO:105, an adenine at a position corresponding to position 720 according to SEQ ID NO:106, and a second primer derived from the 3′ flanking sequence adjacent to an adenine at a position corresponding to position 4,229 according to SEQ ID NO:2, an adenine at a position corresponding to position 706 according to SEQ ID NO:19, an adenine at a position corresponding to position 773 according to SEQ ID NO:20, an adenine at a position corresponding to position 738 according to SEQ ID NO:21, an adenine at a position corresponding to position 720 according to SEQ ID NO:22, an adenine at a position corresponding to position 695 according to SEQ ID NO:23, an adenine at a position corresponding to position 488 according to SEQ ID NO:24, an adenine at a position corresponding to position 481 according to SEQ ID NO:25, an adenine at a position corresponding to position 720 according to SEQ ID NO:26, an adenine at a position corresponding to position 706 according to SEQ ID NO:99, an adenine at a position corresponding to position 773 according to SEQ ID NO:100, an adenine at a position corresponding to position 738 according to SEQ ID NO:101, an adenine at a position corresponding to position 720 according to SEQ ID NO:102, an adenine at a position corresponding to position 695 according to SEQ ID NO:103, an adenine at a position corresponding to position 488 according to SEQ ID NO:104, an adenine at a position corresponding to position 481 according to SEQ ID NO:105, an adenine at a position corresponding to position 720 according to SEQ ID NO:106 to produce an amplicon that is indicative of the presence of the SNP at positions encoding an adenine at a position corresponding to position 4,229 according to SEQ ID NO:2, an adenine at a position corresponding to position 706 according to SEQ ID NO:19, an adenine at a position corresponding to position 773 according to SEQ ID NO:20, an adenine at a position corresponding to position 738 according to SEQ ID NO:21, an adenine at a position corresponding to position 720 according to SEQ ID NO:22, an adenine at a position corresponding to position 695 according to SEQ ID NO:23, an adenine at a position corresponding to position 488 according to SEQ ID NO:24, an adenine at a position corresponding to position 481 according to SEQ ID NO:25, an adenine at a position corresponding to position 720 according to SEQ ID NO:26, an adenine at a position corresponding to position 706 according to SEQ ID NO:99, an adenine at a position corresponding to position 773 according to SEQ ID NO:100, an adenine at a position corresponding to position 738 according to SEQ ID NO:101, an adenine at a position corresponding to position 720 according to SEQ ID NO:102, an adenine at a position corresponding to position 695 according to SEQ ID NO:103, an adenine at a position corresponding to position 488 according to SEQ ID NO:104, an adenine at a position corresponding to position 481 according to SEQ ID NO:105, an adenine at a position corresponding to position 720 according to SEQ ID NO:106. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising an adenine at a position corresponding to position 4,229 according to SEQ ID NO:2, an adenine at a position corresponding to position 706 according to SEQ ID NO:19, an adenine at a position corresponding to position 773 according to SEQ ID NO:20, an adenine at a position corresponding to position 738 according to SEQ ID NO:21, an adenine at a position corresponding to position 720 according to SEQ ID NO:22, an adenine at a position corresponding to position 695 according to SEQ ID NO:23, an adenine at a position corresponding to position 488 according to SEQ ID NO:24, an adenine at a position corresponding to position 481 according to SEQ ID NO:25, an adenine at a position corresponding to position 720 according to SEQ ID NO:26, an adenine at a position corresponding to position 706 according to SEQ ID NO:99, an adenine at a position corresponding to position 773 according to SEQ ID NO:100, an adenine at a position corresponding to position 738 according to SEQ ID NO:101, an adenine at a position corresponding to position 720 according to SEQ ID NO:102, an adenine at a position corresponding to position 695 according to SEQ ID NO:103, an adenine at a position corresponding to position 488 according to SEQ ID NO:104, an adenine at a position corresponding to position 481 according to SEQ ID NO:105, an adenine at a position corresponding to position 720 according to SEQ ID NO:106, and at least 1, 2, 3, 4, 5, 6, 7, 8, 9, 10, or more nucleotides on each side of positions comprising an adenine at a position corresponding to position 4,229 according to SEQ ID NO:2, an adenine at a position corresponding to position 706 according to SEQ ID NO:19, an adenine at a position corresponding to position 773 according to SEQ ID NO:20, an adenine at a position corresponding to position 738 according to SEQ ID NO:21, an adenine at a position corresponding to position 720 according to SEQ ID NO:22, an adenine at a position corresponding to position 695 according to SEQ ID NO:23, an adenine at a position corresponding to position 488 according to SEQ ID NO:24, an adenine at a position corresponding to position 481 according to SEQ ID NO:25, an adenine at a position corresponding to position 720 according to SEQ ID NO:26, an adenine at a position corresponding to position 706 according to SEQ ID NO:99, an adenine at a position corresponding to position 773 according to SEQ ID NO:100, an adenine at a position corresponding to position 738 according to SEQ ID NO:101, an adenine at a position corresponding to position 720 according to SEQ ID NO:102, an adenine at a position corresponding to position 695 according to SEQ ID NO:103, an adenine at a position corresponding to position 488 according to SEQ ID NO:104, an adenine at a position corresponding to position 481 according to SEQ ID NO:105, an adenine at a position corresponding to position 720 according to SEQ ID NO:106.

In some embodiments, to determine whether an ANGPTL7 nucleic acid molecule (genomic nucleic acid molecule, mRNA molecule, or cDNA molecule), or complement thereof, within a biological sample comprises a nucleotide sequence comprising an adenine at a position corresponding to position 4,269 according to SEQ ID NO:3, an adenine at a position corresponding to position 746 according to SEQ ID NO:27, an adenine at a position corresponding to position 812 according to SEQ ID NO:28, an adenine at a position corresponding to position 778 according to SEQ ID NO:29, an adenine at a position corresponding to position 760 according to SEQ ID NO:30, an adenine at a position corresponding to position 735 according to SEQ ID NO:31, an adenine at a position corresponding to position 528 according to SEQ ID NO:32, an adenine at a position corresponding to position 521 according to SEQ ID NO:33, an adenine at a position corresponding to position 760 according to SEQ ID NO:34, an adenine at a position corresponding to position 746 according to SEQ ID NO:107, an adenine at a position corresponding to position 812 according to SEQ ID NO:108, an adenine at a position corresponding to position 778 according to SEQ ID NO:109, an adenine at a position corresponding to position 760 according to SEQ ID NO:110, an adenine at a position corresponding to position 735 according to SEQ ID NO:111, an adenine at a position corresponding to position 528 according to SEQ ID NO:112, an adenine at a position corresponding to position 529 according to SEQ ID NO:113, an adenine at a position corresponding to position 760 according to SEQ ID NO:114, the biological sample can be subjected to an amplification method using a primer pair that includes a first primer derived from the 5′ flanking sequence adjacent to an adenine at a position corresponding to position 4,269 according to SEQ ID NO:3, an adenine at a position corresponding to position 746 according to SEQ ID NO:27, an adenine at a position corresponding to position 812 according to SEQ ID NO:28, an adenine at a position corresponding to position 778 according to SEQ ID NO:29, an adenine at a position corresponding to position 760 according to SEQ ID NO:30, an adenine at a position corresponding to position 735 according to SEQ ID NO:31, an adenine at a position corresponding to position 528 according to SEQ ID NO:32, an adenine at a position corresponding to position 521 according to SEQ ID NO:33, an adenine at a position corresponding to position 760 according to SEQ ID NO:34, an adenine at a position corresponding to position 746 according to SEQ ID NO:107, an adenine at a position corresponding to position 812 according to SEQ ID NO:108, an adenine at a position corresponding to position 778 according to SEQ ID NO:109, an adenine at a position corresponding to position 760 according to SEQ ID NO:110, an adenine at a position corresponding to position 735 according to SEQ ID NO:111, an adenine at a position corresponding to position 528 according to SEQ ID NO:112, an adenine at a position corresponding to position 529 according to SEQ ID NO:113, an adenine at a position corresponding to position 760 according to SEQ ID NO:114, and a second primer derived from the 3′ flanking sequence adjacent to an adenine at a position corresponding to position 4,269 according to SEQ ID NO:3, an adenine at a position corresponding to position 746 according to SEQ ID NO:27, an adenine at a position corresponding to position 812 according to SEQ ID NO:28, an adenine at a position corresponding to position 778 according to SEQ ID NO:29, an adenine at a position corresponding to position 760 according to SEQ ID NO:30, an adenine at a position corresponding to position 735 according to SEQ ID NO:31, an adenine at a position corresponding to position 528 according to SEQ ID NO:32, an adenine at a position corresponding to position 521 according to SEQ ID NO:33, an adenine at a position corresponding to position 760 according to SEQ ID NO:34, an adenine at a position corresponding to position 746 according to SEQ ID NO:107, an adenine at a position corresponding to position 812 according to SEQ ID NO:108, an adenine at a position corresponding to position 778 according to SEQ ID NO:109, an adenine at a position corresponding to position 760 according to SEQ ID NO:110, an adenine at a position corresponding to position 735 according to SEQ ID NO:111, an adenine at a position corresponding to position 528 according to SEQ ID NO:112, an adenine at a position corresponding to position 529 according to SEQ ID NO:113, an adenine at a position corresponding to position 760 according to SEQ ID NO:114 to produce an amplicon that is indicative of the presence of the SNP at positions encoding an adenine at a position corresponding to position 4,269 according to SEQ ID NO:3, an adenine at a position corresponding to position 746 according to SEQ ID NO:27, an adenine at a position corresponding to position 812 according to SEQ ID NO:28, an adenine at a position corresponding to position 778 according to SEQ ID NO:29, an adenine at a position corresponding to position 760 according to SEQ ID NO:30, an adenine at a position corresponding to position 735 according to SEQ ID NO:31, an adenine at a position corresponding to position 528 according to SEQ ID NO:32, an adenine at a position corresponding to position 521 according to SEQ ID NO:33, an adenine at a position corresponding to position 760 according to SEQ ID NO:34, an adenine at a position corresponding to position 746 according to SEQ ID NO:107, an adenine at a position corresponding to position 812 according to SEQ ID NO:108, an adenine at a position corresponding to position 778 according to SEQ ID NO:109, an adenine at a position corresponding to position 760 according to SEQ ID NO:110, an adenine at a position corresponding to position 735 according to SEQ ID NO:111, an adenine at a position corresponding to position 528 according to SEQ ID NO:112, an adenine at a position corresponding to position 529 according to SEQ ID NO:113, an adenine at a position corresponding to position 760 according to SEQ ID NO:114. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising an adenine at a position corresponding to position 4,269 according to SEQ ID NO:3, an adenine at a position corresponding to position 746 according to SEQ ID NO:27, an adenine at a position corresponding to position 812 according to SEQ ID NO:28, an adenine at a position corresponding to position 778 according to SEQ ID NO:29, an adenine at a position corresponding to position 760 according to SEQ ID NO:30, an adenine at a position corresponding to position 735 according to SEQ ID NO:31, an adenine at a position corresponding to position 528 according to SEQ ID NO:32, an adenine at a position corresponding to position 521 according to SEQ ID NO:33, an adenine at a position corresponding to position 760 according to SEQ ID NO:34, an adenine at a position corresponding to position 746 according to SEQ ID NO:107, an adenine at a position corresponding to position 812 according to SEQ ID NO:108, an adenine at a position corresponding to position 778 according to SEQ ID NO:109, an adenine at a position corresponding to position 760 according to SEQ ID NO:110, an adenine at a position corresponding to position 735 according to SEQ ID NO:111, an adenine at a position corresponding to position 528 according to SEQ ID NO:112, an adenine at a position corresponding to position 529 according to SEQ ID NO:113, an adenine at a position corresponding to position 760 according to SEQ ID NO:114 and at least 1, 2, 3, 4, 5, 6, 7, 8, 9, 10, or more nucleotides on each side of positions comprising an adenine at a position corresponding to position 4,269 according to SEQ ID NO:3, an adenine at a position corresponding to position 746 according to SEQ ID NO:27, an adenine at a position corresponding to position 812 according to SEQ ID NO:28, an adenine at a position corresponding to position 778 according to SEQ ID NO:29, an adenine at a position corresponding to position 760 according to SEQ ID NO:30, an adenine at a position corresponding to position 735 according to SEQ ID NO:31, an adenine at a position corresponding to position 528 according to SEQ ID NO:32, an adenine at a position corresponding to position 521 according to SEQ ID NO:33, an adenine at a position corresponding to position 760 according to SEQ ID NO:34, an adenine at a position corresponding to position 746 according to SEQ ID NO:107, an adenine at a position corresponding to position 812 according to SEQ ID NO:108, an adenine at a position corresponding to position 778 according to SEQ ID NO:109, an adenine at a position corresponding to position 760 according to SEQ ID NO:110, an adenine at a position corresponding to position 735 according to SEQ ID NO:111, an adenine at a position corresponding to position 528 according to SEQ ID NO:112, an adenine at a position corresponding to position 529 according to SEQ ID NO:113, an adenine at a position corresponding to position 760 according to SEQ ID NO:114.

In some embodiments, to determine whether an ANGPTL7 nucleic acid molecule (genomic nucleic acid molecule, mRNA molecule, or cDNA molecule), or complement thereof, within a biological sample comprises a nucleotide sequence comprising a thymine at a position corresponding to position 4,273 according to SEQ ID NO:4, a uracil at a position corresponding to position 750 according to SEQ ID NO:35, a uracil at a position corresponding to position 817 according to SEQ ID NO:36, a uracil at a position corresponding to position 782 according to SEQ ID NO:37, a uracil at a position corresponding to position 764 according to SEQ ID NO:38, a uracil at a position corresponding to position 739 according to SEQ ID NO:39, a uracil at a position corresponding to position 532 according to SEQ ID NO:40, a uracil at a position corresponding to position 525 according to SEQ ID NO:41, a uracil at a position corresponding to position 764 according to SEQ ID NO:42, a thymine at a position corresponding to position 750 according to SEQ ID NO:115, a thymine at a position corresponding to position 817 according to SEQ ID NO:116, a thymine at a position corresponding to position 782 according to SEQ ID NO:117, a thymine at a position corresponding to position 764 according to SEQ ID NO:118, a thymine at a position corresponding to position 739 according to SEQ ID NO:119, a thymine at a position corresponding to position 532 according to SEQ ID NO:120, a thymine at a position corresponding to position 525 according to SEQ ID NO:121, a thymine at a position corresponding to position 764 according to SEQ ID NO:122, the biological sample can be subjected to an amplification method using a primer pair that includes a first primer derived from the 5′ flanking sequence adjacent to a thymine at a position corresponding to position 4,273 according to SEQ ID NO:4, a uracil at a position corresponding to position 750 according to SEQ ID NO:35, a uracil at a position corresponding to position 817 according to SEQ ID NO:36, a uracil at a position corresponding to position 782 according to SEQ ID NO:37, a uracil at a position corresponding to position 764 according to SEQ ID NO:38, a uracil at a position corresponding to position 739 according to SEQ ID NO:39, a uracil at a position corresponding to position 532 according to SEQ ID NO:40, a uracil at a position corresponding to position 525 according to SEQ ID NO:41, a uracil at a position corresponding to position 764 according to SEQ ID NO:42, a thymine at a position corresponding to position 750 according to SEQ ID NO:115, a thymine at a position corresponding to position 817 according to SEQ ID NO:116, a thymine at a position corresponding to position 782 according to SEQ ID NO:117, a thymine at a position corresponding to position 764 according to SEQ ID NO:118, a thymine at a position corresponding to position 739 according to SEQ ID NO:119, a thymine at a position corresponding to position 532 according to SEQ ID NO:120, a thymine at a position corresponding to position 525 according to SEQ ID NO:121, a thymine at a position corresponding to position 764 according to SEQ ID NO:122, and a second primer derived from the 3′ flanking sequence adjacent to a thymine at a position corresponding to position 4,273 according to SEQ ID NO:4, a uracil at a position corresponding to position 750 according to SEQ ID NO:35, a uracil at a position corresponding to position 817 according to SEQ ID NO:36, a uracil at a position corresponding to position 782 according to SEQ ID NO:37, a uracil at a position corresponding to position 764 according to SEQ ID NO:38, a uracil at a position corresponding to position 739 according to SEQ ID NO:39, a uracil at a position corresponding to position 532 according to SEQ ID NO:40, a uracil at a position corresponding to position 525 according to SEQ ID NO:41, a uracil at a position corresponding to position 764 according to SEQ ID NO:42, a thymine at a position corresponding to position 750 according to SEQ ID NO:115, a thymine at a position corresponding to position 817 according to SEQ ID NO:116, a thymine at a position corresponding to position 782 according to SEQ ID NO:117, a thymine at a position corresponding to position 764 according to SEQ ID NO:118, a thymine at a position corresponding to position 739 according to SEQ ID NO:119, a thymine at a position corresponding to position 532 according to SEQ ID NO:120, a thymine at a position corresponding to position 525 according to SEQ ID NO:121, a thymine at a position corresponding to position 764 according to SEQ ID NO:122 to produce an amplicon that is indicative of the presence of the SNP at positions encoding a thymine at a position corresponding to position 4,273 according to SEQ ID NO:4, a uracil at a position corresponding to position 750 according to SEQ ID NO:35, a uracil at a position corresponding to position 817 according to SEQ ID NO:36, a uracil at a position corresponding to position 782 according to SEQ ID NO:37, a uracil at a position corresponding to position 764 according to SEQ ID NO:38, a uracil at a position corresponding to position 739 according to SEQ ID NO:39, a uracil at a position corresponding to position 532 according to SEQ ID NO:40, a uracil at a position corresponding to position 525 according to SEQ ID NO:41, a uracil at a position corresponding to position 764 according to SEQ ID NO:42, a thymine at a position corresponding to position 750 according to SEQ ID NO:115, a thymine at a position corresponding to position 817 according to SEQ ID NO:116, a thymine at a position corresponding to position 782 according to SEQ ID NO:117, a thymine at a position corresponding to position 764 according to SEQ ID NO:118, a thymine at a position corresponding to position 739 according to SEQ ID NO:119, a thymine at a position corresponding to position 532 according to SEQ ID NO:120, a thymine at a position corresponding to position 525 according to SEQ ID NO:121, a thymine at a position corresponding to position 764 according to SEQ ID NO:122. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 4,273 according to SEQ ID NO:4, a uracil at a position corresponding to position 750 according to SEQ ID NO:35, a uracil at a position corresponding to position 817 according to SEQ ID NO:36, a uracil at a position corresponding to position 782 according to SEQ ID NO:37, a uracil at a position corresponding to position 764 according to SEQ ID NO:38, a uracil at a position corresponding to position 739 according to SEQ ID NO:39, a uracil at a position corresponding to position 532 according to SEQ ID NO:40, a uracil at a position corresponding to position 525 according to SEQ ID NO:41, a uracil at a position corresponding to position 764 according to SEQ ID NO:42, a thymine at a position corresponding to position 750 according to SEQ ID NO:115, a thymine at a position corresponding to position 817 according to SEQ ID NO:116, a thymine at a position corresponding to position 782 according to SEQ ID NO:117, a thymine at a position corresponding to position 764 according to SEQ ID NO:118, a thymine at a position corresponding to position 739 according to SEQ ID NO:119, a thymine at a position corresponding to position 532 according to SEQ ID NO:120, a thymine at a position corresponding to position 525 according to SEQ ID NO:121, a thymine at a position corresponding to position 764 according to SEQ ID NO:122 and at least 1, 2, 3, 4, 5, 6, 7, 8, 9, 10, or more nucleotides on each side of positions comprising a thymine at a position corresponding to position 4,273 according to SEQ ID NO:4, a uracil at a position corresponding to position 750 according to SEQ ID NO:35, a uracil at a position corresponding to position 817 according to SEQ ID NO:36, a uracil at a position corresponding to position 782 according to SEQ ID NO:37, a uracil at a position corresponding to position 764 according to SEQ ID NO:38, a uracil at a position corresponding to position 739 according to SEQ ID NO:39, a uracil at a position corresponding to position 532 according to SEQ ID NO:40, a uracil at a position corresponding to position 525 according to SEQ ID NO:41, a uracil at a position corresponding to position 764 according to SEQ ID NO:42, a thymine at a position corresponding to position 750 according to SEQ ID NO:115, a thymine at a position corresponding to position 817 according to SEQ ID NO:116, a thymine at a position corresponding to position 782 according to SEQ ID NO:117, a thymine at a position corresponding to position 764 according to SEQ ID NO:118, a thymine at a position corresponding to position 739 according to SEQ ID NO:119, a thymine at a position corresponding to position 532 according to SEQ ID NO:120, a thymine at a position corresponding to position 525 according to SEQ ID NO:121, a thymine at a position corresponding to position 764 according to SEQ ID NO:122.

In some embodiments, to determine whether an ANGPTL7 nucleic acid molecule (genomic nucleic acid molecule, mRNA molecule, or cDNA molecule), or complement thereof, within a biological sample comprises a nucleotide sequence comprising a thymine at a position corresponding to position 4,277 according to SEQ ID NO:5, a uracil at a position corresponding to position 754 according to SEQ ID NO:43, a uracil at a position corresponding to position 821 according to SEQ ID NO:44, a uracil at a position corresponding to position 786 according to SEQ ID NO:45, a uracil at a position corresponding to position 768 according to SEQ ID NO:46, a uracil at a position corresponding to position 743 according to SEQ ID NO:47, a uracil at a position corresponding to position 536 according to SEQ ID NO:48, a uracil at a position corresponding to position 529 according to SEQ ID NO:49, a uracil at a position corresponding to position 768 according to SEQ ID NO:50, a thymine at a position corresponding to position 754 according to SEQ ID NO:123, a thymine at a position corresponding to position 821 according to SEQ ID NO:124, a thymine at a position corresponding to position 786 according to SEQ ID NO:125, a thymine at a position corresponding to position 768 according to SEQ ID NO:126, a thymine at a position corresponding to position 743 according to SEQ ID NO:127, a thymine at a position corresponding to position 536 according to SEQ ID NO:128, a thymine at a position corresponding to position 529 according to SEQ ID NO:129, a thymine at a position corresponding to position 768 according to SEQ ID NO:130, the biological sample can be subjected to an amplification method using a primer pair that includes a first primer derived from the 5′ flanking sequence adjacent to a thymine at a position corresponding to position 4,277 according to SEQ ID NO:5, a uracil at a position corresponding to position 754 according to SEQ ID NO:43, a uracil at a position corresponding to position 821 according to SEQ ID NO:44, a uracil at a position corresponding to position 786 according to SEQ ID NO:45, a uracil at a position corresponding to position 768 according to SEQ ID NO:46, a uracil at a position corresponding to position 743 according to SEQ ID NO:47, a uracil at a position corresponding to position 536 according to SEQ ID NO:48, a uracil at a position corresponding to position 529 according to SEQ ID NO:49, a uracil at a position corresponding to position 768 according to SEQ ID NO:50, a thymine at a position corresponding to position 754 according to SEQ ID NO:123, a thymine at a position corresponding to position 821 according to SEQ ID NO:124, a thymine at a position corresponding to position 786 according to SEQ ID NO:125, a thymine at a position corresponding to position 768 according to SEQ ID NO:126, a thymine at a position corresponding to position 743 according to SEQ ID NO:127, a thymine at a position corresponding to position 536 according to SEQ ID NO:128, a thymine at a position corresponding to position 529 according to SEQ ID NO:129, a thymine at a position corresponding to position 768 according to SEQ ID NO:130, and a second primer derived from the 3′ flanking sequence adjacent to a thymine at a position corresponding to position 4,277 according to SEQ ID NO:5, a uracil at a position corresponding to position 754 according to SEQ ID NO:43, a uracil at a position corresponding to position 821 according to SEQ ID NO:44, a uracil at a position corresponding to position 786 according to SEQ ID NO:45, a uracil at a position corresponding to position 768 according to SEQ ID NO:46, a uracil at a position corresponding to position 743 according to SEQ ID NO:47, a uracil at a position corresponding to position 536 according to SEQ ID NO:48, a uracil at a position corresponding to position 529 according to SEQ ID NO:49, a uracil at a position corresponding to position 768 according to SEQ ID NO:50, a thymine at a position corresponding to position 754 according to SEQ ID NO:123, a thymine at a position corresponding to position 821 according to SEQ ID NO:124, a thymine at a position corresponding to position 786 according to SEQ ID NO:125, a thymine at a position corresponding to position 768 according to SEQ ID NO:126, a thymine at a position corresponding to position 743 according to SEQ ID NO:127, a thymine at a position corresponding to position 536 according to SEQ ID NO:128, a thymine at a position corresponding to position 529 according to SEQ ID NO:129, a thymine at a position corresponding to position 768 according to SEQ ID NO:130 to produce an amplicon that is indicative of the presence of the SNP at positions encoding a thymine at a position corresponding to position 4,277 according to SEQ ID NO:5, a uracil at a position corresponding to position 754 according to SEQ ID NO:43, a uracil at a position corresponding to position 821 according to SEQ ID NO:44, a uracil at a position corresponding to position 786 according to SEQ ID NO:45, a uracil at a position corresponding to position 768 according to SEQ ID NO:46, a uracil at a position corresponding to position 743 according to SEQ ID NO:47, a uracil at a position corresponding to position 536 according to SEQ ID NO:48, a uracil at a position corresponding to position 529 according to SEQ ID NO:49, a uracil at a position corresponding to position 768 according to SEQ ID NO:50, a thymine at a position corresponding to position 754 according to SEQ ID NO:123, a thymine at a position corresponding to position 821 according to SEQ ID NO:124, a thymine at a position corresponding to position 786 according to SEQ ID NO:125, a thymine at a position corresponding to position 768 according to SEQ ID NO:126, a thymine at a position corresponding to position 743 according to SEQ ID NO:127, a thymine at a position corresponding to position 536 according to SEQ ID NO:128, a thymine at a position corresponding to position 529 according to SEQ ID NO:129, a thymine at a position corresponding to position 768 according to SEQ ID NO:130. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 4,277 according to SEQ ID NO:5, a uracil at a position corresponding to position 754 according to SEQ ID NO:43, a uracil at a position corresponding to position 821 according to SEQ ID NO:44, a uracil at a position corresponding to position 786 according to SEQ ID NO:45, a uracil at a position corresponding to position 768 according to SEQ ID NO:46, a uracil at a position corresponding to position 743 according to SEQ ID NO:47, a uracil at a position corresponding to position 536 according to SEQ ID NO:48, a uracil at a position corresponding to position 529 according to SEQ ID NO:49, a uracil at a position corresponding to position 768 according to SEQ ID NO:50, a thymine at a position corresponding to position 754 according to SEQ ID NO:123, a thymine at a position corresponding to position 821 according to SEQ ID NO:124, a thymine at a position corresponding to position 786 according to SEQ ID NO:125, a thymine at a position corresponding to position 768 according to SEQ ID NO:126, a thymine at a position corresponding to position 743 according to SEQ ID NO:127, a thymine at a position corresponding to position 536 according to SEQ ID NO:128, a thymine at a position corresponding to position 529 according to SEQ ID NO:129, a thymine at a position corresponding to position 768 according to SEQ ID NO:130 and at least 1, 2, 3, 4, 5, 6, 7, 8, 9, 10, or more nucleotides on each side of positions comprising a thymine at a position corresponding to position 4,277 according to SEQ ID NO:5, a uracil at a position corresponding to position 754 according to SEQ ID NO:43, a uracil at a position corresponding to position 821 according to SEQ ID NO:44, a uracil at a position corresponding to position 786 according to SEQ ID NO:45, a uracil at a position corresponding to position 768 according to SEQ ID NO:46, a uracil at a position corresponding to position 743 according to SEQ ID NO:47, a uracil at a position corresponding to position 536 according to SEQ ID NO:48, a uracil at a position corresponding to position 529 according to SEQ ID NO:49, a uracil at a position corresponding to position 768 according to SEQ ID NO:50, a thymine at a position corresponding to position 754 according to SEQ ID NO:123, a thymine at a position corresponding to position 821 according to SEQ ID NO:124, a thymine at a position corresponding to position 786 according to SEQ ID NO:125, a thymine at a position corresponding to position 768 according to SEQ ID NO:126, a thymine at a position corresponding to position 743 according to SEQ ID NO:127, a thymine at a position corresponding to position 536 according to SEQ ID NO:128, a thymine at a position corresponding to position 529 according to SEQ ID NO:129, a thymine at a position corresponding to position 768 according to SEQ ID NO:130.

In some embodiments, to determine whether an ANGPTL7 nucleic acid molecule (genomic nucleic acid molecule, mRNA molecule, or cDNA molecule), or complement thereof, within a biological sample comprises a nucleotide sequence comprising an adenine at a position corresponding to position 4,311 according to SEQ ID NO:6, an adenine at a position corresponding to position 788 according to SEQ ID NO:51, an adenine at a position corresponding to position 855 according to SEQ ID NO:52, an adenine at a position corresponding to position 820 according to SEQ ID NO:53, an adenine at a position corresponding to position 802 according to SEQ ID NO:54, an adenine at a position corresponding to position 777 according to SEQ ID NO:55, an adenine at a position corresponding to position 570 according to SEQ ID NO:56, an adenine at a position corresponding to position 563 according to SEQ ID NO:57, an adenine at a position corresponding to position 802 according to SEQ ID NO:58, an adenine at a position corresponding to position 788 according to SEQ ID NO:131, an adenine at a position corresponding to position 855 according to SEQ ID NO:132, an adenine at a position corresponding to position 820 according to SEQ ID NO:133, an adenine at a position corresponding to position 802 according to SEQ ID NO:134, an adenine at a position corresponding to position 777 according to SEQ ID NO:135, an adenine at a position corresponding to position 570 according to SEQ ID NO:136, an adenine at a position corresponding to position 563 according to SEQ ID NO:137, an adenine at a position corresponding to position 802 according to SEQ ID NO:138, the biological sample can be subjected to an amplification method using a primer pair that includes a first primer derived from the 5′ flanking sequence adjacent to an adenine at a position corresponding to position 4,311 according to SEQ ID NO:6, an adenine at a position corresponding to position 788 according to SEQ ID NO:51, an adenine at a position corresponding to position 855 according to SEQ ID NO:52, an adenine at a position corresponding to position 820 according to SEQ ID NO:53, an adenine at a position corresponding to position 802 according to SEQ ID NO:54, an adenine at a position corresponding to position 777 according to SEQ ID NO:55, an adenine at a position corresponding to position 570 according to SEQ ID NO:56, an adenine at a position corresponding to position 563 according to SEQ ID NO:57, an adenine at a position corresponding to position 802 according to SEQ ID NO:58, an adenine at a position corresponding to position 788 according to SEQ ID NO:131, an adenine at a position corresponding to position 855 according to SEQ ID NO:132, an adenine at a position corresponding to position 820 according to SEQ ID NO:133, an adenine at a position corresponding to position 802 according to SEQ ID NO:134, an adenine at a position corresponding to position 777 according to SEQ ID NO:135, an adenine at a position corresponding to position 570 according to SEQ ID NO:136, an adenine at a position corresponding to position 563 according to SEQ ID NO:137, an adenine at a position corresponding to position 802 according to SEQ ID NO:138, and a second primer derived from the 3′ flanking sequence adjacent to an adenine at a position corresponding to position 4,311 according to SEQ ID NO:6, an adenine at a position corresponding to position 788 according to SEQ ID NO:51, an adenine at a position corresponding to position 855 according to SEQ ID NO:52, an adenine at a position corresponding to position 820 according to SEQ ID NO:53, an adenine at a position corresponding to position 802 according to SEQ ID NO:54, an adenine at a position corresponding to position 777 according to SEQ ID NO:55, an adenine at a position corresponding to position 570 according to SEQ ID NO:56, an adenine at a position corresponding to position 563 according to SEQ ID NO:57, an adenine at a position corresponding to position 802 according to SEQ ID NO:58, an adenine at a position corresponding to position 788 according to SEQ ID NO:131, an adenine at a position corresponding to position 855 according to SEQ ID NO:132, an adenine at a position corresponding to position 820 according to SEQ ID NO:133, an adenine at a position corresponding to position 802 according to SEQ ID NO:134, an adenine at a position corresponding to position 777 according to SEQ ID NO:135, an adenine at a position corresponding to position 570 according to SEQ ID NO:136, an adenine at a position corresponding to position 563 according to SEQ ID NO:137, an adenine at a position corresponding to position 802 according to SEQ ID NO:138 to produce an amplicon that is indicative of the presence of the SNP at positions encoding an adenine at a position corresponding to position 4,311 according to SEQ ID NO:6, an adenine at a position corresponding to position 788 according to SEQ ID NO:51, an adenine at a position corresponding to position 855 according to SEQ ID NO:52, an adenine at a position corresponding to position 820 according to SEQ ID NO:53, an adenine at a position corresponding to position 802 according to SEQ ID NO:54, an adenine at a position corresponding to position 777 according to SEQ ID NO:55, an adenine at a position corresponding to position 570 according to SEQ ID NO:56, an adenine at a position corresponding to position 563 according to SEQ ID NO:57, an adenine at a position corresponding to position 802 according to SEQ ID NO:58, an adenine at a position corresponding to position 788 according to SEQ ID NO:131, an adenine at a position corresponding to position 855 according to SEQ ID NO:132, an adenine at a position corresponding to position 820 according to SEQ ID NO:133, an adenine at a position corresponding to position 802 according to SEQ ID NO:134, an adenine at a position corresponding to position 777 according to SEQ ID NO:135, an adenine at a position corresponding to position 570 according to SEQ ID NO:136, an adenine at a position corresponding to position 563 according to SEQ ID NO:137, an adenine at a position corresponding to position 802 according to SEQ ID NO:138. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising an adenine at a position corresponding to position 4,311 according to SEQ ID NO:6, an adenine at a position corresponding to position 788 according to SEQ ID NO:51, an adenine at a position corresponding to position 855 according to SEQ ID NO:52, an adenine at a position corresponding to position 820 according to SEQ ID NO:53, an adenine at a position corresponding to position 802 according to SEQ ID NO:54, an adenine at a position corresponding to position 777 according to SEQ ID NO:55, an adenine at a position corresponding to position 570 according to SEQ ID NO:56, an adenine at a position corresponding to position 563 according to SEQ ID NO:57, an adenine at a position corresponding to position 802 according to SEQ ID NO:58, an adenine at a position corresponding to position 788 according to SEQ ID NO:131, an adenine at a position corresponding to position 855 according to SEQ ID NO:132, an adenine at a position corresponding to position 820 according to SEQ ID NO:133, an adenine at a position corresponding to position 802 according to SEQ ID NO:134, an adenine at a position corresponding to position 777 according to SEQ ID NO:135, an adenine at a position corresponding to position 570 according to SEQ ID NO:136, an adenine at a position corresponding to position 563 according to SEQ ID NO:137, an adenine at a position corresponding to position 802 according to SEQ ID NO:138 and at least 1, 2, 3, 4, 5, 6, 7, 8, 9, 10, or more nucleotides on each side of positions comprising an adenine at a position corresponding to position 4,311 according to SEQ ID NO:6, an adenine at a position corresponding to position 788 according to SEQ ID NO:51, an adenine at a position corresponding to position 855 according to SEQ ID NO:52, an adenine at a position corresponding to position 820 according to SEQ ID NO:53, an adenine at a position corresponding to position 802 according to SEQ ID NO:54, an adenine at a position corresponding to position 777 according to SEQ ID NO:55, an adenine at a position corresponding to position 570 according to SEQ ID NO:56, an adenine at a position corresponding to position 563 according to SEQ ID NO:57, an adenine at a position corresponding to position 802 according to SEQ ID NO:58, an adenine at a position corresponding to position 788 according to SEQ ID NO:131, an adenine at a position corresponding to position 855 according to SEQ ID NO:132, an adenine at a position corresponding to position 820 according to SEQ ID NO:133, an adenine at a position corresponding to position 802 according to SEQ ID NO:134, an adenine at a position corresponding to position 777 according to SEQ ID NO:135, an adenine at a position corresponding to position 570 according to SEQ ID NO:136, an adenine at a position corresponding to position 563 according to SEQ ID NO:137, an adenine at a position corresponding to position 802 according to SEQ ID NO:138.

In some embodiments, to determine whether an ANGPTL7 nucleic acid molecule (genomic nucleic acid molecule, mRNA molecule, or cDNA molecule), or complement thereof, within a biological sample comprises a nucleotide sequence comprising a cytosine at a position corresponding to position 4,322 according to SEQ ID NO:7, a cytosine at a position corresponding to position 799 according to SEQ ID NO:59, a cytosine at a position corresponding to position 866 according to SEQ ID NO:60, a cytosine at a position corresponding to position 831 according to SEQ ID NO:61, a cytosine at a position corresponding to position 813 according to SEQ ID NO:62, a cytosine at a position corresponding to position 788 according to SEQ ID NO:63, a cytosine at a position corresponding to position 581 according to SEQ ID NO:64, a cytosine at a position corresponding to position 574 according to SEQ ID NO:65, a cytosine at a position corresponding to position 813 according to SEQ ID NO:66, a cytosine at a position corresponding to position 799 according to SEQ ID NO:139, a cytosine at a position corresponding to position 866 according to SEQ ID NO:140, a cytosine at a position corresponding to position 831 according to SEQ ID NO:141, a cytosine at a position corresponding to position 813 according to SEQ ID NO:142, a cytosine at a position corresponding to position 788 according to SEQ ID NO:143, a cytosine at a position corresponding to position 581 according to SEQ ID NO:144, a cytosine at a position corresponding to position 574 according to SEQ ID NO:145, a cytosine at a position corresponding to position 813 according to SEQ ID NO:146, the biological sample can be subjected to an amplification method using a primer pair that includes a first primer derived from the 5′ flanking sequence adjacent to a cytosine at a position corresponding to position 4,322 according to SEQ ID NO:7, a cytosine at a position corresponding to position 799 according to SEQ ID NO:59, a cytosine at a position corresponding to position 866 according to SEQ ID NO:60, a cytosine at a position corresponding to position 831 according to SEQ ID NO:61, a cytosine at a position corresponding to position 813 according to SEQ ID NO:62, a cytosine at a position corresponding to position 788 according to SEQ ID NO:63, a cytosine at a position corresponding to position 581 according to SEQ ID NO:64, a cytosine at a position corresponding to position 574 according to SEQ ID NO:65, a cytosine at a position corresponding to position 813 according to SEQ ID NO:66, a cytosine at a position corresponding to position 799 according to SEQ ID NO:139, a cytosine at a position corresponding to position 866 according to SEQ ID NO:140, a cytosine at a position corresponding to position 831 according to SEQ ID NO:141, a cytosine at a position corresponding to position 813 according to SEQ ID NO:142, a cytosine at a position corresponding to position 788 according to SEQ ID NO:143, a cytosine at a position corresponding to position 581 according to SEQ ID NO:144, a cytosine at a position corresponding to position 574 according to SEQ ID NO:145, a cytosine at a position corresponding to position 813 according to SEQ ID NO:146, and a second primer derived from the 3′ flanking sequence adjacent to a cytosine at a position corresponding to position 4,322 according to SEQ ID NO:7, a cytosine at a position corresponding to position 799 according to SEQ ID NO:59, a cytosine at a position corresponding to position 866 according to SEQ ID NO:60, a cytosine at a position corresponding to position 831 according to SEQ ID NO:61, a cytosine at a position corresponding to position 813 according to SEQ ID NO:62, a cytosine at a position corresponding to position 788 according to SEQ ID NO:63, a cytosine at a position corresponding to position 581 according to SEQ ID NO:64, a cytosine at a position corresponding to position 574 according to SEQ ID NO:65, a cytosine at a position corresponding to position 813 according to SEQ ID NO:66, a cytosine at a position corresponding to position 799 according to SEQ ID NO:139, a cytosine at a position corresponding to position 866 according to SEQ ID NO:140, a cytosine at a position corresponding to position 831 according to SEQ ID NO:141, a cytosine at a position corresponding to position 813 according to SEQ ID NO:142, a cytosine at a position corresponding to position 788 according to SEQ ID NO:143, a cytosine at a position corresponding to position 581 according to SEQ ID NO:144, a cytosine at a position corresponding to position 574 according to SEQ ID NO:145, a cytosine at a position corresponding to position 813 according to SEQ ID NO:146 to produce an amplicon that is indicative of the presence of the SNP at positions encoding a cytosine at a position corresponding to position 4,322 according to SEQ ID NO:7, a cytosine at a position corresponding to position 799 according to SEQ ID NO:59, a cytosine at a position corresponding to position 866 according to SEQ ID NO:60, a cytosine at a position corresponding to position 831 according to SEQ ID NO:61, a cytosine at a position corresponding to position 813 according to SEQ ID NO:62, a cytosine at a position corresponding to position 788 according to SEQ ID NO:63, a cytosine at a position corresponding to position 581 according to SEQ ID NO:64, a cytosine at a position corresponding to position 574 according to SEQ ID NO:65, a cytosine at a position corresponding to position 813 according to SEQ ID NO:66, a cytosine at a position corresponding to position 799 according to SEQ ID NO:139, a cytosine at a position corresponding to position 866 according to SEQ ID NO:140, a cytosine at a position corresponding to position 831 according to SEQ ID NO:141, a cytosine at a position corresponding to position 813 according to SEQ ID NO:142, a cytosine at a position corresponding to position 788 according to SEQ ID NO:143, a cytosine at a position corresponding to position 581 according to SEQ ID NO:144, a cytosine at a position corresponding to position 574 according to SEQ ID NO:145, a cytosine at a position corresponding to position 813 according to SEQ ID NO:146. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a cytosine at a position corresponding to position 4,322 according to SEQ ID NO:7, a cytosine at a position corresponding to position 799 according to SEQ ID NO:59, a cytosine at a position corresponding to position 866 according to SEQ ID NO:60, a cytosine at a position corresponding to position 831 according to SEQ ID NO:61, a cytosine at a position corresponding to position 813 according to SEQ ID NO:62, a cytosine at a position corresponding to position 788 according to SEQ ID NO:63, a cytosine at a position corresponding to position 581 according to SEQ ID NO:64, a cytosine at a position corresponding to position 574 according to SEQ ID NO:65, a cytosine at a position corresponding to position 813 according to SEQ ID NO:66, a cytosine at a position corresponding to position 799 according to SEQ ID NO:139, a cytosine at a position corresponding to position 866 according to SEQ ID NO:140, a cytosine at a position corresponding to position 831 according to SEQ ID NO:141, a cytosine at a position corresponding to position 813 according to SEQ ID NO:142, a cytosine at a position corresponding to position 788 according to SEQ ID NO:143, a cytosine at a position corresponding to position 581 according to SEQ ID NO:144, a cytosine at a position corresponding to position 574 according to SEQ ID NO:145, a cytosine at a position corresponding to position 813 according to SEQ ID NO:146 and at least 1, 2, 3, 4, 5, 6, 7, 8, 9, 10, or more nucleotides on each side of positions comprising a cytosine at a position corresponding to position 4,322 according to SEQ ID NO:7, a cytosine at a position corresponding to position 799 according to SEQ ID NO:59, a cytosine at a position corresponding to position 866 according to SEQ ID NO:60, a cytosine at a position corresponding to position 831 according to SEQ ID NO:61, a cytosine at a position corresponding to position 813 according to SEQ ID NO:62, a cytosine at a position corresponding to position 788 according to SEQ ID NO:63, a cytosine at a position corresponding to position 581 according to SEQ ID NO:64, a cytosine at a position corresponding to position 574 according to SEQ ID NO:65, a cytosine at a position corresponding to position 813 according to SEQ ID NO:66, a cytosine at a position corresponding to position 799 according to SEQ ID NO:139, a cytosine at a position corresponding to position 866 according to SEQ ID NO:140, a cytosine at a position corresponding to position 831 according to SEQ ID NO:141, a cytosine at a position corresponding to position 813 according to SEQ ID NO:142, a cytosine at a position corresponding to position 788 according to SEQ ID NO:143, a cytosine at a position corresponding to position 581 according to SEQ ID NO:144, a cytosine at a position corresponding to position 574 according to SEQ ID NO:145, a cytosine at a position corresponding to position 813 according to SEQ ID NO:146.

In some embodiments, to determine whether an ANGPTL7 nucleic acid molecule (genomic nucleic acid molecule, mRNA molecule, or cDNA molecule), or complement thereof, within a biological sample comprises a nucleotide sequence comprising a thymine at a position corresponding to position 5,125 according to SEQ ID NO:8, a uracil at a position corresponding to position 916 according to SEQ ID NO:67, a uracil at a position corresponding to position 983 according to SEQ ID NO:68, a uracil at a position corresponding to position 948 according to SEQ ID NO:69, a uracil at a position corresponding to position 930 according to SEQ ID NO:70, a uracil at a position corresponding to position 905 according to SEQ ID NO:71, a uracil at a position corresponding to position 698 according to SEQ ID NO:72, a uracil at a position corresponding to position 691 according to SEQ ID NO:73, a uracil at a position corresponding to position 930 according to SEQ ID NO:74, a thymine at a position corresponding to position 916 according to SEQ ID NO:147, a thymine at a position corresponding to position 983 according to SEQ ID NO:148, a thymine at a position corresponding to position 948 according to SEQ ID NO:149, a thymine at a position corresponding to position 930 according to SEQ ID NO:150, a thymine at a position corresponding to position 905 according to SEQ ID NO:151, a thymine at a position corresponding to position 698 according to SEQ ID NO:152, a thymine at a position corresponding to position 691 according to SEQ ID NO:153, a thymine at a position corresponding to position 930 according to SEQ ID NO:154, the biological sample can be subjected to an amplification method using a primer pair that includes a first primer derived from the 5′ flanking sequence adjacent to a thymine at a position corresponding to position 5,125 according to SEQ ID NO:8, a uracil at a position corresponding to position 916 according to SEQ ID NO:67, a uracil at a position corresponding to position 983 according to SEQ ID NO:68, a uracil at a position corresponding to position 948 according to SEQ ID NO:69, a uracil at a position corresponding to position 930 according to SEQ ID NO:70, a uracil at a position corresponding to position 905 according to SEQ ID NO:71, a uracil at a position corresponding to position 698 according to SEQ ID NO:72, a uracil at a position corresponding to position 691 according to SEQ ID NO:73, a uracil at a position corresponding to position 930 according to SEQ ID NO:74, a thymine at a position corresponding to position 916 according to SEQ ID NO:147, a thymine at a position corresponding to position 983 according to SEQ ID NO:148, a thymine at a position corresponding to position 948 according to SEQ ID NO:149, a thymine at a position corresponding to position 930 according to SEQ ID NO:150, a thymine at a position corresponding to position 905 according to SEQ ID NO:151, a thymine at a position corresponding to position 698 according to SEQ ID NO:152, a thymine at a position corresponding to position 691 according to SEQ ID NO:153, a thymine at a position corresponding to position 930 according to SEQ ID NO:154, and a second primer derived from the 3′ flanking sequence adjacent to a thymine at a position corresponding to position 5,125 according to SEQ ID NO:8, a uracil at a position corresponding to position 916 according to SEQ ID NO:67, a uracil at a position corresponding to position 983 according to SEQ ID NO:68, a uracil at a position corresponding to position 948 according to SEQ ID NO:69, a uracil at a position corresponding to position 930 according to SEQ ID NO:70, a uracil at a position corresponding to position 905 according to SEQ ID NO:71, a uracil at a position corresponding to position 698 according to SEQ ID NO:72, a uracil at a position corresponding to position 691 according to SEQ ID NO:73, a uracil at a position corresponding to position 930 according to SEQ ID NO:74, a thymine at a position corresponding to position 916 according to SEQ ID NO:147, a thymine at a position corresponding to position 983 according to SEQ ID NO:148, a thymine at a position corresponding to position 948 according to SEQ ID NO:149, a thymine at a position corresponding to position 930 according to SEQ ID NO:150, a thymine at a position corresponding to position 905 according to SEQ ID NO:151, a thymine at a position corresponding to position 698 according to SEQ ID NO:152, a thymine at a position corresponding to position 691 according to SEQ ID NO:153, a thymine at a position corresponding to position 930 according to SEQ ID NO:154 to produce an amplicon that is indicative of the presence of the SNP at positions encoding a thymine at a position corresponding to position 5,125 according to SEQ ID NO:8, a uracil at a position corresponding to position 916 according to SEQ ID NO:67, a uracil at a position corresponding to position 983 according to SEQ ID NO:68, a uracil at a position corresponding to position 948 according to SEQ ID NO:69, a uracil at a position corresponding to position 930 according to SEQ ID NO:70, a uracil at a position corresponding to position 905 according to SEQ ID NO:71, a uracil at a position corresponding to position 698 according to SEQ ID NO:72, a uracil at a position corresponding to position 691 according to SEQ ID NO:73, a uracil at a position corresponding to position 930 according to SEQ ID NO:74, a thymine at a position corresponding to position 916 according to SEQ ID NO:147, a thymine at a position corresponding to position 983 according to SEQ ID NO:148, a thymine at a position corresponding to position 948 according to SEQ ID NO:149, a thymine at a position corresponding to position 930 according to SEQ ID NO:150, a thymine at a position corresponding to position 905 according to SEQ ID NO:151, a thymine at a position corresponding to position 698 according to SEQ ID NO:152, a thymine at a position corresponding to position 691 according to SEQ ID NO:153, a thymine at a position corresponding to position 930 according to SEQ ID NO:154. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 5,125 according to SEQ ID NO:8, a uracil at a position corresponding to position 916 according to SEQ ID NO:67, a uracil at a position corresponding to position 983 according to SEQ ID NO:68, a uracil at a position corresponding to position 948 according to SEQ ID NO:69, a uracil at a position corresponding to position 930 according to SEQ ID NO:70, a uracil at a position corresponding to position 905 according to SEQ ID NO:71, a uracil at a position corresponding to position 698 according to SEQ ID NO:72, a uracil at a position corresponding to position 691 according to SEQ ID NO:73, a uracil at a position corresponding to position 930 according to SEQ ID NO:74, a thymine at a position corresponding to position 916 according to SEQ ID NO:147, a thymine at a position corresponding to position 983 according to SEQ ID NO:148, a thymine at a position corresponding to position 948 according to SEQ ID NO:149, a thymine at a position corresponding to position 930 according to SEQ ID NO:150, a thymine at a position corresponding to position 905 according to SEQ ID NO:151, a thymine at a position corresponding to position 698 according to SEQ ID NO:152, a thymine at a position corresponding to position 691 according to SEQ ID NO:153, a thymine at a position corresponding to position 930 according to SEQ ID NO:154, and at least 1, 2, 3, 4, 5, 6, 7, 8, 9, 10, or more nucleotides on each side of positions comprising a thymine at a position corresponding to position 5,125 according to SEQ ID NO:8, a uracil at a position corresponding to position 916 according to SEQ ID NO:67, a uracil at a position corresponding to position 983 according to SEQ ID NO:68, a uracil at a position corresponding to position 948 according to SEQ ID NO:69, a uracil at a position corresponding to position 930 according to SEQ ID NO:70, a uracil at a position corresponding to position 905 according to SEQ ID NO:71, a uracil at a position corresponding to position 698 according to SEQ ID NO:72, a uracil at a position corresponding to position 691 according to SEQ ID NO:73, a uracil at a position corresponding to position 930 according to SEQ ID NO:74, a thymine at a position corresponding to position 916 according to SEQ ID NO:147, a thymine at a position corresponding to position 983 according to SEQ ID NO:148, a thymine at a position corresponding to position 948 according to SEQ ID NO:149, a thymine at a position corresponding to position 930 according to SEQ ID NO:150, a thymine at a position corresponding to position 905 according to SEQ ID NO:151, a thymine at a position corresponding to position 698 according to SEQ ID NO:152, a thymine at a position corresponding to position 691 according to SEQ ID NO:153, a thymine at a position corresponding to position 930 according to SEQ ID NO:154.

In some embodiments, to determine whether an ANGPTL7 nucleic acid molecule (genomic nucleic acid molecule, mRNA molecule, or cDNA molecule), or complement thereof, within a biological sample comprises a nucleotide sequence comprising a thymine at a position corresponding to position 5,176 according to SEQ ID NO:9, a uracil at a position corresponding to position 967 according to SEQ ID NO:75, a uracil at a position corresponding to position 1,034 according to SEQ ID NO:76, a uracil at a position corresponding to position 999 according to SEQ ID NO:77, a uracil at a position corresponding to position 981 according to SEQ ID NO:78, a uracil at a position corresponding to position 956 according to SEQ ID NO:79, a uracil at a position corresponding to position 749 according to SEQ ID NO:80, a uracil at a position corresponding to position 742 according to SEQ ID NO:81, a uracil at a position corresponding to position 981 according to SEQ ID NO:82, a thymine at a position corresponding to position 967 according to SEQ ID NO:155, a thymine at a position corresponding to position 1,034 according to SEQ ID NO:156, a thymine at a position corresponding to position 999 according to SEQ ID NO:157, a thymine at a position corresponding to position 981 according to SEQ ID NO:158, a thymine at a position corresponding to position 956 according to SEQ ID NO:159, a thymine at a position corresponding to position 749 according to SEQ ID NO:160, a thymine at a position corresponding to position 742 according to SEQ ID NO:161, a thymine at a position corresponding to position 981 according to SEQ ID NO:162, the biological sample can be subjected to an amplification method using a primer pair that includes a first primer derived from the 5′ flanking sequence adjacent to a thymine at a position corresponding to position 5,176 according to SEQ ID NO:9, a uracil at a position corresponding to position 967 according to SEQ ID NO:75, a uracil at a position corresponding to position 1,034 according to SEQ ID NO:76, a uracil at a position corresponding to position 999 according to SEQ ID NO:77, a uracil at a position corresponding to position 981 according to SEQ ID NO:78, a uracil at a position corresponding to position 956 according to SEQ ID NO:79, a uracil at a position corresponding to position 749 according to SEQ ID NO:80, a uracil at a position corresponding to position 742 according to SEQ ID NO:81, a uracil at a position corresponding to position 981 according to SEQ ID NO:82, a thymine at a position corresponding to position 967 according to SEQ ID NO:155, a thymine at a position corresponding to position 1,034 according to SEQ ID NO:156, a thymine at a position corresponding to position 999 according to SEQ ID NO:157, a thymine at a position corresponding to position 981 according to SEQ ID NO:158, a thymine at a position corresponding to position 956 according to SEQ ID NO:159, a thymine at a position corresponding to position 749 according to SEQ ID NO:160, a thymine at a position corresponding to position 742 according to SEQ ID NO:161, a thymine at a position corresponding to position 981 according to SEQ ID NO:162, and a second primer derived from the 3′ flanking sequence adjacent to a thymine at a position corresponding to position 5,176 according to SEQ ID NO:9, a uracil at a position corresponding to position 967 according to SEQ ID NO:75, a uracil at a position corresponding to position 1,034 according to SEQ ID NO:76, a uracil at a position corresponding to position 999 according to SEQ ID NO:77, a uracil at a position corresponding to position 981 according to SEQ ID NO:78, a uracil at a position corresponding to position 956 according to SEQ ID NO:79, a uracil at a position corresponding to position 749 according to SEQ ID NO:80, a uracil at a position corresponding to position 742 according to SEQ ID NO:81, a uracil at a position corresponding to position 981 according to SEQ ID NO:82, a thymine at a position corresponding to position 967 according to SEQ ID NO:155, a thymine at a position corresponding to position 1,034 according to SEQ ID NO:156, a thymine at a position corresponding to position 999 according to SEQ ID NO:157, a thymine at a position corresponding to position 981 according to SEQ ID NO:158, a thymine at a position corresponding to position 956 according to SEQ ID NO:159, a thymine at a position corresponding to position 749 according to SEQ ID NO:160, a thymine at a position corresponding to position 742 according to SEQ ID NO:161, a thymine at a position corresponding to position 981 according to SEQ ID NO:162 to produce an amplicon that is indicative of the presence of the SNP at positions encoding a thymine at a position corresponding to position 5,176 according to SEQ ID NO:9, a uracil at a position corresponding to position 967 according to SEQ ID NO:75, a uracil at a position corresponding to position 1,034 according to SEQ ID NO:76, a uracil at a position corresponding to position 999 according to SEQ ID NO:77, a uracil at a position corresponding to position 981 according to SEQ ID NO:78, a uracil at a position corresponding to position 956 according to SEQ ID NO:79, a uracil at a position corresponding to position 749 according to SEQ ID NO:80, a uracil at a position corresponding to position 742 according to SEQ ID NO:81, a uracil at a position corresponding to position 981 according to SEQ ID NO:82, a thymine at a position corresponding to position 967 according to SEQ ID NO:155, a thymine at a position corresponding to position 1,034 according to SEQ ID NO:156, a thymine at a position corresponding to position 999 according to SEQ ID NO:157, a thymine at a position corresponding to position 981 according to SEQ ID NO:158, a thymine at a position corresponding to position 956 according to SEQ ID NO:159, a thymine at a position corresponding to position 749 according to SEQ ID NO:160, a thymine at a position corresponding to position 742 according to SEQ ID NO:161, a thymine at a position corresponding to position 981 according to SEQ ID NO:162. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 5,176 according to SEQ ID NO:9, a uracil at a position corresponding to position 967 according to SEQ ID NO:75, a uracil at a position corresponding to position 1,034 according to SEQ ID NO:76, a uracil at a position corresponding to position 999 according to SEQ ID NO:77, a uracil at a position corresponding to position 981 according to SEQ ID NO:78, a uracil at a position corresponding to position 956 according to SEQ ID NO:79, a uracil at a position corresponding to position 749 according to SEQ ID NO:80, a uracil at a position corresponding to position 742 according to SEQ ID NO:81, a uracil at a position corresponding to position 981 according to SEQ ID NO:82, a thymine at a position corresponding to position 967 according to SEQ ID NO:155, a thymine at a position corresponding to position 1,034 according to SEQ ID NO:156, a thymine at a position corresponding to position 999 according to SEQ ID NO:157, a thymine at a position corresponding to position 981 according to SEQ ID NO:158, a thymine at a position corresponding to position 956 according to SEQ ID NO:159, a thymine at a position corresponding to position 749 according to SEQ ID NO:160, a thymine at a position corresponding to position 742 according to SEQ ID NO:161, a thymine at a position corresponding to position 981 according to SEQ ID NO:162 and at least 1, 2, 3, 4, 5, 6, 7, 8, 9, 10, or more nucleotides on each side of positions comprising a thymine at a position corresponding to position 5,176 according to SEQ ID NO:9, a uracil at a position corresponding to position 967 according to SEQ ID NO:75, a uracil at a position corresponding to position 1,034 according to SEQ ID NO:76, a uracil at a position corresponding to position 999 according to SEQ ID NO:77, a uracil at a position corresponding to position 981 according to SEQ ID NO:78, a uracil at a position corresponding to position 956 according to SEQ ID NO:79, a uracil at a position corresponding to position 749 according to SEQ ID NO:80, a uracil at a position corresponding to position 742 according to SEQ ID NO:81, a uracil at a position corresponding to position 981 according to SEQ ID NO:82, a thymine at a position corresponding to position 967 according to SEQ ID NO:155, a thymine at a position corresponding to position 1,034 according to SEQ ID NO:156, a thymine at a position corresponding to position 999 according to SEQ ID NO:157, a thymine at a position corresponding to position 981 according to SEQ ID NO:158, a thymine at a position corresponding to position 956 according to SEQ ID NO:159, a thymine at a position corresponding to position 749 according to SEQ ID NO:160, a thymine at a position corresponding to position 742 according to SEQ ID NO:161, a thymine at a position corresponding to position 981 according to SEQ ID NO:162.

In some embodiments, to determine whether an ANGPTL7 nucleic acid molecule (genomic nucleic acid molecule, mRNA molecule, or cDNA molecule), or complement thereof, within a biological sample comprises a nucleotide sequence comprising an adenine at a position corresponding to position 5,232 according to SEQ ID NO:10, an adenine at a position corresponding to position 1,023 according to SEQ ID NO:83, an adenine at a position corresponding to position 1,090 according to SEQ ID NO:84, an adenine at a position corresponding to position 1,055 according to SEQ ID NO:85, an adenine at a position corresponding to position 1,037 according to SEQ ID NO:86, an adenine at a position corresponding to position 1,012 according to SEQ ID NO:87, an adenine at a position corresponding to position 805 according to SEQ ID NO:88, an adenine at a position corresponding to position 798 according to SEQ ID NO:89, or an adenine at a position corresponding to position 1,037 according to SEQ ID NO:90, an adenine at a position corresponding to position 1,023 according to SEQ ID NO:163, an adenine at a position corresponding to position 1,090 according to SEQ ID NO:164, an adenine at a position corresponding to position 1,055 according to SEQ ID NO:165, an adenine at a position corresponding to position 1,037 according to SEQ ID NO:166, an adenine at a position corresponding to position 1,012 according to SEQ ID NO:167, an adenine at a position corresponding to position 805 according to SEQ ID NO:168, an adenine at a position corresponding to position 798 according to SEQ ID NO:169, or an adenine at a position corresponding to position 1,037 according to SEQ ID NO:170, the biological sample can be subjected to an amplification method using a primer pair that includes a first primer derived from the 5′ flanking sequence adjacent to an adenine at a position corresponding to position 5,232 according to SEQ ID NO:10, an adenine at a position corresponding to position 1,023 according to SEQ ID NO:83, an adenine at a position corresponding to position 1,090 according to SEQ ID NO:84, an adenine at a position corresponding to position 1,055 according to SEQ ID NO:85, an adenine at a position corresponding to position 1,037 according to SEQ ID NO:86, an adenine at a position corresponding to position 1,012 according to SEQ ID NO:87, an adenine at a position corresponding to position 805 according to SEQ ID NO:88, an adenine at a position corresponding to position 798 according to SEQ ID NO:89, or an adenine at a position corresponding to position 1,037 according to SEQ ID NO:90, an adenine at a position corresponding to position 1,023 according to SEQ ID NO:163, an adenine at a position corresponding to position 1,090 according to SEQ ID NO:164, an adenine at a position corresponding to position 1,055 according to SEQ ID NO:165, an adenine at a position corresponding to position 1,037 according to SEQ ID NO:166, an adenine at a position corresponding to position 1,012 according to SEQ ID NO:167, an adenine at a position corresponding to position 805 according to SEQ ID NO:168, an adenine at a position corresponding to position 798 according to SEQ ID NO:169, or an adenine at a position corresponding to position 1,037 according to SEQ ID NO:170, and a second primer derived from the 3′ flanking sequence adjacent to an adenine at a position corresponding to position 5,232 according to SEQ ID NO:10, an adenine at a position corresponding to position 1,023 according to SEQ ID NO:83, an adenine at a position corresponding to position 1,090 according to SEQ ID NO:84, an adenine at a position corresponding to position 1,055 according to SEQ ID NO:85, an adenine at a position corresponding to position 1,037 according to SEQ ID NO:86, an adenine at a position corresponding to position 1,012 according to SEQ ID NO:87, an adenine at a position corresponding to position 805 according to SEQ ID NO:88, an adenine at a position corresponding to position 798 according to SEQ ID NO:89, or an adenine at a position corresponding to position 1,037 according to SEQ ID NO:90, an adenine at a position corresponding to position 1,023 according to SEQ ID NO:163, an adenine at a position corresponding to position 1,090 according to SEQ ID NO:164, an adenine at a position corresponding to position 1,055 according to SEQ ID NO:165, an adenine at a position corresponding to position 1,037 according to SEQ ID NO:166, an adenine at a position corresponding to position 1,012 according to SEQ ID NO:167, an adenine at a position corresponding to position 805 according to SEQ ID NO:168, an adenine at a position corresponding to position 798 according to SEQ ID NO:169, or an adenine at a position corresponding to position 1,037 according to SEQ ID NO:170. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising an adenine at a position corresponding to position 5,232 according to SEQ ID NO:10, an adenine at a position corresponding to position 1,023 according to SEQ ID NO:83, an adenine at a position corresponding to position 1,090 according to SEQ ID NO:84, an adenine at a position corresponding to position 1,055 according to SEQ ID NO:85, an adenine at a position corresponding to position 1,037 according to SEQ ID NO:86, an adenine at a position corresponding to position 1,012 according to SEQ ID NO:87, an adenine at a position corresponding to position 805 according to SEQ ID NO:88, an adenine at a position corresponding to position 798 according to SEQ ID NO:89, or an adenine at a position corresponding to position 1,037 according to SEQ ID NO:90, an adenine at a position corresponding to position 1,023 according to SEQ ID NO:163, an adenine at a position corresponding to position 1,090 according to SEQ ID NO:164, an adenine at a position corresponding to position 1,055 according to SEQ ID NO:165, an adenine at a position corresponding to position 1,037 according to SEQ ID NO:166, an adenine at a position corresponding to position 1,012 according to SEQ ID NO:167, an adenine at a position corresponding to position 805 according to SEQ ID NO:168, an adenine at a position corresponding to position 798 according to SEQ ID NO:169, or an adenine at a position corresponding to position 1,037 according to SEQ ID NO:170 and at least 1, 2, 3, 4, 5, 6, 7, 8, 9, 10, or more nucleotides on each side of positions comprising an adenine at a position corresponding to position 5,232 according to SEQ ID NO:10, an adenine at a position corresponding to position 1,023 according to SEQ ID NO:83, an adenine at a position corresponding to position 1,090 according to SEQ ID NO:84, an adenine at a position corresponding to position 1,055 according to SEQ ID NO:85, an adenine at a position corresponding to position 1,037 according to SEQ ID NO:86, an adenine at a position corresponding to position 1,012 according to SEQ ID NO:87, an adenine at a position corresponding to position 805 according to SEQ ID NO:88, an adenine at a position corresponding to position 798 according to SEQ ID NO:89, or an adenine at a position corresponding to position 1,037 according to SEQ ID NO:90, an adenine at a position corresponding to position 1,023 according to SEQ ID NO:163, an adenine at a position corresponding to position 1,090 according to SEQ ID NO:164, an adenine at a position corresponding to position 1,055 according to SEQ ID NO:165, an adenine at a position corresponding to position 1,037 according to SEQ ID NO:166, an adenine at a position corresponding to position 1,012 according to SEQ ID NO:167, an adenine at a position corresponding to position 805 according to SEQ ID NO:168, an adenine at a position corresponding to position 798 according to SEQ ID NO:169, or an adenine at a position corresponding to position 1,037 according to SEQ ID NO:170.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6λ sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of an ANGPTL7 predicted loss-of-function polypeptide comprising performing an assay on a biological sample obtained from the subject to determine whether an ANGPTL7 polypeptide in the biological sample contains one or more variations that causes the polypeptide to have a loss-of-function (partial or complete) or predicted loss-of-function (partial or complete). The ANGPTL7 predicted loss-of-function polypeptide can be any of the ANGPTL7 predicted loss-of-function polypeptides described herein. In some embodiments, the methods detect the presence of ANGPTL7 Phe161Ile, Ile174Asn, Gln175His, Arg177STOP, Trp188STOP, Lys192Gln, Arg220Cys, Arg220His, Arg231Cys, Arg248Cys, His266Gln, Asn302Lys, or Arg340His. In some embodiments, the methods detect the presence of ANGPTL7 Phe161Ile, Ile174Asn, Gln175His, Arg177STOP, Trp188STOP, Lys192Gln, Arg220Cys, Arg220His, Arg231Cys, Arg248Cys, His266Gln, Asn302Lys, or Arg340His.

In some embodiments, the methods comprise performing an assay on a biological sample obtained from a subject to determine whether an ANGPTL7 polypeptide in the biological sample comprises an isoleucine at a position corresponding to position 161 according to SEQ ID NO:172. In some embodiments, the methods comprise performing an assay on a biological sample obtained from a subject to determine whether an ANGPTL7 polypeptide in the biological sample comprises an asparagine at a position corresponding to position 174 according to SEQ ID NO:173. In some embodiments, the methods comprise performing an assay on a biological sample obtained from a subject to determine whether an ANGPTL7 polypeptide in the biological sample comprises a histidine at a position corresponding to position 175 according to SEQ ID NO:174. In some embodiments, the methods comprise performing an assay on a biological sample obtained from a subject to determine whether an ANGPTL7 polypeptide in the biological sample comprises a stop codon at a position corresponding to position 177 according to SEQ ID NO:175. In some embodiments, the methods comprise performing an assay on a biological sample obtained from a subject to determine whether an ANGPTL7 polypeptide in the biological sample comprises a stop codon at a position corresponding to position 188 according to SEQ ID NO:176. In some embodiments, the methods comprise performing an assay on a biological sample obtained from a subject to determine whether an ANGPTL7 polypeptide in the biological sample comprises a glutamine at a position corresponding to position 192 according to SEQ ID NO:177. In some embodiments, the methods comprise performing an assay on a biological sample obtained from a subject to determine whether an ANGPTL7 polypeptide in the biological sample comprises a cysteine at a position corresponding to position 231 according to SEQ ID NO:178. In some embodiments, the methods comprise performing an assay on a biological sample obtained from a subject to determine whether an ANGPTL7 polypeptide in the biological sample comprises a cysteine at a position corresponding to position 248 according to SEQ ID NO:179. In some embodiments, the methods comprise performing an assay on a biological sample obtained from a subject to determine whether an ANGPTL7 polypeptide in the biological sample comprises a glutamine at a position corresponding to position 266 according to SEQ ID NO:180.

In some embodiments, the detecting step comprises sequencing at least a portion of the ANGPTL7 polypeptide that comprises a position corresponding to position 161 according to SEQ ID NO:172. In some embodiments, the detecting step comprises sequencing at least a portion of the ANGPTL7 polypeptide that comprises a position corresponding to position 174 according to SEQ ID NO:173. In some embodiments, the detecting step comprises sequencing at least a portion of the ANGPTL7 polypeptide that comprises a position corresponding to position 175 according to SEQ ID NO:174. In some embodiments, the detecting step comprises sequencing at least a portion of the ANGPTL7 polypeptide that comprises a position corresponding to position 177 according to SEQ ID NO:175. In some embodiments, the detecting step comprises sequencing at least a portion of the ANGPTL7 polypeptide that comprises a position corresponding to position 188 according to SEQ ID NO:176. In some embodiments, the detecting step comprises sequencing at least a portion of the ANGPTL7 polypeptide that comprises a position corresponding to position 192 according to SEQ ID NO:177. In some embodiments, the detecting step comprises sequencing at least a portion of the ANGPTL7 polypeptide that comprises a position corresponding to position 231 according to SEQ ID NO:178. In some embodiments, the detecting step comprises sequencing at least a portion of the ANGPTL7 polypeptide that comprises a position corresponding to position 248 according to SEQ ID NO:179. In some embodiments, the detecting step comprises sequencing at least a portion of the ANGPTL7 polypeptide that comprises a position corresponding to position 266 according to SEQ ID NO:180.

In some embodiments, the detecting step comprises an immunoassay for detecting the presence of an ANGPTL7 polypeptide that comprises a position corresponding to position 161 according to SEQ ID NO:172. In some embodiments, the detecting step comprises an immunoassay for detecting the presence of an ANGPTL7 polypeptide that comprises a position corresponding to position 174 according to SEQ ID NO:173. In some embodiments, the detecting step comprises an immunoassay for detecting the presence of an ANGPTL7 polypeptide that comprises a position corresponding to position 175 according to SEQ ID NO:174. In some embodiments, the detecting step comprises an immunoassay for detecting the presence of an ANGPTL7 polypeptide that comprises a position corresponding to position 177 according to SEQ ID NO:175. In some embodiments, the detecting step comprises an immunoassay for detecting the presence of an ANGPTL7 polypeptide that comprises a position corresponding to position 188 according to SEQ ID NO:176. In some embodiments, the detecting step comprises an immunoassay for detecting the presence of an ANGPTL7 polypeptide that comprises a position corresponding to position 192 according to SEQ ID NO:177. In some embodiments, the detecting step comprises an immunoassay for detecting the presence of an ANGPTL7 polypeptide that comprises a position corresponding to position 231 according to SEQ ID NO:178. In some embodiments, the detecting step comprises an immunoassay for detecting the presence of an ANGPTL7 polypeptide that comprises a position corresponding to position 248 according to SEQ ID NO:179. In some embodiments, the detecting step comprises an immunoassay for detecting the presence of an ANGPTL7 polypeptide that comprises a position corresponding to position 266 according to SEQ ID NO:180.

In some embodiments, when the subject does not have an ANGPTL7 predicted loss-of-function polypeptide, the subject has an increased risk of developing ophthalmic conditions or any of glaucoma, including open-angle glaucoma, angle-closure glaucoma, normal-tension glaucoma, congenital glaucoma, secondary glaucoma, pigmentary glaucoma, pseudoexfoliative glaucoma, traumatic glaucoma, neovascular glaucoma, uveitic glaucoma, or irido corneal endothelial syndrome. In some embodiments, when the subject has an ANGPTL7 predicted loss-of-function polypeptide, the subject has a decreased risk of developing ophthalmic conditions or any of glaucoma, including open-angle glaucoma, angle-closure glaucoma, normal-tension glaucoma, congenital glaucoma, secondary glaucoma, pigmentary glaucoma, pseudoexfoliative glaucoma, traumatic glaucoma, neovascular glaucoma, uveitic glaucoma, or irido corneal endothelial syndrome.

The present disclosure also provides isolated nucleic acid molecules that hybridize to ANGPTL7 missense variant genomic nucleic acid molecules, ANGPTL7 missense variant mRNA molecules, and/or ANGPTL7 missense variant cDNA molecules (such as any of the genomic variant nucleic acid molecules, mRNA variant molecules, and cDNA variant molecules disclosed herein). In some embodiments, such isolated nucleic acid molecules hybridize to ANGPTL7 missense variant nucleic acid molecules under stringent conditions. Such nucleic acid molecules can be used, for example, as probes, primers, alteration-specific probes, or alteration-specific primers as described or exemplified herein.

In some embodiments, the isolated nucleic acid molecules hybridize to a portion of the ANGPTL7 missense nucleic acid molecule that includes a position corresponding to: position 4,229 according to SEQ ID NO:2, position 706 according to SEQ ID NO:19, position 773 according to SEQ ID NO:20, position 738 according to SEQ ID NO:21, position 720 according to SEQ ID NO:22, position 695 according to SEQ ID NO:23, position 488 according to SEQ ID NO:24, position 481 according to SEQ ID NO:25, position 720 according to SEQ ID NO:26, position 706 according to SEQ ID NO:99, position 773 according to SEQ ID NO:100, position 738 according to SEQ ID NO:101, position 720 according to SEQ ID NO:102, position 695 according to SEQ ID NO:103, position 488 according to SEQ ID NO:104, position 481 according to SEQ ID NO:105, or position 720 according to SEQ ID NO:106.

In some embodiments, the isolated nucleic acid molecules hybridize to a portion of the ANGPTL7 missense nucleic acid molecule that includes a position corresponding to: position 4,269 according to SEQ ID NO:3, position 746 according to SEQ ID NO:27, position 812 according to SEQ ID NO:28, position 778 according to SEQ ID NO:29, position 760 according to SEQ ID NO:30, position 735 according to SEQ ID NO:31, position 528 according to SEQ ID NO:32, position 521 according to SEQ ID NO:33, position 760 according to SEQ ID NO:34, position 746 according to SEQ ID NO:107, position 812 according to SEQ ID NO:108, position 778 according to SEQ ID NO:109, position 760 according to SEQ ID NO:110, position 735 according to SEQ ID NO:111, position 528 according to SEQ ID NO:112, position 529 according to SEQ ID NO:113, or position 760 according to SEQ ID NO:114.

In some embodiments, the isolated nucleic acid molecules hybridize to a portion of the ANGPTL7 missense nucleic acid molecule that includes a position corresponding to: position 4,273 according to SEQ ID NO:4, position 750 according to SEQ ID NO:35, position 817 according to SEQ ID NO:36, position 782 according to SEQ ID NO:37, position 764 according to SEQ ID NO:38, position 739 according to SEQ ID NO:39, position 532 according to SEQ ID NO:40, position 525 according to SEQ ID NO:41, position 764 according to SEQ ID NO:42, position 750 according to SEQ ID NO:115, position 817 according to SEQ ID NO:116, position 782 according to SEQ ID NO:117, position 764 according to SEQ ID NO:118, position 739 according to SEQ ID NO:119, position 532 according to SEQ ID NO:120, position 525 according to SEQ ID NO:121, or position 764 according to SEQ ID NO:122.

In some embodiments, the isolated nucleic acid molecules hybridize to a portion of the ANGPTL7 missense nucleic acid molecule that includes a position corresponding to: position 4,277 according to SEQ ID NO:5, position 754 according to SEQ ID NO:43, position 821 according to SEQ ID NO:44, position 786 according to SEQ ID NO:45, position 768 according to SEQ ID NO:46, position 743 according to SEQ ID NO:47, position 536 according to SEQ ID NO:48, position 529 according to SEQ ID NO:49, position 768 according to SEQ ID NO:50, position 754 according to SEQ ID NO:123, position 821 according to SEQ ID NO:124, position 786 according to SEQ ID NO:125, position 768 according to SEQ ID NO:126, position 743 according to SEQ ID NO:127, position 536 according to SEQ ID NO:128, position 529 according to SEQ ID NO:129, or position 768 according to SEQ ID NO:130.

In some embodiments, the isolated nucleic acid molecules hybridize to a portion of the ANGPTL7 missense nucleic acid molecule that includes a position corresponding to: position 4,311 according to SEQ ID NO:6, position 788 according to SEQ ID NO:51, position 855 according to SEQ ID NO:52, position 820 according to SEQ ID NO:53, position 802 according to SEQ ID NO:54, position 777 according to SEQ ID NO:55, position 570 according to SEQ ID NO:56, position 563 according to SEQ ID NO:57, position 802 according to SEQ ID NO:58, position 788 according to SEQ ID NO:131, position 855 according to SEQ ID NO:132, position 820 according to SEQ ID NO:133, position 802 according to SEQ ID NO:134, position 777 according to SEQ ID NO:135, position 570 according to SEQ ID NO:136, position 563 according to SEQ ID NO:137, or position 802 according to SEQ ID NO:138.

In some embodiments, the isolated nucleic acid molecules hybridize to a portion of the ANGPTL7 missense nucleic acid molecule that includes a position corresponding to: position 4,322 according to SEQ ID NO:7, position 799 according to SEQ ID NO:59, position 866 according to SEQ ID NO:60, position 831 according to SEQ ID NO:61, position 813 according to SEQ ID NO:62, position 788 according to SEQ ID NO:63, position 581 according to SEQ ID NO:64, position 574 according to SEQ ID NO:65, position 813 according to SEQ ID NO:66, position 799 according to SEQ ID NO:139, position 866 according to SEQ ID NO:140, position 831 according to SEQ ID NO:141, position 813 according to SEQ ID NO:142, position 788 according to SEQ ID NO:143, position 581 according to SEQ ID NO:144, position 574 according to SEQ ID NO:145, or position 813 according to SEQ ID NO:146.

In some embodiments, the isolated nucleic acid molecules hybridize to a portion of the ANGPTL7 missense nucleic acid molecule that includes a position corresponding to: position 5,125 according to SEQ ID NO:8, position 916 according to SEQ ID NO:67, position 983 according to SEQ ID NO:68, position 948 according to SEQ ID NO:69, position 930 according to SEQ ID NO:70, position 905 according to SEQ ID NO:71, position 698 according to SEQ ID NO:72, position 691 according to SEQ ID NO:73, position 930 according to SEQ ID NO:74, position 916 according to SEQ ID NO:147, position 983 according to SEQ ID NO:148, position 948 according to SEQ ID NO:149, position 930 according to SEQ ID NO:150, position 905 according to SEQ ID NO:151, position 698 according to SEQ ID NO:152, position 691 according to SEQ ID NO:153, or position 930 according to SEQ ID NO:154.

In some embodiments, the isolated nucleic acid molecules hybridize to a portion of the ANGPTL7 missense nucleic acid molecule that includes a position corresponding to: position 5,176 according to SEQ ID NO:9, position 967 according to SEQ ID NO:75, position 1,034 according to SEQ ID NO:76, position 999 according to SEQ ID NO:77, position 981 according to SEQ ID NO:78, position 956 according to SEQ ID NO:79, position 749 according to SEQ ID NO:80, position 742 according to SEQ ID NO:81, position 981 according to SEQ ID NO:82, position 967 according to SEQ ID NO:155, position 1,034 according to SEQ ID NO:156, position 999 according to SEQ ID NO:157, position 981 according to SEQ ID NO:158, position 956 according to SEQ ID NO:159, position 749 according to SEQ ID NO:160, position 742 according to SEQ ID NO:161, or position 981 according to SEQ ID NO:162.

In some embodiments, the isolated nucleic acid molecules hybridize to a portion of the ANGPTL7 missense nucleic acid molecule that includes a position corresponding to: position 1,023 according to SEQ ID NO:83, position 1,090 according to SEQ ID NO:84, position 1,055 according to SEQ ID NO:85, position 1,037 according to SEQ ID NO:86, position 1,012 according to SEQ ID NO:87, position 805 according to SEQ ID NO:88, position 798 according to SEQ ID NO:89, position 1,037 according to SEQ ID NO:90, or the complement thereof, position 1,023 according to SEQ ID NO:163, position 1,090 according to SEQ ID NO:164, position 1,055 according to SEQ ID NO:165, position 1,037 according to SEQ ID NO:166, position 1,012 according to SEQ ID NO:167, position 805 according to SEQ ID NO:168, position 798 according to SEQ ID NO:169, or position 1,037 according to SEQ ID NO:170, or the complement thereof.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to ANGPTL7 missense variant genomic nucleic acid molecules, ANGPTL7 missense variant mRNA molecules, and/or ANGPTL7 missense variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of an ANGPTL7 missense nucleic acid molecule encoding an ANGPTL7 predicted loss-of-function polypeptide, or the complement thereof. In some embodiments, the portion comprises a position corresponding to: position 4,229 according to SEQ ID NO:2, or the complement thereof; position 706 according to SEQ ID NO:19, or the complement thereof; position 773 according to SEQ ID NO:20, or the complement thereof; position 738 according to SEQ ID NO:21, or the complement thereof; position 720 according to SEQ ID NO:22, or the complement thereof; position 695 according to SEQ ID NO:23, or the complement thereof; position 488 according to SEQ ID NO:24, or the complement thereof; position 481 according to SEQ ID NO:25, or the complement thereof; position 720 according to SEQ ID NO:26, or the complement thereof; position 706 according to SEQ ID NO:99, or the complement thereof; position 773 according to SEQ ID NO:100, or the complement thereof; position 738 according to SEQ ID NO:101, or the complement thereof; position 720 according to SEQ ID NO:102, or the complement thereof; position 695 according to SEQ ID NO:103, or the complement thereof; position 488 according to SEQ ID NO:104, or the complement thereof; position 481 according to SEQ ID NO:105, or the complement thereof; position 720 according to SEQ ID NO:106, or the complement thereof. In some embodiments, the portion comprises positions corresponding to: positions 4,229-4,231 according to SEQ ID NO:2, or the complement thereof; positions 706-708 according to SEQ ID NO:19, or the complement thereof; positions 773-775 according to SEQ ID NO:20, or the complement thereof; positions 738-740 according to SEQ ID NO:21, or the complement thereof; positions 720-722 according to SEQ ID NO:22, or the complement thereof; positions 695-697 according to SEQ ID NO:23, or the complement thereof; positions 488-490 according to SEQ ID NO:24, or the complement thereof; positions 481-483 according to SEQ ID NO:25, or the complement thereof; positions 720-722 according to SEQ ID NO:26, or the complement thereof; positions 706-708 according to SEQ ID NO:99, or the complement thereof; positions 773-775 according to SEQ ID NO:100, or the complement thereof; positions 738-740 according to SEQ ID NO:101, or the complement thereof; positions 720-722 according to SEQ ID NO:102, or the complement thereof; positions 695-697 according to SEQ ID NO:103, or the complement thereof; positions 488-490 according to SEQ ID NO:104, or the complement thereof; positions 481-483 according to SEQ ID NO:105, or the complement thereof; positions 720-722 according to SEQ ID NO:106, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of an ANGPTL7 missense nucleic acid molecule encoding an ANGPTL7 predicted loss-of-function polypeptide, or the complement thereof, wherein the portion comprises a position corresponding to: position 4,269 according to SEQ ID NO:3, or the complement thereof; position 746 according to SEQ ID NO:27, or the complement thereof; position 812 according to SEQ ID NO:28, or the complement thereof; position 778 according to SEQ ID NO:29, or the complement thereof; position 760 according to SEQ ID NO:30, or the complement thereof; position 735 according to SEQ ID NO:31, or the complement thereof; position 528 according to SEQ ID NO:32, or the complement thereof; position 521 according to SEQ ID NO:33, or the complement thereof; position 760 according to SEQ ID NO:34, or the complement thereof; position 746 according to SEQ ID NO:107, or the complement thereof; position 812 according to SEQ ID NO:108, or the complement thereof; position 778 according to SEQ ID NO:109, or the complement thereof; position 760 according to SEQ ID NO:110, or the complement thereof; position 735 according to SEQ ID NO:111, or the complement thereof; position 528 according to SEQ ID NO:112, or the complement thereof; position 529 according to SEQ ID NO:113, or the complement thereof; position 760 according to SEQ ID NO:114, or the complement thereof. In some embodiments, the portion comprises positions corresponding to: positions 4,268-4,270 according to SEQ ID NO:3, or the complement thereof; positions 745-747 according to SEQ ID NO:27, or the complement thereof; positions 811-813 according to SEQ ID NO:28, or the complement thereof; positions 777-779 according to SEQ ID NO:29, or the complement thereof; positions 759-761 according to SEQ ID NO:30, or the complement thereof; positions 734-736 according to SEQ ID NO:31, or the complement thereof; positions 527-529 according to SEQ ID NO:32, or the complement thereof; positions 520-522 according to SEQ ID NO:33, or the complement thereof; positions 759-761 according to SEQ ID NO:34, or the complement thereof; positions 745-747 according to SEQ ID NO:107, or the complement thereof; positions 811-813 according to SEQ ID NO:108, or the complement thereof; positions 777-779 according to SEQ ID NO:109, or the complement thereof; positions 759-761 according to SEQ ID NO:110, or the complement thereof; positions 734-736 according to SEQ ID NO:111, or the complement thereof; positions 527-529 according to SEQ ID NO:112, or the complement thereof; positions 520-522 according to SEQ ID NO:113, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of an ANGPTL7 missense nucleic acid molecule encoding an ANGPTL7 predicted loss-of-function polypeptide, or the complement thereof, wherein the portion comprises a position corresponding to: position 4,273 according to SEQ ID NO:4, or the complement thereof; position 750 according to SEQ ID NO:35, or the complement thereof; position 817 according to SEQ ID NO:36, or the complement thereof; position 782 according to SEQ ID NO:37, or the complement thereof; position 764 according to SEQ ID NO:38, or the complement thereof; position 739 according to SEQ ID NO:39, or the complement thereof; position 532 according to SEQ ID NO:40, or the complement thereof; position 525 according to SEQ ID NO:41, or the complement thereof; position 764 according to SEQ ID NO:42, or the complement thereof; position 750 according to SEQ ID NO:115, or the complement thereof; position 817 according to SEQ ID NO:116, or the complement thereof; position 782 according to SEQ ID NO:117, or the complement thereof; position 764 according to SEQ ID NO:118, or the complement thereof; position 739 according to SEQ ID NO:119, or the complement thereof; position 532 according to SEQ ID NO:120, or the complement thereof; position 525 according to SEQ ID NO:121, or the complement thereof; position 764 according to SEQ ID NO:122, or the complement thereof. In some embodiments, the portion comprises positions corresponding to: positions 4,271-4,273 according to SEQ ID NO:4, or the complement thereof; positions 748-750 according to SEQ ID NO:35, or the complement thereof; positions 815-817 according to SEQ ID NO:36, or the complement thereof; positions 780-782 according to SEQ ID NO:37, or the complement thereof; positions 762-764 according to SEQ ID NO:38, or the complement thereof; positions 737-739 according to SEQ ID NO:39, or the complement thereof; positions 530-532 according to SEQ ID NO:40, or the complement thereof; positions 523-525 according to SEQ ID NO:41, or the complement thereof; positions 762-764 according to SEQ ID NO:42, or the complement thereof; positions 748-750 according to SEQ ID NO:115, or the complement thereof; positions 815-817 according to SEQ ID NO:116, or the complement thereof; positions 780-782 according to SEQ ID NO:117, or the complement thereof; positions 762-764 according to SEQ ID NO:118, or the complement thereof; positions 737-739 according to SEQ ID NO:119, or the complement thereof; positions 530-532 according to SEQ ID NO:120, or the complement thereof; positions 523-525 according to SEQ ID NO:121, or the complement thereof; positions 762-764 according to SEQ ID NO:122, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of an ANGPTL7 missense nucleic acid molecule encoding an ANGPTL7 predicted loss-of-function polypeptide, or the complement thereof, wherein the portion comprises a position corresponding to: position 4,277 according to SEQ ID NO:5, or the complement thereof; position 754 according to SEQ ID NO:43, or the complement thereof; position 821 according to SEQ ID NO:44, or the complement thereof; position 786 according to SEQ ID NO:45, or the complement thereof; position 768 according to SEQ ID NO:46, or the complement thereof; position 743 according to SEQ ID NO:47, or the complement thereof; position 536 according to SEQ ID NO:48, or the complement thereof; position 529 according to SEQ ID NO:49, or the complement thereof; position 768 according to SEQ ID NO:50, or the complement thereof; position 754 according to SEQ ID NO:123, or the complement thereof; position 821 according to SEQ ID NO:124, or the complement thereof; position 786 according to SEQ ID NO:125, or the complement thereof; position 768 according to SEQ ID NO:126, or the complement thereof; position 743 according to SEQ ID NO:127, or the complement thereof; position 536 according to SEQ ID NO:128, or the complement thereof; position 529 according to SEQ ID NO:129, or the complement thereof; position 768 according to SEQ ID NO:130, or the complement thereof. In some embodiments, the portion comprises positions corresponding to: positions 4,277-4,279 according to SEQ ID NO:5, or the complement thereof; positions 754-756 according to SEQ ID NO:43, or the complement thereof; positions 821-823 according to SEQ ID NO:44, or the complement thereof; positions 786-788 according to SEQ ID NO:45, or the complement thereof; positions 768-770 according to SEQ ID NO:46, or the complement thereof; positions 743-745 according to SEQ ID NO:47, or the complement thereof; positions 536-538 according to SEQ ID NO:48, or the complement thereof; positions 529-531 according to SEQ ID NO:49, or the complement thereof; positions 768-770 according to SEQ ID NO:50, or the complement thereof; positions 754-756 according to SEQ ID NO:123, or the complement thereof; positions 821-823 according to SEQ ID NO:124, or the complement thereof; positions 786-788 according to SEQ ID NO:125, or the complement thereof; positions 768-770 according to SEQ ID NO:126, or the complement thereof; positions 743-745 according to SEQ ID NO:127, or the complement thereof; positions 536-538 according to SEQ ID NO:128, or the complement thereof; positions 529-531 according to SEQ ID NO:129, or the complement thereof; positions 768-770 according to SEQ ID NO:130, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of an ANGPTL7 missense nucleic acid molecule encoding an ANGPTL7 predicted loss-of-function polypeptide, or the complement thereof, wherein the portion comprises a position corresponding to: position 4,311 according to SEQ ID NO:6, or the complement thereof; position 788 according to SEQ ID NO:51, or the complement thereof; position 855 according to SEQ ID NO:52, or the complement thereof; position 820 according to SEQ ID NO:53, or the complement thereof; position 802 according to SEQ ID NO:54, or the complement thereof; position 777 according to SEQ ID NO:55, or the complement thereof; position 570 according to SEQ ID NO:56, or the complement thereof; position 563 according to SEQ ID NO:57, or the complement thereof; position 802 according to SEQ ID NO:58, or the complement thereof; position 788 according to SEQ ID NO:131, or the complement thereof; position 855 according to SEQ ID NO:132, or the complement thereof; position 820 according to SEQ ID NO:133, or the complement thereof; position 802 according to SEQ ID NO:134, or the complement thereof; position 777 according to SEQ ID NO:135, or the complement thereof; position 570 according to SEQ ID NO:136, or the complement thereof; position 563 according to SEQ ID NO:137, or the complement thereof; position 802 according to SEQ ID NO:138, or the complement thereof. In some embodiments, the portion comprises positions corresponding to: positions 4,310-4,312 according to SEQ ID NO:6, or the complement thereof; positions 787-789 according to SEQ ID NO:51, or the complement thereof; positions 854-856 according to SEQ ID NO:52, or the complement thereof; positions 819-821 according to SEQ ID NO:53, or the complement thereof; positions 801-803 according to SEQ ID NO:54, or the complement thereof; positions 776-778 according to SEQ ID NO:55, or the complement thereof; positions 569-571 according to SEQ ID NO:56, or the complement thereof; positions 562-564 according to SEQ ID NO:57, or the complement thereof; positions 801-803 according to SEQ ID NO:58, or the complement thereof; positions 787-789 according to SEQ ID NO:131, or the complement thereof; positions 854-856 according to SEQ ID NO:132, or the complement thereof; positions 819-821 according to SEQ ID NO:133, or the complement thereof; positions 801-803 according to SEQ ID NO:134, or the complement thereof; positions 776-778 according to SEQ ID NO:135, or the complement thereof; positions 569-571 according to SEQ ID NO:136, or the complement thereof; positions 562-564 according to SEQ ID NO:137, or the complement thereof; positions 801-803 according to SEQ ID NO:138, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of an ANGPTL7 missense nucleic acid molecule encoding an ANGPTL7 predicted loss-of-function polypeptide, or the complement thereof, wherein the portion comprises a position corresponding to: position 4,322 according to SEQ ID NO:7, or the complement thereof; position 799 according to SEQ ID NO:59, or the complement thereof; position 866 according to SEQ ID NO:60, or the complement thereof; position 831 according to SEQ ID NO:61, or the complement thereof; position 813 according to SEQ ID NO:62, or the complement thereof; position 788 according to SEQ ID NO:63, or the complement thereof; position 581 according to SEQ ID NO:64, or the complement thereof; position 574 according to SEQ ID NO:65, or the complement thereof; position 813 according to SEQ ID NO:66, or the complement thereof; position 799 according to SEQ ID NO:139, or the complement thereof; position 866 according to SEQ ID NO:140, or the complement thereof; position 831 according to SEQ ID NO:141, or the complement thereof; position 813 according to SEQ ID NO:142, or the complement thereof; position 788 according to SEQ ID NO:143, or the complement thereof; position 581 according to SEQ ID NO:144, or the complement thereof; position 574 according to SEQ ID NO:145, or the complement thereof; position 813 according to SEQ ID NO:146, or the complement thereof. In some embodiments, the portion comprises positions corresponding to: positions 4,322-4,324 according to SEQ ID NO:7, or the complement thereof; positions 799-801 according to SEQ ID NO:59, or the complement thereof; positions 866-868 according to SEQ ID NO:60, or the complement thereof; positions 831-833 according to SEQ ID NO:61, or the complement thereof; positions 813-815 according to SEQ ID NO:62, or the complement thereof; positions 788-790 according to SEQ ID NO:63, or the complement thereof; positions 581-583 according to SEQ ID NO:64, or the complement thereof; positions 574-576 according to SEQ ID NO:65, or the complement thereof; positions 813-815 according to SEQ ID NO:66, or the complement thereof; positions 799-801 according to SEQ ID NO:139, or the complement thereof; positions 866-868 according to SEQ ID NO:140, or the complement thereof; positions 831-833 according to SEQ ID NO:141, or the complement thereof; positions 813-815 according to SEQ ID NO:142, or the complement thereof; positions 788-790 according to SEQ ID NO:143, or the complement thereof; positions 581-583 according to SEQ ID NO:144, or the complement thereof; positions 574-576 according to SEQ ID NO:145, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of an ANGPTL7 missense nucleic acid molecule encoding an ANGPTL7 predicted loss-of-function polypeptide, or the complement thereof, wherein the portion comprises a position corresponding to: position 5,125 according to SEQ ID NO:8, or the complement thereof; position 916 according to SEQ ID NO:67, or the complement thereof; position 983 according to SEQ ID NO:68, or the complement thereof; position 948 according to SEQ ID NO:69, or the complement thereof; position 930 according to SEQ ID NO:70, or the complement thereof; position 905 according to SEQ ID NO:71, or the complement thereof; position 698 according to SEQ ID NO:72, or the complement thereof; position 691 according to SEQ ID NO:73, or the complement thereof; position 930 according to SEQ ID NO:74, or the complement thereof; position 916 according to SEQ ID NO:147, or the complement thereof; position 983 according to SEQ ID NO:148, or the complement thereof; position 948 according to SEQ ID NO:149, or the complement thereof; position 930 according to SEQ ID NO:150, or the complement thereof; position 905 according to SEQ ID NO:151, or the complement thereof; position 698 according to SEQ ID NO:152, or the complement thereof; position 691 according to SEQ ID NO:153, or the complement thereof; position 930 according to SEQ ID NO:154, or the complement thereof. In some embodiments, the portion comprises positions corresponding to: positions 5,125-5,127 according to SEQ ID NO:8, or the complement thereof; positions 916-918 according to SEQ ID NO:67, or the complement thereof; positions 983-985 according to SEQ ID NO:68, or the complement thereof; positions 948-950 according to SEQ ID NO:69, or the complement thereof; positions 930-932 according to SEQ ID NO:70, or the complement thereof; positions 905-907 according to SEQ ID NO:71, or the complement thereof; positions 698-700 according to SEQ ID NO:72, or the complement thereof; positions 691-693 according to SEQ ID NO:73, or the complement thereof; positions 930-932 according to SEQ ID NO:74, or the complement thereof; positions 916-918 according to SEQ ID NO:147, or the complement thereof; positions 983-985 according to SEQ ID NO:148, or the complement thereof; positions 948-950 according to SEQ ID NO:149, or the complement thereof; positions 930-932 according to SEQ ID NO:150, or the complement thereof; positions 905-907 according to SEQ ID NO:151, or the complement thereof; positions 698-700 according to SEQ ID NO:152, or the complement thereof; positions 691-693 according to SEQ ID NO:153, or the complement thereof; positions 930-932 according to SEQ ID NO:154, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of an ANGPTL7 missense nucleic acid molecule encoding an ANGPTL7 predicted loss-of-function polypeptide, or the complement thereof, wherein the portion comprises a position corresponding to: position 5,176 according to SEQ ID NO:9, or the complement thereof; position 967 according to SEQ ID NO:75, or the complement thereof; position 1,034 according to SEQ ID NO:76, or the complement thereof; position 999 according to SEQ ID NO:77, or the complement thereof; position 981 according to SEQ ID NO:78, or the complement thereof; position 956 according to SEQ ID NO:79, or the complement thereof; position 749 according to SEQ ID NO:80, or the complement thereof; position 742 according to SEQ ID NO:81, or the complement thereof; position 981 according to SEQ ID NO:82, or the complement thereof; position 967 according to SEQ ID NO:155, or the complement thereof; position 1,034 according to SEQ ID NO:156, or the complement thereof; position 999 according to SEQ ID NO:157, or the complement thereof; position 981 according to SEQ ID NO:158, or the complement thereof; position 956 according to SEQ ID NO:159, or the complement thereof; position 749 according to SEQ ID NO:160, or the complement thereof; position 742 according to SEQ ID NO:161, or the complement thereof; position 981 according to SEQ ID NO:162, or the complement thereof. In some embodiments, the portion comprises positions corresponding to: positions 5,176-5,178 according to SEQ ID NO:9, or the complement thereof; positions 967-969 according to SEQ ID NO:75, or the complement thereof; positions 1,034-1,036 according to SEQ ID NO:76, or the complement thereof; positions 999-1,001 according to SEQ ID NO:77, or the complement thereof; positions 981-983 according to SEQ ID NO:78, or the complement thereof; positions 956-958 according to SEQ ID NO:79, or the complement thereof; positions 749-751 according to SEQ ID NO:80, or the complement thereof; positions 742-744 according to SEQ ID NO:81, or the complement thereof; positions 981-983 according to SEQ ID NO:82, or the complement thereof; positions 967-969 according to SEQ ID NO:155, or the complement thereof; positions 1,034-1,036 according to SEQ ID NO:156, or the complement thereof; positions 999-1,001 according to SEQ ID NO:157, or the complement thereof; positions 981-983 according to SEQ ID NO:158, or the complement thereof; positions 956-958 according to SEQ ID NO:159, or the complement thereof; positions 749-751 according to SEQ ID NO:160, or the complement thereof; positions 742-744 according to SEQ ID NO:161, or the complement thereof; positions 981-983 according to SEQ ID NO:162, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of an ANGPTL7 missense nucleic acid molecule encoding an ANGPTL7 predicted loss-of-function polypeptide, or the complement thereof, wherein the portion comprises a position corresponding to: position 1,023 according to SEQ ID NO:83, or the complement thereof; position 1,090 according to SEQ ID NO:84, or the complement thereof; position 1,055 according to SEQ ID NO:85, or the complement thereof; position 1,037 according to SEQ ID NO:86, or the complement thereof; position 1,012 according to SEQ ID NO:87, or the complement thereof; position 805 according to SEQ ID NO:88, or the complement thereof; position 798 according to SEQ ID NO:89, or the complement thereof; or position 1,037 according to SEQ ID NO:90, or the complement thereof; position 1,023 according to SEQ ID NO:163, or the complement thereof; position 1,090 according to SEQ ID NO:164, or the complement thereof; position 1,055 according to SEQ ID NO:165, or the complement thereof; position 1,037 according to SEQ ID NO:166, or the complement thereof; position 1,012 according to SEQ ID NO:167, or the complement thereof; position 805 according to SEQ ID NO:168, or the complement thereof; position 798 according to SEQ ID NO:169, or the complement thereof; or position 1,037 according to SEQ ID NO:170, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of an ANGPTL7 missense nucleic acid molecule encoding an ANGPTL7 predicted loss-of-function polypeptide, or the complement thereof, wherein the portion comprises a position corresponding to: position 1,021-1,023 according to SEQ ID NO:83, or the complement thereof; position 1,088-1,090 according to SEQ ID NO:84, or the complement thereof; position 1,053-1,055 according to SEQ ID NO:85, or the complement thereof; position 1,035-1,037 according to SEQ ID NO:86, or the complement thereof; position 1,010-1,012 according to SEQ ID NO:87, or the complement thereof; position 803-805 according to SEQ ID NO:88, or the complement thereof; position 796-798 according to SEQ ID NO:89, or the complement thereof; or position 1,035-1,037 according to SEQ ID NO:90, or the complement thereof; position 1,021-1,023 according to SEQ ID NO:163, or the complement thereof; position 1,088-1,090 according to SEQ ID NO:164, or the complement thereof; position 1,053-1,055 according to SEQ ID NO:165, or the complement thereof; position 1,035-1,037 according to SEQ ID NO:166, or the complement thereof; position 1,010-1,012 according to SEQ ID NO:167, or the complement thereof; position 803-805 according to SEQ ID NO:168, or the complement thereof; position 796-798 according to SEQ ID NO:169, or the complement thereof; or position 1,035-1,037 according to SEQ ID NO:170, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the ANGPTL7 missense variant genomic nucleic acid molecules, ANGPTL7 missense variant mRNA molecules, and/or ANGPTL7 missense variant cDNA molecules disclosed herein. The primers described herein can be used to amplify the ANGPTL7 missense variant genomic nucleic acid molecules, ANGPTL7 missense variant mRNA molecules, or ANGPTL7 missense variant cDNA molecules, or a fragment thereof.

The present disclosure also provides pairs of primers comprising any of the primers described above. For example, if one of the primers' 3′-ends hybridizes to a thymine at a position corresponding to position 4,229 according to SEQ ID NO:1 (rather than an adenine) in a particular ANGPTL7 nucleic acid molecule, then the presence of the amplified fragment would indicate the presence of an ANGPTL7 reference genomic nucleic acid molecule. Conversely, if one of the primers' 3′-ends hybridizes to an adenine at a position corresponding to position 4,229 according to SEQ ID NO:2 (rather than a thymine) in a particular ANGPTL7 nucleic acid molecule, then the presence of the amplified fragment would indicate the presence of the ANGPTL7 missense variant genomic nucleic acid molecule. In some embodiments, the nucleotide of the primer complementary to the adenine at a position corresponding to position 4,229 according to SEQ ID NO:2 can be at the 3′ end of the primer. In addition, if one of the primers' 3′-ends hybridizes to a uracil at a position corresponding to position 706 according to SEQ ID NO:11 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 706 according to SEQ ID NO:19 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 706 according to SEQ ID NO:19 can be at the 3′ end of the primer. In addition, if one of the primers' 3′-ends hybridizes to a uracil at a position corresponding to position 773 according to SEQ ID NO:12 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 773 according to SEQ ID NO:20 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 773 according to SEQ ID NO:20 can be at the 3′ end of the primer. In addition, if one of the primers' 3′-ends hybridizes to a uracil at a position corresponding to position 738 according to SEQ ID NO:13 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 738 according to SEQ ID NO:21 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 738 according to SEQ ID NO:21 can be at the 3′ end of the primer. In addition, if one of the primers' 3′-ends hybridizes to a uracil at a position corresponding to position 720 according to SEQ ID NO:14 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 720 according to SEQ ID NO:22 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 720 according to SEQ ID NO:22 can be at the 3′ end of the primer. In addition, if one of the primers' 3′-ends hybridizes to a uracil at a position corresponding to position 695 according to SEQ ID NO:15 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 695 according to SEQ ID NO:23 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 695 according to SEQ ID NO:23 can be at the 3′ end of the primer. In addition, if one of the primers' 3′-ends hybridizes to a uracil at a position corresponding to position 488 according to SEQ ID NO:16 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 488 according to SEQ ID NO:24 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 488 according to SEQ ID NO:24 can be at the 3′ end of the primer. In addition, if one of the primers' 3′-ends hybridizes to a uracil at a position corresponding to position 481 according to SEQ ID NO:17 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 481 according to SEQ ID NO:25 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 481 according to SEQ ID NO:25 can be at the 3′ end of the primer. In addition, if one of the primers' 3′-ends hybridizes to a uracil at a position corresponding to position 720 according to SEQ ID NO:18 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 720 according to SEQ ID NO:26 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 720 according to SEQ ID NO:26 can be at the 3′ end of the primer. In addition, if one of the primers' 3′-ends hybridizes to a thymine at a position corresponding to position 706 according to SEQ ID NO:91 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 706 according to SEQ ID NO:99 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 706 according to SEQ ID NO:99 can be at the 3′ end of the primer. In addition, if one of the primers' 3′-ends hybridizes to a thymine at a position corresponding to position 773 according to SEQ ID NO:92 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 773 according to SEQ ID NO:100 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 773 according to SEQ ID NO:100 can be at the 3′ end of the primer. In addition, if one of the primers' 3′-ends hybridizes to a thymine at a position corresponding to position 738 according to SEQ ID NO:93 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 738 according to SEQ ID NO:101 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 738 according to SEQ ID NO:101 can be at the 3′ end of the primer. In addition, if one of the primers' 3′-ends hybridizes to a thymine at a position corresponding to position 720 according to SEQ ID NO:94 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 720 according to SEQ ID NO:102 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 720 according to SEQ ID NO:102 can be at the 3′ end of the primer. In addition, if one of the primers' 3′-ends hybridizes to a thymine at a position corresponding to position 695 according to SEQ ID NO:95 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 695 according to SEQ ID NO:103 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 695 according to SEQ ID NO:103 can be at the 3′ end of the primer. In addition, if one of the primers' 3′-ends hybridizes to a thymine at a position corresponding to position 488 according to SEQ ID NO:96 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 488 according to SEQ ID NO:104 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 488 according to SEQ ID NO:104 can be at the 3′ end of the primer. In addition, if one of the primers' 3′-ends hybridizes to a thymine at a position corresponding to position 481 according to SEQ ID NO:97 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 481 according to SEQ ID NO:105 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 481 according to SEQ ID NO:105 can be at the 3′ end of the primer. In addition, if one of the primers' 3′-ends hybridizes to a thymine at a position corresponding to position 720 according to SEQ ID NO:98 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 720 according to SEQ ID NO:106 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 720 according to SEQ ID NO:106 can be at the 3′ end of the primer.

If one of the primers' 3′-ends hybridizes to a thymine at a position corresponding to position 4,269 according to SEQ ID NO:1 (rather than an adenine) in a particular ANGPTL7 nucleic acid molecule, then the presence of the amplified fragment would indicate the presence of an ANGPTL7 reference genomic nucleic acid molecule. Conversely, if one of the primers' 3′-ends hybridizes to an adenine at a position corresponding to position 4,269 according to SEQ ID NO:3 (rather than a thymine) in a particular ANGPTL7 nucleic acid molecule, then the presence of the amplified fragment would indicate the presence of the ANGPTL7 missense variant genomic nucleic acid molecule. In some embodiments, the nucleotide of the primer complementary to the adenine at a position corresponding to position 4,269 according to SEQ ID NO:3 can be at the 3′ end of the primer. In addition, if one of the primers' 3′-ends hybridizes to a uracil at a position corresponding to position 746 according to SEQ ID NO:11 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 746 according to SEQ ID NO:27 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 746 according to SEQ ID NO:27 can be at the 3′ end of the primer. In addition, if one of the primers' 3′-ends hybridizes to a uracil at a position corresponding to position 812 according to SEQ ID NO:12 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 812 according to SEQ ID NO:28 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 812 according to SEQ ID NO:28 can be at the 3′ end of the primer. In addition, if one of the primers' 3′-ends hybridizes to a uracil at a position corresponding to position 778 according to SEQ ID NO:13 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 778 according to SEQ ID NO:29 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 778 according to SEQ ID NO:29 can be at the 3′ end of the primer. In addition, if one of the primers' 3′-ends hybridizes to a uracil at a position corresponding to position 760 according to SEQ ID NO:14 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 760 according to SEQ ID NO:30 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 760 according to SEQ ID NO:30 can be at the 3′ end of the primer. In addition, if one of the primers' 3′-ends hybridizes to a uracil at a position corresponding to position 735 according to SEQ ID NO:15 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 735 according to SEQ ID NO:31 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 735 according to SEQ ID NO:31 can be at the 3′ end of the primer. In addition, if one of the primers' 3′-ends hybridizes to a uracil at a position corresponding to position 528 according to SEQ ID NO:16 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 528 according to SEQ ID NO:32 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 528 according to SEQ ID NO:32 can be at the 3′ end of the primer. In addition, if one of the primers' 3′-ends hybridizes to a uracil at a position corresponding to position 521 according to SEQ ID NO:17 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 521 according to SEQ ID NO:33 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 521 according to SEQ ID NO:33 can be at the 3′ end of the primer. In addition, if one of the primers' 3′-ends hybridizes to a uracil at a position corresponding to position 760 according to SEQ ID NO:18 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 760 according to SEQ ID NO:34 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 760 according to SEQ ID NO:34 can be at the 3′ end of the primer. In addition, if one of the primers' 3′-ends hybridizes to a thymine at a position corresponding to position 746 according to SEQ ID NO:91 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 746 according to SEQ ID NO:107 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 746 according to SEQ ID NO:107 can be at the 3′ end of the primer. In addition, if one of the primers' 3′-ends hybridizes to a thymine at a position corresponding to position 812 according to SEQ ID NO:92 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 812 according to SEQ ID NO:108 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 812 according to SEQ ID NO:108 can be at the 3′ end of the primer. In addition, if one of the primers' 3′-ends hybridizes to a thymine at a position corresponding to position 778 according to SEQ ID NO:93 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 778 according to SEQ ID NO:109 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 778 according to SEQ ID NO:109 can be at the 3′ end of the primer. In addition, if one of the primers' 3′-ends hybridizes to a thymine at a position corresponding to position 760 according to SEQ ID NO:94 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 760 according to SEQ ID NO:110 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 760 according to SEQ ID NO:110 can be at the 3′ end of the primer. In addition, if one of the primers' 3′-ends hybridizes to a thymine at a position corresponding to position 735 according to SEQ ID NO:95 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 735 according to SEQ ID NO:111 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 735 according to SEQ ID NO:111 can be at the 3′ end of the primer. In addition, if one of the primers' 3′-ends hybridizes to a thymine at a position corresponding to position 528 according to SEQ ID NO:96 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 528 according to SEQ ID NO:112 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 528 according to SEQ ID NO:112 can be at the 3′ end of the primer. In addition, if one of the primers' 3′-ends hybridizes to a thymine at a position corresponding to position 529 according to SEQ ID NO:97 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 529 according to SEQ ID NO:113 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 529 according to SEQ ID NO:113 can be at the 3′ end of the primer. In addition, if one of the primers' 3′-ends hybridizes to a thymine at a position corresponding to position 760 according to SEQ ID NO:98 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 760 according to SEQ ID NO:114 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 760 according to SEQ ID NO:114 can be at the 3′ end of the primer.

If one of the primers' 3′-ends hybridizes to a guanine at a position corresponding to position 4,273 according to SEQ ID NO:1 (rather than a thymine) in a particular ANGPTL7 nucleic acid molecule, then the presence of the amplified fragment would indicate the presence of an ANGPTL7 reference genomic nucleic acid molecule. Conversely, if one of the primers' 3′-ends hybridizes to a thymine at a position corresponding to position 4,273 according to SEQ ID NO:4 (rather than a guanine) in a particular ANGPTL7 nucleic acid molecule, then the presence of the amplified fragment would indicate the presence of the ANGPTL7 missense variant genomic nucleic acid molecule. In some embodiments, the nucleotide of the primer complementary to the thymine at a position corresponding to position 4,273 according to SEQ ID NO:4 can be at the 3′ end of the primer. In addition, if one of the primers' 3′-ends hybridizes to a guanine at a position corresponding to position 750 according to SEQ ID NO:11 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 750 according to SEQ ID NO:35 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 750 according to SEQ ID NO:35 can be at the 3′ end of the primer. In addition, if one of the primers' 3′-ends hybridizes to a guanine at a position corresponding to position 817 according to SEQ ID NO:12 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 817 according to SEQ ID NO:36 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 817 according to SEQ ID NO:36 can be at the 3′ end of the primer. In addition, if one of the primers' 3′-ends hybridizes to a guanine at a position corresponding to position 782 according to SEQ ID NO:13 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 782 according to SEQ ID NO:37 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 782 according to SEQ ID NO:37 can be at the 3′ end of the primer. In addition, if one of the primers' 3′-ends hybridizes to a guanine at a position corresponding to position 764 according to SEQ ID NO:14 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 764 according to SEQ ID NO:38 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 764 according to SEQ ID NO:38 can be at the 3′ end of the primer. In addition, if one of the primers' 3′-ends hybridizes to a guanine at a position corresponding to position 739 according to SEQ ID NO:15 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 739 according to SEQ ID NO:39 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 739 according to SEQ ID NO:39 can be at the 3′ end of the primer. In addition, if one of the primers' 3′-ends hybridizes to a guanine at a position corresponding to position 532 according to SEQ ID NO:16 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 532 according to SEQ ID NO:40 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 532 according to SEQ ID NO:40 can be at the 3′ end of the primer. In addition, if one of the primers' 3′-ends hybridizes to a guanine at a position corresponding to position 525 according to SEQ ID NO:17 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 525 according to SEQ ID NO:41 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 525 according to SEQ ID NO:41 can be at the 3′ end of the primer. In addition, if one of the primers' 3′-ends hybridizes to a guanine at a position corresponding to position 764 according to SEQ ID NO:18 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 764 according to SEQ ID NO:42 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 764 according to SEQ ID NO:42 can be at the 3′ end of the primer. In addition, if one of the primers' 3′-ends hybridizes to a guanine at a position corresponding to position 750 according to SEQ ID NO:91 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 750 according to SEQ ID NO:115 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 750 according to SEQ ID NO:115 can be at the 3′ end of the primer. In addition, if one of the primers' 3′-ends hybridizes to a guanine at a position corresponding to position 817 according to SEQ ID NO:92 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 817 according to SEQ ID NO:116 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 817 according to SEQ ID NO:116 can be at the 3′ end of the primer. In addition, if one of the primers' 3′-ends hybridizes to a guanine at a position corresponding to position 782 according to SEQ ID NO:93 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 782 according to SEQ ID NO:117 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 782 according to SEQ ID NO:117 can be at the 3′ end of the primer. In addition, if one of the primers' 3′-ends hybridizes to a guanine at a position corresponding to position 764 according to SEQ ID NO:94 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 764 according to SEQ ID NO:118 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 764 according to SEQ ID NO:118 can be at the 3′ end of the primer. In addition, if one of the primers' 3′-ends hybridizes to a guanine at a position corresponding to position 739 according to SEQ ID NO:95 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 739 according to SEQ ID NO:119 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 739 according to SEQ ID NO:119 can be at the 3′ end of the primer. In addition, if one of the primers' 3′-ends hybridizes to a guanine at a position corresponding to position 532 according to SEQ ID NO:96 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 532 according to SEQ ID NO:120 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 532 according to SEQ ID NO:120 can be at the 3′ end of the primer. In addition, if one of the primers' 3′-ends hybridizes to a guanine at a position corresponding to position 525 according to SEQ ID NO:97 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 525 according to SEQ ID NO:121 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 525 according to SEQ ID NO:121 can be at the 3′ end of the primer. In addition, if one of the primers' 3′-ends hybridizes to a guanine at a position corresponding to position 764 according to SEQ ID NO:98 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 764 according to SEQ ID NO:122 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 764 according to SEQ ID NO:122 can be at the 3′ end of the primer.

If one of the primers' 3′-ends hybridizes to a cytosine at a position corresponding to position 4,277 according to SEQ ID NO:1 (rather than a thymine) in a particular ANGPTL7 nucleic acid molecule, then the presence of the amplified fragment would indicate the presence of an ANGPTL7 reference genomic nucleic acid molecule. Conversely, if one of the primers' 3′-ends hybridizes to a thymine at a position corresponding to position 4,277 according to SEQ ID NO:5 (rather than a cytosine) in a particular ANGPTL7 nucleic acid molecule, then the presence of the amplified fragment would indicate the presence of the ANGPTL7 missense variant genomic nucleic acid molecule. In some embodiments, the nucleotide of the primer complementary to the thymine at a position corresponding to position 4,277 according to SEQ ID NO:5 can be at the 3′ end of the primer. In addition, if one of the primers' 3′-ends hybridizes to a cytosine at a position corresponding to position 754 according to SEQ ID NO:11 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 754 according to SEQ ID NO:43 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 754 according to SEQ ID NO:43 can be at the 3′ end of the primer. In addition, if one of the primers' 3′-ends hybridizes to a cytosine at a position corresponding to position 821 according to SEQ ID NO:12 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 821 according to SEQ ID NO:44 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 821 according to SEQ ID NO:44 can be at the 3′ end of the primer. In addition, if one of the primers' 3′-ends hybridizes to a cytosine at a position corresponding to position 786 according to SEQ ID NO:13 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 786 according to SEQ ID NO:45 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 786 according to SEQ ID NO:45 can be at the 3′ end of the primer. In addition, if one of the primers' 3′-ends hybridizes to a cytosine at a position corresponding to position 768 according to SEQ ID NO:14 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 768 according to SEQ ID NO:46 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 768 according to SEQ ID NO:46 can be at the 3′ end of the primer. In addition, if one of the primers' 3′-ends hybridizes to a cytosine at a position corresponding to position 743 according to SEQ ID NO:15 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 743 according to SEQ ID NO:47 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 743 according to SEQ ID NO:47 can be at the 3′ end of the primer. In addition, if one of the primers' 3′-ends hybridizes to a cytosine at a position corresponding to position 536 according to SEQ ID NO:16 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 536 according to SEQ ID NO:48 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 536 according to SEQ ID NO:48 can be at the 3′ end of the primer. In addition, if one of the primers' 3′-ends hybridizes to a cytosine at a position corresponding to position 529 according to SEQ ID NO:17 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 529 according to SEQ ID NO:49 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 529 according to SEQ ID NO:49 can be at the 3′ end of the primer. In addition, if one of the primers' 3′-ends hybridizes to a cytosine at a position corresponding to position 768 according to SEQ ID NO:18 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 768 according to SEQ ID NO:50 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 768 according to SEQ ID NO:50 can be at the 3′ end of the primer. In addition, if one of the primers' 3′-ends hybridizes to a cytosine at a position corresponding to position 754 according to SEQ ID NO:91 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 754 according to SEQ ID NO:123 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 754 according to SEQ ID NO:123 can be at the 3′ end of the primer. In addition, if one of the primers' 3′-ends hybridizes to a cytosine at a position corresponding to position 821 according to SEQ ID NO:92 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 821 according to SEQ ID NO:124 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 821 according to SEQ ID NO:124 can be at the 3′ end of the primer. In addition, if one of the primers' 3′-ends hybridizes to a cytosine at a position corresponding to position 786 according to SEQ ID NO:93 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 786 according to SEQ ID NO:125 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 786 according to SEQ ID NO:125 can be at the 3′ end of the primer. In addition, if one of the primers' 3′-ends hybridizes to a cytosine at a position corresponding to position 768 according to SEQ ID NO:94 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 768 according to SEQ ID NO:126 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 768 according to SEQ ID NO:126 can be at the 3′ end of the primer. In addition, if one of the primers' 3′-ends hybridizes to a cytosine at a position corresponding to position 743 according to SEQ ID NO:95 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 743 according to SEQ ID NO:127 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 743 according to SEQ ID NO:127 can be at the 3′ end of the primer. In addition, if one of the primers' 3′-ends hybridizes to a cytosine at a position corresponding to position 536 according to SEQ ID NO:96 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 536 according to SEQ ID NO:128 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 536 according to SEQ ID NO:128 can be at the 3′ end of the primer. In addition, if one of the primers' 3′-ends hybridizes to a cytosine at a position corresponding to position 529 according to SEQ ID NO:97 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 529 according to SEQ ID NO:129 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 529 according to SEQ ID NO:129 can be at the 3′ end of the primer. In addition, if one of the primers' 3′-ends hybridizes to a cytosine at a position corresponding to position 768 according to SEQ ID NO:98 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 768 according to SEQ ID NO:130 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 768 according to SEQ ID NO:130 can be at the 3′ end of the primer.

If one of the primers' 3′-ends hybridizes to a guanine at a position corresponding to position 4,311 according to SEQ ID NO:1 (rather than an adenine) in a particular ANGPTL7 nucleic acid molecule, then the presence of the amplified fragment would indicate the presence of an ANGPTL7 reference genomic nucleic acid molecule. Conversely, if one of the primers' 3′-ends hybridizes to an adenine at a position corresponding to position 4,311 according to SEQ ID NO:6 (rather than a guanine) in a particular ANGPTL7 nucleic acid molecule, then the presence of the amplified fragment would indicate the presence of the ANGPTL7 missense variant genomic nucleic acid molecule. In some embodiments, the nucleotide of the primer complementary to the adenine at a position corresponding to position 4,311 according to SEQ ID NO:6 can be at the 3′ end of the primer. In addition, if one of the primers' 3′-ends hybridizes to a guanine at a position corresponding to position 788 according to SEQ ID NO:11 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 788 according to SEQ ID NO:51 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 788 according to SEQ ID NO:51 can be at the 3′ end of the primer. In addition, if one of the primers' 3′-ends hybridizes to a guanine at a position corresponding to position 855 according to SEQ ID NO:12 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 855 according to SEQ ID NO:52 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 855 according to SEQ ID NO:52 can be at the 3′ end of the primer. In addition, if one of the primers' 3′-ends hybridizes to a guanine at a position corresponding to position 820 according to SEQ ID NO:13 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 820 according to SEQ ID NO:53 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 820 according to SEQ ID NO:53 can be at the 3′ end of the primer. In addition, if one of the primers' 3′-ends hybridizes to a guanine at a position corresponding to position 802 according to SEQ ID NO:14 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 802 according to SEQ ID NO:54 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 802 according to SEQ ID NO:54 can be at the 3′ end of the primer. In addition, if one of the primers' 3′-ends hybridizes to a guanine at a position corresponding to position 777 according to SEQ ID NO:15 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 777 according to SEQ ID NO:55 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 777 according to SEQ ID NO:55 can be at the 3′ end of the primer. In addition, if one of the primers' 3′-ends hybridizes to a guanine at a position corresponding to position 570 according to SEQ ID NO:16 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 570 according to SEQ ID NO:56 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 570 according to SEQ ID NO:56 can be at the 3′ end of the primer. In addition, if one of the primers' 3′-ends hybridizes to a guanine at a position corresponding to position 563 according to SEQ ID NO:17 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 563 according to SEQ ID NO:57 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 563 according to SEQ ID NO:57 can be at the 3′ end of the primer. In addition, if one of the primers' 3′-ends hybridizes to a guanine at a position corresponding to position 802 according to SEQ ID NO:18 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 802 according to SEQ ID NO:58 (rather than a guanine) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 802 according to SEQ ID NO:58 can be at the 3′ end of the primer. In addition, if one of the primers' 3′-ends hybridizes to a guanine at a position corresponding to position 788 according to SEQ ID NO:91 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 788 according to SEQ ID NO:131 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 788 according to SEQ ID NO:131 can be at the 3′ end of the primer. In addition, if one of the primers' 3′-ends hybridizes to a guanine at a position corresponding to position 855 according to SEQ ID NO:92 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 855 according to SEQ ID NO:132 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 855 according to SEQ ID NO:132 can be at the 3′ end of the primer. In addition, if one of the primers' 3′-ends hybridizes to a guanine at a position corresponding to position 820 according to SEQ ID NO:93 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 820 according to SEQ ID NO:133 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 820 according to SEQ ID NO:133 can be at the 3′ end of the primer. In addition, if one of the primers' 3′-ends hybridizes to a guanine at a position corresponding to position 802 according to SEQ ID NO:94 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 802 according to SEQ ID NO:134 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 802 according to SEQ ID NO:134 can be at the 3′ end of the primer. In addition, if one of the primers' 3′-ends hybridizes to a guanine at a position corresponding to position 777 according to SEQ ID NO:95 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 777 according to SEQ ID NO:135 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 777 according to SEQ ID NO:135 can be at the 3′ end of the primer. In addition, if one of the primers' 3′-ends hybridizes to a guanine at a position corresponding to position 570 according to SEQ ID NO:96 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 570 according to SEQ ID NO:136 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 570 according to SEQ ID NO:136 can be at the 3′ end of the primer. In addition, if one of the primers' 3′-ends hybridizes to a guanine at a position corresponding to position 563 according to SEQ ID NO:97 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 563 according to SEQ ID NO:137 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 563 according to SEQ ID NO:137 can be at the 3′ end of the primer. In addition, if one of the primers' 3′-ends hybridizes to a guanine at a position corresponding to position 802 according to SEQ ID NO:98 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 802 according to SEQ ID NO:138 (rather than a guanine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 802 according to SEQ ID NO:138 can be at the 3′ end of the primer.

If one of the primers' 3′-ends hybridizes to an adenine at a position corresponding to position 4,322 according to SEQ ID NO:1 (rather than a cytosine) in a particular ANGPTL7 nucleic acid molecule, then the presence of the amplified fragment would indicate the presence of an ANGPTL7 reference genomic nucleic acid molecule. Conversely, if one of the primers' 3′-ends hybridizes to a cytosine at a position corresponding to position 4,322 according to SEQ ID NO:7 (rather than an adenine) in a particular ANGPTL7 nucleic acid molecule, then the presence of the amplified fragment would indicate the presence of the ANGPTL7 missense variant genomic nucleic acid molecule. In some embodiments, the nucleotide of the primer complementary to the cytosine at a position corresponding to position 4,322 according to SEQ ID NO:7 can be at the 3′ end of the primer. In addition, if one of the primers' 3′-ends hybridizes to an adenine at a position corresponding to position 799 according to SEQ ID NO:11 (rather than a cytosine) in a particular ANGPTL7 nucleic acid molecule, then the presence of the amplified fragment would indicate the presence of an ANGPTL7 reference mRNA molecule. Conversely, if one of the primers' 3′-ends hybridizes to a cytosine at a position corresponding to position 799 according to SEQ ID NO:59 (rather than an adenine) in a particular ANGPTL7 mRNA molecule, then the presence of the amplified fragment would indicate the presence of the ANGPTL7 missense variant mRNA molecule. In some embodiments, the nucleotide of the primer complementary to the cytosine at a position corresponding to position 799 according to SEQ ID NO:59 can be at the 3′ end of the primer. In addition, if one of the primers' 3′-ends hybridizes to an adenine at a position corresponding to position 866 according to SEQ ID NO:12 (rather than a cytosine) in a particular ANGPTL7 nucleic acid molecule, then the presence of the amplified fragment would indicate the presence of an ANGPTL7 reference mRNA molecule. Conversely, if one of the primers' 3′-ends hybridizes to a cytosine at a position corresponding to position 866 according to SEQ ID NO:60 (rather than an adenine) in a particular ANGPTL7 mRNA molecule, then the presence of the amplified fragment would indicate the presence of the ANGPTL7 missense variant mRNA molecule. In some embodiments, the nucleotide of the primer complementary to the cytosine at a position corresponding to position 866 according to SEQ ID NO:60 can be at the 3′ end of the primer. In addition, if one of the primers' 3′-ends hybridizes to an adenine at a position corresponding to position 831 according to SEQ ID NO:13 (rather than a cytosine) in a particular ANGPTL7 nucleic acid molecule, then the presence of the amplified fragment would indicate the presence of an ANGPTL7 reference mRNA molecule. Conversely, if one of the primers' 3′-ends hybridizes to a cytosine at a position corresponding to position 831 according to SEQ ID NO:61 (rather than an adenine) in a particular ANGPTL7 mRNA molecule, then the presence of the amplified fragment would indicate the presence of the ANGPTL7 missense variant mRNA molecule. In some embodiments, the nucleotide of the primer complementary to the cytosine at a position corresponding to position 831 according to SEQ ID NO:61 can be at the 3′ end of the primer. In addition, if one of the primers' 3′-ends hybridizes to an adenine at a position corresponding to position 813 according to SEQ ID NO:14 (rather than a cytosine) in a particular ANGPTL7 nucleic acid molecule, then the presence of the amplified fragment would indicate the presence of an ANGPTL7 reference mRNA molecule. Conversely, if one of the primers' 3′-ends hybridizes to a cytosine at a position corresponding to position 813 according to SEQ ID NO:62 (rather than an adenine) in a particular ANGPTL7 mRNA molecule, then the presence of the amplified fragment would indicate the presence of the ANGPTL7 missense variant mRNA molecule. In some embodiments, the nucleotide of the primer complementary to the cytosine at a position corresponding to position 813 according to SEQ ID NO:62 can be at the 3′ end of the primer. In addition, if one of the primers' 3′-ends hybridizes to an adenine at a position corresponding to position 788 according to SEQ ID NO:15 (rather than a cytosine) in a particular ANGPTL7 nucleic acid molecule, then the presence of the amplified fragment would indicate the presence of an ANGPTL7 reference mRNA molecule. Conversely, if one of the primers' 3′-ends hybridizes to a cytosine at a position corresponding to position 788 according to SEQ ID NO:63 (rather than an adenine) in a particular ANGPTL7 mRNA molecule, then the presence of the amplified fragment would indicate the presence of the ANGPTL7 missense variant mRNA molecule. In some embodiments, the nucleotide of the primer complementary to the cytosine at a position corresponding to position 788 according to SEQ ID NO:63 can be at the 3′ end of the primer. In addition, if one of the primers' 3′-ends hybridizes to an adenine at a position corresponding to position 581 according to SEQ ID NO:16 (rather than a cytosine) in a particular ANGPTL7 nucleic acid molecule, then the presence of the amplified fragment would indicate the presence of an ANGPTL7 reference mRNA molecule. Conversely, if one of the primers' 3′-ends hybridizes to a cytosine at a position corresponding to position 581 according to SEQ ID NO:64 (rather than an adenine) in a particular ANGPTL7 mRNA molecule, then the presence of the amplified fragment would indicate the presence of the ANGPTL7 missense variant mRNA molecule. In some embodiments, the nucleotide of the primer complementary to the cytosine at a position corresponding to position 581 according to SEQ ID NO:64 can be at the 3′ end of the primer. In addition, if one of the primers' 3′-ends hybridizes to an adenine at a position corresponding to position 574 according to SEQ ID NO:17 (rather than a cytosine) in a particular ANGPTL7 nucleic acid molecule, then the presence of the amplified fragment would indicate the presence of an ANGPTL7 reference mRNA molecule. Conversely, if one of the primers' 3′-ends hybridizes to a cytosine at a position corresponding to position 574 according to SEQ ID NO:65 (rather than an adenine) in a particular ANGPTL7 mRNA molecule, then the presence of the amplified fragment would indicate the presence of the ANGPTL7 missense variant mRNA molecule. In some embodiments, the nucleotide of the primer complementary to the cytosine at a position corresponding to position 574 according to SEQ ID NO:65 can be at the 3′ end of the primer. In addition, if one of the primers' 3′-ends hybridizes to an adenine at a position corresponding to position 813 according to SEQ ID NO:18 (rather than a cytosine) in a particular ANGPTL7 nucleic acid molecule, then the presence of the amplified fragment would indicate the presence of an ANGPTL7 reference mRNA molecule. Conversely, if one of the primers' 3′-ends hybridizes to a cytosine at a position corresponding to position 813 according to SEQ ID NO:66 (rather than an adenine) in a particular ANGPTL7 mRNA molecule, then the presence of the amplified fragment would indicate the presence of the ANGPTL7 missense variant mRNA molecule. In some embodiments, the nucleotide of the primer complementary to the cytosine at a position corresponding to position 813 according to SEQ ID NO:66 can be at the 3′ end of the primer. In addition, if one of the primers' 3′-ends hybridizes to an adenine at a position corresponding to position 799 according to SEQ ID NO:91 (rather than a cytosine) in a particular ANGPTL7 nucleic acid molecule, then the presence of the amplified fragment would indicate the presence of an ANGPTL7 reference cDNA molecule. Conversely, if one of the primers' 3′-ends hybridizes to a cytosine at a position corresponding to position 799 according to SEQ ID NO:139 (rather than an adenine) in a particular ANGPTL7 cDNA molecule, then the presence of the amplified fragment would indicate the presence of the ANGPTL7 missense variant cDNA molecule. In some embodiments, the nucleotide of the primer complementary to the cytosine at a position corresponding to position 799 according to SEQ ID NO:139 can be at the 3′ end of the primer. In addition, if one of the primers' 3′-ends hybridizes to an adenine at a position corresponding to position 866 according to SEQ ID NO:92 (rather than a cytosine) in a particular ANGPTL7 nucleic acid molecule, then the presence of the amplified fragment would indicate the presence of an ANGPTL7 reference cDNA molecule. Conversely, if one of the primers' 3′-ends hybridizes to a cytosine at a position corresponding to position 866 according to SEQ ID NO:140 (rather than an adenine) in a particular ANGPTL7 cDNA molecule, then the presence of the amplified fragment would indicate the presence of the ANGPTL7 missense variant cDNA molecule. In some embodiments, the nucleotide of the primer complementary to the cytosine at a position corresponding to position 866 according to SEQ ID NO:140 can be at the 3′ end of the primer. In addition, if one of the primers' 3′-ends hybridizes to an adenine at a position corresponding to position 831 according to SEQ ID NO:93 (rather than a cytosine) in a particular ANGPTL7 nucleic acid molecule, then the presence of the amplified fragment would indicate the presence of an ANGPTL7 reference cDNA molecule. Conversely, if one of the primers' 3′-ends hybridizes to a cytosine at a position corresponding to position 831 according to SEQ ID NO:141 (rather than an adenine) in a particular ANGPTL7 cDNA molecule, then the presence of the amplified fragment would indicate the presence of the ANGPTL7 missense variant cDNA molecule. In some embodiments, the nucleotide of the primer complementary to the cytosine at a position corresponding to position 831 according to SEQ ID NO:141 can be at the 3′ end of the primer. In addition, if one of the primers' 3′-ends hybridizes to an adenine at a position corresponding to position 813 according to SEQ ID NO:94 (rather than a cytosine) in a particular ANGPTL7 nucleic acid molecule, then the presence of the amplified fragment would indicate the presence of an ANGPTL7 reference cDNA molecule. Conversely, if one of the primers' 3′-ends hybridizes to a cytosine at a position corresponding to position 813 according to SEQ ID NO:142 (rather than an adenine) in a particular ANGPTL7 cDNA molecule, then the presence of the amplified fragment would indicate the presence of the ANGPTL7 missense variant cDNA molecule. In some embodiments, the nucleotide of the primer complementary to the cytosine at a position corresponding to position 813 according to SEQ ID NO:142 can be at the 3′ end of the primer. In addition, if one of the primers' 3′-ends hybridizes to an adenine at a position corresponding to position 788 according to SEQ ID NO:95 (rather than a cytosine) in a particular ANGPTL7 nucleic acid molecule, then the presence of the amplified fragment would indicate the presence of an ANGPTL7 reference cDNA molecule. Conversely, if one of the primers' 3′-ends hybridizes to a cytosine at a position corresponding to position 788 according to SEQ ID NO:143 (rather than an adenine) in a particular ANGPTL7 cDNA molecule, then the presence of the amplified fragment would indicate the presence of the ANGPTL7 missense variant cDNA molecule. In some embodiments, the nucleotide of the primer complementary to the cytosine at a position corresponding to position 788 according to SEQ ID NO:143 can be at the 3′ end of the primer. In addition, if one of the primers' 3′-ends hybridizes to an adenine at a position corresponding to position 581 according to SEQ ID NO:96 (rather than a cytosine) in a particular ANGPTL7 nucleic acid molecule, then the presence of the amplified fragment would indicate the presence of an ANGPTL7 reference cDNA molecule. Conversely, if one of the primers' 3′-ends hybridizes to a cytosine at a position corresponding to position 581 according to SEQ ID NO:144 (rather than an adenine) in a particular ANGPTL7 cDNA molecule, then the presence of the amplified fragment would indicate the presence of the ANGPTL7 missense variant cDNA molecule. In some embodiments, the nucleotide of the primer complementary to the cytosine at a position corresponding to position 581 according to SEQ ID NO:144 can be at the 3′ end of the primer. In addition, if one of the primers' 3′-ends hybridizes to an adenine at a position corresponding to position 574 according to SEQ ID NO:97 (rather than a cytosine) in a particular ANGPTL7 nucleic acid molecule, then the presence of the amplified fragment would indicate the presence of an ANGPTL7 reference cDNA molecule. Conversely, if one of the primers' 3′-ends hybridizes to a cytosine at a position corresponding to position 574 according to SEQ ID NO:145 (rather than an adenine) in a particular ANGPTL7 cDNA molecule, then the presence of the amplified fragment would indicate the presence of the ANGPTL7 missense variant cDNA molecule. In some embodiments, the nucleotide of the primer complementary to the cytosine at a position corresponding to position 574 according to SEQ ID NO:145 can be at the 3′ end of the primer. In addition, if one of the primers' 3′-ends hybridizes to an adenine at a position corresponding to position 813 according to SEQ ID NO:98 (rather than a cytosine) in a particular ANGPTL7 nucleic acid molecule, then the presence of the amplified fragment would indicate the presence of an ANGPTL7 reference cDNA molecule. Conversely, if one of the primers' 3′-ends hybridizes to a cytosine at a position corresponding to position 813 according to SEQ ID NO:146 (rather than an adenine) in a particular ANGPTL7 cDNA molecule, then the presence of the amplified fragment would indicate the presence of the ANGPTL7 missense variant cDNA molecule. In some embodiments, the nucleotide of the primer complementary to the cytosine at a position corresponding to position 813 according to SEQ ID NO:146 can be at the 3′ end of the primer.

If one of the primers' 3′-ends hybridizes to a cytosine at a position corresponding to position 5,125 according to SEQ ID NO:1 (rather than a thymine) in a particular ANGPTL7 nucleic acid molecule, then the presence of the amplified fragment would indicate the presence of an ANGPTL7 reference genomic nucleic acid molecule. Conversely, if one of the primers' 3′-ends hybridizes to a thymine at a position corresponding to position 5,125 according to SEQ ID NO:8 (rather than a cytosine) in a particular ANGPTL7 nucleic acid molecule, then the presence of the amplified fragment would indicate the presence of the ANGPTL7 missense variant genomic nucleic acid molecule. In some embodiments, the nucleotide of the primer complementary to the thymine at a position corresponding to position 5,125 according to SEQ ID NO:8 can be at the 3′ end of the primer. In addition, if one of the primers' 3′-ends hybridizes to a cytosine at a position corresponding to position 916 according to SEQ ID NO:11 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 916 according to SEQ ID NO:67 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 916 according to SEQ ID NO:67 can be at the 3′ end of the primer. In addition, if one of the primers' 3′-ends hybridizes to a cytosine at a position corresponding to position 983 according to SEQ ID NO:12 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 983 according to SEQ ID NO:68 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 983 according to SEQ ID NO:68 can be at the 3′ end of the primer. In addition, if one of the primers' 3′-ends hybridizes to a cytosine at a position corresponding to position 948 according to SEQ ID NO:13 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 948 according to SEQ ID NO:69 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 948 according to SEQ ID NO:69 can be at the 3′ end of the primer. In addition, if one of the primers' 3′-ends hybridizes to a cytosine at a position corresponding to position 930 according to SEQ ID NO:14 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 930 according to SEQ ID NO:70 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 930 according to SEQ ID NO:70 can be at the 3′ end of the primer. In addition, if one of the primers' 3′-ends hybridizes to a cytosine at a position corresponding to position 905 according to SEQ ID NO:15 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 905 according to SEQ ID NO:71 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 905 according to SEQ ID NO:71 can be at the 3′ end of the primer. In addition, if one of the primers' 3′-ends hybridizes to a cytosine at a position corresponding to position 698 according to SEQ ID NO:16 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 698 according to SEQ ID NO:72 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 698 according to SEQ ID NO:72 can be at the 3′ end of the primer. In addition, if one of the primers' 3′-ends hybridizes to a cytosine at a position corresponding to position 691 according to SEQ ID NO:17 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 691 according to SEQ ID NO:73 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 691 according to SEQ ID NO:73 can be at the 3′ end of the primer. In addition, if one of the primers' 3′-ends hybridizes to a cytosine at a position corresponding to position 930 according to SEQ ID NO:18 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 930 according to SEQ ID NO:74 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 930 according to SEQ ID NO:74 can be at the 3′ end of the primer. In addition, if one of the primers' 3′-ends hybridizes to a cytosine at a position corresponding to position 916 according to SEQ ID NO:91 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 916 according to SEQ ID NO:147 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 916 according to SEQ ID NO:147 can be at the 3′ end of the primer. In addition, if one of the primers' 3′-ends hybridizes to a cytosine at a position corresponding to position 983 according to SEQ ID NO:92 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 983 according to SEQ ID NO:148 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 983 according to SEQ ID NO:148 can be at the 3′ end of the primer. In addition, if one of the primers' 3′-ends hybridizes to a cytosine at a position corresponding to position 948 according to SEQ ID NO:93 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 948 according to SEQ ID NO:149 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 948 according to SEQ ID NO:149 can be at the 3′ end of the primer. In addition, if one of the primers' 3′-ends hybridizes to a cytosine at a position corresponding to position 930 according to SEQ ID NO:94 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 930 according to SEQ ID NO:150 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 930 according to SEQ ID NO:150 can be at the 3′ end of the primer. In addition, if one of the primers' 3′-ends hybridizes to a cytosine at a position corresponding to position 905 according to SEQ ID NO:95 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 905 according to SEQ ID NO:151 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 905 according to SEQ ID NO:151 can be at the 3′ end of the primer. In addition, if one of the primers' 3′-ends hybridizes to a cytosine at a position corresponding to position 698 according to SEQ ID NO:96 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 698 according to SEQ ID NO:152 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 698 according to SEQ ID NO:152 can be at the 3′ end of the primer. In addition, if one of the primers' 3′-ends hybridizes to a cytosine at a position corresponding to position 691 according to SEQ ID NO:97 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 691 according to SEQ ID NO:153 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 691 according to SEQ ID NO:153 can be at the 3′ end of the primer. In addition, if one of the primers' 3′-ends hybridizes to a cytosine at a position corresponding to position 930 according to SEQ ID NO:98 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 930 according to SEQ ID NO:154 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 930 according to SEQ ID NO:154 can be at the 3′ end of the primer.

If one of the primers' 3′-ends hybridizes to a cytosine at a position corresponding to position 5,176 according to SEQ ID NO:1 (rather than a thymine) in a particular ANGPTL7 nucleic acid molecule, then the presence of the amplified fragment would indicate the presence of an ANGPTL7 reference genomic nucleic acid molecule. Conversely, if one of the primers' 3′-ends hybridizes to a thymine at a position corresponding to position 5,176 according to SEQ ID NO:9 (rather than a cytosine) in a particular ANGPTL7 nucleic acid molecule, then the presence of the amplified fragment would indicate the presence of the ANGPTL7 missense variant genomic nucleic acid molecule. In some embodiments, the nucleotide of the primer complementary to the thymine at a position corresponding to position 5,176 according to SEQ ID NO:9 can be at the 3′ end of the primer. In addition, if one of the primers' 3′-ends hybridizes to a cytosine at a position corresponding to position 967 according to SEQ ID NO:11 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 967 according to SEQ ID NO:75 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 967 according to SEQ ID NO:75 can be at the 3′ end of the primer. In addition, if one of the primers' 3′-ends hybridizes to a cytosine at a position corresponding to position 1,034 according to SEQ ID NO:12 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 1,034 according to SEQ ID NO:76 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 1,034 according to SEQ ID NO:76 can be at the 3′ end of the primer. In addition, if one of the primers' 3′-ends hybridizes to a cytosine at a position corresponding to position 999 according to SEQ ID NO:13 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 999 according to SEQ ID NO:77 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 999 according to SEQ ID NO:77 can be at the 3′ end of the primer. In addition, if one of the primers' 3′-ends hybridizes to a cytosine at a position corresponding to position 981 according to SEQ ID NO:14 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 981 according to SEQ ID NO:78 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 981 according to SEQ ID NO:78 can be at the 3′ end of the primer. In addition, if one of the primers' 3′-ends hybridizes to a cytosine at a position corresponding to position 956 according to SEQ ID NO:15 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 956 according to SEQ ID NO:79 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 956 according to SEQ ID NO:79 can be at the 3′ end of the primer. In addition, if one of the primers' 3′-ends hybridizes to a cytosine at a position corresponding to position 749 according to SEQ ID NO:16 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 749 according to SEQ ID NO:80 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 749 according to SEQ ID NO:80 can be at the 3′ end of the primer. In addition, if one of the primers' 3′-ends hybridizes to a cytosine at a position corresponding to position 742 according to SEQ ID NO:17 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 742 according to SEQ ID NO:81 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 742 according to SEQ ID NO:81 can be at the 3′ end of the primer. In addition, if one of the primers' 3′-ends hybridizes to a cytosine at a position corresponding to position 981 according to SEQ ID NO:18 (rather than a uracil) in a particular ANGPTL7 nucleic acid molecule, then the presence of the amplified fragment would indicate the presence of an ANGPTL7 reference mRNA molecule. Conversely, if one of the primers' 3′-ends hybridizes to a uracil at a position corresponding to position 981 according to SEQ ID NO:82 (rather than a cytosine) in a particular ANGPTL7 mRNA molecule, then the presence of the amplified fragment would indicate the presence of the ANGPTL7 missense variant mRNA molecule. In some embodiments, the nucleotide of the primer complementary to the uracil at a position corresponding to position 981 according to SEQ ID NO:82 can be at the 3′ end of the primer. In addition, if one of the primers' 3′-ends hybridizes to a cytosine at a position corresponding to position 967 according to SEQ ID NO:91 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 967 according to SEQ ID NO:155 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 967 according to SEQ ID NO:155 can be at the 3′ end of the primer. In addition, if one of the primers' 3′-ends hybridizes to a cytosine at a position corresponding to position 1,034 according to SEQ ID NO:92 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 1,034 according to SEQ ID NO:156 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 1,034 according to SEQ ID NO:156 can be at the 3′ end of the primer. In addition, if one of the primers' 3′-ends hybridizes to a cytosine at a position corresponding to position 999 according to SEQ ID NO:93 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 999 according to SEQ ID NO:157 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 999 according to SEQ ID NO:157 can be at the 3′ end of the primer. In addition, if one of the primers' 3′-ends hybridizes to a cytosine at a position corresponding to position 981 according to SEQ ID NO:94 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 981 according to SEQ ID NO:158 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 981 according to SEQ ID NO:158 can be at the 3′ end of the primer. In addition, if one of the primers' 3′-ends hybridizes to a cytosine at a position corresponding to position 956 according to SEQ ID NO:95 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 956 according to SEQ ID NO:159 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 956 according to SEQ ID NO:159 can be at the 3′ end of the primer. In addition, if one of the primers' 3′-ends hybridizes to a cytosine at a position corresponding to position 749 according to SEQ ID NO:96 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 749 according to SEQ ID NO:160 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 749 according to SEQ ID NO:160 can be at the 3′ end of the primer. In addition, if one of the primers' 3′-ends hybridizes to a cytosine at a position corresponding to position 742 according to SEQ ID NO:97 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 742 according to SEQ ID NO:161 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 742 according to SEQ ID NO:161 can be at the 3′ end of the primer. In addition, if one of the primers' 3′-ends hybridizes to a cytosine at a position corresponding to position 981 according to SEQ ID NO:98 (rather than a thymine) in a particular ANGPTL7 nucleic acid molecule, then the presence of the amplified fragment would indicate the presence of an ANGPTL7 reference cDNA molecule. Conversely, if one of the primers' 3′-ends hybridizes to a thymine at a position corresponding to position 981 according to SEQ ID NO:162 (rather than a cytosine) in a particular ANGPTL7 cDNA molecule, then the presence of the amplified fragment would indicate the presence of the ANGPTL7 missense variant cDNA molecule. In some embodiments, the nucleotide of the primer complementary to the thymine at a position corresponding to position 981 according to SEQ ID NO:162 can be at the 3′ end of the primer.

If one of the primers' 3′-ends hybridizes to a thymine at a position corresponding to position 5,232 according to SEQ ID NO:1 (rather than an adenine) in a particular ANGPTL7 nucleic acid molecule, then the presence of the amplified fragment would indicate the presence of an ANGPTL7 reference genomic nucleic acid molecule. Conversely, if one of the primers' 3′-ends hybridizes to an adenine at a position corresponding to position 5,232 according to SEQ ID NO:10 (rather than a thymine) in a particular ANGPTL7 nucleic acid molecule, then the presence of the amplified fragment would indicate the presence of the ANGPTL7 missense variant genomic nucleic acid molecule. In some embodiments, the nucleotide of the primer complementary to the adenine at a position corresponding to position 5,232 according to SEQ ID NO:10 can be at the 3′ end of the primer. In addition, if one of the primers' 3′-ends hybridizes to a uracil at a position corresponding to position 1,023 according to SEQ ID NO:11 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 1,023 according to SEQ ID NO:83 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 1,023 according to SEQ ID NO:83 can be at the 3′ end of the primer. In addition, if one of the primers' 3′-ends hybridizes to a uracil at a position corresponding to position 1,090 according to SEQ ID NO:12 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 1,090 according to SEQ ID NO:84 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 1,090 according to SEQ ID NO:84 can be at the 3′ end of the primer. In addition, if one of the primers' 3′-ends hybridizes to a uracil at a position corresponding to position 1,055 according to SEQ ID NO:13 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 1,055 according to SEQ ID NO:85 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 1,055 according to SEQ ID NO:85 can be at the 3′ end of the primer. In addition, if one of the primers' 3′-ends hybridizes to a uracil at a position corresponding to position 1,037 according to SEQ ID NO:14 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 1,037 according to SEQ ID NO:86 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 1,037 according to SEQ ID NO:86 can be at the 3′ end of the primer. In addition, if one of the primers' 3′-ends hybridizes to a uracil at a position corresponding to position 1,012 according to SEQ ID NO:15 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 1,012 according to SEQ ID NO:87 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 1,012 according to SEQ ID NO:87 can be at the 3′ end of the primer. In addition, if one of the primers' 3′-ends hybridizes to a uracil at a position corresponding to position 805 according to SEQ ID NO:16 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 805 according to SEQ ID NO:88 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 805 according to SEQ ID NO:88 can be at the 3′ end of the primer. In addition, if one of the primers' 3′-ends hybridizes to a uracil at a position corresponding to position 798 according to SEQ ID NO:17 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 798 according to SEQ ID NO:89 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 798 according to SEQ ID NO:89 can be at the 3′ end of the primer. In addition, if one of the primers' 3′-ends hybridizes to a uracil at a position corresponding to position 1,037 according to SEQ ID NO:18 (rather than an adenine) in a particular ANGPTL7 nucleic acid molecule, then the presence of the amplified fragment would indicate the presence of an ANGPTL7 reference mRNA molecule. Conversely, if one of the primers' 3′-ends hybridizes to an adenine at a position corresponding to position 1,037 according to SEQ ID NO:90 (rather than a uracil) in a particular ANGPTL7 mRNA molecule, then the presence of the amplified fragment would indicate the presence of the ANGPTL7 missense variant mRNA molecule. In some embodiments, the nucleotide of the primer complementary to the adenine at a position corresponding to position 1,037 according to SEQ ID NO:90 can be at the 3′ end of the primer. In addition, if one of the primers' 3′-ends hybridizes to a thymine at a position corresponding to position 1,023 according to SEQ ID NO:91 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 1,023 according to SEQ ID NO:163 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 1,023 according to SEQ ID NO:163 can be at the 3′ end of the primer. In addition, if one of the primers' 3′-ends hybridizes to a thymine at a position corresponding to position 1,090 according to SEQ ID NO:92 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 1,090 according to SEQ ID NO:164 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 1,090 according to SEQ ID NO:164 can be at the 3′ end of the primer. In addition, if one of the primers' 3′-ends hybridizes to a thymine at a position corresponding to position 1,055 according to SEQ ID NO:93 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 1,055 according to SEQ ID NO:165 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 1,055 according to SEQ ID NO:165 can be at the 3′ end of the primer. In addition, if one of the primers' 3′-ends hybridizes to a thymine at a position corresponding to position 1,037 according to SEQ ID NO:94 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 1,037 according to SEQ ID NO:166 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 1,037 according to SEQ ID NO:166 can be at the 3′ end of the primer. In addition, if one of the primers' 3′-ends hybridizes to a thymine at a position corresponding to position 1,012 according to SEQ ID NO:95 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 1,012 according to SEQ ID NO:167 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 1,012 according to SEQ ID NO:167 can be at the 3′ end of the primer. In addition, if one of the primers' 3′-ends hybridizes to a thymine at a position corresponding to position 805 according to SEQ ID NO:96 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 805 according to SEQ ID NO:168 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 805 according to SEQ ID NO:168 can be at the 3′ end of the primer. In addition, if one of the primers' 3′-ends hybridizes to a thymine at a position corresponding to position 798 according to SEQ ID NO:97 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 798 according to SEQ ID NO:169 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 798 according to SEQ ID NO:169 can be at the 3′ end of the primer. In addition, if one of the primers' 3′-ends hybridizes to a thymine at a position corresponding to position 1,037 according to SEQ ID NO:98 (rather than an adenine) in a particular ANGPTL7 nucleic acid molecule, then the presence of the amplified fragment would indicate the presence of an ANGPTL7 reference cDNA molecule. Conversely, if one of the primers' 3′-ends hybridizes to an adenine at a position corresponding to position 1,037 according to SEQ ID NO:170 (rather than a thymine) in a particular ANGPTL7 cDNA molecule, then the presence of the amplified fragment would indicate the presence of the ANGPTL7 missense variant cDNA molecule. In some embodiments, the nucleotide of the primer complementary to the adenine at a position corresponding to position 1,037 according to SEQ ID NO:170 can be at the 3′ end of the primer.

In the context of the present disclosure “specifically hybridizes” means that the probe or primer (such as, for example, the alteration-specific probe or alteration-specific primer) does not hybridize to a nucleic acid sequence encoding an ANGPTL7 reference genomic nucleic acid molecule, an ANGPTL7 reference mRNA molecule, and/or an ANGPTL7 reference cDNA molecule.

In any of the embodiments described throughout the present disclosure, the probes (such as, for example, an alteration-specific probe) can comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well. In some embodiments, the support is a microarray.

The present disclosure also provides molecular complexes comprising or consisting of any of the ANGPTL7 missense nucleic acid molecules (genomic nucleic acid molecules, mRNA molecules, or cDNA molecules), or complement thereof, described herein and any of the alteration-specific primers or alteration-specific probes described herein. In some embodiments, the ANGPTL7 missense nucleic acid molecules (genomic nucleic acid molecules, mRNA molecules, or cDNA molecules), or complement thereof, in the molecular complexes are single-stranded. In some embodiments, the ANGPTL7 missense nucleic acid molecule is any of the genomic nucleic acid molecules described herein. In some embodiments, the ANGPTL7 missense nucleic acid molecule is any of the mRNA molecules described herein. In some embodiments, the ANGPTL7 missense nucleic acid molecule is any of the cDNA molecules described herein. In some embodiments, the molecular complex comprises or consists of any of the ANGPTL7 missense nucleic acid molecules (genomic nucleic acid molecules, mRNA molecules, or cDNA molecules), or complement thereof, described herein and any of the alteration-specific primers described herein. In some embodiments, the molecular complex comprises or consists of any of the ANGPTL7 missense nucleic acid molecules (genomic nucleic acid molecules, mRNA molecules, or cDNA molecules), or complement thereof, described herein and any of the alteration-specific probes described herein.

In some embodiments, the molecular complex comprises or consists of an alteration-specific primer or an alteration-specific probe hybridized to an ANGPTL7 genomic nucleic acid molecule encoding an ANGPTL7 predicted loss-of-function polypeptide, wherein the alteration-specific primer or the alteration-specific probe is hybridized to the ANGPTL7 genomic nucleic acid molecule at a position corresponding to: position 4,229 according to SEQ ID NO:2, or the complement thereof; position 4,269 according to SEQ ID NO:3, or the complement thereof; position 4,273 according to SEQ ID NO:4, or the complement thereof; position 4,277 according to SEQ ID NO:5, or the complement thereof; position 4,311 according to SEQ ID NO:6, or the complement thereof; position 4,322 according to SEQ ID NO:7, or the complement thereof; position 5,125 according to SEQ ID NO:8, or the complement thereof; or position 5,176 according to SEQ ID NO:9, or the complement thereof.

In some embodiments, the molecular complex comprises or consists of an alteration-specific primer or an alteration-specific probe that is hybridized to: an ATC codon at positions corresponding to positions 4,229-4,231 according to SEQ ID NO:2, an AAC codon at positions corresponding to positions 4,268-4,270 according to SEQ ID NO:3, a CAT codon at positions corresponding to positions 4,271-4,273 according to SEQ ID NO:4, a TGA codon at positions corresponding to positions 4,277-4,279 according to SEQ ID NO:5, a TAG codon at positions corresponding to positions 4,310-4,312 according to SEQ ID NO:6, a CAG codon at positions corresponding to positions 4,322-4,324 according to SEQ ID NO:7, a TGC codon at positions corresponding to positions 5,125-5,127 according to SEQ ID NO:8, a TGC codon at positions corresponding to positions 5,176-5,178 according to SEQ ID NO:9, or a CAA codon at positions corresponding to positions 5,230-5,232 according to SEQ ID NO:10.

In some embodiments, the molecular complex comprises or consists of a genomic nucleic acid molecule that comprises SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In some embodiments, the molecular complex comprises or consists of an alteration-specific primer or an alteration-specific probe hybridized to an ANGPTL7 mRNA molecule encoding an ANGPTL7 predicted loss-of-function polypeptide, wherein the alteration-specific primer or the alteration-specific probe is hybridized to the ANGPTL7 mRNA molecule at a position corresponding to: position 706 according to SEQ ID NO:19, or the complement thereof; position 773 according to SEQ ID NO:20, or the complement thereof; position 738 according to SEQ ID NO:21, or the complement thereof; position 720 according to SEQ ID NO:22, or the complement thereof; position 695 according to SEQ ID NO:23, or the complement thereof; position 488 according to SEQ ID NO:24, or the complement thereof; position 481 according to SEQ ID NO:25, or the complement thereof; position 720 according to SEQ ID NO:26, or the complement thereof; position 746 according to SEQ ID NO:27, or the complement thereof; position 812 according to SEQ ID NO:28, or the complement thereof; position 778 according to SEQ ID NO:29, or the complement thereof; position 760 according to SEQ ID NO:30, or the complement thereof; position 735 according to SEQ ID NO:31, or the complement thereof; position 528 according to SEQ ID NO:32, or the complement thereof; position 521 according to SEQ ID NO:33, or the complement thereof; position 760 according to SEQ ID NO:34, or the complement thereof; position 750 according to SEQ ID NO:35, or the complement thereof; position 817 according to SEQ ID NO:36, or the complement thereof; position 782 according to SEQ ID NO:37, or the complement thereof; position 764 according to SEQ ID NO:38, or the complement thereof; position 739 according to SEQ ID NO:39, or the complement thereof; position 532 according to SEQ ID NO:40, or the complement thereof; position 525 according to SEQ ID NO:41, or the complement thereof; position 764 according to SEQ ID NO:42, or the complement thereof; position 754 according to SEQ ID NO:43, or the complement thereof; position 821 according to SEQ ID NO:44, or the complement thereof; position 786 according to SEQ ID NO:45, or the complement thereof; position 768 according to SEQ ID NO:46, or the complement thereof; position 743 according to SEQ ID NO:47, or the complement thereof; position 536 according to SEQ ID NO:48, or the complement thereof; position 529 according to SEQ ID NO:49, or the complement thereof; position 768 according to SEQ ID NO:50, or the complement thereof; position 788 according to SEQ ID NO:51, or the complement thereof; position 855 according to SEQ ID NO:52, or the complement thereof; position 820 according to SEQ ID NO:53, or the complement thereof; position 802 according to SEQ ID NO:54, or the complement thereof; position 777 according to SEQ ID NO:55, or the complement thereof; position 570 according to SEQ ID NO:56, or the complement thereof; position 563 according to SEQ ID NO:57, or the complement thereof; position 802 according to SEQ ID NO:58, or the complement thereof; position 799 according to SEQ ID NO:59, or the complement thereof; position 866 according to SEQ ID NO:60, or the complement thereof; position 831 according to SEQ ID NO:61, or the complement thereof; position 813 according to SEQ ID NO:62, or the complement thereof; position 788 according to SEQ ID NO:63, or the complement thereof; position 581 according to SEQ ID NO:64, or the complement thereof; position 574 according to SEQ ID NO:65, or the complement thereof; position 813 according to SEQ ID NO:66, or the complement thereof; position 916 according to SEQ ID NO:67, or the complement thereof; position 983 according to SEQ ID NO:68, or the complement thereof; position 948 according to SEQ ID NO:69, or the complement thereof; position 930 according to SEQ ID NO:70, or the complement thereof; position 905 according to SEQ ID NO:71, or the complement thereof; position 698 according to SEQ ID NO:72, or the complement thereof; position 691 according to SEQ ID NO:73, or the complement thereof; position 930 according to SEQ ID NO:74, or the complement thereof; position 967 according to SEQ ID NO:75, or the complement thereof; position 1,034 according to SEQ ID NO:76, or the complement thereof; position 999 according to SEQ ID NO:77, or the complement thereof; position 981 according to SEQ ID NO:78, or the complement thereof; position 956 according to SEQ ID NO:79, or the complement thereof; position 749 according to SEQ ID NO:80, or the complement thereof; position 742 according to SEQ ID NO:81, or the complement thereof; position 981 according to SEQ ID NO:82, or the complement thereof; position 1,023 according to SEQ ID NO:83, or the complement thereof; position 1,090 according to SEQ ID NO:84, or the complement thereof; position 1,055 according to SEQ ID NO:85, or the complement thereof; position 1,037 according to SEQ ID NO:86, or the complement thereof; position 1,012 according to SEQ ID NO:87, or the complement thereof; position 805 according to SEQ ID NO:88, or the complement thereof; position 798 according to SEQ ID NO:89, or the complement thereof; or position 1,037 according to SEQ ID NO:90, or the complement thereof.

In some embodiments, the molecular complex comprises or consists of an alteration-specific primer or an alteration-specific probe that is hybridized to: an AUC codon at positions corresponding to positions 706-708 according to SEQ ID NO:19, an AUC codon at positions corresponding to positions 773-775 according to SEQ ID NO:20, an AUC codon at positions corresponding to positions 738-740 according to SEQ ID NO:21, an AUC codon at positions corresponding to positions 720-722 according to SEQ ID NO:22, an AUC codon at positions corresponding to positions 695-697 according to SEQ ID NO:23, an AUC codon at positions corresponding to positions 488-490 according to SEQ ID NO:24, an AUC codon at positions corresponding to positions 481-483 according to SEQ ID NO:25, an AUC codon at positions corresponding to positions 720-722 according to SEQ ID NO:26, an AAC codon at positions corresponding to positions 745-747 according to SEQ ID NO:27, an AAC codon at positions corresponding to positions 811-813 according to SEQ ID NO:28, an AAC codon at positions corresponding to positions 777-779 according to SEQ ID NO:29, an AAC codon at positions corresponding to positions 759-761 according to SEQ ID NO:30, an AAC codon at positions corresponding to positions 734-736 according to SEQ ID NO:31, an AAC codon at positions corresponding to positions 527-529 according to SEQ ID NO:32, an AAC codon at positions corresponding to positions 520-522 according to SEQ ID NO:33, an AAC codon at positions corresponding to positions 759-761 according to SEQ ID NO:34, a CAU codon at positions corresponding to positions 748-750 according to SEQ ID NO:35, a CAU codon at positions corresponding to positions 815-817 according to SEQ ID NO:36, a CAU codon at positions corresponding to positions 780-782 according to SEQ ID NO:37, a CAU codon at positions corresponding to positions 762-764 according to SEQ ID NO:38, a CAU codon at positions corresponding to positions 737-739 according to SEQ ID NO:39, a CAU codon at positions corresponding to positions 530-532 according to SEQ ID NO:40, a CAU codon at positions corresponding to positions 523-525 according to SEQ ID NO:41, a CAU codon at positions corresponding to positions 762-764 according to SEQ ID NO:42, a UGA codon at positions corresponding to positions 754-756 according to SEQ ID NO:43, a UGA codon at positions corresponding to positions 821-823 according to SEQ ID NO:44, a UGA codon at positions corresponding to positions 786-788 according to SEQ ID NO:45, a UGA codon at positions corresponding to positions 768-770 according to SEQ ID NO:46, a UGA codon at positions corresponding to positions 743-745 according to SEQ ID NO:47, a UGA codon at positions corresponding to positions 536-538 according to SEQ ID NO:48, a UGA codon at positions corresponding to positions 529-531 according to SEQ ID NO:49, a UGA codon at positions corresponding to positions 768-770 according to SEQ ID NO:50, a UGG codon at positions corresponding to positions 787-789 according to SEQ ID NO:51, a UGG codon at positions corresponding to positions 854-856 according to SEQ ID NO:52, a UGG codon at positions corresponding to positions 819-821 according to SEQ ID NO:53, a UGG codon at positions corresponding to positions 801-803 according to SEQ ID NO:54, a UGG codon at positions corresponding to positions 776-778 according to SEQ ID NO:55, a UGG codon at positions corresponding to positions 569-571 according to SEQ ID NO:56, a UGG codon at positions corresponding to positions 562-564 according to SEQ ID NO:57, a UGG codon at positions corresponding to positions 801-803 according to SEQ ID NO:58, a CAG codon at positions corresponding to positions 799-801 according to SEQ ID NO:59, a CAG codon at positions corresponding to positions 866-868 according to SEQ ID NO:60, a CAG codon at positions corresponding to positions 831-833 according to SEQ ID NO:61, a CAG codon at positions corresponding to positions 813-815 according to SEQ ID NO:62, a CAG codon at positions corresponding to positions 788-790 according to SEQ ID NO:63, a CAG codon at positions corresponding to positions 581-583 according to SEQ ID NO:64, a CAG codon at positions corresponding to positions 574-576 according to SEQ ID NO:65, a CAG codon at positions corresponding to positions 813-815 according to SEQ ID NO:66, a UGC codon at positions corresponding to positions 916-918 according to SEQ ID NO:67, a UGC codon at positions corresponding to positions 983-985 according to SEQ ID NO:68, a UGC codon at positions corresponding to positions 948-950 according to SEQ ID NO:69, a UGC codon at positions corresponding to positions 930-932 according to SEQ ID NO:70, a UGC codon at positions corresponding to positions 905-907 according to SEQ ID NO:71, a UGC codon at positions corresponding to positions 698-700 according to SEQ ID NO:72, a UGC codon at positions corresponding to positions 691-693 according to SEQ ID NO:73, a UGC codon at positions corresponding to positions 930-932 according to SEQ ID NO:74, a UGC codon at positions corresponding to positions 967-969 according to SEQ ID NO:75, a UGC codon at positions corresponding to positions 1,034-1,036 according to SEQ ID NO:76, a UGC codon at positions corresponding to positions 999-1,001 according to SEQ ID NO:77, a UGC codon at positions corresponding to positions 981-983 according to SEQ ID NO:78, a UGC codon at positions corresponding to positions 956-958 according to SEQ ID NO:79, a UGC codon at positions corresponding to positions 749-751 according to SEQ ID NO:80, a UGC codon at positions corresponding to positions 742-744 according to SEQ ID NO:81, a UGC codon at positions corresponding to positions 981-983 according to SEQ ID NO:82, a CAA codon at positions corresponding to positions 1,021-1,023 according to SEQ ID NO:83, a CAA codon at positions corresponding to positions 1,088-1,090 according to SEQ ID NO:84, a CAA codon at positions corresponding to positions 1,053-1,055 according to SEQ ID NO:85, a CAA codon at positions corresponding to positions 1,035-1,037 according to SEQ ID NO:86, a CAA codon at positions corresponding to positions 1,010-1,012 according to SEQ ID NO:87, a CAA codon at positions corresponding to positions 803-805 according to SEQ ID NO:88, a CAA codon at positions corresponding to positions 796-798 according to SEQ ID NO:89, a CAA codon at positions corresponding to positions 1,035-1,037 according to SEQ ID NO:90.

In some embodiments, the molecular complex comprises or consists of an mRNA molecule that comprises SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:87, SEQ ID NO:88, SEQ ID NO:89, SEQ ID NO:90.

In some embodiments, the molecular complex comprises or consists of an alteration-specific primer or an alteration-specific probe hybridized to an ANGPTL7 cDNA molecule encoding an ANGPTL7 predicted loss-of-function polypeptide, wherein the alteration-specific primer or the alteration-specific probe is hybridized to the ANGPTL7 cDNA molecule at a position corresponding to: position 706 according to SEQ ID NO:99, or the complement thereof; position 773 according to SEQ ID NO:100, or the complement thereof; position 738 according to SEQ ID NO:101, or the complement thereof; position 720 according to SEQ ID NO:102, or the complement thereof; position 695 according to SEQ ID NO:103, or the complement thereof; position 488 according to SEQ ID NO:104, or the complement thereof; position 481 according to SEQ ID NO:105, or the complement thereof; position 720 according to SEQ ID NO:106, or the complement thereof; position 746 according to SEQ ID NO:107, or the complement thereof; position 812 according to SEQ ID NO:108, or the complement thereof; position 778 according to SEQ ID NO:109, or the complement thereof; position 760 according to SEQ ID NO:110, or the complement thereof; position 735 according to SEQ ID NO:111, or the complement thereof; position 528 according to SEQ ID NO:112, or the complement thereof; position 529 according to SEQ ID NO:113, or the complement thereof; position 760 according to SEQ ID NO:114, or the complement thereof; position 750 according to SEQ ID NO:115, or the complement thereof; position 817 according to SEQ ID NO:116, or the complement thereof; position 782 according to SEQ ID NO:117, or the complement thereof; position 764 according to SEQ ID NO:118, or the complement thereof; position 739 according to SEQ ID NO:119, or the complement thereof; position 532 according to SEQ ID NO:120, or the complement thereof; position 525 according to SEQ ID NO:121, or the complement thereof; position 764 according to SEQ ID NO:122, or the complement thereof; position 754 according to SEQ ID NO:123, or the complement thereof; position 821 according to SEQ ID NO:124, or the complement thereof; position 786 according to SEQ ID NO:125, or the complement thereof; position 768 according to SEQ ID NO:126, or the complement thereof; position 743 according to SEQ ID NO:127, or the complement thereof; position 536 according to SEQ ID NO:128, or the complement thereof; position 529 according to SEQ ID NO:129, or the complement thereof; position 768 according to SEQ ID NO:130, or the complement thereof; position 788 according to SEQ ID NO:131, or the complement thereof; position 855 according to SEQ ID NO:132, or the complement thereof; position 820 according to SEQ ID NO:133, or the complement thereof; position 802 according to SEQ ID NO:134, or the complement thereof; position 777 according to SEQ ID NO:135, or the complement thereof; position 570 according to SEQ ID NO:136, or the complement thereof; position 563 according to SEQ ID NO:137, or the complement thereof; position 802 according to SEQ ID NO:138, or the complement thereof; position 799 according to SEQ ID NO:139, or the complement thereof; position 866 according to SEQ ID NO:140, or the complement thereof; position 831 according to SEQ ID NO:141, or the complement thereof; position 813 according to SEQ ID NO:142, or the complement thereof; position 788 according to SEQ ID NO:143, or the complement thereof; position 581 according to SEQ ID NO:144, or the complement thereof; position 574 according to SEQ ID NO:145, or the complement thereof; position 813 according to SEQ ID NO:146, or the complement thereof; position 916 according to SEQ ID NO:147, or the complement thereof; position 983 according to SEQ ID NO:148, or the complement thereof; position 948 according to SEQ ID NO:149, or the complement thereof; position 930 according to SEQ ID NO:150, or the complement thereof; position 905 according to SEQ ID NO:151, or the complement thereof; position 698 according to SEQ ID NO:152, or the complement thereof; position 691 according to SEQ ID NO:153, or the complement thereof; position 930 according to SEQ ID NO:154, or the complement thereof; position 967 according to SEQ ID NO:155, or the complement thereof; position 1,034 according to SEQ ID NO:156, or the complement thereof; position 999 according to SEQ ID NO:157, or the complement thereof; position 981 according to SEQ ID NO:158, or the complement thereof; position 956 according to SEQ ID NO:159, or the complement thereof; position 749 according to SEQ ID NO:160, or the complement thereof; position 742 according to SEQ ID NO:161, or the complement thereof; position 981 according to SEQ ID NO:162, or the complement thereof; position 1,023 according to SEQ ID NO:163, or the complement thereof; position 1,090 according to SEQ ID NO:164, or the complement thereof; position 1,055 according to SEQ ID NO:165, or the complement thereof; position 1,037 according to SEQ ID NO:166, or the complement thereof; position 1,012 according to SEQ ID NO:167, or the complement thereof; position 805 according to SEQ ID NO:168, or the complement thereof; position 798 according to SEQ ID NO:169, or the complement thereof; or position 1,037 according to SEQ ID NO:170, or the complement thereof.

In some embodiments, the molecular complex comprises or consists of an alteration-specific primer or an alteration-specific probe that is hybridized to: an ATC codon at positions corresponding to positions 706-708 according to SEQ ID NO:99, an ATC codon at positions corresponding to positions 773-775 according to SEQ ID NO:100, an ATC codon at positions corresponding to positions 738-740 according to SEQ ID NO:101, an ATC codon at positions corresponding to positions 720-722 according to SEQ ID NO:102, an ATC codon at positions corresponding to positions 695-697 according to SEQ ID NO:103, an ATC codon at positions corresponding to positions 488-490 according to SEQ ID NO:104, an ATC codon at positions corresponding to positions 481-483 according to SEQ ID NO:105, an ATC codon at positions corresponding to positions 720-722 according to SEQ ID NO:106, an AAC codon at positions corresponding to positions 745-747 according to SEQ ID NO:107, an AAC codon at positions corresponding to positions 811-813 according to SEQ ID NO:108, an AAC codon at positions corresponding to positions 777-779 according to SEQ ID NO:109, an AAC codon at positions corresponding to positions 759-761 according to SEQ ID NO:110, an AAC codon at positions corresponding to positions 734-736 according to SEQ ID NO:111, an AAC codon at positions corresponding to positions 527-529 according to SEQ ID NO:112, an AAC codon at positions corresponding to positions 520-522 according to SEQ ID NO:113, an AAC codon at positions corresponding to positions 759-761 according to SEQ ID NO:114, a CAT codon at positions corresponding to positions 748-750 according to SEQ ID NO:115, a CAT codon at positions corresponding to positions 815-817 according to SEQ ID NO:116, a CAT codon at positions corresponding to positions 780-782 according to SEQ ID NO:117, a CAT codon at positions corresponding to positions 762-764 according to SEQ ID NO:118, a CAT codon at positions corresponding to positions 737-739 according to SEQ ID NO:119, a CAT codon at positions corresponding to positions 530-532 according to SEQ ID NO:120, a CAT codon at positions corresponding to positions 523-525 according to SEQ ID NO:121, a CAT codon at positions corresponding to positions 762-764 according to SEQ ID NO:122, a TGA codon at positions corresponding to positions 754-756 according to SEQ ID NO:123, a TGA codon at positions corresponding to positions 821-823 according to SEQ ID NO:124, a TGA codon at positions corresponding to positions 786-788 according to SEQ ID NO:125, a TGA codon at positions corresponding to positions 768-770 according to SEQ ID NO:126, a TGA codon at positions corresponding to positions 743-745 according to SEQ ID NO:127, a TGA codon at positions corresponding to positions 536-538 according to SEQ ID NO:128, a TGA codon at positions corresponding to positions 529-531 according to SEQ ID NO:129, a TGA codon at positions corresponding to positions 768-770 according to SEQ ID NO:130, a TAG codon at positions corresponding to positions 787-789 according to SEQ ID NO:131, a TAG codon at positions corresponding to positions 854-856 according to SEQ ID NO:132, a TAG codon at positions corresponding to positions 819-821 according to SEQ ID NO:133, a TAG codon at positions corresponding to positions 801-803 according to SEQ ID NO:134, a TAG codon at positions corresponding to positions 776-778 according to SEQ ID NO:135, a TAG codon at positions corresponding to positions 569-571 according to SEQ ID NO:136, a TAG codon at positions corresponding to positions 562-564 according to SEQ ID NO:137, a TAG codon at positions corresponding to positions 801-803 according to SEQ ID NO:138, a CAG codon at positions corresponding to positions 799-801 according to SEQ ID NO:139, a CAG codon at positions corresponding to positions 866-868 according to SEQ ID NO:140, a CAG codon at positions corresponding to positions 831-833 according to SEQ ID NO:141, a CAG codon at positions corresponding to positions 813-815 according to SEQ ID NO:142, a CAG codon at positions corresponding to positions 788-790 according to SEQ ID NO:143, a CAG codon at positions corresponding to positions 581-583 according to SEQ ID NO:144, a CAG codon at positions corresponding to positions 574-576 according to SEQ ID NO:145, a CAG codon at positions corresponding to positions 813-815 according to SEQ ID NO:146, a TGC codon at positions corresponding to positions 916-918 according to SEQ ID NO:147, a TGC codon at positions corresponding to positions 983-985 according to SEQ ID NO:148, a TGC codon at positions corresponding to positions 948-950 according to SEQ ID NO:149, a TGC codon at positions corresponding to positions 930-932 according to SEQ ID NO:150, a TGC codon at positions corresponding to positions 905-907 according to SEQ ID NO:151, a TGC codon at positions corresponding to positions 698-700 according to SEQ ID NO:152, a TGC codon at positions corresponding to positions 691-693 according to SEQ ID NO:153, a TGC codon at positions corresponding to positions 930-932 according to SEQ ID NO:154, a TGC codon at positions corresponding to positions 967-969 according to SEQ ID NO:155, a TGC codon at positions corresponding to positions 1,034-1,036 according to SEQ ID NO:156, a TGC codon at positions corresponding to positions 999-1,001 according to SEQ ID NO:157, a TGC codon at positions corresponding to positions 981-983 according to SEQ ID NO:158, a TGC codon at positions corresponding to positions 956-958 according to SEQ ID NO:159, a TGC codon at positions corresponding to positions 749-751 according to SEQ ID NO:160, a TGC codon at positions corresponding to positions 742-744 according to SEQ ID NO:161, a TGC codon at positions corresponding to positions 981-983 according to SEQ ID NO:162 a CAA codon at positions corresponding to positions 1,021-1,023 according to SEQ ID NO:163, a CAA codon at positions corresponding to positions 1,088-1,090 according to SEQ ID NO:164, a CAA codon at positions corresponding to positions 1,053-1,055 according to SEQ ID NO:165, a CAA codon at positions corresponding to positions 1,035-1,037 according to SEQ ID NO:166, a CAA codon at positions corresponding to positions 1,010-1,012 according to SEQ ID NO:167, a CAA codon at positions corresponding to positions 803-805 according to SEQ ID NO:168, a CAA codon at positions corresponding to positions 796-798 according to SEQ ID NO:169, a CAA codon at positions corresponding to positions 1,035-1,037 according to SEQ ID NO:170.

In some embodiments, the molecular complex comprises or consists of an cDNA molecule that comprises SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170.

In some embodiments, the molecular complex comprises an alteration-specific probe or an alteration-specific primer comprising a label. In some embodiments, the label is a fluorescent label, a radiolabel, or biotin. In some embodiments, the molecular complex further comprises a non-human polymerase.

In some embodiments, any of the methods described herein can further comprise determining the subject's aggregate gene burden of having an ANGPTL7 missense variant nucleic acid molecule encoding an ANGPTL7 predicted loss-of-function polypeptide, and/or an ANGPTL7 predicted loss-of-function variant polypeptide associated with a decreased risk of developing an ophthalmic condition. The aggregate gene burden is the aggregate of all variants in the ANGPTL7 gene, which can be carried out in an association analysis with an ophthalmic condition. In some embodiments, the subject is homozygous for one or more ANGPTL7 missense variant nucleic acid molecules encoding an ANGPTL7 predicted loss-of-function polypeptide associated with a decreased risk of developing an ophthalmic condition. In some embodiments, the subject is heterozygous for one or more ANGPTL7 missense variant nucleic acid molecules encoding an ANGPTL7 predicted loss-of-function polypeptide associated with a decreased risk of developing an ophthalmic condition. The result of the association analysis suggests that ANGPTL7 missense variant nucleic acid molecules encoding an ANGPTL7 predicted loss-of-function polypeptide are associated with decreased risk of developing an ophthalmic condition. When the subject has a lower aggregate burden, the subject is at a higher risk of developing an ophthalmic condition and the subject is administered or continued to be administered the therapeutic agent that treats, prevents, or inhibits an ophthalmic condition in a standard dosage amount, and/or an ANGPTL7 inhibitor. When the subject has a greater aggregate burden, the subject is at a lower risk of developing an ophthalmic condition and the subject is administered or continued to be administered the therapeutic agent that treats, prevents, or inhibits an ophthalmic condition in an amount that is the same as or less than the standard dosage amount. The greater the aggregate burden, the lower the risk of developing an ophthalmic condition.

In some embodiments, the subject's aggregate burden of having any one or more ANGPTL7 missense variant nucleic acid molecules encoding an ANGPTL7 predicted loss-of-function polypeptide represents a weighted sum of a plurality of any of the ANGPTL7 missense variant nucleic acid molecules encoding an ANGPTL7 predicted loss-of-function polypeptide. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the ANGPTL7 gene where the genetic burden is the number of alleles multiplied by the association estimate with an ophthalmic condition or related outcome for each allele (e.g., a weighted polygenic burden score). This can include any genetic variants, regardless of their genomic annotation, in proximity to the ANGPTL7 gene (up to 10 Mb around the gene) that show a non-zero association with an ophthalmic condition-related traits in a genetic association analysis. In some embodiments, when the subject has an aggregate burden above a desired threshold score, the subject has a decreased risk of developing an ophthalmic condition. In some embodiments, when the subject has an aggregate burden below a desired threshold score, the subject has an increased risk of developing an ophthalmic condition.

In some embodiments, the aggregate burden may be divided into quintiles, e.g., top quintile, intermediate quintile, and bottom quintile, wherein the top quintile of aggregate burden corresponds to the lowest risk group and the bottom quintile of aggregate burden corresponds to the highest risk group. In some embodiments, a subject having a greater aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with an ophthalmic condition in the top 10%, top 20%, top 30%, top 40%, or top 50% of p-value range for the association. In some embodiments, each of the identified genetic variants comprise the genetic variants having association with an ophthalmic condition with p-value of no more than about 10⁻², about 10⁻³, about 10⁻⁴, about 10⁻⁵, about 10⁻¹, about 10⁻⁷, about 10⁻¹, about 10⁻¹, about 10⁻¹⁰, about 10-11 about 10⁻¹², about 10⁻¹³, about 10⁻¹⁴, about or 10⁻¹⁵. In some embodiments, the identified genetic variants comprise the genetic variants having association with an ophthalmic condition with p-value of less than 5×10⁻⁸. In some embodiments, the identified genetic variants comprise genetic variants having association with an ophthalmic condition in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having aggregate burdens in the bottom decile, quintile, or tertile in a reference population. The threshold of the aggregate burden is determined on the basis of the nature of the intended practical application and the risk difference that would be considered meaningful for that practical application.

In some embodiments, when a subject is identified as having an increased risk of developing an ophthalmic condition, the subject is further administered a therapeutic agent that treats, prevents, or inhibits an ophthalmic condition, and/or an ANGPTL7 inhibitor, as described herein. For example, when the subject is ANGPTL7 reference, and therefore has an increased risk of developing an ophthalmic condition, the subject is administered an ANGPTL7 inhibitor. In some embodiments, such a subject is also administered a therapeutic agent that treats, prevents, or inhibits an ophthalmic condition. In some embodiments, when the subject is heterozygous for an ANGPTL7 missense variant nucleic acid molecule encoding an ANGPTL7 predicted loss-of-function polypeptide, the subject is administered the therapeutic agent that treats, prevents, or inhibits an ophthalmic condition in a dosage amount that is the same as or less than a standard dosage amount, and is also administered an ANGPTL7 inhibitor. In some embodiments, the subject is ANGPTL7 reference. In some embodiments, the subject is heterozygous for an ANGPTL7 missense variant nucleic acid molecule encoding an ANGPTL7 predicted loss-of-function polypeptide. Furthermore, when the subject has a lower aggregate burden for having an ANGPTL7 missense variant nucleic acid molecule encoding an ANGPTL7 predicted loss-of-function polypeptide, and therefore has an increased risk of developing an ophthalmic condition, the subject is administered a therapeutic agent that treats, prevents, or inhibits an ophthalmic condition. In some embodiments, when the subject has a lower aggregate burden for having an ANGPTL7 missense variant nucleic acid molecule encoding an ANGPTL7 predicted loss-of-function polypeptide, the subject is administered the therapeutic agent that treats, prevents, or inhibits an ophthalmic condition in a dosage amount that is the same as or greater than the standard dosage amount administered to a subject who has a greater aggregate burden for having an ANGPTL7 missense variant nucleic acid molecule encoding an ANGPTL7 predicted loss-of-function polypeptide.

The nucleotide sequence of an ANGPTL7 reference genomic nucleic acid molecule is set forth in SEQ ID NO:1. Referring to SEQ ID NO:1, position 4,229 is a thymine. Referring to SEQ ID NO:1, position 4,269 is a thymine. Referring to SEQ ID NO:1, position 4,273 is a guanine. Referring to SEQ ID NO:1, position 4,277 is a cytosine. Referring to SEQ ID NO:1, position 4,311 is a guanine. Referring to SEQ ID NO:1, position 4,322 is an adenine. Referring to SEQ ID NO:1, position 5,125 is a cytosine. Referring to SEQ ID NO:1, position 5,176 is a cytosine. Referring to SEQ ID NO:1, position 5,232 is a thymine.

An ANGPTL7 missense variant genomic nucleic acid molecule exists, wherein the thymine at position 4,229 is replaced with an adenine. The nucleotide sequence of this ANGPTL7 missense variant genomic nucleic acid molecule is set forth in SEQ ID NO:2.

Another ANGPTL7 missense variant genomic nucleic acid molecule exists, wherein the thymine at position 4,269 is replaced with an adenine. The nucleotide sequence of this ANGPTL7 missense variant genomic nucleic acid molecule is set forth in SEQ ID NO:3.

Another ANGPTL7 missense variant genomic nucleic acid molecule exists, wherein the guanine at position 4,273 is replaced with a thymine. The nucleotide sequence of this ANGPTL7 missense variant genomic nucleic acid molecule is set forth in SEQ ID NO:4.

Another ANGPTL7 missense variant genomic nucleic acid molecule exists, wherein the cytosine at position 4,277 is replaced with a thymine. The nucleotide sequence of this ANGPTL7 missense variant genomic nucleic acid molecule is set forth in SEQ ID NO:5.

Another ANGPTL7 missense variant genomic nucleic acid molecule exists, wherein the guanine at position 4,311 is replaced with an adenine. The nucleotide sequence of this ANGPTL7 missense variant genomic nucleic acid molecule is set forth in SEQ ID NO:6.

Another ANGPTL7 missense variant genomic nucleic acid molecule exists, wherein the adenine at position 4,322 is replaced with a cytosine. The nucleotide sequence of this ANGPTL7 missense variant genomic nucleic acid molecule is set forth in SEQ ID NO:7.

Another ANGPTL7 missense variant genomic nucleic acid molecule exists, wherein the cytosine at position 5,125 is replaced with a thymine. The nucleotide sequence of this ANGPTL7 missense variant genomic nucleic acid molecule is set forth in SEQ ID NO:8.

Another ANGPTL7 missense variant genomic nucleic acid molecule exists, wherein the cytosine at position 5,176 is replaced with a thymine. The nucleotide sequence of this ANGPTL7 missense variant genomic nucleic acid molecule is set forth in SEQ ID NO:9.

Another ANGPTL7 missense variant genomic nucleic acid molecule exists, wherein the thymine at position 5,232 is replaced with an adenine. The nucleotide sequence of this ANGPTL7 missense variant genomic nucleic acid molecule is set forth in SEQ ID NO:10.

The nucleotide sequence of an ANGPTL7 reference mRNA molecule is set forth in SEQ ID NO:11. Referring to SEQ ID NO:11, position 706 is a uracil. Referring to SEQ ID NO:11, position 746 is a uracil. Referring to SEQ ID NO:11, position 750 is a guanine. Referring to SEQ ID NO:11, position 754 is a cytosine. Referring to SEQ ID NO:11, position 788 is a guanine. Referring to SEQ ID NO:11, position 799 is an adenine. Referring to SEQ ID NO:11, position 916 is a cytosine. Referring to SEQ ID NO:11, position 967 is a cytosine. Referring to SEQ ID NO:11, position 1,023 is a uracil.

The nucleotide sequence of another ANGPTL7 reference mRNA molecule is set forth in SEQ ID NO:12. Referring to SEQ ID NO:12, position 773 is a uracil. Referring to SEQ ID NO:12, position 812 is a uracil. Referring to SEQ ID NO:12, position 817 is a guanine. Referring to SEQ ID NO:12, position 821 is a cytosine. Referring to SEQ ID NO:12, position 855 is a guanine. Referring to SEQ ID NO:12, position 866 is an adenine. Referring to SEQ ID NO:12, position 983 is a cytosine. Referring to SEQ ID NO:12, position 1,034 is a cytosine. Referring to SEQ ID NO:12, position 1,090 is a uracil.

The nucleotide sequence of another ANGPTL7 reference mRNA molecule is set forth in SEQ ID NO:13. Referring to SEQ ID NO:13, position 738 is a uracil. Referring to SEQ ID NO:13, position 778 is a uracil. Referring to SEQ ID NO:13, position 782 is a guanine. Referring to SEQ ID NO:13, position 786 is a cytosine. Referring to SEQ ID NO:13, position 820 is a guanine. Referring to SEQ ID NO:13, position 831 is an adenine. Referring to SEQ ID NO:13, position 948 is a cytosine. Referring to SEQ ID NO:13, position 999 is a cytosine. Referring to SEQ ID NO:13, position 1,055 is a uracil.

The nucleotide sequence of another ANGPTL7 reference mRNA molecule is set forth in SEQ ID NO:14. Referring to SEQ ID NO:14, position 720 is a uracil. Referring to SEQ ID NO:14, position 760 is a uracil. Referring to SEQ ID NO:14, position 764 is a guanine. Referring to SEQ ID NO:14, position 768 is a cytosine. Referring to SEQ ID NO:14, position 802 is a guanine. Referring to SEQ ID NO:14, position 813 is an adenine. Referring to SEQ ID NO:14, position 948 is a cytosine. Referring to SEQ ID NO:14, position 981 is a cytosine. Referring to SEQ ID NO:14, position 1,037 is a uracil.

The nucleotide sequence of another ANGPTL7 reference mRNA molecule is set forth in SEQ ID NO:15. Referring to SEQ ID NO:15, position 695 is a uracil. Referring to SEQ ID NO:15, position 735 is a uracil. Referring to SEQ ID NO:15, position 739 is a guanine. Referring to SEQ ID NO:15, position 743 is a cytosine. Referring to SEQ ID NO:15, position 777 is a guanine. Referring to SEQ ID NO:15, position 788 is an adenine. Referring to SEQ ID NO:15, position 905 is a cytosine. Referring to SEQ ID NO:15, position 956 is a cytosine. Referring to SEQ ID NO:15, position 1,012 is a uracil.

The nucleotide sequence of another ANGPTL7 reference mRNA molecule is set forth in SEQ ID NO:16. Referring to SEQ ID NO:16, position 488 is a uracil. Referring to SEQ ID NO:16, position 528 is a uracil. Referring to SEQ ID NO:16, position 532 is a guanine. Referring to SEQ ID NO:16, position 536 is a cytosine. Referring to SEQ ID NO:16, position 570 is a guanine. Referring to SEQ ID NO:16, position 581 is an adenine. Referring to SEQ ID NO:16, position 698 is a cytosine. Referring to SEQ ID NO:16, position 749 is a cytosine. Referring to SEQ ID NO:16, position 805 is a uracil.

The nucleotide sequence of another ANGPTL7 reference mRNA molecule is set forth in SEQ ID NO:17. Referring to SEQ ID NO:17, position 481 is a uracil. Referring to SEQ ID NO:17, position 521 is a uracil. Referring to SEQ ID NO:17, position 525 is a guanine. Referring to SEQ ID NO:17, position 529 is a cytosine. Referring to SEQ ID NO:17, position 563 is a guanine. Referring to SEQ ID NO:17, position 574 is an adenine. Referring to SEQ ID NO:17, position 691 is a cytosine. Referring to SEQ ID NO:17, position 742 is a cytosine. Referring to SEQ ID NO:17, position 798 is a uracil.

The nucleotide sequence of another ANGPTL7 reference mRNA molecule is set forth in SEQ ID NO:18. Referring to SEQ ID NO:18, position 720 is a uracil. Referring to SEQ ID NO:18, position 760 is a uracil. Referring to SEQ ID NO:18, position 764 is a guanine. Referring to SEQ ID NO:18, position 768 is a cytosine. Referring to SEQ ID NO:18, position 802 is a guanine. Referring to SEQ ID NO:18, position 813 is an adenine. Referring to SEQ ID NO:18, position 930 is a cytosine. Referring to SEQ ID NO:18, position 981 is a cytosine. Referring to SEQ ID NO:18, position 1,037 is a uracil.

An ANGPTL7 missense variant mRNA molecule exists, wherein the uracil at position 706 is replaced with an adenine. The nucleotide sequence of this ANGPTL7 missense variant mRNA molecule is set forth in SEQ ID NO:19.

Another ANGPTL7 missense variant mRNA molecule exists, wherein the uracil at position 746 is replaced with an adenine. The nucleotide sequence of this ANGPTL7 missense variant mRNA molecule is set forth in SEQ ID NO:20.

Another ANGPTL7 missense variant mRNA molecule exists, wherein the uracil at position 750 is replaced with an adenine. The nucleotide sequence of this ANGPTL7 missense variant mRNA molecule is set forth in SEQ ID NO:21.

Another ANGPTL7 missense variant mRNA molecule exists, wherein the uracil at position 754 is replaced with an adenine. The nucleotide sequence of this ANGPTL7 missense variant mRNA molecule is set forth in SEQ ID NO:22.

Another ANGPTL7 missense variant mRNA molecule exists, wherein the uracil at position 788 is replaced with an adenine. The nucleotide sequence of this ANGPTL7 missense variant mRNA molecule is set forth in SEQ ID NO:23.

Another ANGPTL7 missense variant mRNA molecule exists, wherein the uracil at position 799 is replaced with an adenine. The nucleotide sequence of this ANGPTL7 missense variant mRNA molecule is set forth in SEQ ID NO:24.

Another ANGPTL7 missense variant mRNA molecule exists, wherein the uracil at position 916 is replaced with an adenine. The nucleotide sequence of this ANGPTL7 missense variant mRNA molecule is set forth in SEQ ID NO:25.

Another ANGPTL7 missense variant mRNA molecule exists, wherein the uracil at position 967 is replaced with an adenine. The nucleotide sequence of this ANGPTL7 missense variant mRNA molecule is set forth in SEQ ID NO:26.

Another ANGPTL7 missense variant mRNA molecule exists, wherein the uracil at position 773 is replaced with an adenine. The nucleotide sequence of this ANGPTL7 missense variant mRNA molecule is set forth in SEQ ID NO:27.

Another ANGPTL7 missense variant mRNA molecule exists, wherein the uracil at position 812 is replaced with an adenine. The nucleotide sequence of this ANGPTL7 missense variant mRNA molecule is set forth in SEQ ID NO:28.

Another ANGPTL7 missense variant mRNA molecule exists, wherein the uracil at position 817 is replaced with an adenine. The nucleotide sequence of this ANGPTL7 missense variant mRNA molecule is set forth in SEQ ID NO:29.

Another ANGPTL7 missense variant mRNA molecule exists, wherein the uracil at position 821 is replaced with an adenine. The nucleotide sequence of this ANGPTL7 missense variant mRNA molecule is set forth in SEQ ID NO:30.

Another ANGPTL7 missense variant mRNA molecule exists, wherein the uracil at position 855 is replaced with an adenine. The nucleotide sequence of this ANGPTL7 missense variant mRNA molecule is set forth in SEQ ID NO:31.

Another ANGPTL7 missense variant mRNA molecule exists, wherein the uracil at position 866 is replaced with an adenine. The nucleotide sequence of this ANGPTL7 missense variant mRNA molecule is set forth in SEQ ID NO:32.

Another ANGPTL7 missense variant mRNA molecule exists, wherein the uracil at position 983 is replaced with an adenine. The nucleotide sequence of this ANGPTL7 missense variant mRNA molecule is set forth in SEQ ID NO:33.

Another ANGPTL7 missense variant mRNA molecule exists, wherein the uracil at position 1,034 is replaced with an adenine. The nucleotide sequence of this ANGPTL7 missense variant mRNA molecule is set forth in SEQ ID NO:34.

Another ANGPTL7 missense variant mRNA molecule exists, wherein the guanine at position 738 is replaced with a uracil. The nucleotide sequence of this ANGPTL7 missense variant mRNA molecule is set forth in SEQ ID NO:35.

Another ANGPTL7 missense variant mRNA molecule exists, wherein the guanine at position 778 is replaced with a uracil. The nucleotide sequence of this ANGPTL7 missense variant mRNA molecule is set forth in SEQ ID NO:36.

Another ANGPTL7 missense variant mRNA molecule exists, wherein the guanine at position 782 is replaced with a uracil. The nucleotide sequence of this ANGPTL7 missense variant mRNA molecule is set forth in SEQ ID NO:37.

Another ANGPTL7 missense variant mRNA molecule exists, wherein the guanine at position 786 is replaced with a uracil. The nucleotide sequence of this ANGPTL7 missense variant mRNA molecule is set forth in SEQ ID NO:38.

Another ANGPTL7 missense variant mRNA molecule exists, wherein the guanine at position 820 is replaced with a uracil. The nucleotide sequence of this ANGPTL7 missense variant mRNA molecule is set forth in SEQ ID NO:39.

Another ANGPTL7 missense variant mRNA molecule exists, wherein the guanine at position 831 is replaced with a uracil. The nucleotide sequence of this ANGPTL7 missense variant mRNA molecule is set forth in SEQ ID NO:40.

Another ANGPTL7 missense variant mRNA molecule exists, wherein the guanine at position 948 is replaced with a uracil. The nucleotide sequence of this ANGPTL7 missense variant mRNA molecule is set forth in SEQ ID NO:41.

Another ANGPTL7 missense variant mRNA molecule exists, wherein the guanine at position 999 is replaced with a uracil. The nucleotide sequence of this ANGPTL7 missense variant mRNA molecule is set forth in SEQ ID NO:42.

Another ANGPTL7 missense variant mRNA molecule exists, wherein the cytosine at position 720 is replaced with a uracil. The nucleotide sequence of this ANGPTL7 missense variant mRNA molecule is set forth in SEQ ID NO:43.

Another ANGPTL7 missense variant mRNA molecule exists, wherein the cytosine at position 760 is replaced with a uracil. The nucleotide sequence of this ANGPTL7 missense variant mRNA molecule is set forth in SEQ ID NO:44.

Another ANGPTL7 missense variant mRNA molecule exists, wherein the cytosine at position 764 is replaced with a uracil. The nucleotide sequence of this ANGPTL7 missense variant mRNA molecule is set forth in SEQ ID NO:45.

Another ANGPTL7 missense variant mRNA molecule exists, wherein the cytosine at position 768 is replaced with a uracil. The nucleotide sequence of this ANGPTL7 missense variant mRNA molecule is set forth in SEQ ID NO:46.

Another ANGPTL7 missense variant mRNA molecule exists, wherein the cytosine at position 802 is replaced with a uracil. The nucleotide sequence of this ANGPTL7 missense variant mRNA molecule is set forth in SEQ ID NO:47.

Another ANGPTL7 missense variant mRNA molecule exists, wherein the cytosine at position 813 is replaced with a uracil. The nucleotide sequence of this ANGPTL7 missense variant mRNA molecule is set forth in SEQ ID NO:48.

Another ANGPTL7 missense variant mRNA molecule exists, wherein the cytosine at position 930 is replaced with a uracil. The nucleotide sequence of this ANGPTL7 missense variant mRNA molecule is set forth in SEQ ID NO:49.

Another ANGPTL7 missense variant mRNA molecule exists, wherein the cytosine at position 981 is replaced with a uracil. The nucleotide sequence of this ANGPTL7 missense variant mRNA molecule is set forth in SEQ ID NO:50.

Another ANGPTL7 missense variant mRNA molecule exists, wherein the guanine at position 695 is replaced with an adenine. The nucleotide sequence of this ANGPTL7 missense variant mRNA molecule is set forth in SEQ ID NO:51.

Another ANGPTL7 missense variant mRNA molecule exists, wherein the guanine at position 735 is replaced with an adenine. The nucleotide sequence of this ANGPTL7 missense variant mRNA molecule is set forth in SEQ ID NO:52.

Another ANGPTL7 missense variant mRNA molecule exists, wherein the guanine at position 739 is replaced with an adenine. The nucleotide sequence of this ANGPTL7 missense variant mRNA molecule is set forth in SEQ ID NO:53.

Another ANGPTL7 missense variant mRNA molecule exists, wherein the guanine at position 743 is replaced with an adenine. The nucleotide sequence of this ANGPTL7 missense variant mRNA molecule is set forth in SEQ ID NO:54.

Another ANGPTL7 missense variant mRNA molecule exists, wherein the guanine at position 777 is replaced with an adenine. The nucleotide sequence of this ANGPTL7 missense variant mRNA molecule is set forth in SEQ ID NO:55.

Another ANGPTL7 missense variant mRNA molecule exists, wherein the guanine at position 788 is replaced with an adenine. The nucleotide sequence of this ANGPTL7 missense variant mRNA molecule is set forth in SEQ ID NO:56.

Another ANGPTL7 missense variant mRNA molecule exists, wherein the guanine at position 905 is replaced with an adenine. The nucleotide sequence of this ANGPTL7 missense variant mRNA molecule is set forth in SEQ ID NO:57.

Another ANGPTL7 missense variant mRNA molecule exists, wherein the guanine at position 956 is replaced with an adenine. The nucleotide sequence of this ANGPTL7 missense variant mRNA molecule is set forth in SEQ ID NO:58.

Another ANGPTL7 missense variant mRNA molecule exists, wherein the adenine at position 488 is replaced with a cytosine. The nucleotide sequence of this ANGPTL7 missense variant mRNA molecule is set forth in SEQ ID NO:59.

Another ANGPTL7 missense variant mRNA molecule exists, wherein the adenine at position 538 is replaced with a cytosine. The nucleotide sequence of this ANGPTL7 missense variant mRNA molecule is set forth in SEQ ID NO:60.

Another ANGPTL7 missense variant mRNA molecule exists, wherein the adenine at position 532 is replaced with a cytosine. The nucleotide sequence of this ANGPTL7 missense variant mRNA molecule is set forth in SEQ ID NO:61.

Another ANGPTL7 missense variant mRNA molecule exists, wherein the adenine at position 536 is replaced with a cytosine. The nucleotide sequence of this ANGPTL7 missense variant mRNA molecule is set forth in SEQ ID NO:62.

Another ANGPTL7 missense variant mRNA molecule exists, wherein the adenine at position 570 is replaced with a cytosine. The nucleotide sequence of this ANGPTL7 missense variant mRNA molecule is set forth in SEQ ID NO:63.

Another ANGPTL7 missense variant mRNA molecule exists, wherein the adenine at position 581 is replaced with a cytosine. The nucleotide sequence of this ANGPTL7 missense variant mRNA molecule is set forth in SEQ ID NO:64.

Another ANGPTL7 missense variant mRNA molecule exists, wherein the adenine at position 698 is replaced with a cytosine. The nucleotide sequence of this ANGPTL7 missense variant mRNA molecule is set forth in SEQ ID NO:65.

Another ANGPTL7 missense variant mRNA molecule exists, wherein the adenine at position 749 is replaced with a cytosine. The nucleotide sequence of this ANGPTL7 missense variant mRNA molecule is set forth in SEQ ID NO:66.

Another ANGPTL7 missense variant mRNA molecule exists, wherein the cytosine at position 481 is replaced with a uracil. The nucleotide sequence of this ANGPTL7 missense variant mRNA molecule is set forth in SEQ ID NO:67.

Another ANGPTL7 missense variant mRNA molecule exists, wherein the cytosine at position 521 is replaced with a uracil. The nucleotide sequence of this ANGPTL7 missense variant mRNA molecule is set forth in SEQ ID NO:68.

Another ANGPTL7 missense variant mRNA molecule exists, wherein the cytosine at position 525 is replaced with a uracil. The nucleotide sequence of this ANGPTL7 missense variant mRNA molecule is set forth in SEQ ID NO:69.

Another ANGPTL7 missense variant mRNA molecule exists, wherein the cytosine at position 529 is replaced with a uracil. The nucleotide sequence of this ANGPTL7 missense variant mRNA molecule is set forth in SEQ ID NO:70.

Another ANGPTL7 missense variant mRNA molecule exists, wherein the cytosine at position 563 is replaced with a uracil. The nucleotide sequence of this ANGPTL7 missense variant mRNA molecule is set forth in SEQ ID NO:71.

Another ANGPTL7 missense variant mRNA molecule exists, wherein the cytosine at position 574 is replaced with a uracil. The nucleotide sequence of this ANGPTL7 missense variant mRNA molecule is set forth in SEQ ID NO:72.

Another ANGPTL7 missense variant mRNA molecule exists, wherein the cytosine at position 691 is replaced with a uracil. The nucleotide sequence of this ANGPTL7 missense variant mRNA molecule is set forth in SEQ ID NO:73.

Another ANGPTL7 missense variant mRNA molecule exists, wherein the cytosine at position 742 is replaced with a uracil. The nucleotide sequence of this ANGPTL7 missense variant mRNA molecule is set forth in SEQ ID NO:74.

Another ANGPTL7 missense variant mRNA molecule exists, wherein the cytosine at position 720 is replaced with a uracil. The nucleotide sequence of this ANGPTL7 missense variant mRNA molecule is set forth in SEQ ID NO:75.

Another ANGPTL7 missense variant mRNA molecule exists, wherein the cytosine at position 760 is replaced with a uracil. The nucleotide sequence of this ANGPTL7 missense variant mRNA molecule is set forth in SEQ ID NO:76.

Another ANGPTL7 missense variant mRNA molecule exists, wherein the cytosine at position 764 is replaced with a uracil. The nucleotide sequence of this ANGPTL7 missense variant mRNA molecule is set forth in SEQ ID NO:77.

Another ANGPTL7 missense variant mRNA molecule exists, wherein the cytosine at position 768 is replaced with a uracil. The nucleotide sequence of this ANGPTL7 missense variant mRNA molecule is set forth in SEQ ID NO:78.

Another ANGPTL7 missense variant mRNA molecule exists, wherein the cytosine at position 802 is replaced with a uracil. The nucleotide sequence of this ANGPTL7 missense variant mRNA molecule is set forth in SEQ ID NO:79.

Another ANGPTL7 missense variant mRNA molecule exists, wherein the cytosine at position 813 is replaced with a uracil. The nucleotide sequence of this ANGPTL7 missense variant mRNA molecule is set forth in SEQ ID NO:80.

Another ANGPTL7 missense variant mRNA molecule exists, wherein the cytosine at position 930 is replaced with a uracil. The nucleotide sequence of this ANGPTL7 missense variant mRNA molecule is set forth in SEQ ID NO:81.

Another ANGPTL7 missense variant mRNA molecule exists, wherein the cytosine at position 981 is replaced with a uracil. The nucleotide sequence of this ANGPTL7 missense variant mRNA molecule is set forth in SEQ ID NO:82.

Another ANGPTL7 missense variant mRNA molecule exists, wherein the uracil at position 1,023 is replaced with an adenine. The nucleotide sequence of this ANGPTL7 missense variant mRNA molecule is set forth in SEQ ID NO:83.

Another ANGPTL7 missense variant mRNA molecule exists, wherein the uracil at position 1,090 is replaced with an adenine. The nucleotide sequence of this ANGPTL7 missense variant mRNA molecule is set forth in SEQ ID NO:84.

Another ANGPTL7 missense variant mRNA molecule exists, wherein the uracil at position 1,055 is replaced with an adenine. The nucleotide sequence of this ANGPTL7 missense variant mRNA molecule is set forth in SEQ ID NO:85.

Another ANGPTL7 missense variant mRNA molecule exists, wherein the uracil at position 1,037 is replaced with an adenine. The nucleotide sequence of this ANGPTL7 missense variant mRNA molecule is set forth in SEQ ID NO:86.

Another ANGPTL7 missense variant mRNA molecule exists, wherein the uracil at position 1,012 is replaced with an adenine. The nucleotide sequence of this ANGPTL7 missense variant mRNA molecule is set forth in SEQ ID NO:87.

Another ANGPTL7 missense variant mRNA molecule exists, wherein the uracil at position 805 is replaced with an adenine. The nucleotide sequence of this ANGPTL7 missense variant mRNA molecule is set forth in SEQ ID NO:88.

Another ANGPTL7 missense variant mRNA molecule exists, wherein the uracil at position 798 is replaced with an adenine. The nucleotide sequence of this ANGPTL7 missense variant mRNA molecule is set forth in SEQ ID NO:89.

Another ANGPTL7 missense variant mRNA molecule exists, wherein the uracil at position 1,037 is replaced with an adenine. The nucleotide sequence of this ANGPTL7 missense variant mRNA molecule is set forth in SEQ ID NO:90.

The nucleotide sequence of an ANGPTL7 reference cDNA molecule is set forth in SEQ ID NO:91. Referring to SEQ ID NO:91, position 706 is a thymine. Referring to SEQ ID NO:91, position 746 is a thymine. Referring to SEQ ID NO:91, position 750 is a guanine. Referring to SEQ ID NO:91, position 754 is a cytosine. Referring to SEQ ID NO:91, position 788 is a guanine. Referring to SEQ ID NO:91, position 799 is an adenine. Referring to SEQ ID NO:91, position 916 is a cytosine. Referring to SEQ ID NO:91, position 967 is a cytosine. Referring to SEQ ID NO:91, position 1,023 is a thymine.

The nucleotide sequence of another ANGPTL7 reference cDNA molecule is set forth in SEQ ID NO:92. Referring to SEQ ID NO:92, position 773 is a thymine. Referring to SEQ ID NO:92, position 812 is a thymine. Referring to SEQ ID NO:92, position 817 is a guanine. Referring to SEQ ID NO:92, position 821 is a cytosine. Referring to SEQ ID NO:92, position 855 is a guanine. Referring to SEQ ID NO:92, position 866 is an adenine. Referring to SEQ ID NO:92, position 983 is a cytosine. Referring to SEQ ID NO:92, position 1,034 is a cytosine. Referring to SEQ ID NO:92, position 1,090 is a thymine.

The nucleotide sequence of another ANGPTL7 reference cDNA molecule is set forth in SEQ ID NO:93. Referring to SEQ ID NO:93, position 738 is a thymine. Referring to SEQ ID NO:93, position 778 is a thymine. Referring to SEQ ID NO:93, position 782 is a guanine. Referring to SEQ ID NO:93, position 786 is a cytosine. Referring to SEQ ID NO:93, position 820 is a guanine. Referring to SEQ ID NO:93, position 831 is an adenine. Referring to SEQ ID NO:93, position 948 is a cytosine. Referring to SEQ ID NO:93, position 999 is a cytosine. Referring to SEQ ID NO:93, position 1,055 is a thymine.

The nucleotide sequence of another ANGPTL7 reference cDNA molecule is set forth in SEQ ID NO:94. Referring to SEQ ID NO:94, position 720 is a thymine. Referring to SEQ ID NO:94, position 760 is a thymine. Referring to SEQ ID NO:94, position 764 is a guanine. Referring to SEQ ID NO:94, position 768 is a cytosine. Referring to SEQ ID NO:94, position 802 is a guanine. Referring to SEQ ID NO:94, position 813 is an adenine. Referring to SEQ ID NO:94, position 948 is a cytosine. Referring to SEQ ID NO:94, position 981 is a cytosine. Referring to SEQ ID NO:94, position 1,037 is a thymine.

The nucleotide sequence of another ANGPTL7 reference cDNA molecule is set forth in SEQ ID NO:95. Referring to SEQ ID NO:95, position 695 is a thymine. Referring to SEQ ID NO:95, position 735 is a thymine. Referring to SEQ ID NO:95, position 739 is a guanine. Referring to SEQ ID NO:95, position 743 is a cytosine. Referring to SEQ ID NO:95, position 777 is a guanine. Referring to SEQ ID NO:95, position 788 is an adenine. Referring to SEQ ID NO:95, position 905 is a cytosine. Referring to SEQ ID NO:95, position 956 is a cytosine. Referring to SEQ ID NO:95, position 1,012 is a thymine.

The nucleotide sequence of another ANGPTL7 reference cDNA molecule is set forth in SEQ ID NO:96. Referring to SEQ ID NO:96, position 488 is a thymine. Referring to SEQ ID NO:96, position 528 is a thymine. Referring to SEQ ID NO:96, position 532 is a guanine. Referring to SEQ ID NO:96, position 536 is a cytosine. Referring to SEQ ID NO:96, position 570 is a guanine. Referring to SEQ ID NO:96, position 581 is an adenine. Referring to SEQ ID NO:96, position 698 is a cytosine. Referring to SEQ ID NO:96, position 749 is a cytosine. Referring to SEQ ID NO:96, position 805 is a thymine.

The nucleotide sequence of another ANGPTL7 reference cDNA molecule is set forth in SEQ ID NO:97. Referring to SEQ ID NO:97, position 481 is a thymine. Referring to SEQ ID NO:97, position 521 is a thymine. Referring to SEQ ID NO:97, position 525 is a guanine. Referring to SEQ ID NO:97, position 529 is a cytosine. Referring to SEQ ID NO:97, position 563 is a guanine. Referring to SEQ ID NO:97, position 574 is an adenine. Referring to SEQ ID NO:97, position 691 is a cytosine. Referring to SEQ ID NO:97, position 742 is a cytosine. Referring to SEQ ID NO:97, position 798 is a thymine.

The nucleotide sequence of another ANGPTL7 reference cDNA molecule is set forth in SEQ ID NO:98. Referring to SEQ ID NO:98, position 720 is a thymine. Referring to SEQ ID NO:98, position 760 is a thymine. Referring to SEQ ID NO:98, position 764 is a guanine. Referring to SEQ ID NO:98, position 768 is a cytosine. Referring to SEQ ID NO:98, position 802 is a guanine. Referring to SEQ ID NO:98, position 813 is an adenine. Referring to SEQ ID NO:98, position 930 is a cytosine. Referring to SEQ ID NO:98, position 981 is a cytosine. Referring to SEQ ID NO:98, position 1,037 is a thymine.

An ANGPTL7 missense variant cDNA molecule exists, wherein the thymine at position 706 is replaced with an adenine. The nucleotide sequence of this ANGPTL7 missense variant cDNA molecule is set forth in SEQ ID NO:99.

Another ANGPTL7 missense variant cDNA molecule exists, wherein the thymine at position 746 is replaced with an adenine. The nucleotide sequence of this ANGPTL7 missense variant cDNA molecule is set forth in SEQ ID NO:100.

Another ANGPTL7 missense variant cDNA molecule exists, wherein the thymine at position 750 is replaced with an adenine. The nucleotide sequence of this ANGPTL7 missense variant cDNA molecule is set forth in SEQ ID NO:101.

Another ANGPTL7 missense variant cDNA molecule exists, wherein the thymine at position 754 is replaced with an adenine. The nucleotide sequence of this ANGPTL7 missense variant cDNA molecule is set forth in SEQ ID NO:102.

Another ANGPTL7 missense variant cDNA molecule exists, wherein the thymine at position 788 is replaced with an adenine. The nucleotide sequence of this ANGPTL7 missense variant cDNA molecule is set forth in SEQ ID NO:103.

Another ANGPTL7 missense variant cDNA molecule exists, wherein the thymine at position 799 is replaced with an adenine. The nucleotide sequence of this ANGPTL7 missense variant cDNA molecule is set forth in SEQ ID NO:104.

Another ANGPTL7 missense variant cDNA molecule exists, wherein the thymine at position 916 is replaced with an adenine. The nucleotide sequence of this ANGPTL7 missense variant cDNA molecule is set forth in SEQ ID NO:105.

Another ANGPTL7 missense variant cDNA molecule exists, wherein the thymine at position 967 is replaced with an adenine. The nucleotide sequence of this ANGPTL7 missense variant cDNA molecule is set forth in SEQ ID NO:106.

Another ANGPTL7 missense variant cDNA molecule exists, wherein the thymine at position 773 is replaced with an adenine. The nucleotide sequence of this ANGPTL7 missense variant cDNA molecule is set forth in SEQ ID NO:107.

Another ANGPTL7 missense variant cDNA molecule exists, wherein the thymine at position 812 is replaced with an adenine. The nucleotide sequence of this ANGPTL7 missense variant cDNA molecule is set forth in SEQ ID NO:108.

Another ANGPTL7 missense variant cDNA molecule exists, wherein the thymine at position 817 is replaced with an adenine. The nucleotide sequence of this ANGPTL7 missense variant cDNA molecule is set forth in SEQ ID NO:109.

Another ANGPTL7 missense variant cDNA molecule exists, wherein the thymine at position 821 is replaced with an adenine. The nucleotide sequence of this ANGPTL7 missense variant cDNA molecule is set forth in SEQ ID NO:110.

Another ANGPTL7 missense variant cDNA molecule exists, wherein the thymine at position 855 is replaced with an adenine. The nucleotide sequence of this ANGPTL7 missense variant cDNA molecule is set forth in SEQ ID NO:111.

Another ANGPTL7 missense variant cDNA molecule exists, wherein the thymine at position 866 is replaced with an adenine. The nucleotide sequence of this ANGPTL7 missense variant cDNA molecule is set forth in SEQ ID NO:112.

Another ANGPTL7 missense variant cDNA molecule exists, wherein the thymine at position 983 is replaced with an adenine. The nucleotide sequence of this ANGPTL7 missense variant cDNA molecule is set forth in SEQ ID NO:113.

Another ANGPTL7 missense variant cDNA molecule exists, wherein the thymine at position 1,034 is replaced with an adenine. The nucleotide sequence of this ANGPTL7 missense variant cDNA molecule is set forth in SEQ ID NO:114.

Another ANGPTL7 missense variant cDNA molecule exists, wherein the guanine at position 738 is replaced with a thymine. The nucleotide sequence of this ANGPTL7 missense variant cDNA molecule is set forth in SEQ ID NO:115.

Another ANGPTL7 missense variant cDNA molecule exists, wherein the guanine at position 778 is replaced with a thymine. The nucleotide sequence of this ANGPTL7 missense variant cDNA molecule is set forth in SEQ ID NO:116.

Another ANGPTL7 missense variant cDNA molecule exists, wherein the guanine at position 782 is replaced with a thymine. The nucleotide sequence of this ANGPTL7 missense variant cDNA molecule is set forth in SEQ ID NO:117.

Another ANGPTL7 missense variant cDNA molecule exists, wherein the guanine at position 786 is replaced with a thymine. The nucleotide sequence of this ANGPTL7 missense variant cDNA molecule is set forth in SEQ ID NO:118.

Another ANGPTL7 missense variant cDNA molecule exists, wherein the guanine at position 820 is replaced with a thymine. The nucleotide sequence of this ANGPTL7 missense variant cDNA molecule is set forth in SEQ ID NO:119.

Another ANGPTL7 missense variant cDNA molecule exists, wherein the guanine at position 831 is replaced with a thymine. The nucleotide sequence of this ANGPTL7 missense variant cDNA molecule is set forth in SEQ ID NO:120.

Another ANGPTL7 missense variant cDNA molecule exists, wherein the guanine at position 948 is replaced with a thymine. The nucleotide sequence of this ANGPTL7 missense variant cDNA molecule is set forth in SEQ ID NO:121.

Another ANGPTL7 missense variant cDNA molecule exists, wherein the guanine at position 999 is replaced with a thymine. The nucleotide sequence of this ANGPTL7 missense variant cDNA molecule is set forth in SEQ ID NO:122.

Another ANGPTL7 missense variant cDNA molecule exists, wherein the cytosine at position 720 is replaced with a thymine. The nucleotide sequence of this ANGPTL7 missense variant cDNA molecule is set forth in SEQ ID NO:123.

Another ANGPTL7 missense variant cDNA molecule exists, wherein the cytosine at position 760 is replaced with a thymine. The nucleotide sequence of this ANGPTL7 missense variant cDNA molecule is set forth in SEQ ID NO:124.

Another ANGPTL7 missense variant cDNA molecule exists, wherein the cytosine at position 764 is replaced with a thymine. The nucleotide sequence of this ANGPTL7 missense variant cDNA molecule is set forth in SEQ ID NO:125.

Another ANGPTL7 missense variant cDNA molecule exists, wherein the cytosine at position 768 is replaced with a thymine. The nucleotide sequence of this ANGPTL7 missense variant cDNA molecule is set forth in SEQ ID NO:126.

Another ANGPTL7 missense variant cDNA molecule exists, wherein the cytosine at position 802 is replaced with a thymine. The nucleotide sequence of this ANGPTL7 missense variant cDNA molecule is set forth in SEQ ID NO:127.

Another ANGPTL7 missense variant cDNA molecule exists, wherein the cytosine at position 813 is replaced with a thymine. The nucleotide sequence of this ANGPTL7 missense variant cDNA molecule is set forth in SEQ ID NO:128.

Another ANGPTL7 missense variant cDNA molecule exists, wherein the cytosine at position 930 is replaced with a thymine. The nucleotide sequence of this ANGPTL7 missense variant cDNA molecule is set forth in SEQ ID NO:129.

Another ANGPTL7 missense variant cDNA molecule exists, wherein the cytosine at position 981 is replaced with a thymine. The nucleotide sequence of this ANGPTL7 missense variant cDNA molecule is set forth in SEQ ID NO:130.

Another ANGPTL7 missense variant cDNA molecule exists, wherein the guanine at position 695 is replaced with an adenine. The nucleotide sequence of this ANGPTL7 missense variant cDNA molecule is set forth in SEQ ID NO:131.

Another ANGPTL7 missense variant cDNA molecule exists, wherein the guanine at position 735 is replaced with an adenine. The nucleotide sequence of this ANGPTL7 missense variant cDNA molecule is set forth in SEQ ID NO:132.

Another ANGPTL7 missense variant cDNA molecule exists, wherein the guanine at position 739 is replaced with an adenine. The nucleotide sequence of this ANGPTL7 missense variant cDNA molecule is set forth in SEQ ID NO:133.

Another ANGPTL7 missense variant cDNA molecule exists, wherein the guanine at position 743 is replaced with an adenine. The nucleotide sequence of this ANGPTL7 missense variant cDNA molecule is set forth in SEQ ID NO:134.

Another ANGPTL7 missense variant cDNA molecule exists, wherein the guanine at position 777 is replaced with an adenine. The nucleotide sequence of this ANGPTL7 missense variant cDNA molecule is set forth in SEQ ID NO:135.

Another ANGPTL7 missense variant cDNA molecule exists, wherein the guanine at position 788 is replaced with an adenine. The nucleotide sequence of this ANGPTL7 missense variant cDNA molecule is set forth in SEQ ID NO:136.

Another ANGPTL7 missense variant cDNA molecule exists, wherein the guanine at position 905 is replaced with an adenine. The nucleotide sequence of this ANGPTL7 missense variant cDNA molecule is set forth in SEQ ID NO:137.

Another ANGPTL7 missense variant cDNA molecule exists, wherein the guanine at position 956 is replaced with an adenine. The nucleotide sequence of this ANGPTL7 missense variant cDNA molecule is set forth in SEQ ID NO:138.

Another ANGPTL7 missense variant cDNA molecule exists, wherein the adenine at position 488 is replaced with a cytosine. The nucleotide sequence of this ANGPTL7 missense variant cDNA molecule is set forth in SEQ ID NO:139.

Another ANGPTL7 missense variant cDNA molecule exists, wherein the adenine at position 528 is replaced with a cytosine. The nucleotide sequence of this ANGPTL7 missense variant cDNA molecule is set forth in SEQ ID NO:140.

Another ANGPTL7 missense variant cDNA molecule exists, wherein the adenine at position 532 is replaced with a cytosine. The nucleotide sequence of this ANGPTL7 missense variant cDNA molecule is set forth in SEQ ID NO:141.

Another ANGPTL7 missense variant cDNA molecule exists, wherein the adenine at position 536 is replaced with a cytosine. The nucleotide sequence of this ANGPTL7 missense variant cDNA molecule is set forth in SEQ ID NO:142.

Another ANGPTL7 missense variant cDNA molecule exists, wherein the adenine at position 570 is replaced with a cytosine. The nucleotide sequence of this ANGPTL7 missense variant cDNA molecule is set forth in SEQ ID NO:143.

Another ANGPTL7 missense variant cDNA molecule exists, wherein the adenine at position 581 is replaced with a cytosine. The nucleotide sequence of this ANGPTL7 missense variant cDNA molecule is set forth in SEQ ID NO:144.

Another ANGPTL7 missense variant cDNA molecule exists, wherein the adenine at position 698 is replaced with a cytosine. The nucleotide sequence of this ANGPTL7 missense variant cDNA molecule is set forth in SEQ ID NO:145.

Another ANGPTL7 missense variant cDNA molecule exists, wherein the adenine at position 749 is replaced with a cytosine. The nucleotide sequence of this ANGPTL7 missense variant cDNA molecule is set forth in SEQ ID NO:146.

Another ANGPTL7 missense variant cDNA molecule exists, wherein the cytosine at position 481 is replaced with a thymine. The nucleotide sequence of this ANGPTL7 missense variant cDNA molecule is set forth in SEQ ID NO:147.

Another ANGPTL7 missense variant cDNA molecule exists, wherein the cytosine at position 521 is replaced with a thymine. The nucleotide sequence of this ANGPTL7 missense variant cDNA molecule is set forth in SEQ ID NO:148.

Another ANGPTL7 missense variant cDNA molecule exists, wherein the cytosine at position 525 is replaced with a thymine. The nucleotide sequence of this ANGPTL7 missense variant cDNA molecule is set forth in SEQ ID NO:149.

Another ANGPTL7 missense variant cDNA molecule exists, wherein the cytosine at position 529 is replaced with a thymine. The nucleotide sequence of this ANGPTL7 missense variant cDNA molecule is set forth in SEQ ID NO:150.

Another ANGPTL7 missense variant cDNA molecule exists, wherein the cytosine at position 563 is replaced with a thymine. The nucleotide sequence of this ANGPTL7 missense variant cDNA molecule is set forth in SEQ ID NO:151.

Another ANGPTL7 missense variant cDNA molecule exists, wherein the cytosine at position 574 is replaced with a thymine. The nucleotide sequence of this ANGPTL7 missense variant cDNA molecule is set forth in SEQ ID NO:152.

Another ANGPTL7 missense variant cDNA molecule exists, wherein the cytosine at position 691 is replaced with a thymine. The nucleotide sequence of this ANGPTL7 missense variant cDNA molecule is set forth in SEQ ID NO:153.

Another ANGPTL7 missense variant cDNA molecule exists, wherein the cytosine at position 742 is replaced with a thymine. The nucleotide sequence of this ANGPTL7 missense variant cDNA molecule is set forth in SEQ ID NO:154.

Another ANGPTL7 missense variant cDNA molecule exists, wherein the cytosine at position 720 is replaced with a thymine. The nucleotide sequence of this ANGPTL7 missense variant cDNA molecule is set forth in SEQ ID NO:155.

Another ANGPTL7 missense variant cDNA molecule exists, wherein the cytosine at position 760 is replaced with a thymine. The nucleotide sequence of this ANGPTL7 missense variant cDNA molecule is set forth in SEQ ID NO:156.

Another ANGPTL7 missense variant cDNA molecule exists, wherein the cytosine at position 764 is replaced with a thymine. The nucleotide sequence of this ANGPTL7 missense variant cDNA molecule is set forth in SEQ ID NO:157.

Another ANGPTL7 missense variant cDNA molecule exists, wherein the cytosine at position 768 is replaced with a thymine. The nucleotide sequence of this ANGPTL7 missense variant cDNA molecule is set forth in SEQ ID NO:158.

Another ANGPTL7 missense variant cDNA molecule exists, wherein the cytosine at position 802 is replaced with a thymine. The nucleotide sequence of this ANGPTL7 missense variant cDNA molecule is set forth in SEQ ID NO:159.

Another ANGPTL7 missense variant cDNA molecule exists, wherein the cytosine at position 813 is replaced with a thymine. The nucleotide sequence of this ANGPTL7 missense variant cDNA molecule is set forth in SEQ ID NO:160.

Another ANGPTL7 missense variant cDNA molecule exists, wherein the cytosine at position 930 is replaced with a thymine. The nucleotide sequence of this ANGPTL7 missense variant cDNA molecule is set forth in SEQ ID NO:161.

Another ANGPTL7 missense variant cDNA molecule exists, wherein the cytosine at position 981 is replaced with a thymine. The nucleotide sequence of this ANGPTL7 missense variant cDNA molecule is set forth in SEQ ID NO:162.

Another ANGPTL7 missense variant cDNA molecule exists, wherein the thymine at position 1,023 is replaced with an adenine. The nucleotide sequence of this ANGPTL7 missense variant cDNA molecule is set forth in SEQ ID NO:163.

Another ANGPTL7 missense variant cDNA molecule exists, wherein the thymine at position 1,090 is replaced with an adenine. The nucleotide sequence of this ANGPTL7 missense variant cDNA molecule is set forth in SEQ ID NO:164.

Another ANGPTL7 missense variant cDNA molecule exists, wherein the thymine at position 1,055 is replaced with an adenine. The nucleotide sequence of this ANGPTL7 missense variant cDNA molecule is set forth in SEQ ID NO:165.

Another ANGPTL7 missense variant cDNA molecule exists, wherein the thymine at position 1,037 is replaced with an adenine. The nucleotide sequence of this ANGPTL7 missense variant cDNA molecule is set forth in SEQ ID NO:166.

Another ANGPTL7 missense variant cDNA molecule exists, wherein the thymine at position 1,012 is replaced with an adenine. The nucleotide sequence of this ANGPTL7 missense variant cDNA molecule is set forth in SEQ ID NO:167.

Another ANGPTL7 missense variant cDNA molecule exists, wherein the thymine at position 805 is replaced with an adenine. The nucleotide sequence of this ANGPTL7 missense variant cDNA molecule is set forth in SEQ ID NO:168.

Another ANGPTL7 missense variant cDNA molecule exists, wherein the thymine at position 798 is replaced with an adenine. The nucleotide sequence of this ANGPTL7 missense variant cDNA molecule is set forth in SEQ ID NO:169.

Another ANGPTL7 missense variant cDNA molecule exists, wherein the thymine at position 1,037 is replaced with an adenine. The nucleotide sequence of this ANGPTL7 missense variant cDNA molecule is set forth in SEQ ID NO:170.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The isolated nucleic acid molecules, or the complement thereof, can also be present within a host cell. In some embodiments, the host cell can comprise the vector that comprises any of the nucleic acid molecules described herein, or the complement thereof. In some embodiments, the nucleic acid molecule is operably linked to a promoter active in the host cell. In some embodiments, the promoter is an exogenous promoter. In some embodiments, the promoter is an inducible promoter. In some embodiments, the host cell is a bacterial cell, a yeast cell, an insect cell, or a mammalian cell. In some embodiments, the host cell is a bacterial cell. In some embodiments, the host cell is a yeast cell. In some embodiments, the host cell is an insect cell. In some embodiments, the host cell is a mammalian cell.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (1), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:11, or SEQ ID NO:91). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, an ANGPTL7 missense nucleic acid molecule comprising a nucleotide sequence encoding an ANGPTL7 predicted loss-of-function polypeptide, wherein the nucleotide sequence comprises an adenine at a position corresponding to position 4,229 according to SEQ ID NO:2 means that if the nucleotide sequence of the ANGPTL7 genomic nucleic acid molecule is aligned to the sequence of SEQ ID NO:2, the ANGPTL7 sequence has an adenine residue at the position that corresponds to position 4,229 of SEQ ID NO:2. The same applies for an ANGPTL7 missense mRNA molecules comprising a nucleotide sequence encoding an ANGPTL7 predicted loss-of-function polypeptide, wherein the nucleotide sequence comprises an adenine at a position corresponding to position 706 according to SEQ ID NO:19, and an ANGPTL7 missense cDNA molecules comprising a nucleotide sequence encoding an ANGPTL7 predicted loss-of-function polypeptide, wherein the nucleotide sequence comprises an adenine at a position corresponding to position 706 according to SEQ ID NO:99. In other words, these phrases refer to a nucleic acid molecule encoding an ANGPTL7 polypeptide, wherein the genomic nucleic acid molecule has a nucleotide sequence that comprises an adenine residue that is homologous to the adenine residue at position 4,229 of SEQ ID NO:2 (or wherein the mRNA molecule has a nucleotide sequence that comprises an adenine residue that is homologous to the adenine residue at position 706 of SEQ ID NO:19, or wherein the cDNA molecule has a nucleotide sequence that comprises an adenine residue that is homologous to the adenine residue at position 706 of SEQ ID NO:99).

As described herein, a position within an ANGPTL7 missense genomic nucleic acid molecule that corresponds to position 4,229 according to SEQ ID NO:2, for example, can be identified by performing a sequence alignment between the nucleotide sequence of a particular ANGPTL7 nucleic acid molecule and the nucleotide sequence of SEQ ID NO:2. A variety of computational algorithms exist that can be used for performing a sequence alignment to identify a nucleotide position that corresponds to, for example, position 4,229 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequence of an ANGPTL7 reference polypeptide is set forth in SEQ ID NO:171 (Isoform 1). Referring to SEQ ID NO:171 (Isoform 1), the ANGPTL7 reference polypeptide is 346 amino acids in length. Referring to SEQ ID NO:171, position 161 is a phenylalanine. Referring to SEQ ID NO:171, position 174 is an isoleucine. Referring to SEQ ID NO:171, position 175 is a glutamine. Referring to SEQ ID NO:171, position 177 is an arginine. Referring to SEQ ID NO:171, position 188 is a tryptophan. Referring to SEQ ID NO:171, position 192 is a lysine. Referring to SEQ ID NO:171, position 231 is an arginine. Referring to SEQ ID NO:171, position 248 is an arginine. Referring to SEQ ID NO:171, position 266 is a histidine.

The amino acid sequences of ANGPTL7 predicted loss-of-function polypeptides are set forth in SEQ ID NO:172, SEQ ID NO:173, SEQ ID NO:174, in SEQ ID NO:175, SEQ ID NO:176, SEQ ID NO:177, SEQ ID NO:178, SEQ ID NO:179, and SEQ ID NO:180.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or inhibit an ophthalmic condition for use in the treatment of an ophthalmic condition (or for use in the preparation of a medicament for treating an ophthalmic condition) in a subject, wherein the subject has any of the ANGPTL7 missense variant genomic nucleic acid molecules, missense variant mRNA molecules, and/or missense variant cDNA molecules encoding an ANGPTL7 predicted loss-of-function polypeptide described herein. The therapeutic agents that treat or inhibit an ophthalmic condition can be any of the therapeutic agents that treat or inhibit an ophthalmic condition described herein.

In some embodiments, the subject is identified as having a genomic nucleic acid molecule encoding an ANGPTL7 predicted loss-of-function polypeptide, wherein the genomic nucleic acid molecule has a nucleotide sequence comprising: an adenine at a position corresponding to position 4,229 according to SEQ ID NO:2, or the complement thereof; an adenine at a position corresponding to position 4,269 according to SEQ ID NO:3, or the complement thereof; a thymine at a position corresponding to position 4,273 according to SEQ ID NO:4, or the complement thereof; a thymine at a position corresponding to position 4,277 according to SEQ ID NO:5, or the complement thereof; an adenine at a position corresponding to position 4,311 according to SEQ ID NO:6, or the complement thereof; a cytosine at a position corresponding to position 4,322 according to SEQ ID NO:7, or the complement thereof; a thymine at a position corresponding to position 5,125 according to SEQ ID NO:8, or the complement thereof; a thymine at a position corresponding to position 5,176 according to SEQ ID NO:9, or the complement thereof; or an adenine at a position corresponding to position 5,232 according to SEQ ID NO:10, or the complement thereof.

In some embodiments, the subject is identified as having an mRNA molecule encoding an ANGPTL7 predicted loss-of-function polypeptide, wherein the mRNA molecule has a nucleotide sequence comprising: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof; a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a uracil at a position corresponding to position 768 according to SEQ ID NO:50, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof; a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; a uracil at a position corresponding to position 981 according to SEQ ID NO:82, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof.

In some embodiments, the subject is identified as having a cDNA molecule encoding an ANGPTL7 predicted loss-of-function polypeptide, wherein the cDNA molecule has a nucleotide sequence comprising: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; a thymine at a position corresponding to position 764 according to SEQ ID NO:122, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; a thymine at a position corresponding to position 768 according to SEQ ID NO:130, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; an adenine at a position corresponding to position 802 according to SEQ ID NO:138, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; a cytosine at a position corresponding to position 813 according to SEQ ID NO:146, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; a thymine at a position corresponding to position 930 according to SEQ ID NO:154, or the complement thereof; a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; a thymine at a position corresponding to position 981 according to SEQ ID NO:162, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof.

In some embodiments, the subject is identified as having: i) a genomic nucleic acid molecule having a nucleotide sequence encoding an ANGPTL7 predicted loss-of-function polypeptide, wherein the nucleotide sequence comprises an adenine at a position corresponding to position 4,229 according to SEQ ID NO:2, or the complement thereof; ii) an mRNA molecule having a nucleotide sequence encoding an ANGPTL7 predicted loss-of-function polypeptide, wherein the nucleotide sequence comprises: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; or iii) a cDNA molecule having a nucleotide sequence encoding an ANGPTL7 predicted loss-of-function polypeptide, wherein the nucleotide sequence comprises: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof.

In some embodiments, the subject is identified as having a genomic nucleic acid molecule having a nucleotide sequence encoding an ANGPTL7 predicted loss-of-function polypeptide, wherein the nucleotide sequence comprises an adenine at a position corresponding to position 4,229 according to SEQ ID NO:2, or the complement thereof.

In some embodiments, the subject is identified as having an mRNA molecule having a nucleotide sequence encoding an ANGPTL7 predicted loss-of-function polypeptide, wherein the nucleotide sequence comprises: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; or an adenine at a position corresponding to position 720 according to SEQ ID NO:26, or the complement thereof.

In some embodiments, the subject is identified as having a cDNA molecule having a nucleotide sequence encoding an ANGPTL7 predicted loss-of-function polypeptide, wherein the nucleotide sequence comprises: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; or an adenine at a position corresponding to position 720 according to SEQ ID NO:106, or the complement thereof.

In some embodiments, the subject is identified as having: i) a genomic nucleic acid molecule having a nucleotide sequence encoding an ANGPTL7 predicted loss-of-function polypeptide, wherein the nucleotide sequence comprises an adenine at a position corresponding to position 4,269 according to SEQ ID NO:3, or the complement thereof; ii) an mRNA molecule having a nucleotide sequence encoding an ANGPTL7 predicted loss-of-function polypeptide, wherein the nucleotide sequence comprises: an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; or an adenine at a position corresponding to position 760 according to SEQ ID NO:34, or the complement thereof; or iii) a cDNA molecule having a nucleotide sequence encoding an ANGPTL7 predicted loss-of-function polypeptide, wherein the nucleotide sequence comprises: an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; or an adenine at a position corresponding to position 760 according to SEQ ID NO:114, or the complement thereof.

In some embodiments, the subject is identified as having a genomic nucleic acid molecule having a nucleotide sequence encoding an ANGPTL7 predicted loss-of-function polypeptide, wherein the nucleotide sequence comprises an adenine at a position corresponding to position 4,269 according to SEQ ID NO:3, or the complement thereof.

In some embodiments, the subject is identified as having an mRNA molecule having a nucleotide sequence encoding an ANGPTL7 predicted loss-of-function polypeptide, wherein the nucleotide sequence comprises: an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; or an adenine at a position corresponding to position 760 according to SEQ ID NO:34, or the complement thereof.

In some embodiments, the subject is identified as having a cDNA molecule having a nucleotide sequence encoding an ANGPTL7 predicted loss-of-function polypeptide, wherein the nucleotide sequence comprises: an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; or an adenine at a position corresponding to position 760 according to SEQ ID NO:114, or the complement thereof.

In some embodiments, the subject is identified as having: i) a genomic nucleic acid molecule having a nucleotide sequence encoding an ANGPTL7 predicted loss-of-function polypeptide, wherein the nucleotide sequence comprises a thymine at a position corresponding to position 4,273 according to SEQ ID NO:4, or the complement thereof; ii) an mRNA molecule having a nucleotide sequence encoding an ANGPTL7 predicted loss-of-function polypeptide, wherein the nucleotide sequence comprises: a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; or a uracil at a position corresponding to position 764 according to SEQ ID NO:42, or the complement thereof; or iii) a cDNA molecule having a nucleotide sequence encoding an ANGPTL7 predicted loss-of-function polypeptide, wherein the nucleotide sequence comprises: a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; or a thymine at a position corresponding to position 764 according to SEQ ID NO:122, or the complement thereof.

In some embodiments, the subject is identified as having a genomic nucleic acid molecule having a nucleotide sequence encoding an ANGPTL7 predicted loss-of-function polypeptide, wherein the nucleotide sequence comprises a thymine at a position corresponding to position 4,273 according to SEQ ID NO:4, or the complement thereof.

In some embodiments, the subject is identified as having an mRNA molecule having a nucleotide sequence encoding an ANGPTL7 predicted loss-of-function polypeptide, wherein the nucleotide sequence comprises: a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; or a uracil at a position corresponding to position 764 according to SEQ ID NO:42, or the complement thereof.

In some embodiments, the subject is identified as having a cDNA molecule having a nucleotide sequence encoding an ANGPTL7 predicted loss-of-function polypeptide, wherein the nucleotide sequence comprises: a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; or a thymine at a position corresponding to position 764 according to SEQ ID NO:122, or the complement thereof.

In some embodiments, the subject is identified as having: i) a genomic nucleic acid molecule having a nucleotide sequence encoding an ANGPTL7 predicted loss-of-function polypeptide, wherein the nucleotide sequence comprises a thymine at a position corresponding to position 4,277 according to SEQ ID NO:5, or the complement thereof; ii) an mRNA molecule having a nucleotide sequence encoding an ANGPTL7 predicted loss-of-function polypeptide, wherein the nucleotide sequence comprises: a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a or uracil at a position corresponding to position 768 according to SEQ ID NO:50, or the complement thereof; or iii) a cDNA molecule having a nucleotide sequence encoding an ANGPTL7 predicted loss-of-function polypeptide, wherein the nucleotide sequence comprises: a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; or a thymine at a position corresponding to position 768 according to SEQ ID NO:130, or the complement thereof.

In some embodiments, the subject is identified as having a genomic nucleic acid molecule having a nucleotide sequence encoding an ANGPTL7 predicted loss-of-function polypeptide, wherein the nucleotide sequence comprises a thymine at a position corresponding to position 4,277 according to SEQ ID NO:5, or the complement thereof.

In some embodiments, the subject is identified as having an mRNA molecule having a nucleotide sequence encoding an ANGPTL7 predicted loss-of-function polypeptide, wherein the nucleotide sequence comprises: a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; or a uracil at a position corresponding to position 768 according to SEQ ID NO:50, or the complement thereof.

In some embodiments, the subject is identified as having a cDNA molecule having a nucleotide sequence encoding an ANGPTL7 predicted loss-of-function polypeptide, wherein the nucleotide sequence comprises: a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; or a thymine at a position corresponding to position 768 according to SEQ ID NO:130, or the complement thereof.

In some embodiments, the subject is identified as having: i) a genomic nucleic acid molecule having a nucleotide sequence encoding an ANGPTL7 predicted loss-of-function polypeptide, wherein the nucleotide sequence comprises an adenine at a position corresponding to position 4,311 according to SEQ ID NO:6, or the complement thereof; ii) an mRNA molecule having a nucleotide sequence encoding an ANGPTL7 predicted loss-of-function polypeptide, wherein the nucleotide sequence comprises: an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; or an adenine at a position corresponding to position 802 according to SEQ ID NO:58, or the complement thereof; or iii) a cDNA molecule having a nucleotide sequence encoding an ANGPTL7 predicted loss-of-function polypeptide, wherein the nucleotide sequence comprises: an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; or an adenine at a position corresponding to position 802 according to SEQ ID NO:138, or the complement thereof.

In some embodiments, the subject is identified as having a genomic nucleic acid molecule having a nucleotide sequence encoding an ANGPTL7 predicted loss-of-function polypeptide, wherein the nucleotide sequence comprises an adenine at a position corresponding to position 4,311 according to SEQ ID NO:6, or the complement thereof.

In some embodiments, the subject is identified as having an mRNA molecule having a nucleotide sequence encoding an ANGPTL7 predicted loss-of-function polypeptide, wherein the nucleotide sequence comprises: an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; or an adenine at a position corresponding to position 802 according to SEQ ID NO:58, or the complement thereof.

In some embodiments, the subject is identified as having a cDNA molecule having a nucleotide sequence encoding an ANGPTL7 predicted loss-of-function polypeptide, wherein the nucleotide sequence comprises: an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; or an adenine at a position corresponding to position 802 according to SEQ ID NO:138, or the complement thereof.

In some embodiments, the subject is identified as having: i) a genomic nucleic acid molecule having a nucleotide sequence encoding an ANGPTL7 predicted loss-of-function polypeptide, wherein the nucleotide sequence comprises a cytosine at a position corresponding to position 4,322 according to SEQ ID NO:7, or the complement thereof; ii) an mRNA molecule having a nucleotide sequence encoding an ANGPTL7 predicted loss-of-function polypeptide, wherein the nucleotide sequence comprises: a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; or a cytosine at a position corresponding to position 813 according to SEQ ID NO:66, or the complement thereof; or iii) a cDNA molecule having a nucleotide sequence encoding an ANGPTL7 predicted loss-of-function polypeptide, wherein the nucleotide sequence comprises: a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; or a cytosine at a position corresponding to position 813 according to SEQ ID NO:146, or the complement thereof.

In some embodiments, the subject is identified as having a genomic nucleic acid molecule having a nucleotide sequence encoding an ANGPTL7 predicted loss-of-function polypeptide, wherein the nucleotide sequence comprises a cytosine at a position corresponding to position 4,322 according to SEQ ID NO:7, or the complement thereof.

In some embodiments, the subject is identified as having an mRNA molecule having a nucleotide sequence encoding an ANGPTL7 predicted loss-of-function polypeptide, wherein the nucleotide sequence comprises: a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; or a cytosine at a position corresponding to position 813 according to SEQ ID NO:66, or the complement thereof.

In some embodiments, the subject is identified as having a cDNA molecule having a nucleotide sequence encoding an ANGPTL7 predicted loss-of-function polypeptide, wherein the nucleotide sequence comprises: a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; or a cytosine at a position corresponding to position 813 according to SEQ ID NO:146, or the complement thereof.

In some embodiments, the subject is identified as having: i) a genomic nucleic acid molecule having a nucleotide sequence encoding an ANGPTL7 predicted loss-of-function polypeptide, wherein the nucleotide sequence comprises a thymine at a position corresponding to position 5,125 according to SEQ ID NO:8, or the complement thereof; ii) an mRNA molecule having a nucleotide sequence encoding an ANGPTL7 predicted loss-of-function polypeptide, wherein the nucleotide sequence comprises: a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; or a uracil at a position corresponding to position 930 according to SEQ ID NO:74, or the complement thereof; or iii) a cDNA molecule having a nucleotide sequence encoding an ANGPTL7 predicted loss-of-function polypeptide, wherein the nucleotide sequence comprises: a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; or a thymine at a position corresponding to position 930 according to SEQ ID NO:154, or the complement thereof.

In some embodiments, the subject is identified as having a genomic nucleic acid molecule having a nucleotide sequence encoding an ANGPTL7 predicted loss-of-function polypeptide, wherein the nucleotide sequence comprises a thymine at a position corresponding to position 5,125 according to SEQ ID NO:8, or the complement thereof.

In some embodiments, the subject is identified as having an mRNA molecule having a nucleotide sequence encoding an ANGPTL7 predicted loss-of-function polypeptide, wherein the nucleotide sequence comprises: a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; or a uracil at a position corresponding to position 930 according to SEQ ID NO:74, or the complement thereof.

In some embodiments, the subject is identified as having a cDNA molecule having a nucleotide sequence encoding an ANGPTL7 predicted loss-of-function polypeptide, wherein the nucleotide sequence comprises: a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; or a thymine at a position corresponding to position 930 according to SEQ ID NO:154, or the complement thereof.

In some embodiments, the subject is identified as having: i) a genomic nucleic acid molecule having a nucleotide sequence encoding an ANGPTL7 predicted loss-of-function polypeptide, wherein the nucleotide sequence comprises a thymine at a position corresponding to position 5,176 according to SEQ ID NO:9, or the complement thereof; ii) an mRNA molecule having a nucleotide sequence encoding an ANGPTL7 predicted loss-of-function polypeptide, wherein the nucleotide sequence comprises: a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; or a uracil at a position corresponding to position 981 according to SEQ ID NO:82, or the complement thereof; or iii) a cDNA molecule having a nucleotide sequence encoding an ANGPTL7 predicted loss-of-function polypeptide, wherein the nucleotide sequence comprises: a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; or a thymine at a position corresponding to position 981 according to SEQ ID NO:162, or the complement thereof.

In some embodiments, the subject is identified as having a genomic nucleic acid molecule having a nucleotide sequence encoding an ANGPTL7 predicted loss-of-function polypeptide, wherein the nucleotide sequence comprises a thymine at a position corresponding to position 5,176 according to SEQ ID NO:9, or the complement thereof.

In some embodiments, the subject is identified as having an mRNA molecule having a nucleotide sequence encoding an ANGPTL7 predicted loss-of-function polypeptide, wherein the nucleotide sequence comprises: a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; or a uracil at a position corresponding to position 981 according to SEQ ID NO:82, or the complement thereof.

In some embodiments, the subject is identified as having a cDNA molecule having a nucleotide sequence encoding an ANGPTL7 predicted loss-of-function polypeptide, wherein the nucleotide sequence comprises: a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; or a thymine at a position corresponding to position 981 according to SEQ ID NO:162, or the complement thereof.

In some embodiments, the subject is identified as having: i) a genomic nucleic acid molecule having a nucleotide sequence encoding an ANGPTL7 predicted loss-of-function polypeptide, wherein the nucleotide sequence comprises an adenine at a position corresponding to position 5,232 according to SEQ ID NO:10, or the complement thereof; ii) an mRNA molecule having a nucleotide sequence encoding an ANGPTL7 predicted loss-of-function polypeptide, wherein the nucleotide sequence comprises: an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof; or iii) a cDNA molecule having a nucleotide sequence encoding an ANGPTL7 predicted loss-of-function polypeptide, wherein the nucleotide sequence comprises: an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof.

In some embodiments, the subject is identified as having a genomic nucleic acid molecule having a nucleotide sequence encoding an ANGPTL7 predicted loss-of-function polypeptide, wherein the nucleotide sequence comprises an adenine at a position corresponding to position 5,232 according to SEQ ID NO:10, or the complement thereof.

In some embodiments, the subject is identified as having an mRNA molecule having a nucleotide sequence encoding an ANGPTL7 predicted loss-of-function polypeptide, wherein the nucleotide sequence comprises: an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof.

In some embodiments, the subject is identified as having a cDNA molecule having a nucleotide sequence encoding an ANGPTL7 predicted loss-of-function polypeptide, wherein the nucleotide sequence comprises: an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof.

In some embodiments, the subject is identified as having an ANGPTL7 predicted loss-of-function polypeptide that comprises an isoleucine at a position corresponding to position 161 according to SEQ ID NO:172. In some embodiments, the subject is identified as having an ANGPTL7 predicted loss-of-function polypeptide that comprises an asparagine at a position corresponding to position 174 according to SEQ ID NO:173. In some embodiments, the subject is identified as having an ANGPTL7 predicted loss-of-function polypeptide that comprises a histidine at a position corresponding to position 175 according to SEQ ID NO:174. In some embodiments, the subject is identified as having an ANGPTL7 predicted loss-of-function polypeptide that comprises a truncation at a position corresponding to position 176 according to SEQ ID NO:175. In some embodiments, the subject is identified as having an ANGPTL7 predicted loss-of-function polypeptide that comprises a truncation at a position corresponding to position 187 according to SEQ ID NO:176. In some embodiments, the subject is identified as having an ANGPTL7 predicted loss-of-function polypeptide that comprises a glutamine at a position corresponding to position 192 according to SEQ ID NO:177. In some embodiments, the subject is identified as having an ANGPTL7 predicted loss-of-function polypeptide that comprises a cysteine at a position corresponding to position 231 according to SEQ ID NO:178. In some embodiments, the subject is identified as having an ANGPTL7 predicted loss-of-function polypeptide that comprises a cysteine at a position corresponding to position 248 according to SEQ ID NO:179. In some embodiments, the subject is identified as having an ANGPTL7 predicted loss-of-function polypeptide that comprises a glutamine at a position corresponding to position 266 according to SEQ ID NO:180.

The present disclosure also provides ANGPTL7 inhibitors for use in the treatment of an ophthalmic condition (or for use in the preparation of a medicament for treating an ophthalmic condition) in a subject, wherein the subject is heterozygous for any of the ANGPTL7 missense variant genomic nucleic acid molecules, missense variant mRNA molecules, and/or missense variant cDNA molecules encoding an ANGPTL7 predicted loss-of-function polypeptide described herein, or wherein the subject is reference for an ANGPTL7 genomic nucleic acid molecule, mRNA molecule, or cDNA molecule. The ANGPTL7 inhibitors can be any of the ANGPTL7 inhibitors described herein.

In some embodiments, the subject is reference for an ANGPTL7 genomic nucleic acid molecule, an ANGPTL7 mRNA molecule, or an ANGPTL7 cDNA molecule. In some embodiments, the subject is reference for an ANGPTL7 genomic nucleic acid molecule. In some embodiments, the subject is reference for an ANGPTL7 mRNA molecule. In some embodiments, the subject is reference for an ANGPTL7 cDNA molecule.

In some embodiments, the subject is identified as being heterozygous for a genomic nucleic acid molecule encoding an ANGPTL7 predicted loss-of-function polypeptide, wherein the genomic nucleic acid molecule has a nucleotide sequence comprising: an adenine at a position corresponding to position 4,229 according to SEQ ID NO:2, or the complement thereof; an adenine at a position corresponding to position 4,269 according to SEQ ID NO:3, or the complement thereof; a thymine at a position corresponding to position 4,273 according to SEQ ID NO:4, or the complement thereof; a thymine at a position corresponding to position 4,277 according to SEQ ID NO:5, or the complement thereof; an adenine at a position corresponding to position 4,311 according to SEQ ID NO:6, or the complement thereof; a cytosine at a position corresponding to position 4,322 according to SEQ ID NO:7, or the complement thereof; a thymine at a position corresponding to position 5,125 according to SEQ ID NO:8, or the complement thereof; a thymine at a position corresponding to position 5,176 according to SEQ ID NO:9, or the complement thereof; or an adenine at a position corresponding to position 5,232 according to SEQ ID NO:10, or the complement thereof.

In some embodiments, the subject is identified as being heterozygous for an mRNA molecule encoding an ANGPTL7 predicted loss-of-function polypeptide, wherein the mRNA molecule has a nucleotide sequence comprising: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; an adenine at a position corresponding to position 720 according to SEQ ID NO:26, or the complement thereof; an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; an adenine at a position corresponding to position 760 according to SEQ ID NO:34, or the complement thereof; a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; a uracil at a position corresponding to position 764 according to SEQ ID NO:42, or the complement thereof; a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; a uracil at a position corresponding to position 768 according to SEQ ID NO:50, or the complement thereof; an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; an adenine at a position corresponding to position 802 according to SEQ ID NO:58, or the complement thereof; a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; a cytosine at a position corresponding to position 813 according to SEQ ID NO:66, or the complement thereof; a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; a uracil at a position corresponding to position 930 according to SEQ ID NO:74, or the complement thereof; a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; a uracil at a position corresponding to position 981 according to SEQ ID NO:82, or the complement thereof; an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof.

In some embodiments, the subject is identified as being heterozygous for a cDNA molecule encoding an ANGPTL7 predicted loss-of-function polypeptide, wherein the cDNA molecule has a nucleotide sequence comprising: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; an adenine at a position corresponding to position 720 according to SEQ ID NO:106, or the complement thereof; an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; an adenine at a position corresponding to position 760 according to SEQ ID NO:114, or the complement thereof; a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; a thymine at a position corresponding to position 764 according to SEQ ID NO:122, or the complement thereof; a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; a thymine at a position corresponding to position 768 according to SEQ ID NO:130, or the complement thereof; an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; an adenine at a position corresponding to position 802 according to SEQ ID NO:138, or the complement thereof; a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; a cytosine at a position corresponding to position 813 according to SEQ ID NO:146, or the complement thereof; a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; a thymine at a position corresponding to position 930 according to SEQ ID NO:154, or the complement thereof; a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; a thymine at a position corresponding to position 981 according to SEQ ID NO:162, or the complement thereof; an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof.

In some embodiments, the subject is identified as being heterozygous for: i) a genomic nucleic acid molecule having a nucleotide sequence encoding an ANGPTL7 predicted loss-of-function polypeptide, wherein the nucleotide sequence comprises an adenine at a position corresponding to position 4,229 according to SEQ ID NO:2, or the complement thereof; ii) an mRNA molecule having a nucleotide sequence encoding an ANGPTL7 predicted loss-of-function polypeptide, wherein the nucleotide sequence comprises: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; or an adenine at a position corresponding to position 720 according to SEQ ID NO:26, or the complement thereof; or iii) a cDNA molecule having a nucleotide sequence encoding an ANGPTL7 predicted loss-of-function polypeptide, wherein the nucleotide sequence comprises: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; or an adenine at a position corresponding to position 720 according to SEQ ID NO:106, or the complement thereof.

In some embodiments, the subject is identified as being heterozygous for a genomic nucleic acid molecule having a nucleotide sequence encoding an ANGPTL7 predicted loss-of-function polypeptide, wherein the nucleotide sequence comprises an adenine at a position corresponding to position 4,229 according to SEQ ID NO:2, or the complement thereof.

In some embodiments, the subject is identified as being heterozygous for an mRNA molecule having a nucleotide sequence encoding an ANGPTL7 predicted loss-of-function polypeptide, wherein the nucleotide sequence comprises: an adenine at a position corresponding to position 706 according to SEQ ID NO:19, or the complement thereof; an adenine at a position corresponding to position 773 according to SEQ ID NO:20, or the complement thereof; an adenine at a position corresponding to position 738 according to SEQ ID NO:21, or the complement thereof; an adenine at a position corresponding to position 720 according to SEQ ID NO:22, or the complement thereof; an adenine at a position corresponding to position 695 according to SEQ ID NO:23, or the complement thereof; an adenine at a position corresponding to position 488 according to SEQ ID NO:24, or the complement thereof; an adenine at a position corresponding to position 481 according to SEQ ID NO:25, or the complement thereof; or an adenine at a position corresponding to position 720 according to SEQ ID NO:26, or the complement thereof.

In some embodiments, the subject is identified as being heterozygous for a cDNA molecule having a nucleotide sequence encoding an ANGPTL7 predicted loss-of-function polypeptide, wherein the nucleotide sequence comprises: an adenine at a position corresponding to position 706 according to SEQ ID NO:99, or the complement thereof; an adenine at a position corresponding to position 773 according to SEQ ID NO:100, or the complement thereof; an adenine at a position corresponding to position 738 according to SEQ ID NO:101, or the complement thereof; an adenine at a position corresponding to position 720 according to SEQ ID NO:102, or the complement thereof; an adenine at a position corresponding to position 695 according to SEQ ID NO:103, or the complement thereof; an adenine at a position corresponding to position 488 according to SEQ ID NO:104, or the complement thereof; an adenine at a position corresponding to position 481 according to SEQ ID NO:105, or the complement thereof; or an adenine at a position corresponding to position 720 according to SEQ ID NO:106, or the complement thereof.

In some embodiments, the subject is identified as being heterozygous for: i) a genomic nucleic acid molecule having a nucleotide sequence encoding an ANGPTL7 predicted loss-of-function polypeptide, wherein the nucleotide sequence comprises an adenine at a position corresponding to position 4,269 according to SEQ ID NO:3, or the complement thereof; ii) an mRNA molecule having a nucleotide sequence encoding an ANGPTL7 predicted loss-of-function polypeptide, wherein the nucleotide sequence comprises: an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; or an adenine at a position corresponding to position 760 according to SEQ ID NO:34, or the complement thereof; or iii) a cDNA molecule having a nucleotide sequence encoding an ANGPTL7 predicted loss-of-function polypeptide, wherein the nucleotide sequence comprises: an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; or an adenine at a position corresponding to position 760 according to SEQ ID NO:114, or the complement thereof.

In some embodiments, the subject is identified as being heterozygous for a genomic nucleic acid molecule having a nucleotide sequence encoding an ANGPTL7 predicted loss-of-function polypeptide, wherein the nucleotide sequence comprises an adenine at a position corresponding to position 4,269 according to SEQ ID NO:3, or the complement thereof.

In some embodiments, the subject is identified as being heterozygous for an mRNA molecule having a nucleotide sequence encoding an ANGPTL7 predicted loss-of-function polypeptide, wherein the nucleotide sequence comprises: an adenine at a position corresponding to position 746 according to SEQ ID NO:27, or the complement thereof; an adenine at a position corresponding to position 812 according to SEQ ID NO:28, or the complement thereof; an adenine at a position corresponding to position 778 according to SEQ ID NO:29, or the complement thereof; an adenine at a position corresponding to position 760 according to SEQ ID NO:30, or the complement thereof; an adenine at a position corresponding to position 735 according to SEQ ID NO:31, or the complement thereof; an adenine at a position corresponding to position 528 according to SEQ ID NO:32, or the complement thereof; an adenine at a position corresponding to position 521 according to SEQ ID NO:33, or the complement thereof; or an adenine at a position corresponding to position 760 according to SEQ ID NO:34, or the complement thereof.

In some embodiments, the subject is identified as being heterozygous for a cDNA molecule having a nucleotide sequence encoding an ANGPTL7 predicted loss-of-function polypeptide, wherein the nucleotide sequence comprises: an adenine at a position corresponding to position 746 according to SEQ ID NO:107, or the complement thereof; an adenine at a position corresponding to position 812 according to SEQ ID NO:108, or the complement thereof; an adenine at a position corresponding to position 778 according to SEQ ID NO:109, or the complement thereof; an adenine at a position corresponding to position 760 according to SEQ ID NO:110, or the complement thereof; an adenine at a position corresponding to position 735 according to SEQ ID NO:111, or the complement thereof; an adenine at a position corresponding to position 528 according to SEQ ID NO:112, or the complement thereof; an adenine at a position corresponding to position 529 according to SEQ ID NO:113, or the complement thereof; or an adenine at a position corresponding to position 760 according to SEQ ID NO:114, or the complement thereof.

In some embodiments, the subject is identified as being heterozygous for: i) a genomic nucleic acid molecule having a nucleotide sequence encoding an ANGPTL7 predicted loss-of-function polypeptide, wherein the nucleotide sequence comprises a thymine at a position corresponding to position 4,273 according to SEQ ID NO:4, or the complement thereof; ii) an mRNA molecule having a nucleotide sequence encoding an ANGPTL7 predicted loss-of-function polypeptide, wherein the nucleotide sequence comprises: a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; or a uracil at a position corresponding to position 764 according to SEQ ID NO:42, or the complement thereof; or iii) a cDNA molecule having a nucleotide sequence encoding an ANGPTL7 predicted loss-of-function polypeptide, wherein the nucleotide sequence comprises: a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; or a thymine at a position corresponding to position 764 according to SEQ ID NO:122, or the complement thereof.

In some embodiments, the subject is identified as being heterozygous for a genomic nucleic acid molecule having a nucleotide sequence encoding an ANGPTL7 predicted loss-of-function polypeptide, wherein the nucleotide sequence comprises a thymine at a position corresponding to position 4,273 according to SEQ ID NO:4, or the complement thereof.

In some embodiments, the subject is identified as being heterozygous for an mRNA molecule having a nucleotide sequence encoding an ANGPTL7 predicted loss-of-function polypeptide, wherein the nucleotide sequence comprises: a uracil at a position corresponding to position 750 according to SEQ ID NO:35, or the complement thereof; a uracil at a position corresponding to position 817 according to SEQ ID NO:36, or the complement thereof; a uracil at a position corresponding to position 782 according to SEQ ID NO:37, or the complement thereof; a uracil at a position corresponding to position 764 according to SEQ ID NO:38, or the complement thereof; a uracil at a position corresponding to position 739 according to SEQ ID NO:39, or the complement thereof; a uracil at a position corresponding to position 532 according to SEQ ID NO:40, or the complement thereof; a uracil at a position corresponding to position 525 according to SEQ ID NO:41, or the complement thereof; or a uracil at a position corresponding to position 764 according to SEQ ID NO:42, or the complement thereof.

In some embodiments, the subject is identified as being heterozygous for a cDNA molecule having a nucleotide sequence encoding an ANGPTL7 predicted loss-of-function polypeptide, wherein the nucleotide sequence comprises: a thymine at a position corresponding to position 750 according to SEQ ID NO:115, or the complement thereof; a thymine at a position corresponding to position 817 according to SEQ ID NO:116, or the complement thereof; a thymine at a position corresponding to position 782 according to SEQ ID NO:117, or the complement thereof; a thymine at a position corresponding to position 764 according to SEQ ID NO:118, or the complement thereof; a thymine at a position corresponding to position 739 according to SEQ ID NO:119, or the complement thereof; a thymine at a position corresponding to position 532 according to SEQ ID NO:120, or the complement thereof; a thymine at a position corresponding to position 525 according to SEQ ID NO:121, or the complement thereof; or a thymine at a position corresponding to position 764 according to SEQ ID NO:122, or the complement thereof.

In some embodiments, the subject is identified as being heterozygous for: i) a genomic nucleic acid molecule having a nucleotide sequence encoding an ANGPTL7 predicted loss-of-function polypeptide, wherein the nucleotide sequence comprises a thymine at a position corresponding to position 4,277 according to SEQ ID NO:5, or the complement thereof; ii) an mRNA molecule having a nucleotide sequence encoding an ANGPTL7 predicted loss-of-function polypeptide, wherein the nucleotide sequence comprises: a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; a uracil at a position corresponding to position 529 according to SEQ ID NO:49, or the complement thereof; or a uracil at a position corresponding to position 768 according to SEQ ID NO:50, or the complement thereof; or iii) a cDNA molecule having a nucleotide sequence encoding an ANGPTL7 predicted loss-of-function polypeptide, wherein the nucleotide sequence comprises: a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; or a thymine at a position corresponding to position 768 according to SEQ ID NO:130, or the complement thereof.

In some embodiments, the subject is identified as being heterozygous for a genomic nucleic acid molecule having a nucleotide sequence encoding an ANGPTL7 predicted loss-of-function polypeptide, wherein the nucleotide sequence comprises a thymine at a position corresponding to position 4,277 according to SEQ ID NO:5, or the complement thereof.

In some embodiments, the subject is identified as being heterozygous for an mRNA molecule having a nucleotide sequence encoding an ANGPTL7 predicted loss-of-function polypeptide, wherein the nucleotide sequence comprises: a uracil at a position corresponding to position 754 according to SEQ ID NO:43, or the complement thereof; a uracil at a position corresponding to position 821 according to SEQ ID NO:44, or the complement thereof; a uracil at a position corresponding to position 786 according to SEQ ID NO:45, or the complement thereof; a uracil at a position corresponding to position 768 according to SEQ ID NO:46, or the complement thereof; a uracil at a position corresponding to position 743 according to SEQ ID NO:47, or the complement thereof; a uracil at a position corresponding to position 536 according to SEQ ID NO:48, or the complement thereof; or a uracil at a position corresponding to position 529 according to SEQ ID NO:49, or the complement thereof.

In some embodiments, the subject is identified as being heterozygous for a cDNA molecule having a nucleotide sequence encoding an ANGPTL7 predicted loss-of-function polypeptide, wherein the nucleotide sequence comprises: a thymine at a position corresponding to position 754 according to SEQ ID NO:123, or the complement thereof; a thymine at a position corresponding to position 821 according to SEQ ID NO:124, or the complement thereof; a thymine at a position corresponding to position 786 according to SEQ ID NO:125, or the complement thereof; a thymine at a position corresponding to position 768 according to SEQ ID NO:126, or the complement thereof; a thymine at a position corresponding to position 743 according to SEQ ID NO:127, or the complement thereof; a thymine at a position corresponding to position 536 according to SEQ ID NO:128, or the complement thereof; a thymine at a position corresponding to position 529 according to SEQ ID NO:129, or the complement thereof; or a thymine at a position corresponding to position 768 according to SEQ ID NO:130, or the complement thereof.

In some embodiments, the subject is identified as being heterozygous for: i) a genomic nucleic acid molecule having a nucleotide sequence encoding an ANGPTL7 predicted loss-of-function polypeptide, wherein the nucleotide sequence comprises an adenine at a position corresponding to position 4,311 according to SEQ ID NO:6, or the complement thereof; ii) an mRNA molecule having a nucleotide sequence encoding an ANGPTL7 predicted loss-of-function polypeptide, wherein the nucleotide sequence comprises: an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; or an adenine at a position corresponding to position 802 according to SEQ ID NO:58, or the complement thereof; or iii) a cDNA molecule having a nucleotide sequence encoding an ANGPTL7 predicted loss-of-function polypeptide, wherein the nucleotide sequence comprises: an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; or an adenine at a position corresponding to position 802 according to SEQ ID NO:138, or the complement thereof.

In some embodiments, the subject is identified as being heterozygous for a genomic nucleic acid molecule having a nucleotide sequence encoding an ANGPTL7 predicted loss-of-function polypeptide, wherein the nucleotide sequence comprises an adenine at a position corresponding to position 4,311 according to SEQ ID NO:6, or the complement thereof.

In some embodiments, the subject is identified as being heterozygous for an mRNA molecule having a nucleotide sequence encoding an ANGPTL7 predicted loss-of-function polypeptide, wherein the nucleotide sequence comprises: an adenine at a position corresponding to position 788 according to SEQ ID NO:51, or the complement thereof; an adenine at a position corresponding to position 855 according to SEQ ID NO:52, or the complement thereof; an adenine at a position corresponding to position 820 according to SEQ ID NO:53, or the complement thereof; an adenine at a position corresponding to position 802 according to SEQ ID NO:54, or the complement thereof; an adenine at a position corresponding to position 777 according to SEQ ID NO:55, or the complement thereof; an adenine at a position corresponding to position 570 according to SEQ ID NO:56, or the complement thereof; an adenine at a position corresponding to position 563 according to SEQ ID NO:57, or the complement thereof; or an adenine at a position corresponding to position 802 according to SEQ ID NO:58, or the complement thereof.

In some embodiments, the subject is identified as being heterozygous for a cDNA molecule having a nucleotide sequence encoding an ANGPTL7 predicted loss-of-function polypeptide, wherein the nucleotide sequence comprises: an adenine at a position corresponding to position 788 according to SEQ ID NO:131, or the complement thereof; an adenine at a position corresponding to position 855 according to SEQ ID NO:132, or the complement thereof; an adenine at a position corresponding to position 820 according to SEQ ID NO:133, or the complement thereof; an adenine at a position corresponding to position 802 according to SEQ ID NO:134, or the complement thereof; an adenine at a position corresponding to position 777 according to SEQ ID NO:135, or the complement thereof; an adenine at a position corresponding to position 570 according to SEQ ID NO:136, or the complement thereof; an adenine at a position corresponding to position 563 according to SEQ ID NO:137, or the complement thereof; or an adenine at a position corresponding to position 802 according to SEQ ID NO:138, or the complement thereof.

In some embodiments, the subject is identified as being heterozygous for: i) a genomic nucleic acid molecule having a nucleotide sequence encoding an ANGPTL7 predicted loss-of-function polypeptide, wherein the nucleotide sequence comprises a cytosine at a position corresponding to position 4,322 according to SEQ ID NO:7, or the complement thereof; ii) an mRNA molecule having a nucleotide sequence encoding an ANGPTL7 predicted loss-of-function polypeptide, wherein the nucleotide sequence comprises: a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; or a cytosine at a position corresponding to position 813 according to SEQ ID NO:66, or the complement thereof; or iii) a cDNA molecule having a nucleotide sequence encoding an ANGPTL7 predicted loss-of-function polypeptide, wherein the nucleotide sequence comprises: a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; or a cytosine at a position corresponding to position 813 according to SEQ ID NO:146, or the complement thereof.

In some embodiments, the subject is identified as being heterozygous for a genomic nucleic acid molecule having a nucleotide sequence encoding an ANGPTL7 predicted loss-of-function polypeptide, wherein the nucleotide sequence comprises a cytosine at a position corresponding to position 4,322 according to SEQ ID NO:7, or the complement thereof.

In some embodiments, the subject is identified as being heterozygous for an mRNA molecule having a nucleotide sequence encoding an ANGPTL7 predicted loss-of-function polypeptide, wherein the nucleotide sequence comprises: a cytosine at a position corresponding to position 799 according to SEQ ID NO:59, or the complement thereof; a cytosine at a position corresponding to position 866 according to SEQ ID NO:60, or the complement thereof; a cytosine at a position corresponding to position 831 according to SEQ ID NO:61, or the complement thereof; a cytosine at a position corresponding to position 813 according to SEQ ID NO:62, or the complement thereof; a cytosine at a position corresponding to position 788 according to SEQ ID NO:63, or the complement thereof; a cytosine at a position corresponding to position 581 according to SEQ ID NO:64, or the complement thereof; a cytosine at a position corresponding to position 574 according to SEQ ID NO:65, or the complement thereof; or a cytosine at a position corresponding to position 813 according to SEQ ID NO:66, or the complement thereof.

In some embodiments, the subject is identified as being heterozygous for a cDNA molecule having a nucleotide sequence encoding an ANGPTL7 predicted loss-of-function polypeptide, wherein the nucleotide sequence comprises: a cytosine at a position corresponding to position 799 according to SEQ ID NO:139, or the complement thereof; a cytosine at a position corresponding to position 866 according to SEQ ID NO:140, or the complement thereof; a cytosine at a position corresponding to position 831 according to SEQ ID NO:141, or the complement thereof; a cytosine at a position corresponding to position 813 according to SEQ ID NO:142, or the complement thereof; a cytosine at a position corresponding to position 788 according to SEQ ID NO:143, or the complement thereof; a cytosine at a position corresponding to position 581 according to SEQ ID NO:144, or the complement thereof; a cytosine at a position corresponding to position 574 according to SEQ ID NO:145, or the complement thereof; or a cytosine at a position corresponding to position 813 according to SEQ ID NO:146, or the complement thereof.

In some embodiments, the subject is identified as being heterozygous for: i) a genomic nucleic acid molecule having a nucleotide sequence encoding an ANGPTL7 predicted loss-of-function polypeptide, wherein the nucleotide sequence comprises a thymine at a position corresponding to position 5,125 according to SEQ ID NO:8, or the complement thereof; ii) an mRNA molecule having a nucleotide sequence encoding an ANGPTL7 predicted loss-of-function polypeptide, wherein the nucleotide sequence comprises: a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; or a uracil at a position corresponding to position 930 according to SEQ ID NO:74, or the complement thereof; or iii) a cDNA molecule having a nucleotide sequence encoding an ANGPTL7 predicted loss-of-function polypeptide, wherein the nucleotide sequence comprises: a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; or a thymine at a position corresponding to position 930 according to SEQ ID NO:154, or the complement thereof.

In some embodiments, the subject is identified as being heterozygous for a genomic nucleic acid molecule having a nucleotide sequence encoding an ANGPTL7 predicted loss-of-function polypeptide, wherein the nucleotide sequence comprises a thymine at a position corresponding to position 5,125 according to SEQ ID NO:8, or the complement thereof.

In some embodiments, the subject is identified as being heterozygous for an mRNA molecule having a nucleotide sequence encoding an ANGPTL7 predicted loss-of-function polypeptide, wherein the nucleotide sequence comprises: a uracil at a position corresponding to position 916 according to SEQ ID NO:67, or the complement thereof; a uracil at a position corresponding to position 983 according to SEQ ID NO:68, or the complement thereof; a uracil at a position corresponding to position 948 according to SEQ ID NO:69, or the complement thereof; a uracil at a position corresponding to position 930 according to SEQ ID NO:70, or the complement thereof; a uracil at a position corresponding to position 905 according to SEQ ID NO:71, or the complement thereof; a uracil at a position corresponding to position 698 according to SEQ ID NO:72, or the complement thereof; a uracil at a position corresponding to position 691 according to SEQ ID NO:73, or the complement thereof; or a uracil at a position corresponding to position 930 according to SEQ ID NO:74, or the complement thereof.

In some embodiments, the subject is identified as being heterozygous for a cDNA molecule having a nucleotide sequence encoding an ANGPTL7 predicted loss-of-function polypeptide, wherein the nucleotide sequence comprises: a thymine at a position corresponding to position 916 according to SEQ ID NO:147, or the complement thereof; a thymine at a position corresponding to position 983 according to SEQ ID NO:148, or the complement thereof; a thymine at a position corresponding to position 948 according to SEQ ID NO:149, or the complement thereof; a thymine at a position corresponding to position 930 according to SEQ ID NO:150, or the complement thereof; a thymine at a position corresponding to position 905 according to SEQ ID NO:151, or the complement thereof; a thymine at a position corresponding to position 698 according to SEQ ID NO:152, or the complement thereof; a thymine at a position corresponding to position 691 according to SEQ ID NO:153, or the complement thereof; or a thymine at a position corresponding to position 930 according to SEQ ID NO:154, or the complement thereof.

In some embodiments, the subject is identified as being heterozygous for: i) a genomic nucleic acid molecule having a nucleotide sequence encoding an ANGPTL7 predicted loss-of-function polypeptide, wherein the nucleotide sequence comprises a thymine at a position corresponding to position 5,176 according to SEQ ID NO:9, or the complement thereof; ii) an mRNA molecule having a nucleotide sequence encoding an ANGPTL7 predicted loss-of-function polypeptide, wherein the nucleotide sequence comprises: a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; or a uracil at a position corresponding to position 742 according to SEQ ID NO:81, or the complement thereof; or iii) a cDNA molecule having a nucleotide sequence encoding an ANGPTL7 predicted loss-of-function polypeptide, wherein the nucleotide sequence comprises: a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; or a thymine at a position corresponding to position 981 according to SEQ ID NO:162, or the complement thereof.

In some embodiments, the subject is identified as being heterozygous for a genomic nucleic acid molecule having a nucleotide sequence encoding an ANGPTL7 predicted loss-of-function polypeptide, wherein the nucleotide sequence comprises a thymine at a position corresponding to position 5,176 according to SEQ ID NO:9, or the complement thereof.

In some embodiments, the subject is identified as being heterozygous for an mRNA molecule having a nucleotide sequence encoding an ANGPTL7 predicted loss-of-function polypeptide, wherein the nucleotide sequence comprises: a uracil at a position corresponding to position 967 according to SEQ ID NO:75, or the complement thereof; a uracil at a position corresponding to position 1,034 according to SEQ ID NO:76, or the complement thereof; a uracil at a position corresponding to position 999 according to SEQ ID NO:77, or the complement thereof; a uracil at a position corresponding to position 981 according to SEQ ID NO:78, or the complement thereof; a uracil at a position corresponding to position 956 according to SEQ ID NO:79, or the complement thereof; a uracil at a position corresponding to position 749 according to SEQ ID NO:80, or the complement thereof; a uracil at a position corresponding to position 742 according to SEQ ID NO:81, or the complement thereof; or a uracil at a position corresponding to position 981 according to SEQ ID NO:82, or the complement thereof.

In some embodiments, the subject is identified as being heterozygous for a cDNA molecule having a nucleotide sequence encoding an ANGPTL7 predicted loss-of-function polypeptide, wherein the nucleotide sequence comprises: a thymine at a position corresponding to position 967 according to SEQ ID NO:155, or the complement thereof; a thymine at a position corresponding to position 1,034 according to SEQ ID NO:156, or the complement thereof; a thymine at a position corresponding to position 999 according to SEQ ID NO:157, or the complement thereof; a thymine at a position corresponding to position 981 according to SEQ ID NO:158, or the complement thereof; a thymine at a position corresponding to position 956 according to SEQ ID NO:159, or the complement thereof; a thymine at a position corresponding to position 749 according to SEQ ID NO:160, or the complement thereof; a thymine at a position corresponding to position 742 according to SEQ ID NO:161, or the complement thereof; or a thymine at a position corresponding to position 981 according to SEQ ID NO:162, or the complement thereof.

In some embodiments, the subject is identified as being heterozygous for: i) a genomic nucleic acid molecule having a nucleotide sequence encoding an ANGPTL7 predicted loss-of-function polypeptide, wherein the nucleotide sequence comprises an adenine at a position corresponding to position 5,232 according to SEQ ID NO:10, or the complement thereof; ii) an mRNA molecule having a nucleotide sequence encoding an ANGPTL7 predicted loss-of-function polypeptide, wherein the nucleotide sequence comprises: an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof; or iii) a cDNA molecule having a nucleotide sequence encoding an ANGPTL7 predicted loss-of-function polypeptide, wherein the nucleotide sequence comprises: an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof.

In some embodiments, the subject is identified as being heterozygous for a genomic nucleic acid molecule having a nucleotide sequence encoding an ANGPTL7 predicted loss-of-function polypeptide, wherein the nucleotide sequence comprises an adenine at a position corresponding to position 5,232 according to SEQ ID NO:10, or the complement thereof.

In some embodiments, the subject is identified as being heterozygous for an mRNA molecule having a nucleotide sequence encoding an ANGPTL7 predicted loss-of-function polypeptide, wherein the nucleotide sequence comprises: an adenine at a position corresponding to position 1,023 according to SEQ ID NO:83, or the complement thereof; an adenine at a position corresponding to position 1,090 according to SEQ ID NO:84, or the complement thereof; an adenine at a position corresponding to position 1,055 according to SEQ ID NO:85, or the complement thereof; an adenine at a position corresponding to position 1,037 according to SEQ ID NO:86, or the complement thereof; an adenine at a position corresponding to position 1,012 according to SEQ ID NO:87, or the complement thereof; an adenine at a position corresponding to position 805 according to SEQ ID NO:88, or the complement thereof; an adenine at a position corresponding to position 798 according to SEQ ID NO:89, or the complement thereof; or an adenine at a position corresponding to position 1,037 according to SEQ ID NO:90, or the complement thereof.

In some embodiments, the subject is identified as being heterozygous for a cDNA molecule having a nucleotide sequence encoding an ANGPTL7 predicted loss-of-function polypeptide, wherein the nucleotide sequence comprises: an adenine at a position corresponding to position 1,023 according to SEQ ID NO:163, or the complement thereof; an adenine at a position corresponding to position 1,090 according to SEQ ID NO:164, or the complement thereof; an adenine at a position corresponding to position 1,055 according to SEQ ID NO:165, or the complement thereof; an adenine at a position corresponding to position 1,037 according to SEQ ID NO:166, or the complement thereof; an adenine at a position corresponding to position 1,012 according to SEQ ID NO:167, or the complement thereof; an adenine at a position corresponding to position 805 according to SEQ ID NO:168, or the complement thereof; an adenine at a position corresponding to position 798 according to SEQ ID NO:169, or the complement thereof; or an adenine at a position corresponding to position 1,037 according to SEQ ID NO:170, or the complement thereof.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Methods

Participating cohorts Association with IOP was tested on a total of 101,678 individuals and 27,529 individuals of European ancestry from the United Kingdom Biobank (UKB) and the MyCode Community Health Initiative cohort from Geisinger Health System (GHS), respectively. The UKB is a population-based cohort study of people aged between 40 and 69 years recruited through 22 testing centers in the UK between 2006-2010. The GHS MyCode study is a health system-based cohort of patients from Central and Eastern Pennsylvania (USA) recruited in 2007-2019. For IOP association tests in African ancestry individuals, 4,114 individuals from UKB and 3,167 individuals from the Primary Open Angle African-American Glaucoma Genetics (POAAGG) study conducted at the University of Pennsylvania Perelman School of Medicine were included. All participants with a glaucoma diagnosis code (ICD-10 H40) or self-reported glaucoma (UKB field IDs: 6148 and 20002) were excluded from IOP analyses.

Association with glaucoma was tested in 8 studies: UKB, GHS, Mt. Sinai Biome cohort (SINAI), the Malmö Diet and Cancer study (MALMO), the Estonia Biobank (EstBB), The Trøndelag Heath Study (HUNT), FinnGen, a study from Finland, and the Copenhagen General Population Study and the Copenhagen City Heart Study (CGPS-CCHS). In total, there were 36,550 cases (UKB: 11,502, GHS: 7,562, SINAI: 409, MALMO: 2,344, EstBB: 7,629, HUNT: 3,874; CPGS-CCHS: 465) and 743,109 controls of European ancestry individuals, and 5,153 cases (UKB: 448, POAAGG: 3,444, SINAI: 1,261) and 21,650 controls of African ancestry individuals in glaucoma analyses.

### Phenotype Definition

IOP in UKB was measured in each eye using the Ocular Response Analyzer (ORA, Reichert Corp., Buffalo, N.Y.). Participants were excluded from this test if they reported having eye surgery in the preceding 4 weeks or having an eye infection. The ORA calculates two forms of IOP, a Goldmann-correlated IOP (IOPg) and a corneal compensated IOP (IOPcc). IOPg most closely approximates the IOP measured by the Goldmann Applanation Tonometer (GAT), which has been the gold standard for measuring IOP, while IOPcc provides a measure of IOP that is adjusted to remove the influence of corneal biomechanics. For this study, the focus was on IOPg as this measurement is the most comparable to IOP measurements in other cohorts, and herein IOPg will be referred to as IOP. IOP in POAAGG was measured using a GAT. In GHS, IOP measurements were obtained from several instruments including GAT, Tono-pen and I-Care, which are correlated with IOPg readings from the ORA. For GHS individuals who were not prescribed any IOP medications, the median of all IOP measurements available was used. For individuals who had an IOP medication prescribed, the median of IOP measurements available preceding the start date for IOP medications (if available) was used. Individuals for whom non-medicated IOP values were not available were excluded from the IOP genetic analyses. For association analyses of IOP, the individuals with: 1) a glaucoma diagnosis; 2) IOP measures that were more than 5 standard deviations away from the mean; 3) more than a 10-mmHg difference between both eyes, were excluded. A mean IOP measure between both eyes for each individual was derived. IOP of only one eye was used in instances where IOP measures for both eyes were not available.

Glaucoma cases in GHS, SINAI, MALMO, HUNT, EstBB, FinnGen and CGPS-CCHS were defined by the presence of an ICD-10 H40 diagnosis code in either outpatient or inpatient electronic health records. In UKB, glaucoma cases were defined as individuals with either an ICD-10 H40 diagnosis or self-reported glaucoma (UKB field ID: 6148 or 20002). In the POAAGG cohort, glaucoma cases and controls were classified based on an ophthalmic examination by glaucoma specialists.

### Statistical Analysis

High coverage whole exome sequencing and genotyping was performed. The association with IOP and glaucoma of genetic variants or their gene burden was estimated using REGENIE v1.0.43 (Mbatchou et al., Nat. Genet., 2021, doi:10.1038/s41588-021-00870-7) (UKB, GHS, MALMO, SINAI), SAIGE (Zhou et al., Nat. Genet., 2018, 50, 1335-1341) (HUNT, EstBB, FinnGen) or logistic regression (CGPS-CCHS). Analyses were adjusted for age, age², sex, an age-by-sex interaction term, experimental batch-related covariates, and genetic principal components, where appropriate. Results across cohorts were pooled using inverse-variance weighted meta-analysis.

### Functional Studies

In vitro characterization was conducted for the WT ANGPTL7 and three ANGPTL7 variants: Gln175His, Arg177* and Trp188*. Briefly, WT ANGPTL7 and the three variants in a pcDNA 3.1(+) vector backbone were transiently transfected with FuGENE6 (Promega) into HEK293 cells. Protein and mRNA levels of the transfectants in the cell lysate and supernatant were measured via ELISA and TaqMan, respectively, and visualized by western blotting.

Angptl7−/− mice were generated by using the VelociMouse® technology. Heterozygous mice (Angptl7+/−) were bred to generate age-matched wild-type, het and KO littermates that were used for experimentation. Ocular anatomy in these mice was characterized using optical coherence tomography (OCT).

IOP was measured. Briefly, mice were anesthetized and IOP was measured in both eyes using a TonoLab rebound tonometer (Colonial Medical Supply, Franconia, N.H.) before the start of Angptl7 injection and every day afterwards for six days. When testing Angptl7 siRNAs, IOPs were measured in each eye before then start of experiment and then every week until end of study. IOP measurements for both eyes were completed within 3-5 minutes.

### Glaucoma Phenotype Definition

GHS, MALMO, SINAI: ICD-based glaucoma case definition in GHS, MDCS and MSSM required an in-patient diagnosis or ≥2 outpatient diagnoses of ICD10-H40 in the EHR. ICD-based excludes had ≥1 primary or ≥2 secondary diagnoses in the code range (H40-H42). ICD-based controls for glaucoma were defined as individuals who were not cases or excluded.

UKB: Glaucoma ICD-based definitions of cases in UKB required one primary diagnosis or ≥2 secondary diagnoses of ICD10-H40 in the in-patient Health Episode Statistics (HES) records. Since the self-reported diagnoses were available for glaucoma in UKB, the ICD-based and self-reported glaucoma were combined to define cases. Individuals were considered cases if they: identified ‘glaucoma’ from the eye problems or disorders list in the touchscreen questionnaire (UKB field ID: 6148) or, stated they had glaucoma in the verbal interview (UKB field ID: 20002) or were a case for ICD10 H40 glaucoma. Normal controls for glaucoma in UKB were defined as individuals who did not report having glaucoma in the touchscreen (UKB field ID: 6148) or the verbal interview (UKB field ID: 20002), and were defined as controls for ICD-based glaucoma as described above.

POAAGG: In brief, POAG cases were defined as having an open iridocorneal angle and characteristic glaucomatous optic nerve findings in one or both eyes, characteristic visual field defects and all secondary causes of glaucoma excluded. Controls in POAAGG were defined as subjects older than 35, without high myopia (greater than −8.00 diopters) or presbyopia (+8.00 diopters), a family history of POAG, abnormal visual field, IOP greater than 21 mmHg, neuroretinal rim thinning, excavation, notching or nerve fiber layer defects, optic nerves asymmetry or a cup to disc ratio between eyes greater than 0.2. Additional controls for POAAGG were identified from the Penn Medicine Biobank as individuals without ICD9 diagnoses for glaucoma.

EstBB: Glaucoma cases were defined as individuals with at least 2 records of ICD-10 H40 (and its descendants) and controls were individuals that without a diagnosis for ICD-10 H40-H42, ICD-10 H44.5 and ICD-10 Q15.0. Individuals with only 1 ICD-10 H40 code were excluded from the analysis.

HUNT: Glaucoma cases were defined as individuals with an ICD-10 H40 or an ICD-9 365 in-patient diagnosis code. Individuals were excluded from controls if they had any of the following codes: ICD10 H40-H42, ICD10 H44.51, ICD10 Q15.0/ICD9 365, ICD9 377.14 and ICD9 360.42.

FinnGen: Glaucoma cases were defined based on the presence of ICD-10 H40 in the electronic health records.

### Exome Sequencing in UKB, GHS, MALMO and SINAI

High coverage whole exome sequencing was performed. NimbleGen probes (VCRome) or a modified version of the xGen design available from Integrated DNA Technologies (IDT) were used for target sequence capture. Sequencing was performed using 75 bp paired-end reads on Illumina v4 HiSeq 2500 or NovaSeq instruments. Sequencing had a coverage depth (i.e., number of sequence-reads covering each nucleotide in the target areas of the genome) sufficient to provide greater than 20× coverage over 85% of targeted bases in 96% of VCRome samples and 20× coverage over 90% of targeted bases in 99% of IDT samples. Sequence read alignment and variant calling was based on the GRCh38 Human Genome reference sequence. Ensembl v85 gene definitions were used to determine the functional impact of single nucleotide variants and insertion-deletions. Predicted LOF genetic variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Missense variants were classified for likely functional impact according to the number of in silico prediction algorithms that predicted deleteriousness using SIFT, Polyphen2_HDIV and Polyphen2_HVAR (Adzhubei et al., Nat. Methods, 2010, 7, 248-249), LRT and MutationTaster. For each gene, the alternative allele frequency (AAF) and functional annotation of each variant determined inclusion into these 7 gene burden exposures: 1) pLOF variants with AAF<1%; 2) pLOF or missense variants predicted deleterious by 5/5 algorithms with AAF<1%; 3) pLOF or missense variants predicted deleterious by 5/5 algorithms with AAF<0.1%; 4) pLOF or missense variants predicted deleterious by at least 1/5 algorithms with AAF<1%; 5) pLOF or missense variants predicted deleterious by at least 1/5 algorithms with AAF<0.1%; 6) pLOF or any missense with AAF<1%; and 7) pLOF or any missense variants with AAF<0.1%. Each of these 7 gene burden exposures was tested for association with IOP and glaucoma risk using REGENIE, as described below.

### Genotyping

UKB: DNA samples were genotyped as described previously (Bycroft et al., Nature, 2018, 562, 203-209) using the Applied Biosystems UK BiLEVE Axiom Array (N=49,950) or the closely related Applied Biosystems UK Biobank Axiom Array (N=438,427). Genotype data for variants not included in the arrays were inferred using three reference panels (Haplotype Reference Consortium, UK10K and 1000 Genomes Project phase 3).

GHS, SINAI and MALMO: For SINAI and MALMO, DNA from participants was genotyped on the Global Screening Array (GSA) and for GHS, genotyping was performed on either the Illumina OmniExpress Exome (OMNI) or GSA. Both cohorts were imputed to the TOPMed reference panel (stratified by array for GHS) using the TOPMed Imputation Server. Prior to imputation, variants that had a MAF>=0.1%, missingness <1% and HWE p-value >10-15 were retained. Following imputation for GHS, data from the OMNI and GSA datasets were merged for subsequent association analyses, which included an OMNI/GSA batch covariate, in addition to other covariates described below.

Genetic association analyses in UKB, GHS, SINAI and MALMO Association analyses in each study were performed using the genome-wide linear (for IOP) or Firth logistic (for glaucoma) regression test implemented in REGENIE. Step 1 of REGENIE (i.e., prediction of individual trait values based on the genetic data) included directly genotyped variants with a minor allele frequency (MAF)>1%, <10% missingness, Hardy-Weinberg equilibrium test P-value>10⁴⁵ and linkage-disequilibrium (LD) pruning (1000 variant windows, 100 variant sliding windows and r²<0.9). The association model used in step 2 of REGENIE included as covariates (i) age, age², sex, age-by-sex and age²-by-sex; (ii) 10 ancestry-informative principal components (PCs) derived from the analysis of a set of LD-pruned (50 variant windows, 5 variant sliding windows and r²<0.5) common variants from the array (imputed for the GHS study) data generated separately for each ancestry; (iii) an indicator for exome sequencing batch (GHS: three batches; UKB: six IDT batches); and (iv) 20 PCs derived from the analysis of exome variants with a MAF<1% also generated separately for each ancestry.

Within each study, association analyses were performed separately for individuals of African (AFR) and European (EUR) ancestry, when available. Continental ancestries were determined by projecting each sample onto reference principal components calculated from the HapMap3 reference panel. Briefly, the samples were merged with HapMap3 samples and kept only SNPs in common between the two datasets. Further, SNPs with MAF<10%, genotype missingness >5% or Hardy-Weinberg Equilibrium test p-value <10-5 were excluded. PCs for the HapMap3 samples were calculated and projected each of the samples onto those PCs. To assign a continental ancestry group to each non-HapMap3 sample, a kernel density estimator (KDE) was trained using the HapMap3 PCs and used the KDEs to calculate the likelihood of a given sample belonging to each of the five continental ancestry groups. When the likelihood for a given ancestry group was >0.3, the sample was assigned to that ancestry group. When two ancestry groups had a likelihood >0.3, AFR over EUR, Admixed American (AMR) over EUR, AMR over East Asian (EAS), South Asian (SAS) over EUR, and AMR over AFR were arbitrarily assigned. Samples were excluded from analysis if no ancestry likelihoods were >0.3, or if more than three ancestry likelihoods were >0.3. Results were subsequently meta-analyzed across studies and ancestries using an inverse variance-weighed fixed-effects meta-analysis.

### Phenome-Wide Association Analysis for ANGPTL7 pLOF and Missense Variants

A phenome-wide analysis of the association of an aggregate of pLOF and missense variants in ANGPTL7 with hundreds of continuous traits or disease outcomes in the GHS and UKB studies was undertaken. Results were available for 24,082 outcomes across the two cohorts. To control for the number of statistical tests performed, associations were considered statistically significant if the association p-value met a Bonferroni correction for 24,082 tests, that is p<2E-06 (corresponding to a p-value threshold of 0.05 divided by 24,082 statistical tests).

Continuous traits and disease outcomes were defined as described below. In the UKB study, for continuous traits, the values of biomarker, imaging variables or other continuous traits measured during one of the UKB visits or their averages within a given study visit or across study visits were used as outcomes. For binary disease outcomes, case status definition required one or more of the following criteria to apply: a) self-reported disease status or use of medication at digital questionnaire or interview with a trained nurse; or b) EHR of inpatient encounters from the UK National Health Service Hospital Episode Statistics database coded using the ICD-10 coding system. For each binary outcome, controls were individuals without any of the criteria for case definition. In the GHS study, for binary disease outcomes, case status definition required one or more of the following criteria to apply: 1) a problem-list entry of the ICD-10 diagnosis code, 2) an inpatient hospitalization-discharge ICD-10 diagnosis code, or 3) an encounter ICD-10 diagnosis code entered for 2 separate outpatient visits on separate calendar days. Controls were individuals without any of the criteria for case definition. Individuals were excluded if they had the relevant ICD-10 code associated with only one outpatient encounter. For continuous traits, data cleaning was performed by removing non-physiologic lab values, invalid or contaminated specimens, and those that were over 5× upper limit of normal. Then the minimum, median, and maximum laboratory result values over the duration of follow-up were derived for each patient and used as outcomes.

### Small Interfering RNA Molecules

Small interfering RNAs molecules used in this study were synthesized by Alnylam Pharmaceuticals, Inc. (Cambridge, Mass.) as described (Nair et al., J. Am. Chem. Soc., 2014, 136, 16958-16961). The identities and purities of all oligonucleotides were confirmed by electrospray ionization mass spectroscopy and ion exchange high-performance liquid chromatography, respectively. These are siRNA molecules that contain modified bases (2′ mods) and such molecules are conjugated to a proprietary ocular targeting agent/moiety. Concentration of siRNA molecules used in this study were 15 mg/ml.

### Derivation of Mean Corneal Refractive Power and Astigmatism from Refractometry Traits

Corneal refractive power and corneal astigmatism were derived from the autorefractometry and keratometry data available in UKB as previously described (Pontikos et al., PLoS One, 2019, 14, e0218144). Briefly, corneal astigmatism was defined corneal power along strong meridian minus corneal power along weak meridian at 3 mm diameter, whereas the corneal power was the average of these two values for each eye. Refractive astigmatism is defined as the mean cylindrical power between both eyes.

### Generation of Angptl7−/− Mice

The genetically engineered Angptl7−/− mouse strain was created using VelociGene technology. Briefly, mouse embryonic stem cells (50% C57BL/6NTac; 50% 129S6/SvEvTac; and Crb1+/+) were targeted for ablation of a 571 base pair region of the Angptl7 locus, beginning 153 base pairs upstream of the start ATG (mm10 chr4:148,499,872-148,500,442). A self-deleting Hygromycin selection cassette was targeted to the deletion for selection in embryonic stem cells. Heterozygous targeted cells were microinjected into 8-cell embryos from Charles River Laboratories Swiss Webster albino mice, yielding FO VelociMice that were 100% derived from the targeted cells. These mice were subsequently bred to homozygosity and maintained in an animal facility during the study period. The resistance cassette was removed during FO breeding using self-deleting technology.

### Anterior Segment Imaging Using Optical Coherence Tomography

Mice were anesthetized with 0.1 mg/kg of a ketamine/xylazine mixture (12 mg/ml and 0.5 mg/ml, respectively) and one drop of topical proparacaine (0.05%, sterile) on the eyes. After a minute, proparacaine was wiped off of the eyes and images of anterior segment of mice were collected using the infrared (IR) and optical coherence tomography+IR (OCT+IR) options on the Heidelberg Spectralis machine. Following parameters were used to capture images: sensitivity (42), position (−0.00 mm), ART (6 frames), size of scan (large), width and height (15 degrees×10 degrees), and number of sections (81) were the same for all OCT and OCT+IR images. In addition, the sclera option, was used for capturing OCT+IR images of mouse eyes. After acquiring images, mice were put on a warming station and monitored until they were fully awake and exhibiting normal behavior. Corneal thickness was measured using the Heidelberg Eye Explorer (version 1.5.9.0) by three experts in mouse eye anatomy from OCT images of the center of the cornea (section 41/81 at zoom 800%). The measurements from all three individuals were collated in the final plot.

### IOP Measurements in Mice

IOPs were measured in mice. Briefly, mice were anesthetized and IOP was measured in both eyes using a TonoLab rebound tonometer (Colonial Medical Supply, Franconia, N.H.) before the start of Angptl7 injection and every day afterwards for six days. When testing Angptl7 siRNAs, IOPs were measured in each eye before then start of experiment and then every week until end of study. IOP measurements for both eyes were completed in 3-5 minutes.

### Injection of Angptl7 Protein and siRNA into Mouse Eyes

A 33-gauge needle with a glass microsyringe (5-μL volume; Hamilton Company) was used for injections of Angptl7 protein/siRNA into mice eyes. For intravitreal injections, the eye was proptosed, and the needle was inserted through the equatorial sclera and into the vitreous chamber at an angle of approximately 45 degrees, taking care to avoid touching the posterior part of the lens or the retina. Angptl7 protein (catalog #4960-AN-025; R&D Systems, Minneapolis, Minn.) or siRNA (from Alnylam Pharmaceuticals, Supplementary methods) or PBS (1 μL) was injected into the vitreous over the course of 1 minute. The needle was then left in place for a further 45 seconds (to facilitate mixing), before being rapidly withdrawn. Before and during intracameral injections of Angptl7 protein, mice were anesthetized with isoflurane (2.5%) containing oxygen (0.8 L/min). For topical anesthesia, both eyes received one to two drops of 0.5% proparacaine HCl (Akorn, Inc.). Each eye was proptosed and the needle was inserted through the cornea just above the limbal region and into the anterior chamber at an angle parallel to the cornea, taking care to avoid touching the iris, anterior lens capsule epithelium, or corneal endothelium. Up to 1 μL of Angptl7 protein or PBS was injected into each eye over a 30-second period before the needle was withdrawn. Only one injection was administered at day 0.

### In Vitro Characterization

HEK293 cell line was cultured in DMEM media (4.5 g/L D-Glucose, (+) L-Glutamine, (−) Sodium Phosphate, (−) Sodium Pyruvate supplemented with 10% FBS and 1% Penicillin-Streptomycin-Glutamine (Invitrogen), at 37° C. in a humidified atmosphere under 5% CO₂. The day before transfection, HEK293 cells were seeded in OptiMEM supplemented with 10% FBS. After 24 hours, the cells were transfected with FuGENE 6, and 10 μg of pcDNA 3.1(+) encoding the following proteins: ANGPTL7 wild type, Gln175His, Arg177* and Trp188*. After 24 hours, the media was changed with 2% FBS OptiMEM. The following day, the cells were collected in RIPA buffer, supplemented with protease and phosphatase inhibitors (BRAND) or TRizol reagent (Invitrogen) for protein and RNA analysis, respectively. The supernatants were transferred to an Eppendorf tube and immediately flash frozen for downstream protein analysis. Western blot analysis was performed using a rabbit polyclonal antibody against ANGPTL7 at 1:1,000 dilution (10396-1-AP ProteinTech), using standard procedures. ANGPTL7 was quantified by ELISA according to manufacturer's instructions (LS-F50425 Life Sciences). The cell lysates were diluted 1:1,000. The supernatants were diluted 1:10,000. The ELISA plate was read at 450 nm via SpectraMax M4 plate reader (Molecular Devices).

Total RNA was extracted using TRizol reagent (Invitrogen) and RNeasy kit (Qiagen) according to manufacturer's instructions and treated with RNase-free DNase I (Promega). cDNA was synthesized using Superscript VILO cDNA synthesis kit (Invitrogen). Taqman analysis was performed using TaqMan Fast Advanced Master Mix (Applied Biosystems) in a QuantStudio 6 Flex (Applied Biosystems) and commercially available primers and probes for ANGPTL7 (Hs00221727—Applied Biosystem) and GAPDH (Hs02786624_g1—Applied Biosystem).

### In Situ Hybridization Using RNAScope

The expression pattern of TM single cell cluster specific gene expression in the human donor eye was determined by in situ hybridization using RNAScope® according to manufacturer's specifications (Advanced Cell Diagnostics). Briefly, 10% NBF fixed and paraffin embedded human donor eye cups were cut into 5 to 10 m sections and mounted on SUPERFROST® Plus glass slides. For RNAScope, slides were baked on slide warmer for 1 hour at 60° C. and deparaffinized for 20 minutes. Tissue sections then underwent 10 minutes of Pretreat 1—RNAScope hydrogen peroxide treatment (ACD, 320037) at room temperature, followed by 20 minutes of boiling at 90° C. in Pretreat 2—target retrieval treatment (ACD, 320043) in Oster Steamer (IHC World, LLC, Model 5709) and 30 minutes of Pretreat 3-RNAScope protease plus treatment (ACD, 320037) at 40° C. in a HybEZ Oven (ACD, 310010). Tissue sections were then incubated with DNaseI for 10 minutes at 40° C. to reduce potential background from probes binding to genomic DNA. Tissue sections were then washed five times with water, hybridized with RNAScope probes for 2 hours at 40° C. and the remainder of the manufacturer's assay protocol was implemented (ACD, 322360) from Amplified 1 to Amplified 6. The slides were washed twice (two minutes each at room temperature) with RNAScope wash buffer (ACD, 310091). Signal was detected by incubation with Red working solution (1:60 ratio of Red B to Red A) at room temperature for 10 minutes in the absence of light, followed by washing the slides in water several times and viewing under microscope. In some experiments, fluorescent signals were visualized and captured using an open-field Nikon Eclipse Ti-E microscope.

### Example 2: Coding Variants in ANGPTL7 are Associated with Reduced IOP

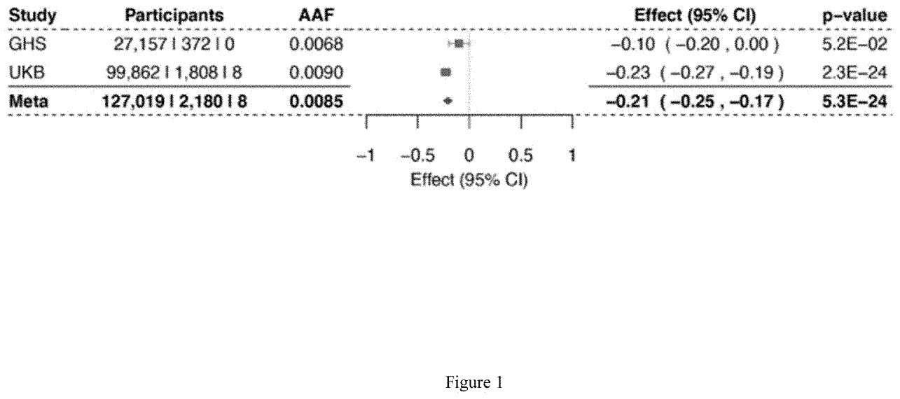

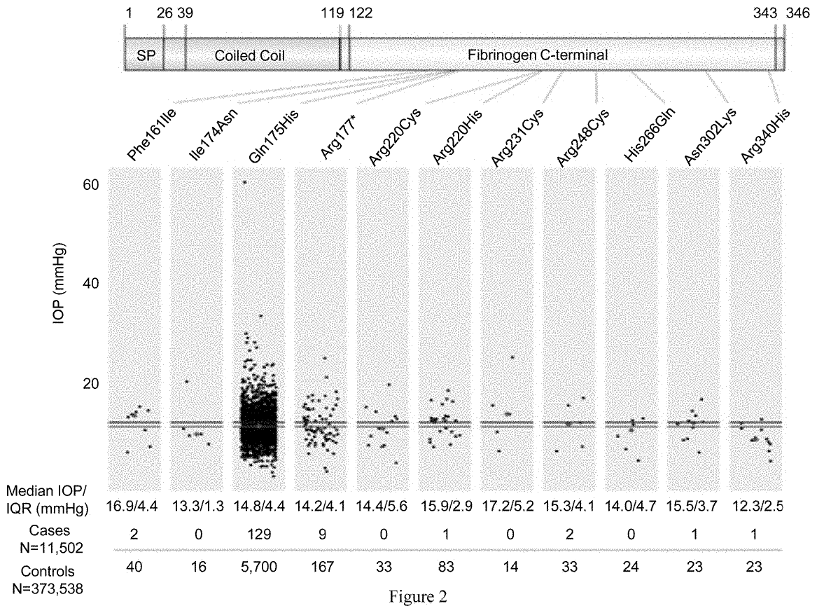

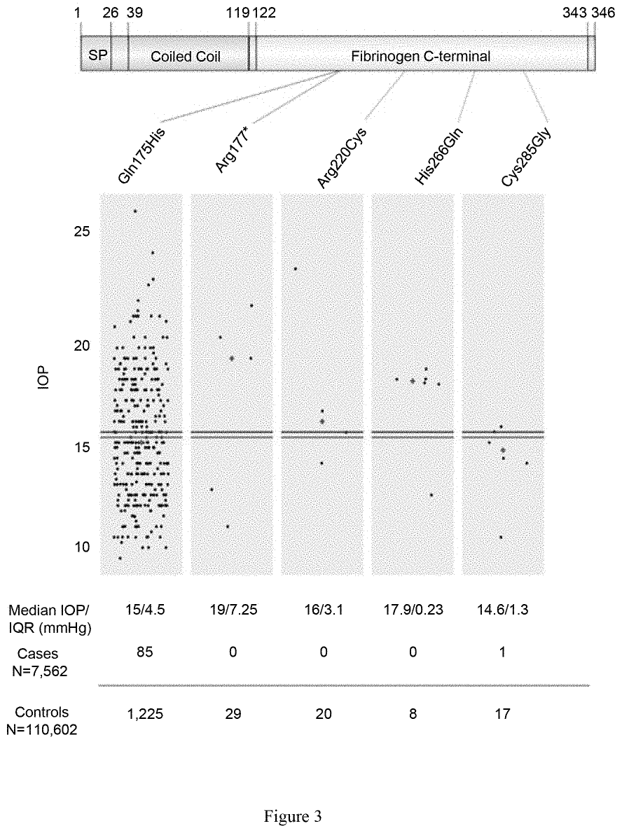

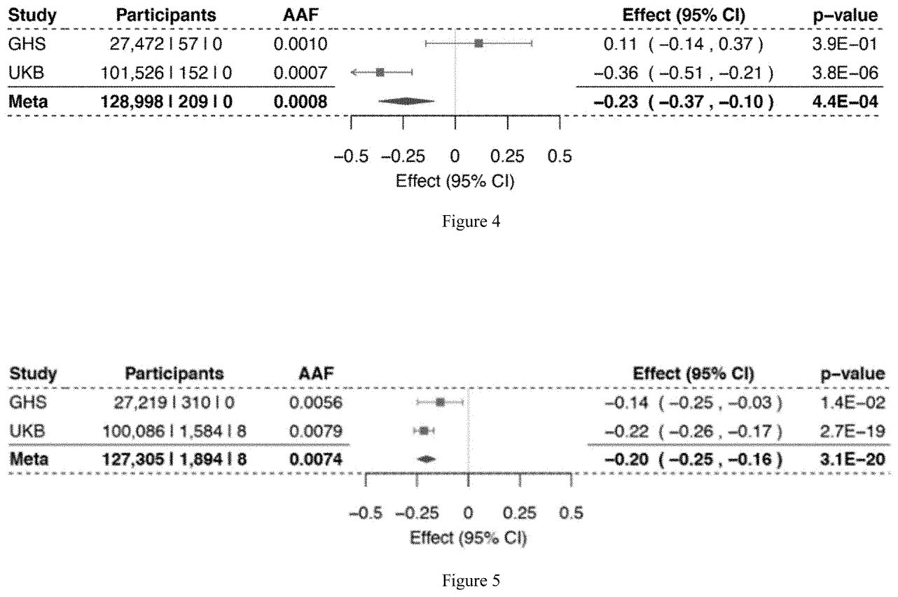

The effect of rare, protein-altering variation on IOP were studied across two large cohorts, UK Biobank (UKB) and Geisinger DiscovEHR (GHS), on 129,207 individuals of European descent after exclusion of cases with a glaucoma diagnosis (see, Methods and Tables 2-5). To increase the power to detect associations with rare variants, burden tests were performed by aggregating for each gene all (minor allele frequency [MAF]<1%) predicted loss-of-function (pLOF, defined as stop-gain, frameshift, splice donor, splice acceptor, start-loss, and stop-loss) and missense (predicted deleterious by 5 algorithms) variants. A genome-wide significant association (p-value<5E-08) of variants in ANGPTL7 with reduced IOP (beta_{allelic}=−0.21, p-value=5.3E-24; FIG. 1) was observed. The gene burden included 99 rare variants, but was dominated by two: a missense (Gln175His, MAF=0.7%) and a stop-gain (Arg177*, MAF=0.03%) variant, which accounted for 1,902 and 82 individuals out of a total of 2,188 carriers, respectively (FIG. 2 and FIG. 3). Exclusion of Gln175His and Arg177* from the burden meta-analysis between UKB and GHS did not eliminate the signal completely (beta_{allelic}=−0.23, p-value=4.4E-04; FIG. 4), suggesting that other ultra-rare variants in ANGPTL7 are also associated with reduced IOP.

TABLE 2
Number of samples across cohorts included in European
ancestry IOP analysis
IOP (EUR)

| Cohort | Data Type | # Samples |
|---|---|---|
| UKB | Array | 101,590 |
| | Imputed | 108,120 |
| | Exome | 101,678 |
| GHS | Imputed | 28,977 |
| | Exome | 27,529 |

TABLE 3
Number of samples across cohorts included in European
ancestry glaucoma analyses
Glaucoma (EUR)

| Cohort | Data Type | # Cases | # Controls |
|---|---|---|---|
| UKB | Array | 11,494 | 373,246 |
| | Imputed | 12,377 | 400,978 |
| | Exome | 11,502 | 373,538 |
| GHS | Imputed | 8,032 | 114,171 |
| | Exome | 7,562 | 110,602 |
| SINAI | Array | 409 | 9,178 |
| | Imputed | 409 | 9,178 |
| | Exome | 409 | 9,178 |
| MALMO | Array | 2,347 | 25,998 |
| | Imputed | 2,342 | 25,973 |
| | Exome | 2,344 | 25,975 |
| FinnGen | Array/Imputed | 5,177 | 130,461 |
| EstBB | Array/Imputed | 7,629 | 128,075 |
| HUNT | Array/Imputed | 3,874 | 64,541 |
| CGPS-CCHS | Array/Imputed | 465 | 19,354 |

TABLE 4
Number of samples across cohorts included in African
ancestry IOP analyses
IOP (AFR)

| Cohort | Data Type | # Samples |
|---|---|---|
| UKB | Array | 4,132 |
| | Imputed | 4,405 |
| | Exome | 4,114 |
| POAAGG | Array | 3,282 |
| | Imputed | 3,282 |
| | Exome | 3,167 |

TABLE 5
Number of samples across cohorts included in African
ancestry glaucoma analyses
Glaucoma (AFR)

| Cohort | Data Type | # Cases | # Controls |
|---|---|---|---|
| UKB | Array | 449 | 7,374 |
| | Imputed | 481 | 7,922 |
| | Exome | 448 | 7,328 |
| SINAI | Array | 1,261 | 10,270 |
| | Imputed | 1,261 | 10,270 |
| | Exome | 1,261 | 10,270 |
| POAAGG | Array | 3,590 | 4,184 |
| | Imputed | 3,590 | 4,184 |
| | Exome | 3,444 | 4,052 |

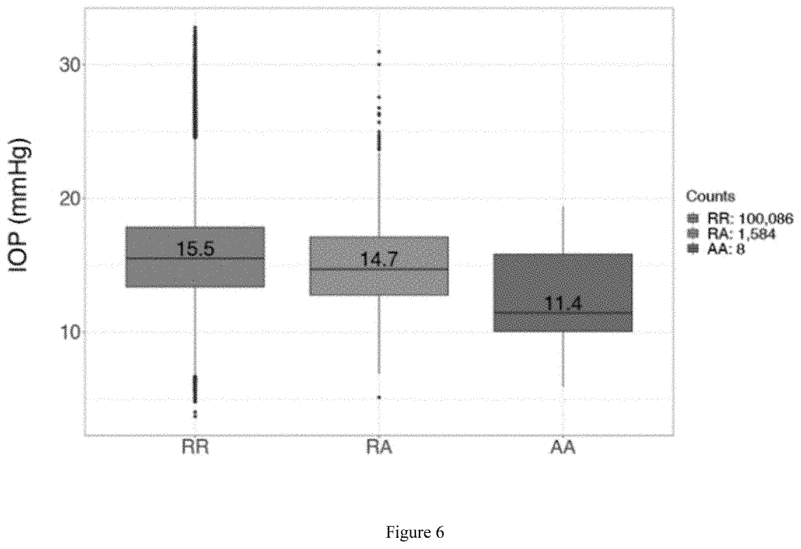

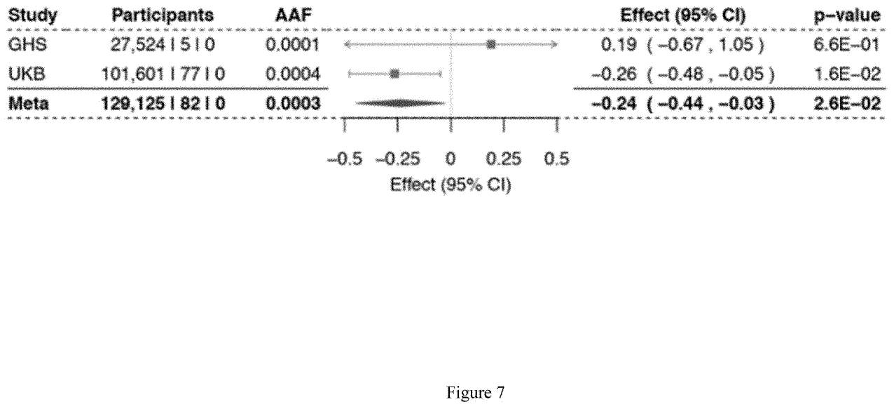

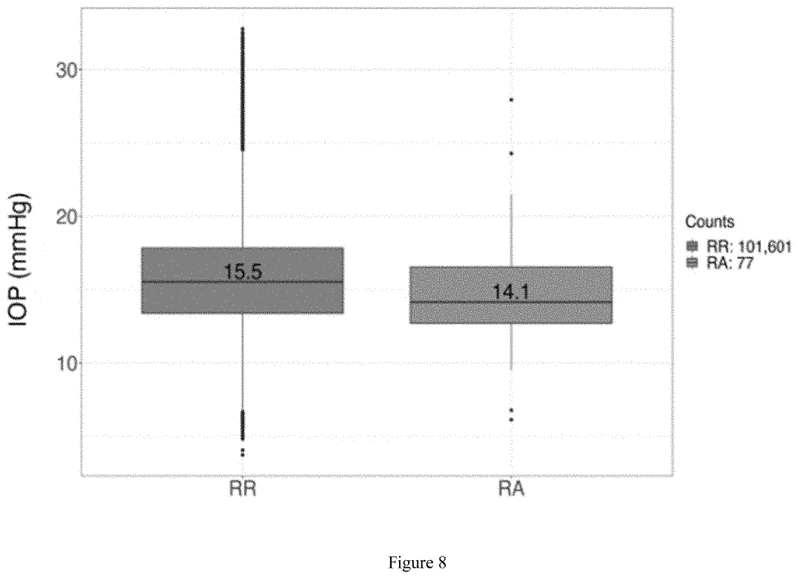

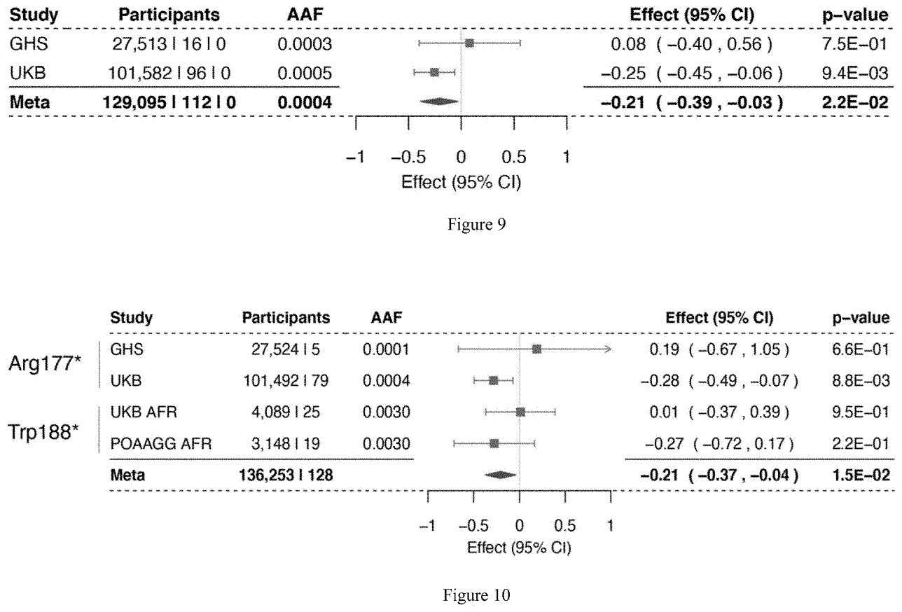

In single variant analyses, Gln175His was associated with reduced IOP at a genome-wide significant level (beta_{allelic}=−0.20 SD, p-value=3E-20, FIG. 5). Heterozygous and homozygous carriers of Gln175His in ANGPTL7 have a 5.2% (0.8 mmHg) and 26.5% (4.1 mmHg) reduction in median IOP in UKB, respectively (FIG. 6). The Arg177* variant was also nominally associated with reduced IOP with an effect size similar to that of Gln175His (beta_{allelic}=−0.24 SD, p-value=2.6E-02, FIG. 7), and 77 heterozygous Arg177* carriers had a 9% (1.4 mmHg) median IOP decrease (FIG. 8). Arg177* appears to be the predominant pLOF variant in European populations; a burden test restricted to 12 pLOF variants was dominated by Arg177* (82 of 112 total carriers) and was comparable (beta_{allelic}=−0.21 SD, p-value=2.2E-02; FIG. 9) to the single-variant association of Arg177* with IOP.

Other ancestries were searched for additional pLOFs in ANGPTL7 and identified Trp188*, which is enriched in individuals of African descent (MAF=0.3%) compared to Europeans (MAF=0.0013%). an association of Trp188* with IOP was performed in African ancestry individuals from UKB and the Primary Open Angle African American Glaucoma Genetics (POAAGG) study, followed by meta-analysis. Trp188* showed a trend towards reduced IOP, similar to Arg177* and Gln175His, but this was not statistically significant (beta_{allelic}=−0.11 SD, p-value=5E-01). A cross-ancestry meta-analysis of Arg177* and Trp188* variants showed a nominally significant association with reduced IOP (beta_{allelic}=−0.21 SD, p-value=1.5E-02; FIG. 10).

In summary, a significant association of Gln175His in ANGPTL7 with reduced IOP was observed and a sub-threshold association, in the same direction and of similar magnitude, with pLOF variants in ANGPTL7. Assuming that the pLOF variants indeed cause a loss of protein function, these data suggests that loss of ANGPTL7 can lead to lower IOP.

### Example 3: IOP-Associated Variants in ANGPTL7 are Protective Against Glaucoma

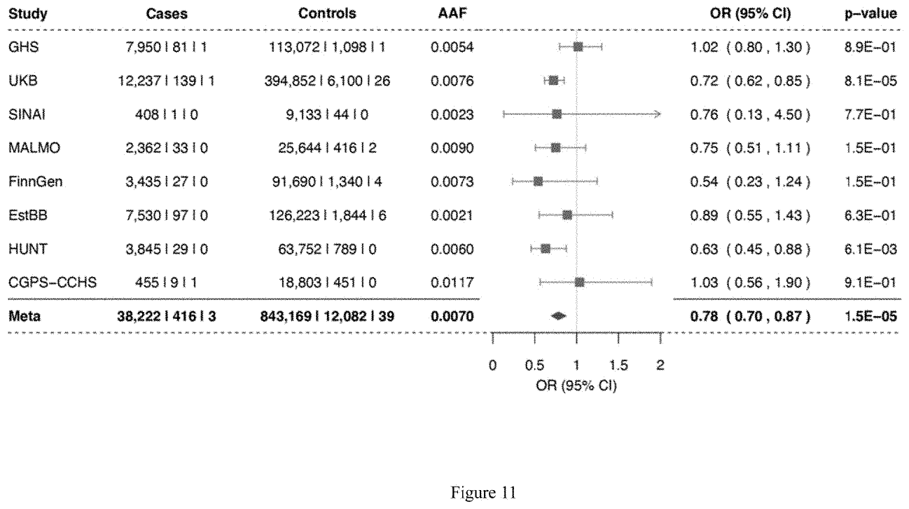

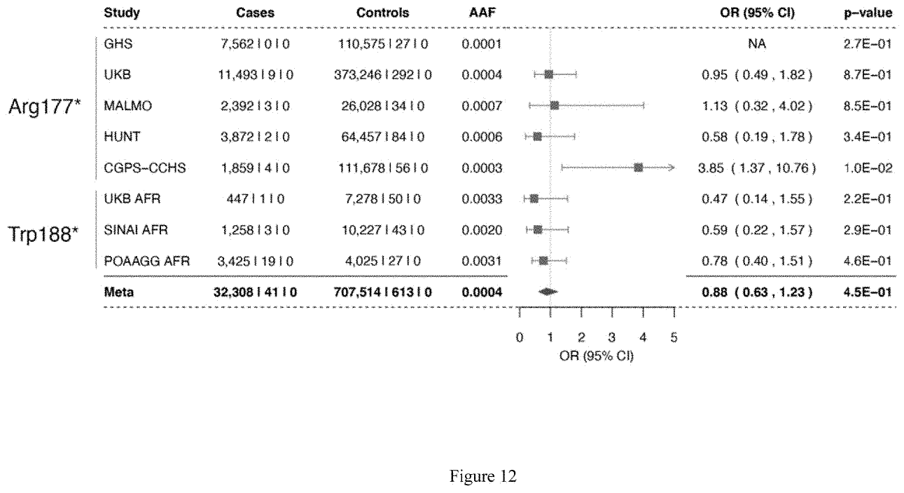

To understand if carriers of variants in ANGPTL7 would also be protected against glaucoma, an association analysis of Gln175His with glaucoma was performed in UKB, GHS, and six additional studies: Mount Sinai's BioMe Personalized Medicine Cohort from Mount Sinai Health System, New York (SINAI), the Malmö diet and cancer study from Malmö, Sweden (MALMO), the FinnGen cohort from Finland, the Estonia Biobank at the University of Tartu, Estonia (EstBB), the HUNT study from Nord-Trøndelag, Norway (HUNT), and the Copenhagen General Population Study/Copenhagen City Heart Study from Copenhagen, Denmark (CGPS-CCHS). A meta-analysis across these eight cohorts showed a significant reduction in glaucoma risk for Gln175His carriers (odds ratio (OR_{allelic})=0.78, p-value=1.5E-05, FIG. 11). Glaucoma risk in carriers of the rarer Arg177*/Trp188* variants was also analyzed in a cross-ancestry meta-analysis and observed a consistent trend towards reduction in risk (OR_{allelic}=0.88, p-value=4.5E-01, FIG. 12). Taken together, the associations of missense and pLOF variants in ANGPTL7 with reduced IOP and the association of the missense variant with reduced glaucoma risk suggest the hypothesis that loss of ANGPTL7 confers protection against glaucoma, and that this effect is mediated through the regulation of IOP.

### Example 4: ANGPTL7 Variants are Associated with Corneal Measures

A phenome-wide association analysis (PheWAS) was performed to understand whether other traits were associated with a burden of loss-of-function and deleterious missense variants in ANGPTL7. The ANGPTL7 variant aggregate was tested for association with 14,050 and 10,032 binary and quantitative traits in UKB and GHS, respectively. No associations reached phenome-wide significance (p-value<2E-06 after multiple testing correction for 24,082 total traits) in GHS. The only significant associations in UKB were with ocular traits (Table 6), specifically with decreased IOPcc, decreased corneal resistance factor (CRF) and increased corneal refractive power along both weak and strong meridians measured at 3 and 6 mm diameters. The effect of these variants on IOPcc was slightly attenuated (−0.17 SD) compared to that on IOPg (−0.22 SD in UKB), which suggests that ANGPTL7 has some impact on corneal properties that are known to affect the IOPg measurements. The association observed with decreased CRF is also consistent with a corneal effect of ANGPTL7.

TABLE 6
Statistically significant (P-value < 2E−06) results from
PheWAS of an aggregate of 110 pLOF and deleterious
missense variants (MAF < 1%) in ANGPTL7 in UKB

| | | OR/Effect |
|---|---|---|
| | | in SD |
| Trait | P-value | LCl/UCI |
| Intraocular pressure Corneal | 6.32E−14 | −0.17 |
| Compensated (IOPcc) (mean of both eyes) | | (−0.21/−0.13) |
| CRF (right eye) | 5.20E−13 | −0.16 |
| | | (−0.20/−0.11) |
| CRF (left eye) | 4.5E−12 | −0.15 |
| | | (−0.20/−0.11) |
| 6 mm weak meridian (left eye) | 4.00E−20 | 0.21 |
| | | (0.16/0.25) |
| 6 mm weak meridian (right eye) | 4.20E−18 | 0.19 |
| | | (0.15/0.24) |
| 6 mm strong meridian (left eye) | 3.10E−17 | 0.19 |
| | | (0.14/0.23) |
| 6 mm strong meridian (right eye) | 7.60E−17 | 0.18 |
| | | (0.14/0.23) |
| 3 mm weak meridian (right eye) | 2.00E−14 | 0.16 |
| | | (0.12/0.20) |
| 3 mm weak meridian (left eye) | 1.60E−13 | 0.15 |
| | | (0.11/0.20) |
| 3 mm strong meridian (left eye) | 1.30E−12 | 0.15 |
| | | (0.11/0.19) |
| 3 mm strong meridian (right eye) | 1.80E−12 | 0.15 |
| | | (0.11/0.19) |
| Corneal Power (mean of both eyes) | 1.10E−13 | 0.16 |
| | | (0.11/0.20) |

CRF = corneal resistance factor.

The autorefraction measurements at 3 mm diameter were used to derive measures of clinical interest, namely, mean corneal refractive power (mCRP), corneal astigmatism and refractive astigmatism and checked for association with ANGPTL7. A significant association with increased mCRP (beta_{allelic}=0.16, p-value=1.1E-13) was observed but no association with corneal or refractive astigmatism. Also, no associations was observed with the mean spherical equivalent (MSE; measure of refractive error) or myopia (either derived from MSE or via ICD-10 diagnosis), which could result from increased mCRP. Overall, these PheWAS results show that while ANGPTL7 is associated with changes in corneal anatomy/biomechanics-related quantitative measures, an increased risk was not detected for any related disease outcomes that could be tested. In addition, pLOF and deleterious missense variants in ANGPTL7 are not associated with any systemic quantitative traits or binary outcomes.

### Example 5: Gln175His, Arg177* and Trp188* are Defective in Secretion

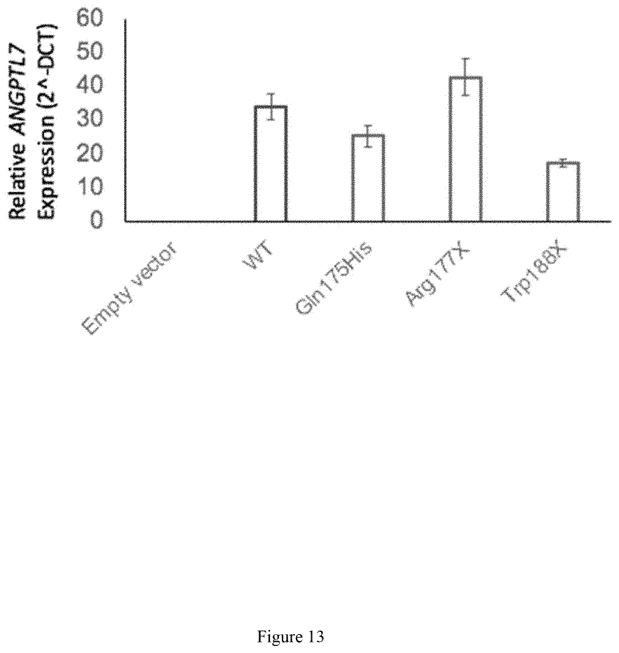

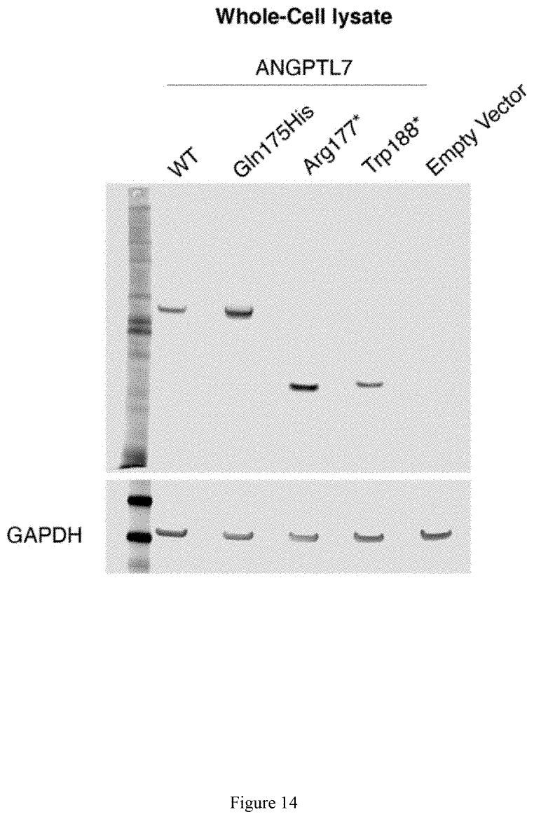

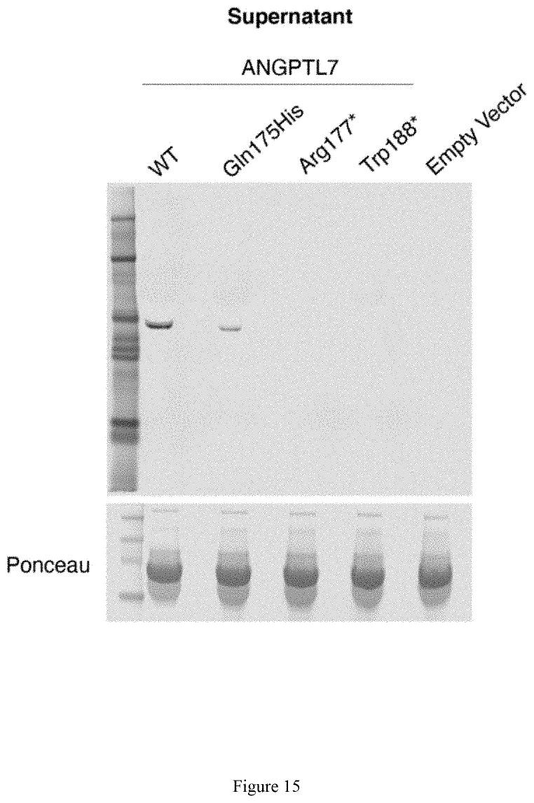

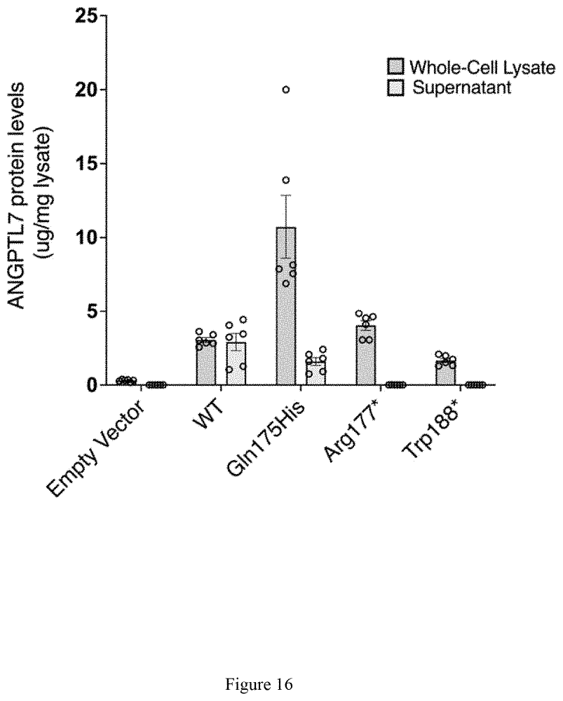

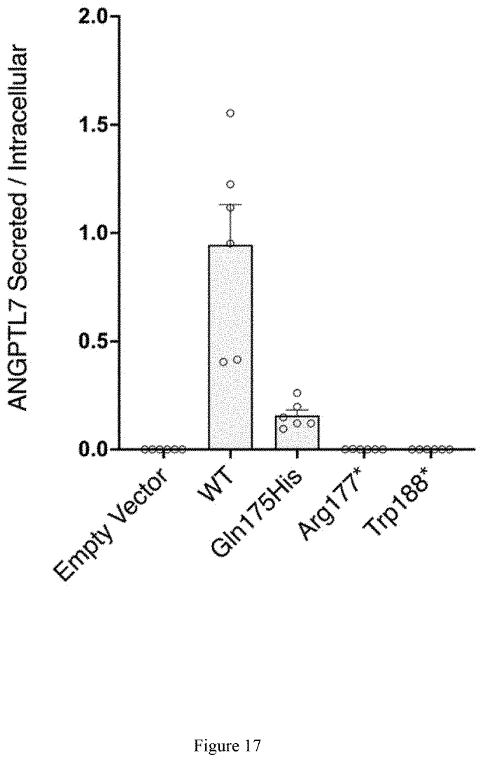

To understand the impact of Gln175His, Arg177*, and Trp188* variants on the expression and secretion of ANGPTL7, constructs expressing the wild type (WT), Gln175His, Arg177*, and Trp188* variant proteins were transiently transfected in HEK293 cells. mRNA levels were measured by Taqman, which showed similar Gln175His and Arg177* transcript levels and a significant decrease in the Trp188* transcript levels compared to WT (FIG. 13). However, analysis of intracellular, steady-state protein in whole-cell lysate by western blotting and ELISA revealed increased levels of Gln175His compared to WT. As expected, Arg177* and Trp188* encoded lower molecular weight proteins (˜30-32 kDa), and while Arg177* showed similar levels of expression compared to WT, Trp188* showed lower levels of protein expression (FIG. 14). Because ANGPTL7 is a secreted protein, the levels of wild type, Gln175His, Arg177*, and Trp188* were determined in the cellular supernatant. The Arg177* and Trp188* variants were not detectable and the Gln175His was drastically reduced in the supernatant compared to WT (FIG. 15). ELISA was used to quantify the protein levels confirming the severely reduced levels of Gln175His and the inability of Arg177* and Trp188* to reach the extracellular space (FIG. 16 and FIG. 17).

### Example 6: ANGPTL7 is Expressed in Cornea, TM, and Sclera Across Species

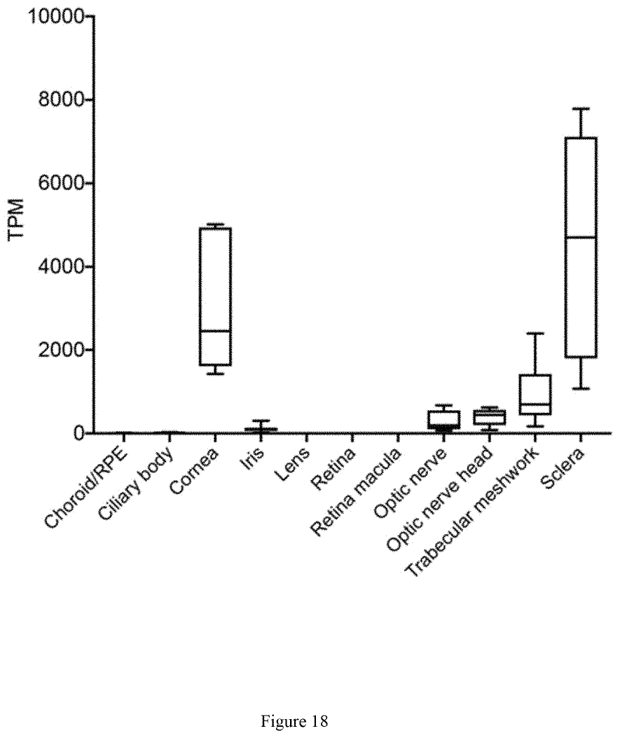

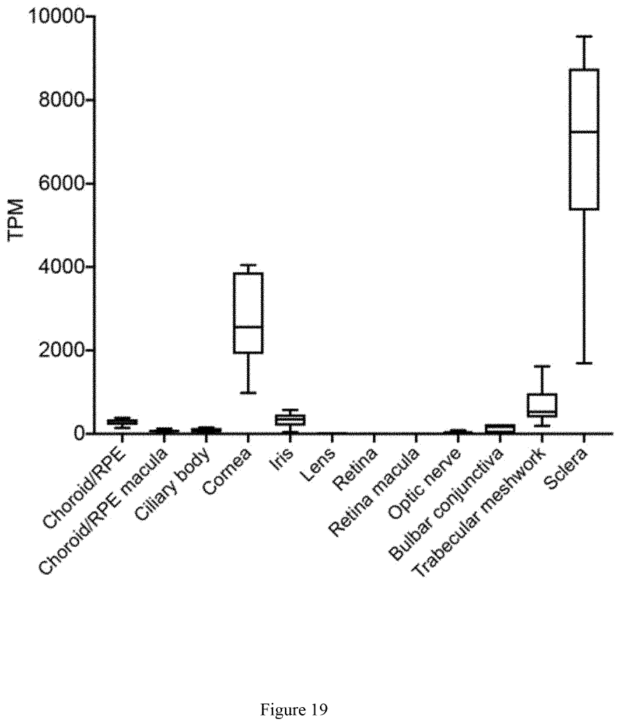

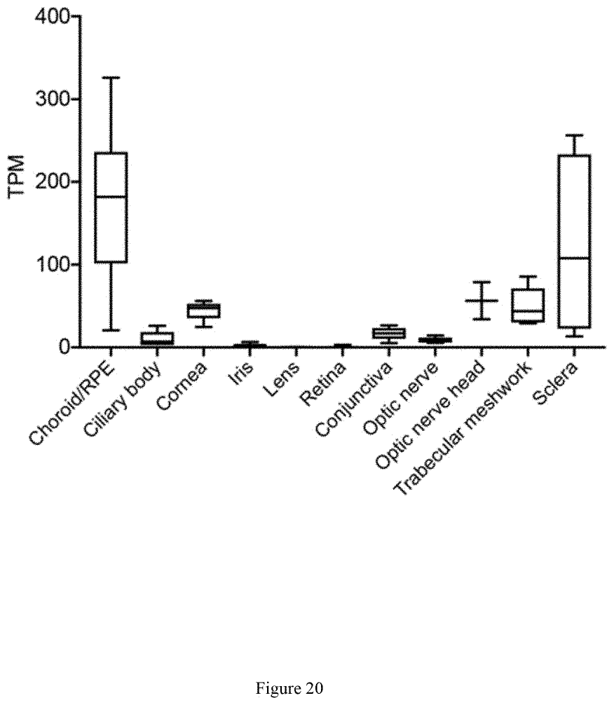

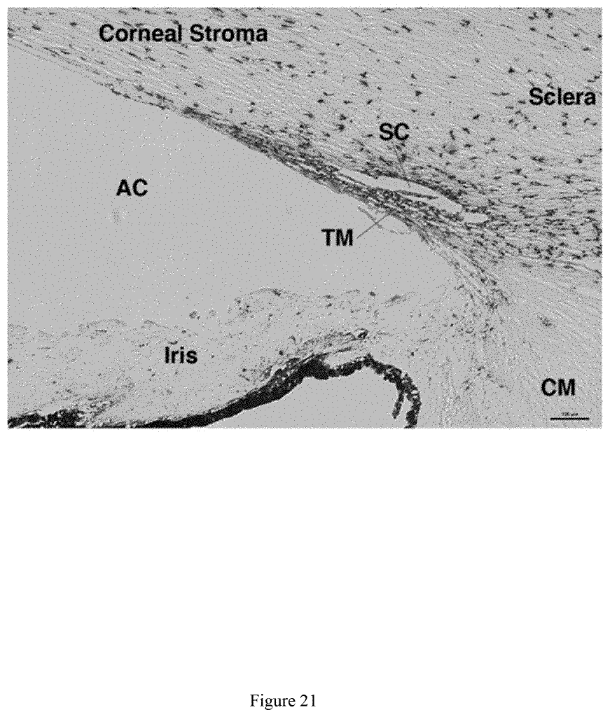

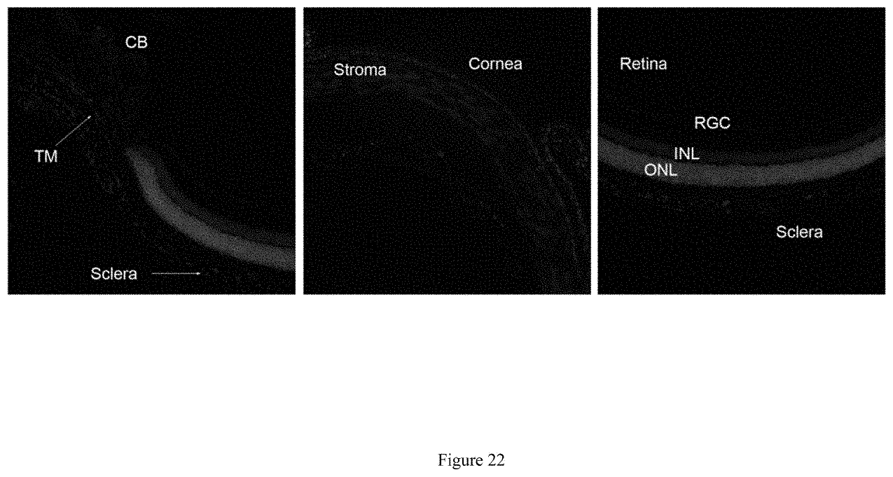

To identify expression of ANGPTL7 in ocular tissues across different species, transcriptome profiles from different parts of eye were generated (Supplementary methods). High ANGPTL7 expression was observed in cornea, TM, and sclera in human and African green monkey eyes (FIG. 18 and FIG. 19). High Angptl7 expression was also observed in cornea, TM, sclera, optic nerve, and choroid/RPE in eyes of C57BL/6J mice (FIG. 20). In situ hybridization on human donor and mouse eyes using RNAScope probes for human ANGPTL7 and mouse Angptl7 showed ANGPTL7/Angptl7 expression in TM, cornea stroma, and sclera (FIG. 21 and FIG. 22).

### Example 7: Increasing Levels of Angptl7 in Mouse Eyes Increases IOP

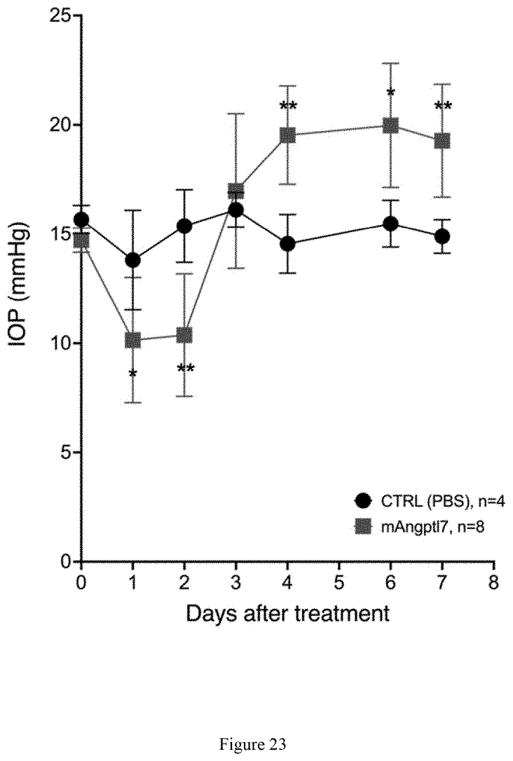

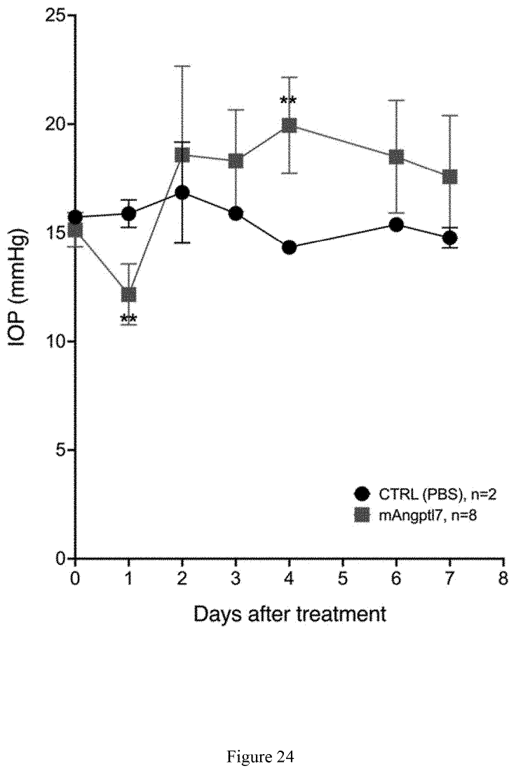

Previous studies showed that overexpression of ANGPTL7 in TM cells leads to changes in extracellular matrix (ECM) deposition and reorganization and that ANGPTL7 is increased in aqueous humor of glaucoma patients, however, the role of ANGPTL7 in IOP regulation is not clear. To investigate this, Angptl7 protein was injected in mice via intravitreal and intracameral routes and measured IOP over time. Intravitreal injection of Angptl7 protein in mice led to an initial drop in IOP followed by, starting on day 4, an elevation in IOP of 4-5 mmHg, a 22-25% increase compared to baseline, that lasted until the end of the experiment on day 7 (FIG. 23). Similarly, intracameral injection of Angptl7 protein in mice led to an initial drop and subsequent elevation (by 2-5 mmHg) of IOP, starting on day 3 until the end of the experiment on day 7 (FIG. 24). Vehicle-injected mice did not show an increase in IOP in either route of administration.

### Example 8: Angptl7 KO Mice have Lower Basal IOP than WT

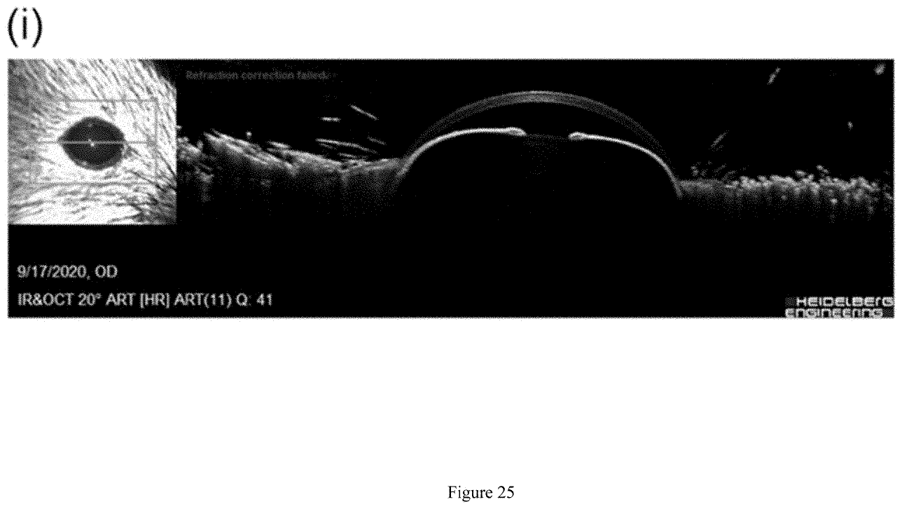

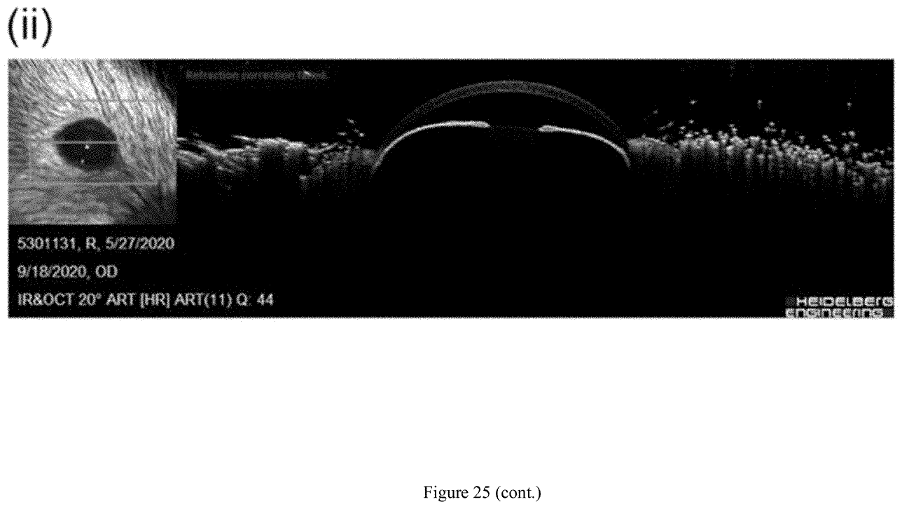

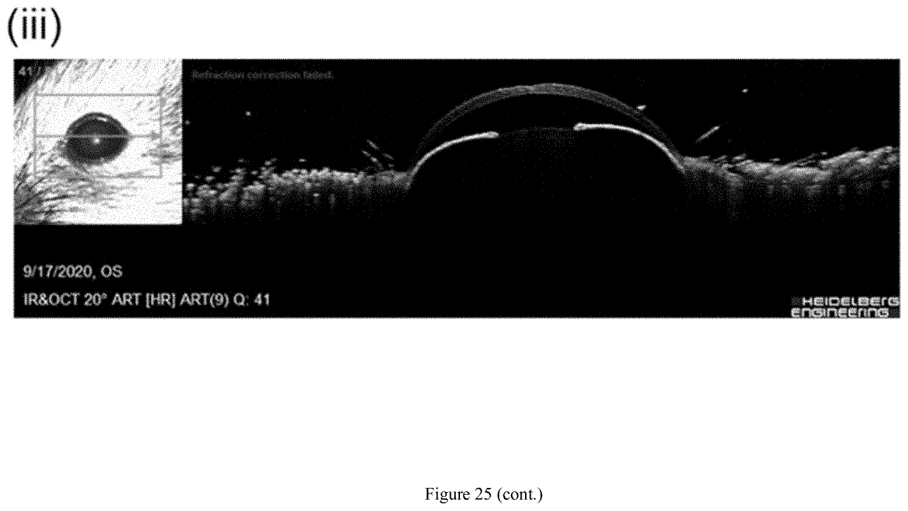

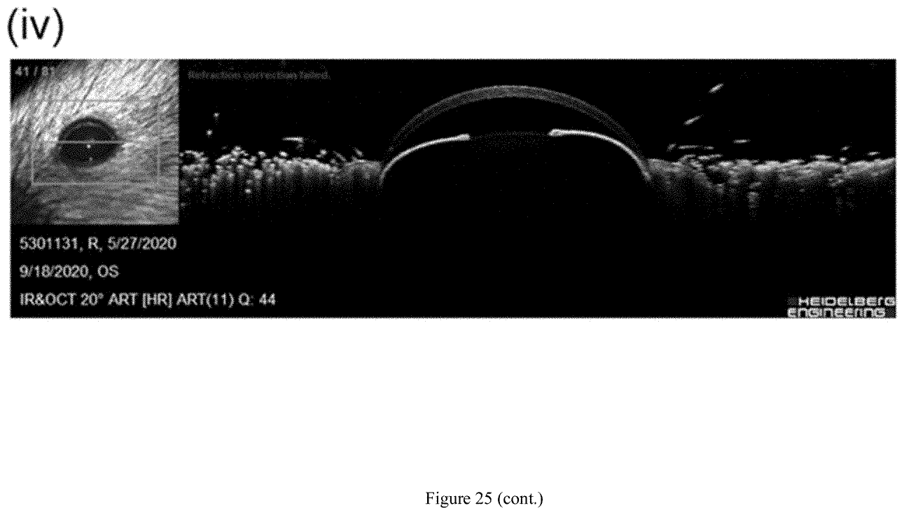

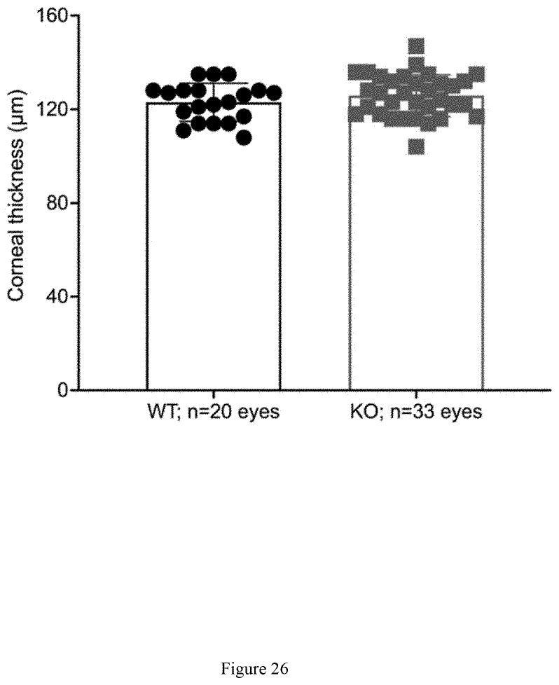

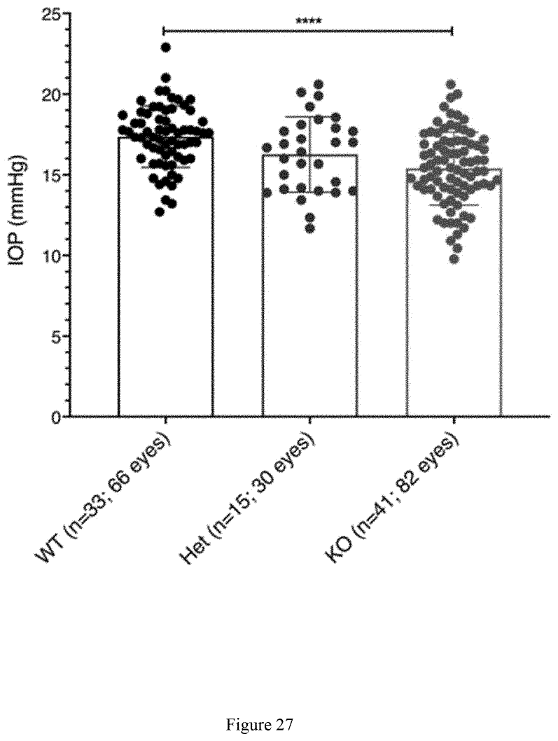

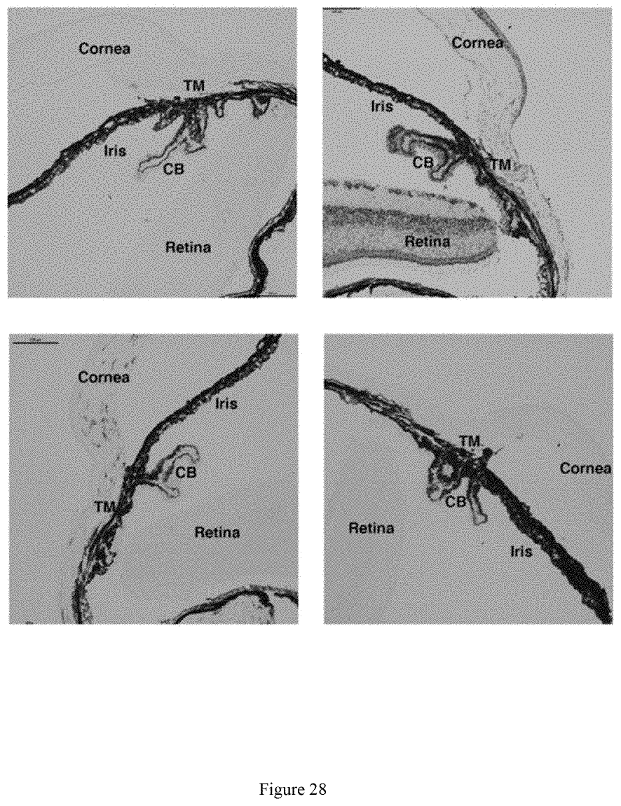

Angptl7−/− (KO) mice were generated and characterized. No ocular changes on anterior segment optical coherence tomography (OCT), or a difference in corneal thickness were observed between the two genotypes (FIG. 25 and FIG. 26). The IOP was also monitored in KO, Angptl7+/−(Het) and WT mice showing a dose-dependent decrease in IOP across the three genotypes (FIG. 27). The mean IOP was lowered in KO mice (mean (SD): 15.39 (2.3) mmHg) by 11% (1.96 mmHg, P<0.0001) compared to WT (17.36 (1.9) mmHg). Het mice (16.26 (2.3) mmHg) showed a smaller (6%, 1.1 mmHg, P=0.02) but significant reduction in IOP compared to WT. These results were confirmed via RNAscope that Angptl7 mRNA was not expressed in any ocular tissue in KO mice whereas it was expressed in TM, cornea, and sclera of WT mice (FIG. 28).

### Example 9: siRNA Induced Knockdown of Angptl7 mRNA and Lowering of IOP in WT Mice

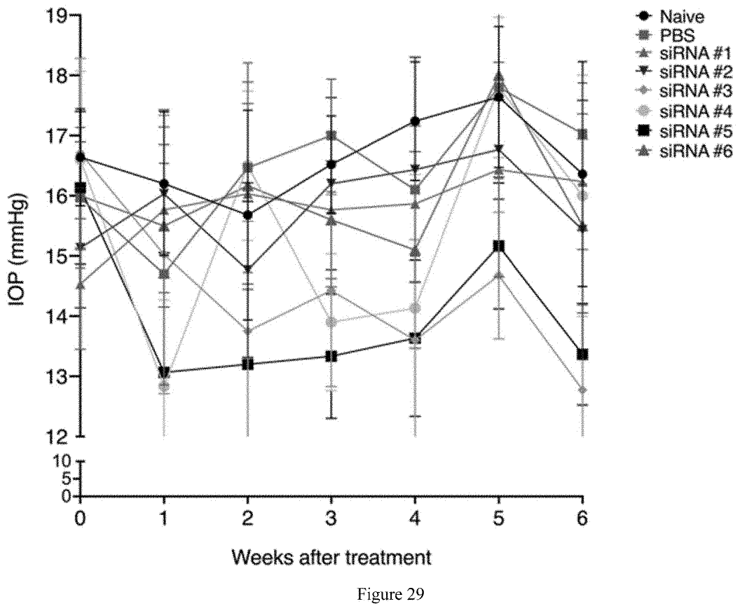

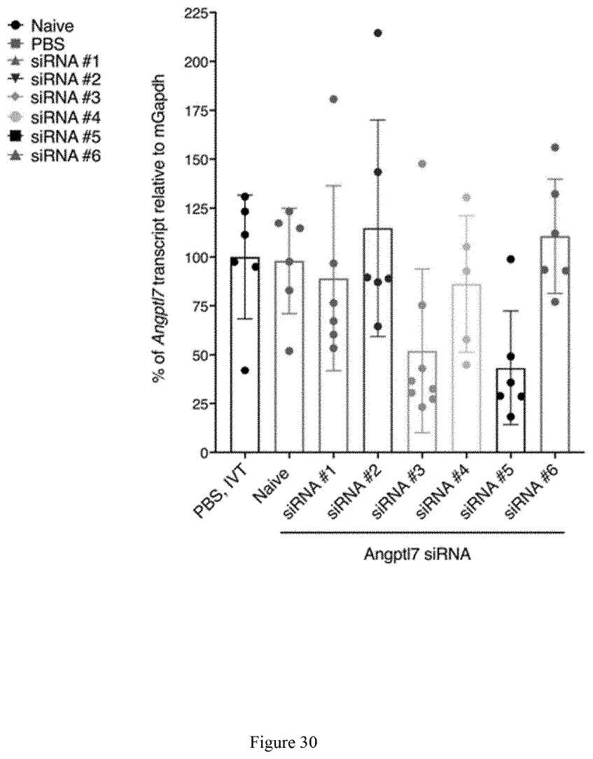

To investigate whether knockdown of Angptl7 with small interfering RNA (siRNA) can also lower IOP, six different siRNAs targeting Angptl7 in C57BL/6J mice were tested and IOP was monitored over time. C57BL/6J mice were injected intravitreally with 15 μg of siRNAs and performed qPCR six weeks later on limbal rings dissected from mouse eyes enriched for the TM. IOP was significantly lowered 2 weeks post-injection in mice treated with two of the six siRNAs compared to the PBS and Naïve (no injection) groups. Naïve and PBS-treated animals maintained their IOPs at baseline for the duration of the study (weeks 0-6). In mice treated with siRNA #3 and #5, IOP was lowered by 2-4 mmHg starting at week 2 compared to PBS-treated mice (FIG. 29). At the end of the study, the eyes were collected, carefully micro-dissected the limbal ring and performed qPCR. The highest level of knockdown (>50%) of Angptl7 mRNA was observed with siRNAs #3 and #5 compared to PBS-treated mice, which is consistent with the IOP lowering observed in mice injected with these two siRNAs (FIG. 30). These results suggest that acute inhibition of Angptl7 expression also lowers IOP.

### Example 10: Gene Expression Changes in Human TM Cells Upon Dexamethasone Treatment

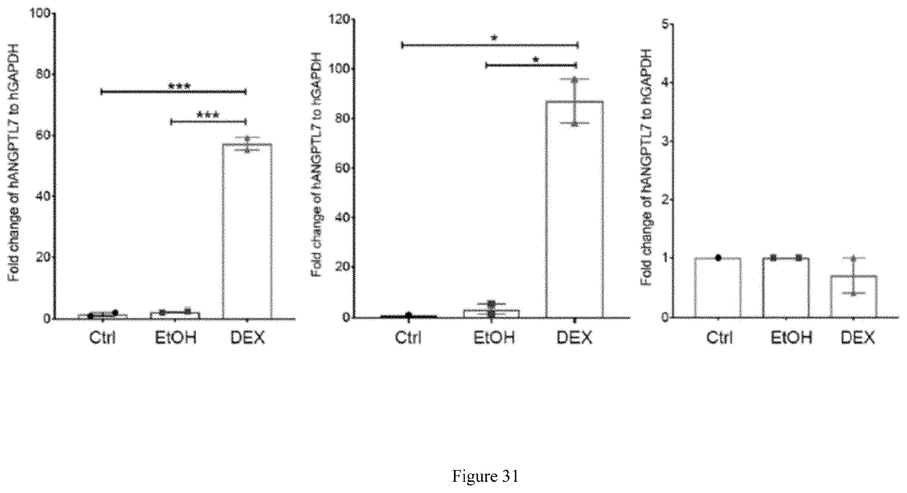

Dexamethasone (DEX) treatment is known to lead to many biochemical changes at the gene expression level in the TM, including upregulation ANGPTL7. To further characterize these previous findings, quantitative PCR (qPCR) was performed on three human TM primary cell lines from three independent human eyes treated with vehicle (0.1% ethanol) or DEX (100 nM) for 72 hours. qPCR analysis revealed increased expression of ANGPTL7 expression in two out of three (FIG. 31), suggesting some degree of variability in the DEX-induced upregulation of ANGTPL7, consistent with the observed variation in response to steroid treatment in the general population.

### Example 11: Discussion

In this study, genetic and functional evidence for a role for ANGPTL7 in the physiological control of IOP and as a potential target for glaucoma therapy is presented. Through genetic association analyses in Europeans, a rare missense variant, Gln175His (rs28991009) was identified, in ANGPTL7 associated with a decrease in IOP and with decreased risk for glaucoma, consistent with previously reported findings. pLOF variants in ANGPTL7, Arg177* (rs143435072) and African ancestry-enriched Trp188* (rs145750805) were further identified, that also associated with a decrease in IOP, suggesting that Gln175His carriers are protected from glaucoma through a loss or reduction in ANGPTL7 activity. Through cell-based expression assays, it was found that Gln175His, Arg177*, and Trp188* were severely defective in secretion when compared to wild-type and, while not proof, this observation is consistent with the hypothesis that they result in a loss of protein function. Also were identified predicted-deleterious ANGPTL7 variants in burden analyses associated with reduced IOP, indicating that there may be other ultra-rare ANGPTL7 variants that confer protection from glaucoma. Supporting this hypothesis, a recent report described the association of one of these ANGPTL7 variants enriched in Finnish individuals (Arg220Cys) with reduced IOP and decreased risk for glaucoma.

Within the eye, the present in situ hybridization results show that ANGPTL7 is expressed most strongly in the cornea and TM, consistent with previous findings. This study has also shown using single-cell RNAseq that ANGPTL7 expression is particularly enriched in the juxtacanalicular tissue (JCT), a region of the TM most important for IOP regulation and generation of aqueous humor outflow resistance. In addition to being expressed in tissues directly relevant in glaucoma, several lines of evidence have implicated ANGPTL7 in glaucoma pathophysiology. First, elevated levels of ANGPTL7 mRNA and protein were observed in eye tissues from glaucoma patients compared to controls, and under conditions of increased IOP simulated by perfusion of eye anterior segment explants. Second, ANGPTL7 is one of the most highly upregulated genes in response to corticosteroid treatment, which can cause increased IOP in ˜40% of general population and ˜90% of individuals with POAG. Third, ANGPTL7 levels are also increased in response to TGF-beta, a growth factor that is thought to modulate the ECM and lead to increased IOP. Fourth, ANGPTL7 itself can modulate the expression of components of the TM ECM.

In mice, where reciprocal experiments were performed: measuring IOP after increasing ANGPTL7 levels via injection of mAngptl7 into mouse eyes, and after removing all mAngptl7 protein by generating Angtpl7 KO mice. The findings show that increasing mAngptl7 results in increased (˜2-4 mmHg) IOP and decreasing mAngptl7 through KO mice reduces basal IOP levels (˜2 mmHg), establishing that ANGPTL7 functions in vivo to maintain IOP homeostasis. In addition, the reduction in IOP observed in KO mice was recapitulated by injecting WT mouse eyes with siRNA against mAngptl7, which not only replicates the observation in genetic mutant mice but also illustrates that the effect of mAngptl7 on IOP continues post-development and is amenable to modulation by therapeutics in adulthood. These results of lower IOP in Angptl7 KO mice are highly consistent with observations from human genetics. Based on this, the siRNA knockdown findings could be extended to humans and surmise that inhibition of ANGPTL7 in adulthood could be an efficacious way to lower IOP and, eventually, the risk for glaucoma.

An independent contribution of ANGPTL7 to IOP homeostasis is likely based on the following: 1) The persistent association with reduced IOPcc suggests that there is IOP reduction even after controlling for corneal properties; 2) Angptl7 KO mice eyes show reduced basal IOP without evidence of corneal thinning or other corneal abnormalities; and 3) the association of ANGPTL7 variants with glaucoma protection is a result that would not be expected if the reduction of IOP was purely due to ANGPTL7's effect on corneal anatomy. Therefore, it is believed that ANGPTL7 likely has a pleiotropic effect on both IOP and corneal anatomy/biomechanics in humans.

In summary, these genetic and pharmacological results indicate that ANGPTL7 participates in the normal physiological regulation of IOP in humans and mice. Since excessive amounts of ANGPTL7 protein in the eyes of experimental animals cause IOP to elevate to pathological levels, upregulation of ANGPTL7 in humans may be responsible for the elevated IOP that leads to POAG. Therefore, ANGPTL7 appears to be an excellent candidate to explore as a therapeutic target for POAG.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having an ophthalmic condition, the method comprising administering an Angiopoietin-Like 7 (ANGPTL7) inhibitor to the subject, wherein the inhibitor comprises a Cas protein and guide RNA (gRNA) that hybridizes to a gRNA recognition sequence that includes or is proximate to a position corresponding to: position 4,269 according to SEQ ID NO:1, position 5,125 according to SEQ ID NO:1, position 5,176 according to SEQ ID NO:1, or position 5,232 according to SEQ ID NO: 1.

2. A method of treating a subject having increased intraocular pressure (IOP), the method comprising administering an Angiopoietin-Like 7 (ANGPTL7) inhibitor to the subject, wherein the inhibitor comprises a Cas protein and guide RNA (gRNA) that hybridizes to a gRNA recognition sequence that includes or is proximate to a position corresponding to: position 4,269 according to SEQ ID NO:1, position 5,125 according to SEQ ID NO:1, position 5,176 according to SEQ ID NO:1, or position 5,232 according to SEQ ID NO:1.

3. A method of treating a subject having glaucoma, the method comprising administering an Angiopoietin-Like 7 (ANGPTL7) inhibitor to the subject, wherein the inhibitor comprises a Cas protein and guide RNA (gRNA) that hybridizes to a gRNA recognition sequence that includes or is proximate to a position corresponding to: position 4,269 according to SEQ ID NO:1, position 5,125 according to SEQ ID NO:1, position 5,176 according to SEQ ID NO:1, or position 5,232 according to SEQ ID NO: 1.

4. A method of treating a subject having open angle glaucoma, the method comprising administering an Angiopoietin-Like 7 (ANGPTL7) inhibitor to the subject, wherein the inhibitor comprises a Cas protein and guide RNA (gRNA) that hybridizes to a gRNA recognition sequence that includes or is proximate to a position corresponding to: position 4,269 according to SEQ ID NO:1, position 5,125 according to SEQ ID NO:1, position 5,176 according to SEQ ID NO:1, or position 5,232 according to SEQ ID NO: 1.

5. A method of treating a subject having angle-closure glaucoma, the method comprising administering an Angiopoietin-Like 7 (ANGPTL7) inhibitor to the subject, wherein the inhibitor comprises a Cas protein and guide RNA (gRNA) that hybridizes to a gRNA recognition sequence that includes or is proximate to a position corresponding to: position 4,269 according to SEQ ID NO:1, position 5,125 according to SEQ ID NO:1, position 5,176 according to SEQ ID NO:1, or position 5,232 according to SEQ ID NO: 1.

6-14. (canceled)

15. The method according to claim 1, further comprising detecting the presence or absence of an ANGPTL7 missense variant nucleic acid molecule encoding an ANGPTL7 predicted loss-of-function polypeptide comprising one or more Ile174Asn, Arg231Cys, Arg248Cys, or His266Gln variants in a biological sample obtained from the subject.

16. The method according to claim 15, further comprising administering a therapeutic agent that treats or inhibits an ophthalmic condition in a standard dosage amount to a subject wherein the ANGPTL7 missense variant nucleic acid molecule is absent from the biological sample

17. The method according to claim 15, further comprising administering a therapeutic agent that treats or inhibits an ophthalmic condition in a dosage amount that is the same as or less than a standard dosage amount to a subject that is heterozygous for the ANGPTL7 missense variant nucleic acid molecule.

18-35. (canceled)

36. A method of treating a subject with a therapeutic agent that treats or inhibits an ophthalmic condition, wherein the subject has an ophthalmic condition, the method comprising:
determining whether the subject has an Angiopoietin-Like 7 (ANGPTL7) missense variant nucleic acid molecule encoding an ANGPTL7 predicted loss-of-function polypeptide comprising one or more Ile174Asn, Arg231Cys, Arg248Cys, or His266Gln variants by:
obtaining or having obtained a biological sample from the subject; and
performing or having performed a sequence analysis on the biological sample to determine if the subject has a genotype comprising the ANGPTL7 missense variant nucleic acid molecule encoding the ANGPTL7 predicted loss-of-function polypeptide comprising one or more Ile174Asn, Arg231Cys, Arg248Cys, or His266Gln variants; and
administering or continuing to administer the therapeutic agent that treats or inhibits the ophthalmic condition in a standard dosage amount to a subject that is ANGPTL7 reference, and administering an ANGPTL7 inhibitor to the subject; and
administering or continuing to administer the therapeutic agent that treats or inhibits the ophthalmic condition in an amount that is the same as or less than a standard dosage amount to a subject that is heterozygous for the ANGPTL7 missense variant nucleic acid molecule encoding the ANGPTL7 predicted loss-of-function polypeptide comprising one or more Ile174Asn, Arg231Cys, Arg248Cys, or His266Gln variants, and administering an ANGPTL7 inhibitor to the subject;
wherein the presence of a genotype having the ANGPTL7 missense variant nucleic acid molecule encoding the ANGPTL7 predicted loss-of-function polypeptide comprising one or more Ile174Asn, Arg231Cys, Arg248Cys, or His266Gln variants indicates the subject has a reduced risk of developing ophthalmic conditions.

37. The method according to claim 36, wherein the subject is ANGPTL7 reference, and the subject is administered or continued to be administered the therapeutic agent that treats or inhibits the ophthalmic condition in a standard dosage amount, and is administered an ANGPTL7 inhibitor.

38. The method according to claim 36, wherein the subject is heterozygous for an ANGPTL7 missense variant nucleic acid molecule, and the subject is administered or continued to be administered the therapeutic agent that treats or inhibits the ophthalmic condition in an amount that is the same as or less than a standard dosage amount, and is administered an ANGPTL7 inhibitor.

39-58. (canceled)

59. The method according to claim 36, wherein the ANGPTL7 inhibitor comprises a Cas protein and guide RNA (gRNA) that hybridizes to a gRNA recognition sequence that includes or is proximate to a position corresponding to: position 4,269 according to SEQ ID NO:1, position 5,125 according to SEQ ID NO:1, position 5,176 according to SEQ ID NO:1, or position 5,232 according to SEQ ID NO:10.

60-65. (canceled)

66. A method of identifying a subject having an increased risk of developing an ophthalmic condition, the method comprising:
determining or having determined the presence or absence of an Angiopoietin-Like 7 (ANGPTL7) missense variant nucleic acid molecule encoding an ANGPTL7 predicted loss-of-function polypeptide comprising one or more Ile174Asn, Arg231Cys, Arg248Cys, or His266Gln variants in a biological sample obtained from the subject;
wherein:
when the subject is ANGPTL7 reference, then the subject has an increased risk of developing ophthalmic conditions; and
when the subject is heterozygous or homozygous for an ANGPTL7 missense variant nucleic acid molecule encoding the ANGPTL7 predicted loss-of-function polypeptide comprising one or more Ile174Asn, Arg231Cys, Arg248Cys, or His266Gln variants, then the subject has a decreased risk of developing an ophthalmic condition.

67-84. (canceled)

85. The method according to claim 66, wherein the subject is ANGPTL7 reference, and the subject is administered a therapeutic agent that treats or inhibits an ophthalmic condition in a standard dosage amount, and is administered an ANGPTL7 inhibitor.

86. The method according to claim 66, wherein the subject is heterozygous for an ANGPTL7 predicted loss-of-function variant, and the subject is administered a therapeutic agent that treats or inhibits an ophthalmic condition in an amount that is the same as or lower than a standard dosage amount, and is administered an ANGPTL7 inhibitor.

87. A method of detecting an Angiopoietin-Like 7 (ANGPTL7) missense variant nucleic acid molecule, or the complement thereof, encoding an ANGPTL7 predicted loss-of-function polypeptide in a subject, the method comprising assaying a biological sample obtained from the subject to determine whether a nucleic acid molecule in the biological sample encodes an ANGPTL7 predicted loss-of-function polypeptide comprising one or more Ile174Asn, Arg231Cys, Arg248Cys, or His266Gln variants

88-103. (canceled)

104. A method of detecting the presence of an Angiopoietin-Like 7 (ANGPTL7) Ile174Asn, Arg231Cys, Arg248Cys, or His266Gln polypeptide, comprising performing an assay on a biological sample obtained from a subject to determine whether an ANGPTL7 polypeptide in the biological sample comprises: an asparagine at a position corresponding to position 174 according to SEQ ID NO:173, a cysteine at a position corresponding to position 231 according to SEQ ID NO:178, a cysteine at a position corresponding to position 248 according to SEQ ID NO:179, or a glutamine at a position corresponding to position 266 according to SEQ ID NO:180.

105-117. (canceled)
